(19) United States
(12) Patent Application Publication
(10) Pub. No.: US 2015/0072969 A1
(43) Pub. Date: Mar. 12, 2015

(54) PHENYL ALKANOIC ACID DERIVATIVES AS GPR AGONISTS

(71) Applicant: PIRAMAL ENTERPRISES LIMITED, Mumbai (IN)

(72) Inventors: Sanjay Kumar, Mumbai (IN); Rajiv Sharma, Mumbai (IN); Vishal Ashok Mahajan, Mumbai (IN); Sangameshwar Prabhakar Sawargave, Mumbai (IN)

(73) Assignee: PIRAMAL ENTERPRISES LIMITED, Mumbai (IN)

(21) Appl. No.: 14/381,696

(22) PCT Filed: Feb. 27, 2013
(86) PCT No.: PCT/IB2013/051555

§ 371 Date: Aug. 28, 2014

Related U.S. Application Data
(60) Provisional application No. 61/603,988, filed on Feb. 28, 2012.
(60) Provisional application No. 61/736,622, filed on Dec. 13, 2012.

(51) Int. Cl. C07D 413/12; C07D 405/12; C07D 409/12; C07D 205/04; C07D 305/06; C07D 407/12

(52) U.S. Cl. CPC C07D 413/12; C07D 305/06; C07D 407/12; C07D 409/12; C07D 205/04; C07D 405/12
USPC 514/210.01; 549/510; 514/449; 549/473; 514/461; 549/414; 514/451; 549/13; 514/432; 549/60; 514/444; 548/235; 514/374; 548/950; 546/281.7; 514/336; 544/122; 514/235.8

(57) ABSTRACT

The present invention relates to phenyl alkanoic acid derivatives (the compounds of Formula (I)); and their isotopic forms, stereoisomeric and tautomeric forms and mixtures thereof in all ratios, or pharmaceutically acceptable salts, pharmaceutically acceptable solvates, prodrugs, polymorphs, N-oxides, S-oxides or carboxylic acid isosteres thereof. The invention also relates to processes for the preparation of compounds of Formula (I)

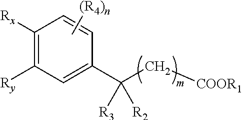

and pharmaceutical compositions comprising one or more of the compounds of Formula (I). The said compounds and the pharmaceutical composition function as GPR (G-protein coupled receptor) agonists, particularly as GPR40 agonists, and are useful in the treatment of diseases or conditions mediated by GPR40. The present invention further relates to a method of treatment of diseases or conditions mediated by GPR40 comprising administering to a subject in need thereof a therapeutically effective amount of the compounds of Formula (I).

### FIELD OF THE INVENTION

The present invention relates to phenyl alkanoic acid derivatives (the compounds of Formula (I)), processes for their preparation, pharmaceutical compositions containing said compounds, their use as GPR (G-protein coupled receptor) agonists, particularly as GPR40 agonists and methods of using these compounds in the treatment of GPR40 mediated diseases or conditions.

### BRIEF DESCRIPTION OF THE INVENTION

Obesity is a major health problem throughout the world. It is a risk factor for developing insulin resistance, type 2 diabetes, hypertension, and cardiovascular diseases (Circulation, 2003, 107:1448-1453). In the United States, only about a third of adults are considered to be of ‘normal’ weight and similar trends are being increasingly observed worldwide (Nature, 2006, 444(14):840-46). Obesity is typically associated with elevated levels of free fatty acids (FFAs) and is linked to glucose intolerance and type 2 diabetes (Cell Metab., 2005, 1(4):245-58).

According to one report, the prevalence of diabetes was 171 million patients worldwide in the year 2000, and is expected to grow to 366 million patients (thirty million in the United States alone) by 2030 (Diabetes Care, 2004, 27(5):1047-53). The increasing incidence is largely driven by the dramatic rise in obesity, especially in Western societies. Type 2 diabetes accounts for 90-95% of all diabetes. Complex networks of signaling pathways are activated when the insulin receptor is stimulated, but in patients who suffer from type 2 diabetes, those receptors on cells in tissues such as muscle, fat and liver become less responsive or resistant to insulin. In addition, patients with type 2 diabetes are typically characterized by reduced glucose stimulated insulin secretion (GSIS) (Expert Opin. Ther. Patents, 2009, 19(2): 237-264).

Metabolic syndrome, also known as Syndrome-X, is characterized by a cluster of conditions, including insulin resistance, obesity, hypertension and dyslipidaemia. Persistent obesity disregulates metabolic processes including action of insulin on glucose-lipid-free fatty acid metabolism and severely affects processes controlling blood glucose, blood pressure, and lipids. It is also well recognized that people with obesity and metabolic syndrome are at an increased risk of developing type 2 diabetes and cardiovascular diseases. Prevalence of obesity and metabolic syndrome has shown a rapid rise in developing countries in the past few decades and has led to increased risk of cardiovascular diseases and consequent morbidity and mortality (JRAAS, 2006, 7(1):S12-S18; J. Clin. Endocrinol. Metab., 2008, 93(11):S9-S30).

It is well-known that the production of insulin is essential for carbohydrate and lipid metabolism and that insulin imbalance leads to conditions such as type 2 diabetes mellitus, which is a serious metabolic disease as discussed above. Relatively recently the function of the G-protein coupled receptor, particularly G-protein coupled receptor (GPR40) is recognised in modulating insulin secretion, which has provided insight into regulation of carbohydrate and lipid metabolism. This has lead to the targets for the development of therapeutic agents for disorders such as obesity, diabetes, cardiovascular disease and dyslipidemia.

G-protein coupled receptors (GPCRs) constitute a super family of membrane proteins activated by a variety of endogenous ligands such as hormones, neurotransmitters, peptides, proteins, steroids as wells as fatty acids (FAs) and other lipids (Diabetes Obes. Metab., 2009, 11(4):1-18). Impaired GSIS are a prominent feature of overt type 2 diabetes and FFAs are known to influence insulin secretion from β-cells primarily by enhancing GSIS. G-protein coupled receptors (GPCRs) such as GPR40, whose endogenous ligands are medium and long chain free fatty acids, are known to play an important role in insulin release.

The G-protein coupled receptor, GPR40, alternatively called the FFA receptor 1, is G_{α}q-coupled Class 1 GPCR and a member of a small family of fatty acid sensing GPCRs. GPR40 is preferentially expressed in β-cells and is activated by medium to long chain FFAs, thereby triggering a signaling cascade that results in increased levels of [Ca²⁺] in β-cell lines (Diabetes, 2008, 57:2280-87 and Bioorganic & Medicinal Chemistry Letters, 2012, 22:1267-1270).

Studies conducted in animals (mice) further established that loss of GPR40 protects mice from obesity induced hyperglycemia, glucose intolerance, hyperinsulinemia, fatty liver development, hepatic glucose output and hypertriglyceridemia (Diabetes, 2008, 57:2280-87).

The identification of the function of G-protein coupled receptor GPR40 in modulating insulin secretion and their role in lipid metabolism has therefore sparked interest in GPR40 agonists being considered as potential targets for the development of therapeutic agents which can be useful to treat metabolic disorders such as obesity, type 2 diabetes, cardiovascular diseases and hypertriglyceridemia.

Several small molecule GPR agonists are known and have been reported in various publications and patents. PCT published application WO2005086661A2, discloses compounds capable of modulating the G-protein coupled receptor GPR40, compositions comprising the compounds and methods for their use in controlling insulin levels in vivo and for the treatment of conditions such as type 2 diabetes, hypertension, ketoacidosis, obesity, glucose intolerance and hypercholesterolemia and related disorders associated with abnormally high or low plasma lipoprotein, triglyceride or glucose levels.

PCT published application WO200801931A1, discloses fused cyclic compounds which are useful as insulin secretagogues or agents for the prophylaxis or treatment of diabetes and related disorders. PCT published applications WO2009111056 A1 and WO2010045258A2 disclose spirocyclic compounds which act as GPR40 modulators, compositions comprising the compounds and methods for their use in the treatment or prevention of metabolic disorders, especially type 2 diabetes, obesity and related disorders. PCT published application WO2010123016A1 discloses carboxylic acid compounds which have GPR40 agonist activity and are useful as insulin secretion promoter and as a prophylactic and therapeutic agent for diabetes or borderline diabetes (abnormal glucose tolerance and fasting blood sugar). PCT published application WO2012011125A1 discloses compounds that have the ability to modulate the activity of GPR40, compositions comprising these compounds and their use in the treatment of disorders related to GPR40 activity, especially metabolic conditions, such as diabetes, obesity, hyperglycemia, insulin resistance, hypercholesteremia and related disorders.

Thus, in view of the role of GPR such as GPR40 in the pathophysiology of metabolic disorders, there exists a continuing medical need for safe and efficacious compounds which can function as GPR agonists.

### SUMMARY OF THE INVENTION

In one aspect, the present invention relates to a compound of Formula (I) (as described herein) in an isotopic form, or a stereoisomer or a tautomer or a pharmaceutically acceptable salt, a pharmaceutically acceptable solvate, a prodrug, a polymorph, N-oxide, S-oxide, or a carboxylic acid isostere thereof.

In another aspect of the present invention, there is provided a process for the preparation of the compound of Formula (I).

In a further aspect, the present invention relates to a pharmaceutical composition comprising a therapeutically effective amount of a compound of Formula (I) or a pharmaceutically acceptable salt thereof; and at least one pharmaceutically acceptable carrier or excipient.

In a further aspect, the present invention relates to a pharmaceutical composition comprising a therapeutically effective amount of a compound of Formula (I) or a pharmaceutically acceptable salt thereof; and one further therapeutically active agent and at least one pharmaceutically acceptable carrier or excipient.

In another further aspect, the present invention relates to a method for modulating GPR40 function in a cell.

In yet another aspect, the present invention provides a compound of Formula (I) for use in the treatment or prophylaxis of a disease or a condition mediated by GPR40.

In yet another further aspect, the present invention provides a method for the treatment or prophylaxis of a disease or a condition mediated by GPR40, comprising administering to a subject in need thereof a therapeutically effective amount of the compound of Formula (I) or a pharmaceutically acceptable salt thereof.

In a still further aspect, the present invention relates to use of the compound of Formula (I) or a pharmaceutically acceptable salt thereof in the manufacture of a medicament, for the treatment or prophylaxis of a disease or a condition mediated by GPR40.

In another further aspect, the present invention relates to use of the compound of Formula (I) or a pharmaceutically acceptable salt thereof in combination with one further therapeutically active agent for the treatment or prophylaxis of a disease or a condition mediated by GPR40.

These and other objectives and advantages of the present invention will be apparent to those skilled in the art from the following description.

### DETAILED DESCRIPTION OF THE INVENTION

The present invention relates to a compound of Formula (I):

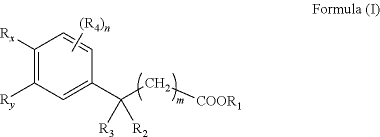

wherein,
R₁ is hydrogen or (C₁-C₆) alkyl;
R₂ and R₃ together form a saturated or a partially unsaturated 3- to 9-membered heterocyclyl containing one or two heteroatoms selected from O, N or S; or R₂ and
R₃ together form a saturated or a partially unsaturated (C₄-C₈) cycloalkyl;
R₄ at each occurrence is independently selected from hydrogen, (C₁-C₆)alkyl, halogen, halo(C₁-C₆) alkyl, hydroxy, —O(C₁-C₆)alkyl, (C₆-C₁₀)aryl, amino, cyano, nitro, —C(O)R₉ or —S(O)ₚR₆;
Rₓ and R_{y} are independently selected from A-CH(R₇)—X or R₅; provided that at least one of Rₓ and R_{y} is A-CH(R₇)—X;
R₅ is selected from hydrogen, (C₁-C₆) alkyl, halogen, halo(C₁-C₆)alkyl, hydroxy, —O(C₁-C₆)alkyl, (C₆-C₁₀)aryl, amino, cyano, nitro, —C(O)R₉ or —S(O)ₚR₆;
R₆ is selected from hydrogen, (C₁-C₆)alkyl or amino;
R₇ is hydrogen or (C₁-C₆)alkyl;
X is selected from O, NR₈ or S;
R₈ is selected from hydrogen, (C₁-C₆)alkyl, (C₃-C₈)cycloalkyl, (C₆-C₁₀)aryl, heterocyclyl, heteroaryl, cyano, —C(O)(C₁-C₆)alkyl, —C(O)O(C₁-C₆)alkyl, —C(O)NH₂ or —S(O)ₚR₆; wherein R₆ is as defined above;
R₉ is selected from (C₁-C₆) alkyl, —O(C₁-C₆)alkyl, hydroxy or amino;
A is selected from (C₃-C₈)cycloalkyl, (C₆-C₁₀)aryl, heterocyclyl, heteroaryl,

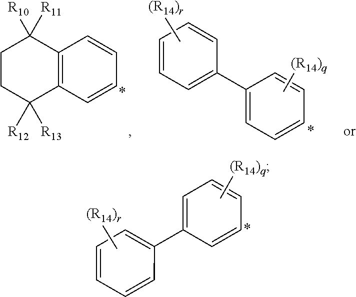

R₁₀, R₁₁, R₁₂ and R₁₃ are independently selected from hydrogen and (C₁-C₆) alkyl; or R₁₀ and R₁₁ can together form a (C₃-C₈)cycloalkyl ring and R₁₂ and R₁₃ are hydrogen; or R₁₂ and R₁₃ can together form a (C₃-C₈)cycloalkyl ring and R₁₀ and R₁₁ are hydrogen;
R₁₄ at each occurrence is independently selected from hydrogen, (C₁-C₆) alkyl, halogen, halo(C₁-C₆)alkyl, hydroxy, —O(C₁-C₆)alkyl, —O(C₃-C₈)cycloalkyl, —O(C₁-C₆)alkylheterocyclyl, —O-heterocyclyl, halo(C₁-C₆) alkoxy, —O(C₁-C₆)alkyl-S(O)ₚR₆, (C₆-C₁₀)aryl, amino, cyano, nitro, —C(O)R₉, —S(O)ₚR₆, —(CH₂)ₛNR₁₅R₁₆ and —X(CH₂)ₛNR₁₅R₁₆; wherein X, R₆ and R₉ are as defined above;
R₁₅ and R₁₆ are independently selected from hydrogen, (C₁-C₆)alkyl and —(CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4;
t is an integer from 1 to 4;
* indicates the point of attachment to —CH of CH(R₇)—X;
wherein,

(C₁-C₆)alkyl is unsubstituted or substituted with one or more groups independently selected from (C₁-C₆)alkyl, (C₂-C₈)alkenyl, (C₂-C₈)alkynyl, halogen, halo(C₁-C₆)alkyl, hydroxy, —O(C₁-C₆)alkyl, (C₃-C₈)cycloalkyl, (C₆-C₁₀)aryl, heterocyclyl, heteroaryl, amino, cyano, nitro, —C(O)R₉ and —O(C₁-C₆) alkyl-S(O)ₚR₆; wherein R₆, R₉, and p are as defined above;

—O(C₁-C₆)alkyl is unsubstituted or substituted with one or more groups independently selected from (C₁-C₆)alkyl, (C₃-C₈)cycloalkyl, heterocyclyl, hydroxy, halogen, amino, cyano, —(C₁-C₆)alkyl-S(O)ₚR₆, —S(O)ₚR₆, —NR₁₅R₁₆ and —(CH₂)ₛNR₁₅R₁₆; wherein R₆, R₁₅, R₁₆, p and s are as defined above;

(C₆-C₁₀)aryl is unsubstituted or substituted with one or more groups independently selected from (C₁-C₆)alkyl, (C₂-C₈)alkenyl, (C₂-C₈)alkynyl, halogen, halo(C₁-C₆) alkyl, hydroxy, —O(C₁-C₆)alkyl, halo(C₁-C₆) alkoxy, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

heterocyclyl is a 3- to 9-membered ring, which is unsubstituted or substituted with one or more groups independently selected from (C₁-C₆)alkyl, (C₂-C₈)alkenyl, (C₂-C₈)alkynyl, halogen, halo(C₁-C₆) alkyl, hydroxy, —O(C₁-C₆)alkyl, halo(C₁-C₆) alkoxy, (C₃-C₈)cycloalkyl, (C₆-C₁₀)aryl, heterocyclyl, heteroaryl, amino, cyano, nitro, —(C₁-C₆)alkyl-OH, (C₁-C₆)alkyl-O—(C₁-C₆)alkyl, —C(O)R₉ and —O(C₁-C₆)alkyl-S(O)ₚR₆; wherein R₆, R₉, and p are as defined above;

heteroaryl is a 3- to 10-membered ring, which is unsubstituted or substituted with one or more groups independently selected from (C₁-C₆)alkyl, (C₂-C₈)alkenyl, (C₂-C₈)alkynyl, halogen, halo(C₁-C₆)alkyl, hydroxy, —O(C₁-C₆)alkyl, halo(C₁-C₆) alkoxy, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

halogen is selected from chlorine, bromine, iodine or fluorine; or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

### DEFINITIONS

Unless otherwise indicated, the following definitions are set forth to illustrate and define the meaning and scope of the various terms used to describe the invention herein and the appended claims. These definitions should not be interpreted in the literal sense as they are not general definitions and are relevant only for this application.

It will be understood that “substitution,” “substituted” or “substituted with” means that one or more hydrogens of the specified moiety are replaced with a suitable substituent and includes the implicit proviso that such substitution is in accordance with permitted valence of the substituted atom and the substituent, and results in a stable compound.

The terms “a”, “an” and “the” refers to “one or more” when used in the subject specification, including the claims. Thus, for example, reference to “a compound” may include a plurality of such compounds, or reference to “a disease” or “a condition” includes a plurality of diseases or disorders.

Within the context of the present invention, the term “(C₁-C₆)alkyl” or “alkyl”, as used herein, alone or as part of a substitutent group refers to an aliphatic group, including straight or branched chain alkyl group. A straight-chain or branched chain alkyl has six or fewer carbon atoms in its backbone, for instance, C₁-C₆ for straight-chain and C₃-C₆ for branched chain. Suitable alkyl groups containing from one to six carbon atoms include, but are not limited to, methyl, ethyl, propyl, butyl, pentyl, hexyl, isopropyl, isobutyl, 1-methylbutyl, secondary butyl, tertiary pentyl, neopentyl, 2,2-dimethylbutyl, 2-methylpentyl, 3-methylpentyl or 3-methylpentyl.

Furthermore, unless stated otherwise, the alkyl groups may be unsubstituted or substituted with one or more substituents, for instance, from one to five identical or different substituents, for example, (C₁-C₆) alkyl, (C₂-C₈)alkenyl, (C₂-C₈) alkynyl, halogen, halo(C₁-C₆)alkyl, hydroxy, —O(C₁-C₆) alkyl, (C₃-C₈)cycloalkyl, (C₆-C₁₀)aryl, heterocyclyl, -heterocyclyl-(C₁-C₆)alkyl-OH, heteroaryl, amino, cyano, nitro, —S(O)ₚR₆, —C(O)R₉ or —O(C₁-C₆)alkyl-S(O)ₚR₆; wherein R₆, R₉, and p are as defined above. Examples of substituted alkyl include but not limited to hydroxymethyl, 2-chlorobutyl, trifluoromethyl, aminoethyl or benzyl.

Within the context of the present application, the term “(C₂-C₈)alkenyl” or “alkenyl”, as used herein, alone or as part of a substituent group, refers to an unsaturated straight or branched chain hydrocarbon radical containing at least one carbon-carbon double bond (two adjacent sp² carbon atoms). For example, the term “(C₂-C₈)alkenyl” refers to an alkenyl group having two to eight carbon atoms. Depending upon the placement of double bond and substituents if any, the geometry of the double bond may be entgegen (E), or zusammen (Z), cis or trans. Examples of alkenyl include, but are not limited to, vinyl, allyl or 2-propenyl. Unless indicated otherwise, the alkenyl groups may be unsubstituted or substituted with one or more substituents independently selected from (C₁-C₆)alkyl, halogen, halo(C₁-C₆) alkyl, hydroxy, —O(C₁-C₆)alkyl, (C₃-C₈)cycloalkyl, (C₆-C₆)aryl, heterocyclyl, heteroaryl, amino, nitro, cyano, —C(O)R₉ and —OC(O)CH₃; wherein R₉ is as defined above.

Within the context of the present application, and as used herein, the term “(C₂-C₈)alkynyl” or “alkynyl” refers to an unsaturated, branched or straight chain having from two to eight carbon atoms and at least one carbon-carbon triple bond (two adjacent sp carbon atoms). Examples of alkynyl include, but not limited to, ethynyl, 1-propynyl, 3-propynyl and 4-butynyl. Unless stated otherwise, the alkynyl groups may be unsubstituted or substituted with one or more substituents independently selected from (C₁-C₆)alkyl, halogen, halo(C₁-C₆)alkyl, hydroxy, —O(C₁-C₆)alkyl, (C₃-C₈)cycloalkyl, (C₆-C₁₀)aryl, heterocyclyl, heteroaryl, amino, nitro, cyano, —C(O)R₉ and —OC(O)CH₃; wherein R₉ is as defined above.

Within the context of the present application and as used herein, the term “haloalkyl” or “halo(C₁-C₆)alkyl” refers to radicals wherein one or more of the hydrogen atoms of the alkyl group are substituted with one or more halogens. A monohaloalkyl radical, for example, may have a chlorine, bromine, iodine or fluorine atom. Dihalo and polyhaloalkyl radicals may have two or more of the same or different halogen atoms. Examples of “haloalkyl” or “halo(C₁-C₆) alkyl” include, but are not limited to, chloromethyl, dichloromethyl, trichloromethyl, dichloroethyl, dichloropropyl, fluoromethyl, difluoromethyl, trifluoromethyl, pentafluoroethyl, hepta fluoropropyl, difluorochloromethyl, dichlorofluoro methyl, difluoroethyl or difluoropropyl.

Within the context of the present application and as used herein, the term “alkoxy” refers to (C₁-C₆)alkyl group having an oxygen radical attached thereto. The terms alkoxy or —O(C₁-C₆)alkyl wherever used in this specification have the same meaning. Representative alkoxy groups include, but not limited to, methoxy, ethoxy, propoxy, isopropoxy, isobutoxy and tert-butoxy. —O(C₁-C₆)alkyl is unsubstituted or substituted with one or more groups independently selected from (C₁-C₆)alkyl, (C₃-C₈)cycloalkyl, heterocyclyl, hydroxy, halogen, amino, cyano, —(C₁-C₆)alkyl-S(O)ₚR₆, heterocyclyl-(C₁-C₆)alkyl-OH, —S(O)ₚR₆, —NR₁₅R₁₆, and —(CH₂)ₛNR₁₅R₁₆; wherein R₆, R₁₅, R₁₆, p and s are as defined above.

Within the context of the present application and as used herein, the term “haloalkoxy” or “halo(C₁-C₆)alkoxy” refers to radicals wherein one or more of the hydrogen atoms of the alkoxy group are substituted with one or more halogens. Representative examples of “haloalkoxy” or “halo(C₁-C₆)alkoxy” groups include, but not limited to, difluoromethoxy (OCHF₂), trifluoromethoxy (OCF₃) or trifluoroethoxy (OCH₂CF₃).

Within the context of the present application and as used herein, the term “(C₃-C₈)cycloalkyl” or “cycloalkyl” refers to a monocyclic hydrocarbon ring containing three to eight carbon atoms. Representative (C₃-C₈)cycloalkyl groups include, but not limited to, cyclopropyl, cyclobutyl, cyclopentyl, cyclohexyl, cycloheptyl or cyclooctyl. Unless stated otherwise, (C₃-C₈)cycloalkyl may be unsubstituted or substituted with one or more substituents independently selected from (C₁-C₆) alkyl, halogen, halo(C₁-C₆) alkyl, hydroxy, —O(C₁-C₆)alkyl, (C₃-C₈)cycloalkyl, (C₆-C₁₀)aryl, heterocyclyl, heteroaryl, amino, cyano, nitro, —C(O)R₉ or —OC(O)CH₃, wherein R₉ is as defined above. Cycloalkyl group comprises a saturated cycloalkyl ring system which does not contain any double bond within the ring or a partially unsaturated cycloalkyl ring system which may contain one or more double bonds within the ring system that is stable, and do not form an aromatic ring system.

Within the context of the present application and as used herein, the term “C₆-C₁₀ aryl” or “aryl” refers to a monocyclic or bicyclic hydrocarbon ring system having up to ten ring carbon atoms, wherein at least one carbocyclic ring is having a π electron system. Examples of (C₆-C₁₀) aryl ring systems include, but not limited to, phenyl or naphthyl. Unless indicated otherwise, aryl group may be unsubstituted or substituted with one or more substituents independently selected from (C₁-C₆)alkyl, (C₂-C₈) alkenyl, (C₂-C₈) alkynyl, halogen, halo(C₁-C₆) alkyl, hydroxy, thiol, —O(C₁-C₆) alkyl, halo(C₁-C₆)alkoxy, (C₃-C₈)cycloalkyl, (C₆-C₁₀)aryl, heterocyclyl, heteroaryl, amino, cyano, nitro, —C(O)R₉, —OC(O)CH₃, —S(O)ₚR₆ and —O(C₁-C₆)alkyl-S(O)ₚR₆; wherein R₆, R₉, and p are as defined above;

Aryl groups can be substituted in any desired position. For example, in monosubstituted phenyl, the substitutent may be located in the 2-position, the 3-position, the 4-position or the 5-position. If the phenyl carries two substituents, they can be located in 2,3-position, 2,4-position, 2,5-position, 2,6-position, 3,4-position or 3,5-position. Examples of monosubstituted phenyl groups include, but not limited to, 3-trifluoromethylphenyl, 4-chlorophenyl, 4-cyanophenyl or the like groups. Examples of disubstituted phenyl groups include, but not limited to, are 4-methoxy-3-trifluoromethylphenyl, 2-methyl-5-trifluoromethyl, 2-methoxy-5-trifluoromethylphenyl, 4-methyl-3-trifluoromethylphenyl, 3-methoxy-4-trifluoromethylphenyl, 3-fluoro-4-tri fluoromethylphenyl, 3-fluoro-5-trifluoromethoxyphenyl, 3-fluoro-4-trifluoromethoxy phenyl or 2-fluoro-3-trifluoromethylphenyl.

Within the context of the present application and as used herein, the term “heterocyclyl” refers to 3- to 9-membered saturated or partially unsaturated monocyclic or bicyclic ring system containing one to four identical or different hetero atoms selected from: a nitrogen (N), a sulphur (S) or an oxygen (O) atom. Heterocyclyl includes saturated heterocyclic ring systems, which do not contain any double bond. Partially unsaturated heterocyclic ring systems, contain at least one double bond, but do not form an aromatic system containing hetero atom. Suitable saturated and partially unsaturated non-aromatic heterocyclic groups include but are not limited to, oxetane, azetidine, thietane, tetrahydrofuran, tetrahydrothiophene, pyrrolidine, dihydropyran, tetrahydropyran, thio-dihydropyran, thio-tetrahydropyran, piperidine, piperazine, morpholine, 1,3-oxazinane, 1,3-thiazinane, 4,5,6-tetra hydropyrimidine, 2,3-dihydrofuran, dihydrothiene, dihydropyridine, tetrahydro pyridine, isoxazolidine or pyrazolidine.

Unless stated otherwise, heterocyclyl may be unsubstituted or substituted with one or more substituents independently selected from (C₁-C₆)alkyl, (C₂-C₈)alkenyl, (C₂-C₈)alkynyl, halogen, halo(C₁-C₆) alkyl, hydroxy, —O(C₁-C₆)alkyl, halo(C₁-C₆)alkoxy, (C₃-C₈)cycloalkyl, (C₆-C₁₀)aryl, heterocyclyl, heteroaryl, amino, cyano, nitro, —(C₁-C₆)alkyl-OH, (C₁-C₆)alkyl-O—(C₁-C₆)alkyl, —C(O)R₉, —OC(O)CH₃, and O(C₁-C₆)alkyl-S(O)ₚR₆; wherein R₆, R₉ and p are as defined above.

Heterocyclyl monocyclic or bicyclic ring systems having an aromatic ring containing hetero atom/s are herein referred to as “heteroaryl”. Within the context of the present invention and as used herein, the term “heteroaryl” refers to 3- to 10-membered aromatic monocyclic or bicyclic ring system containing one to four identical or different hetero atoms selected from: a nitrogen (N), a sulphur (S) or an oxygen (O) atom. Representative examples of heteroaryl include but not limited to thiene, furan, pyridine, oxazole, thiazole, pyrazine, pyrimidine, pyrrole, pyrazole, isooxazole, triazole, tetrazole, pyridazine, isothiazole, benzothiazole, benzooxazole, benzimidazole, quinoline or isoquinoline. Heteroaryl group may be unsubstituted or substituted with one or more substituents independently selected from (C₁-C₆)alkyl, (C₂-C₈)alkenyl, (C₂-C₈)alkynyl, halogen, halo(C₁-C₆) alkyl, hydroxy, thiol, —O(C₁-C₆)alkyl, halo(C₁-C₆)alkoxy, (C₃-C₈)cycloalkyl, (C₆-C₁₀)aryl, heterocyclyl, heteroaryl, amino, cyano, nitro, —(C₁-C₆)alkyl-OH, —(C₁-C₆)alkyl-O—(C₁-C₆)alkyl, —C(O)R₉, —OC(O)CH₃, —S(O)ₚR₆ and —O(C₁-C₆)alkyl-S(O)ₚR₆; wherein R₆, R₉, and p are as defined above. The nitrogen or sulfur atom of the “heterocyclyl” or “heteroaryl” can be optionally oxidized to the corresponding N-oxide, S-oxide or S,S-dioxide.

The term “heteroatom” as used herein, includes nitrogen (N), oxygen (O) and sulfur (S). Any heteroatom with unsatisfied valency is assumed to have a hydrogen atom to satisfy the valency or when the heteroatom is N, it may be substituted with a group selected from (C₁-C₆)alkyl, —C(O)(C₁-C₆)alkyl or —S(O)₂(C₁-C₆)alkyl. Suitable (C₁-C₆)alkyl groups may be selected from, but not limited to methyl, ethyl, propyl, butyl, pentyl, hexyl, isopropyl or isobutyl.

The term “halogen” or “halo” as used herein, unless otherwise indicated refer to bromine, chlorine, fluorine or iodine atom.

The term “amino” refers to the group “NH₂” which may be unsubstituted or substituted by one or more substituents. Examples of substituents include, but not limited to, (C₁-C₄)alkyl, (C₆-C₁₀) aryl or the like groups.

Within the context of the present invention and as used herein interchangeably throughout this application, the terms “compounds of Formula (I)”, “phenyl alkanoic acid derivatives of Formula (I)” and “compounds of the present invention” include all the isotopic forms, stereoisomeric and tautomeric forms and mixtures thereof in all ratios, and their pharmaceutically acceptable salts, solvates, polymorphs, prodrugs, carboxylic acid isosteres, N-oxides and S-oxides. Further, in the context of the present invention, reference to the compounds of Formula (I) may include reference to the compounds represented herein by the compounds of Formula (Ia) and/or the compounds represented herein by the compounds of Formula (Ib).

Within the context of this present application and as used herein the term “isotopic forms” or “isotopically labeled forms” is a general term used for isotopic forms of compounds of Formula (I), wherein one or more atoms of compounds of Formula (I) are replaced by their respective isotopes. All isotopes of any particular atom or element as specified are contemplated within the scope of the compounds of the invention. Examples of isotopes that may be incorporated into the compounds disclosed herein include, but are not limited to, isotopes of hydrogen such as ²H (deuterium or D) and ³H, carbon such as ¹¹C, ¹³C and ¹⁴C, nitrogen such as ¹³N and ¹⁵N, oxygen such as ¹⁵O, ¹⁷O and ¹⁸O, chlorine such as ³⁶Cl, fluorine such as ¹⁸F and sulphur such as ³⁵S. Substitution with heavier isotopes, for example, replacing one or more key carbon-hydrogen bonds with carbon-deuterium bond may show certain therapeutic advantages, resulting from longer metabolism cycles, (e.g., increased in vivo half life or reduced dosage requirements), improved safety or greater effectiveness and hence may be preferred in certain circumstances.

Representative examples of isotopic forms of the compounds of Formula (I) may include, without limitation, deuterated compounds of Formula (I). The term “deuterated” as used herein, by itself or used to modify a compound or group, refers to replacement of one or more hydrogen atom(s), which is attached to carbon(s), with a deuterium atom. For example, the compounds of Formula (I) may include in the definitions of one or more of the various variables R₁, R₄, R₅, R₆, R₇, R₈, R₉, R₁₀, R₁₁, R₁₂, R₁₃, R₁₄, R₁₅ and R₁₆, wherever applicable, deuterium, deuterated-alkyl, deuterated-alkoxy, deuterated-cycloalkyl, deuterated-heterocyclyl, deuterated-aryl, deuterated-heteroaryl and the like.

The term “deuterated-alkyl” refers to an (C₁-C₆)alkyl group as defined herein, wherein at least one hydrogen atom bound to carbon is replaced by a deuterium. That is, in a deuterated alkyl group, at least one carbon atom is bound to a deuterium. In a deuterated alkyl group, it is possible for a carbon atom to be bound to more than one deuterium; it is also possible that more than one carbon atom in the alkyl group is bound to a deuterium. Analogously, the term “deuterated” and the terms deuterated-heterocyclyl, deuterated-heteroaryl, deuterated-cycloalkyl, deute rated-aryl, “deuterated-alkoxy” each refer to the corresponding chemical moiety wherein at least one carbon is bound to a deuterium.

Within the context of the present invention and as used herein, the term “stereoisomer” is a general term used for all isomers of individual compounds that differ only in the orientation of their atoms in space. The term stereoisomer includes mirror image isomers (enantiomers), mixtures of mirror image isomers (racemates, racemic mixtures), geometric (cis/trans or E/Z) isomers, and isomers of compounds with more than one chiral center that are not mirror images of one another (diastereoisomers).

Within the context of the present invention and as used herein, the term “tautomer” refers to the coexistence of two (or more) compounds that differ from each other only in the position of one (or more) mobile atoms and in electron distribution, for example, keto-enol tautomers.

The term “pharmaceutically acceptable salts” as used herein includes salts of the active compounds i.e. the compounds of Formula (I) which are prepared by treating said compounds with a suitable acid or a base, depending on the particular substituents found on the compounds described herein.

Within the context of the present invention and as used herein “N-oxide” refers to the oxide of the nitrogen atom of a nitrogen-containing heteroaryl or heterocycle. N-oxide can be formed in the presence of an oxidizing agent for example peroxide such as m-chloro-perbenzoic acid or hydrogen peroxide. N-oxide refers to an amine oxide, also known as amine-N-oxide, and is a chemical compound that contains N→O bond.

Within the context of the present invention and as used herein “S-oxide” refers to the oxide of the sulfur atom (S-oxide) or dioxide of the sulfur atom (S,S-dioxide) of a sulfur-containing heteroaryl or heterocycle. S-oxide and S,S-dioxides can be formed in the presence of an oxidizing agent for example peroxide such as m-chloro-perbenzoic acid or oxone.

Within the context of the present invention and as used herein, the term “solvate” or “solvates” describe a complex wherein the compound of Formula (I) of the present invention, is coordinated with a proportional amount of a solvent molecule. Specific solvates, wherein the solvent is water, are referred to as hydrates.

Within the context of the present invention and as used herein the term “prodrug” or “prodrugs” refer to the compounds that are drug precursors, which following administration, release the drug in vivo via a chemical or metabolic process, for example, a prodrug on being brought to the physiological pH or through an enzyme action is converted to the desired drug.

Within the context of the present invention and as used herein the term “polymorph” or “polymorphic form” or “polymorphs” refer to crystals of the same compound that differs only in the arrangement and/or conformation of the molecule in the crystal lattice.

Within the context of the present invention and as used herein the term “carboxylic acid isosteres” refer to groups or molecules that have physical and chemical similarities to a carboxylic acid group, producing similar biological effects as those produced by a carboxylic acid group. Examples of carboxylic acid isosteres include groups selected from hydroxamic, acylcyanamide, phosphonate, sulfonate, sulfonamide, tetrazole, hydroxylisoxazole and oxadiazolone (The Practice of Medicinal Chemistry, Edited by Camille G. Wermuth, Second Edition, 2003, 189-214).

Within the context of the present invention and as used herein, the term “GPR agonist” or “GPR agonists” refer to the compound(s) of Formula (I) of the present invention which binds to, activates, increases, stimulates, potentiates, sensitizes or upregulates one or more of the G-protein coupled receptors which are reported to play an important physiological role in insulin release. For instance, the G-protein coupled receptor may be GPR40 that has been reported to play a physiological role in insulin release.

Within the context of the present invention and as used herein, the term “GPR40 agonist” or “GPR40 agonists” refer to the compound(s) of Formula (I) of the present invention which binds to, activates, increases, stimulates, potentiates, sensitizes or upregulates GPR40 receptor and promotes glucose induced insulin secretion.

The term “therapeutically effective amount” as used herein in the present invention generally refers to the amount of the compound (e.g. the compound of Formula (I)) or a composition containing the said compound that will elicit the biological or medical response of a tissue or a subject when treated with the compound. Particularly, the term “therapeutically effective amount” includes the amount of a compound, when administered, that induces a positive modification in the disease or condition to be treated or is sufficient to prevent development of, or alleviate to some extent, one or more of the symptoms of the condition or disorder being treated in a subject. In respect of the therapeutic amount of the compound, consideration is also given that the amount of the compound used for the treatment of a subject is low enough to avoid undue or severe side effects, within the scope of sound medical judgement. The therapeutically effective amount of the compound or composition will vary with the particular condition being treated, the age and physical condition of the end user, the severity of the condition being treated or prevented, the duration of the treatment, the nature of concurrent therapy, the specific compound or composition employed, the particular pharmaceutically acceptable carrier utilized and other factors.

The term “treatment”, “treat” and “therapy” as used herein and the like refer to alleviate, slow the progression, prophylaxis, attenuation or cure of existing disease (for example, metabolic disorders). Treatment also includes preventing development of, or alleviating to some extent, one or more of the symptoms of the disease or condition being treated.

As used herein, the term “prophylaxis” covers within its purview the preventive treatment of a subclinical disease-state or a condition in a subject (e.g. a human), aimed at reducing the probability of the occurrence of a clinical disease-state. Subjects are selected for preventative therapy based on factors that are known to increase risk of suffering a clinical disease state or a condition compared to the general population. “Prophylaxis” therapies can be divided into (a) primary prevention and (b) secondary prevention. Primary prevention is defined as treatment in a subject that has not yet presented with a clinical disease state or a condition, whereas secondary prevention is defined as preventing a second occurrence of the same or similar clinical disease state.

The term “subject” as used herein refers to an animal, preferably a mammal, and most preferably a human.

The term “mammal” used herein refers to warm-blooded vertebrate animals of the class Mammalia, including humans, characterized by a covering of hair on the skin and, in the female, milk-producing mammary glands for nourishing the young. The term mammal includes animals such as cat, dog, rabbit, bear, fox, wolf, monkey, deer, mouse, pig as well as human.

### EMBODIMENTS

In an embodiment, the present invention encompasses a compound of Formula (I), wherein R₁ is selected from hydrogen, methyl, ethyl or propyl.

In one embodiment, the present invention encompasses a compound of Formula (I), wherein R₂ and R₃ together form a saturated or a partially unsaturated 3- to 9-membered heterocyclyl ring containing one or two heteroatoms independently selected from O, N or S.

In another embodiment, the present invention encompasses a compound of Formula (I), wherein R₂ and R₃ together form a saturated or a partially unsaturated 3- to 9-membered heterocyclyl ring containing one or two O atoms.

In yet another embodiment, the present invention encompasses a compound of Formula (I), wherein R₂ and R₃ together form an oxetane ring.

In another embodiment, the present invention encompasses a compound of Formula (I), wherein R₂ and R₃ together form a saturated or a partially unsaturated heterocyclyl ring containing one or two heteroatoms independently selected from N or S atoms; when the heteroatom is N, it is substituted with hydrogen, (C₁-C₆)alkyl, —C(O)(C₁-C₆)alkyl or —S(O)₂(C₁-C₆)alkyl.

In another embodiment, the present invention encompasses a compound of Formula (I), wherein R₂ and R₃ together form a saturated or a partially unsaturated (C₄-C₈)cycloalkyl ring.

In another embodiment, the present invention encompasses a compound of Formula (I), wherein Rₓ is A-CH(R₇)—X and R_{y} is R₅; wherein X, R₅, R₇ and A are as defined above.

In yet another embodiment, the present invention encompasses a compound of Formula (I), wherein both Rₓ and R_{y} represent A-CH(R₇)—X; wherein X, R₇ and A are as defined above.

In yet another further embodiment, the present invention encompasses a compound of Formula (I), wherein Rₓ is R₅ and R_{y} is A-CH(R₇)—X; wherein X, R₅, R₇ and A are as defined above.

In another embodiment, the present invention encompasses a compound of Formula (I), wherein Rₓ is A-CH(R₇)—X and R_{y} is R₅; wherein X is O and R₅, R₇ and A are as defined above.

In yet another embodiment, the present invention encompasses a compound of Formula (I), wherein both Rₓ and R_{y} represent A-CH(R₇)—X; wherein X is O and R₇ and A are as defined above.

In yet another further embodiment, the present invention encompasses a compound of Formula (I), wherein Rₓ is R₅ and R_{y} is A-CH(R₇)—X; wherein X is O and R₅, R₇ and A are as defined above.

In a further embodiment, the present invention encompasses a compound of Formula (I), wherein Rₓ is A-CH(R₇)—X and R_{y} is R₅; and wherein X is S or NR₈, wherein R₈ is selected from hydrogen, (C₁-C₆) alkyl, —C(O)(C₁-C₆)alkyl, —C(O)O(C₁-C₆)alkyl, —C(O)NH₂ or —S(O)ₚR₆, wherein R₅, R₆, R₇, A and p are as defined above.

In yet another embodiment, the present invention encompasses a compound of Formula (I), wherein both Rₓ and R_{y} represent A-CH(R₇)—X; and wherein X is S or NR₈, wherein R₈ is selected from hydrogen, (C₁-C₆)alkyl, —C(O)(C₁-C₆)alkyl, —C(O)O(C₁-C₆)alkyl, —C(O)NH₂ or —S(O)ₚR₆, wherein R₆, R₇, A and p are as defined above.

In yet another further embodiment, the present invention encompasses a compound of Formula (I), wherein Rₓ is R₅ and R_{y} is A-CH(R₇)—X; and wherein X is S or NR₈, wherein R₈ is selected from hydrogen, (C₁-C₆)alkyl, —C(O)(C₁-C₆)alkyl, —C(O)O(C₁-C₆)alkyl, —C(O)NH₂ or —S(O)ₚR₆, wherein R₅, R₆, R₇, A and p are as defined above.

In one embodiment, the present invention encompasses a compound of Formula (I), wherein A is selected from:

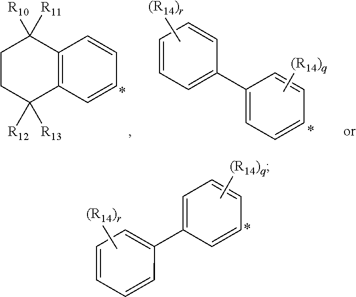

wherein R₁₀, R₁₁, R₁₂, R₁₃, R₁₄, q, r and * are as defined above.

In yet another embodiment, the present invention encompasses a compound of Formula (I), wherein A is

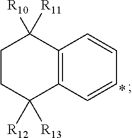

wherein R₁₀, R₁₁, R₁₂ and R₁₃ represent (C₁-C₆) alkyl; and * is as defined above.

In another embodiment, the present invention encompasses a compound of Formula (I), wherein A is selected from:

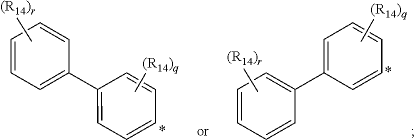

wherein R₁₄ at each occurrence is selected from hydrogen, (C₁-C₆) alkyl, halogen, halo(C₁-C₆) alkyl, —O(C₁-C₆)alkyl, halo(C₁-C₆)alkoxy, —O(C₁-C₆)alkyl-S(O)ₚR₆, —O(C₁-C₆)alkyl-heterocyclyl, —O-heterocyclyl, cyano, —S(O)ₚR₆, —(CH₂)ₛNR₁₅R₁₆ or —X(CH₂)ₛNR₁₅R₁₆, wherein X, R₆, R₁₅, R₁₆, p, q, r, s and * are as defined above; (C₁-C₆)alkyl may be unsubstituted or substituted with one or more groups independently selected from (C₁-C₆)alkyl, halogen, halo(C₁-C₆) alkyl, hydroxy, —O(C₁-C₆)alkyl, (C₆-C₁₀)aryl, heterocyclyl, amino, cyano, nitro, —C(O)R₉ and —O(C₁-C₆)alkyl-S(O)ₚR₆, wherein R₆, R₉, and p are as defined above; and heterocyclyl may be unsubstituted or substituted with one or more groups independently selected from (C₁-C₆)alkyl, halogen, halo(C₁-C₆)alkyl, hydroxy, —O(C₁-C₆)alkyl, halo(C₁-C₆)alkoxy, (C₆-C₁₀)aryl, amino, cyano, nitro, —(C₁-C₆)alkyl-OH, —(C₁-C₆)alkyl-O—(C₁-C₆)alkyl, —C(O)R₉ and —O(C₁-C₆)alkyl-S(O)ₚR₆, wherein R₆, R₉ and p are as defined above.

In one embodiment, the present invention encompasses a compound of Formula (I), wherein A is selected from (C₆-C₁₀)aryl or heteroaryl; wherein (C₆-C₁₀)aryl is unsubstituted or substituted with one or more groups independently selected from (C₁-C₆) alkyl, halogen, halo(C₁-C₆)alkyl, hydroxy, —O(C₁-C₆)alkyl, halo(C₁-C₆)alkoxy, (C₆-C₁₀)aryl, heteroaryl, amino, cyano, nitro, —C(O)R₉ and —O(C₁-C₆)alkyl-S(O)ₚR₆, wherein R₆, R₉, and p are as defined above; heteroaryl is unsubstituted or substituted with one or more groups independently selected from (C₁-C₆) alkyl, halogen, halo(C₁-C₆) alkyl, hydroxy, —O(C₁-C₆)alkyl, halo(C₁-C₆)alkoxy, (C₆-C₁₀)aryl, heterocyclyl, amino, cyano, nitro, —C(O)R₉ and —O(C₁-C₆)alkyl-S(O)ₚR₆, wherein R₆, R₉, and p are as defined above.

In an embodiment, the compounds of Formula (I) encompasses a compound of Formula (Ia),

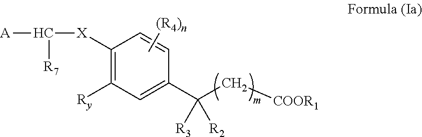

wherein,
R₁ is hydrogen or (C₁-C₆) alkyl;
R₂ and R₃ together form a saturated or a partially unsaturated 3- to 9-membered heterocyclyl ring containing one or two heteroatoms independently selected from O, N and S; or R₂ and R₃ together form a saturated or a partially unsaturated (C₄-C₈) cycloalkyl ring;
R₄ at each occurrence is independently selected from hydrogen, (C₁-C₆)alkyl, halogen, halo(C₁-C₆) alkyl, hydroxy, —O(C₁-C₆)alkyl, (C₆-C₁₀)aryl, amino, cyano, nitro, —C(O)R₉ and —S(O)ₚR₆;

### R_{y} is A-CH(R₇)—X or R₅;

R₅ is selected from hydrogen, (C₁-C₆) alkyl, halogen, halo(C₁-C₆)alkyl, hydroxy, —O(C₁-C₆)alkyl, (C₆-C₁₀)aryl, amino, cyano, nitro, —C(O)R₉ or —S(O)ₚR₆;
R₆ is selected from hydrogen, (C₁-C₆) alkyl or amino;
R₇ is hydrogen or (C₁-C₆)alkyl;
X is selected from O, NR₈ or S;
R₈ is selected from hydrogen, (C₁-C₆)alkyl, (C₃-C₈)cycloalkyl, (C₆-C₁₀)aryl, heterocyclyl, heteroaryl, cyano, —C(O)(C₁-C₆)alkyl, —C(O)O(C₁-C₆)alkyl, —C(O)NH₂ or —S(O)ₚR₆; wherein R₆ is as defined above;
R₉ is selected from (C₁-C₆) alkyl, O(C₁-C₆)alkyl, hydroxy or amino;
A is selected from (C₃-C₈)cycloalkyl, (C₆-C₁₀)aryl, heterocyclyl, heteroaryl,

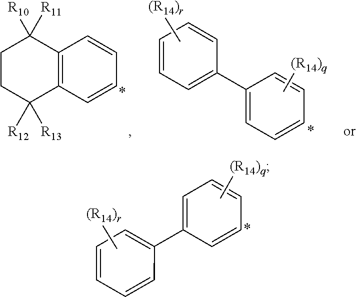

R₁₀, R₁₁, R₁₂ and R₁₃ are independently selected from hydrogen and (C₁-C₆) alkyl; or R₁₀ and R₁₁ can together form a (C₃-C₈)cycloalkyl ring and R₁₂ and R₁₃ are hydrogen; or R₁₂ and R₁₃ can together form a (C₃-C₈) cycloalkyl ring; and R₁₀ and R₁₁ are hydrogen;
R₁₄ at each occurrence is independently selected from hydrogen, (C₁-C₆) alkyl, halogen, halo(C₁-C₆) alkyl, hydroxy, —O(C₁-C₆)alkyl, —O(C₃-C₈)cycloalkyl, halo(C₁-C₆)alkoxy, —O(C₁-C₆)alkyl-S(O)ₚR₆, —O(C₁-C₆)alkyl-heterocyclyl, —O-heterocyclyl, (C₆-C₁₀)aryl, amino, cyano, nitro, —C(O)R₉, —S(O)ₚR₆, —(CH₂)ₛNR₁₅R₁₆ and —X(CH₂)ₛNR₁₅R₁₆; wherein X, R₆ and R₉ are as defined above;
R₁₅ and R₁₆ are independently selected from hydrogen, (C₁-C₆)alkyl and —(CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4;
t is an integer from 1 to 4;
* indicates the point of attachment to —CH of CH(R₇)—X;
wherein,

(C₁-C₆)alkyl is unsubstituted or substituted with one or more groups selected from (C₁-C₆)alkyl, (C₂-C₈)alkenyl, (C₂-C₈)alkynyl, halogen, halo(C₁-C₆)alkyl, hydroxy, —O(C₁-C₆)alkyl, (C₃-C₈)cycloalkyl, (C₆-C₁₀)aryl, heterocyclyl, heteroaryl, amino, cyano, nitro, —C(O)R₉ or —O(C₁-C₆)alkyl-S(O)ₚR₆; wherein R₆, R₉, and p are as defined above;

—O(C₁-C₆)alkyl is unsubstituted or substituted with one or more groups selected from (C₁-C₆)alkyl, (C₃-C₈)cycloalkyl, heterocyclyl, hydroxy, halogen, amino, cyano, —(C₁-C₆)alkyl-S(O)ₚR₆, —S(O)ₚR₆, —NR₁₅R₁₆ or —(CH₂)ₛNR₁₅R₁₆; wherein R₆, R₁₅, R₁₆, p and s are as defined above;

(C₆-C₁₀)aryl is unsubstituted or substituted with one or more groups selected from (C₁-C₆)alkyl, (C₂-C₈) alkenyl, (C₂-C₈) alkynyl, halogen, halo(C₁-C₆) alkyl, hydroxy, —O(C₁-C₆) alkyl, halo(C₁-C₆)alkoxy, (C₃-C₈)cycloalkyl, (C₆-C₁₀)aryl, heterocyclyl, heteroaryl, amino, cyano, nitro, —C(O)R₉ or —O(C₁-C₆)alkyl-S(O)ₚR₆; wherein R₆, R₉, and p are as defined above;

heterocyclyl is a 3- to 9-membered ring, which is unsubstituted or substituted with one or more groups selected from (C₁-C₆)alkyl, (C₂-C₈)alkenyl, (C₂-C₈)alkynyl, halogen, halo(C₁-C₆)alkyl, hydroxy, —O(C₁-C₆)alkyl, halo(C₁-C₆)alkoxy, (C₃-C₈)cycloalkyl, (C₆-C₁₀)aryl, heterocyclyl, heteroaryl, amino, cyano, nitro, —(C₁-C₆)alkyl-OH, (C₁-C₆)alkyl-O—(C₁-C₆)alkyl, C(O)R₉ or O(C₁-C₆)alkyl-S(O)ₚR₆; wherein R₆, R₉, and p are as defined above;

heteroaryl is a 3- to 10-membered ring, which is unsubstituted or substituted with one or more groups selected from (C₁-C₆)alkyl, (C₂-C₈)alkenyl, (C₂-C₈)alkynyl, halogen, halo(C₁-C₆) alkyl, hydroxy, —O(C₁-C₆)alkyl, halo(C₁-C₆)alkoxy, (C₃-C₈)cycloalkyl, (C₆-C₁₀)aryl, heterocyclyl, heteroaryl, amino, cyano, nitro, —C(O)R₉ or —O(C₁-C₆)alkyl-S(O)ₚR₆; wherein R₆, R₉, and p are as defined above;

halogen is selected from chlorine, bromine, iodine or fluorine;

or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

In an embodiment, the compounds of Formula (I) encompasses a compound of Formula (Ia);

wherein,
R₁ is hydrogen or (C₁-C₆) alkyl;
R₂ and R₃ together form a saturated or a partially unsaturated 3- to 9-membered heterocyclyl ring containing one or two heteroatoms independently selected from O, N or S; or R₂ and R₃ together form a saturated or a partially unsaturated (C₄-C₈)cycloalkyl ring;
R₄ at each occurrence is independently selected from hydrogen, (C₁-C₆)alkyl, halogen, halo(C₁-C₆) alkyl, hydroxy, —O(C₁-C₆)alkyl, (C₆-C₁₀)aryl, amino, cyano, nitro, —C(O)R₉ and —S(O)R₆;

### R_{y} is R₅;

R₅ is selected from hydrogen, (C₁-C₆)alkyl, halogen, halo(C₁-C₆)alkyl, hydroxy, —O(C₁-C₆)alkyl, (C₆-C₁₀)aryl, amino, cyano, nitro, —C(O)R₉ or —S(O)ₚR₆;
R₆ is selected from hydrogen, (C₁-C₆) alkyl or amino;
R₇ is hydrogen or (C₁-C₆)alkyl;
X is selected from O, NR₈ or S;
R₈ is selected from hydrogen, (C₁-C₆) alkyl, (C₃-C₈)cycloalkyl, (C₆-C₁₀)aryl, heterocyclyl, heteroaryl, cyano, —C(O)(C₁-C₆)alkyl, —C(O)O(C₁-C₆)alkyl, —C(O)NH₂ or —S(O)ₚR₆; wherein R₆ is as defined above;
R₉ is selected from (C₁-C₆) alkyl, —O(C₁-C₆)alkyl, hydroxy or amino;
A is selected from (C₃-C₈)cycloalkyl, (C₆-C₁₀)aryl, heterocyclyl, heteroaryl,

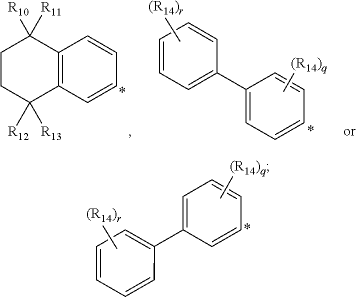

R₁₀, R₁₁, R₁₂ and R₁₃ are independently selected from hydrogen and (C₁-C₆) alkyl; or R₁₀ and R₁₁ can together form a (C₃-C₈)cycloalkyl ring and R₁₂ and R₁₃ are hydrogen; or R₁₂ and R₁₃ can together form a (C₃-C₈) cycloalkyl ring and R₁₀ and R₁₁ are hydrogen;
R₁₄ at each occurrence is independently selected from hydrogen, (C₁-C₆) alkyl, halogen, halo(C₁-C₆)alkyl, hydroxy, —O(C₁-C₆)alkyl, —O(C₃-C₈)cycloalkyl, halo(C₁-C₆)alkoxy, —O(C₁-C₆)alkyl-S(O)ₚR₆, —O(C₁-C₆)alkyl-heterocyclyl, —O-heterocyclyl, (C₆-C₁₀)aryl, amino, cyano, nitro, —C(O)R₉, —S(O)ₚR₆, —(CH₂)ₛNR₁₅R₁₆ and —X(CH₂)ₛNR₁₅R₁₆; wherein X, R₆ and R₉ are as defined above;
R₁₅ and R₁₆ are independently selected from hydrogen, (C₁-C₆)alkyl and —(CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4;
t is an integer from 1 to 4;
* indicates the point of attachment to —CH of CH(R₇)—X;
wherein,

(C₁-C₆)alkyl is unsubstituted or substituted with one or more groups independently selected from (C₁-C₆)alkyl, (C₂-C₈) alkenyl, (C₂-C₈) alkynyl, halogen, halo(C₁-C₆) alkyl, hydroxy, —O(C₁-C₆)alkyl, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

—O(C₁-C₆)alkyl is unsubstituted or substituted with one or more groups independently selected from (C₁-C₆)alkyl, (C₃-C₈)cycloalkyl, heterocyclyl, hydroxy, halogen, amino, cyano, —(C₁-C₆)alkyl-S(O)ₚR₆, —S(O)ₚR₆, —NR₁₅R₁₆ and —(CH₂)ₛNR₁₅R₁₆; wherein R₆, R₁₅, R₁₆, p and s are as defined above;

(C₆-C₁₀)aryl is unsubstituted or substituted with one or more groups independently selected from (C₁-C₆)alkyl, (C₂-C₈) alkenyl, (C₂-C₈) alkynyl, halogen, halo(C₁-C₆) alkyl, hydroxy, —O(C₁-C₆) alkyl, halo(C₁-C₆)alkoxy, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

heterocyclyl is a 3- to 9-membered ring, which is unsubstituted or substituted with one or more groups independently selected from (C₁-C₆)alkyl, (C₂-C₈) alkenyl, (C₂-C₈) alkynyl, halogen, halo(C₁-C₆) alkyl, hydroxy, —O(C₁-C₆) alkyl, halo(C₁-C₆)alkoxy, (C₃-C₈)cycloalkyl, (C₆-C₁₀)aryl, heterocyclyl, heteroaryl, amino, cyano, nitro, —(C₁-C₆)alkyl-OH, (C₁-C₆)alkyl-O—(C₁-C₆)alkyl, —C(O)R₉ and —O(C₁-C₆)alkyl-S(O)ₚR₆; wherein R₆, R₉, and p are as defined above;

heteroaryl is a 3- to 10-membered ring, which is unsubstituted or substituted with one or more groups independently selected from (C₁-C₆)alkyl, (C₂-C₈) alkenyl, (C₂-C₈) alkynyl, halogen, halo(C₁-C₆) alkyl, hydroxy, —O(C₁-C₆) alkyl, halo(C₁-C₆)alkoxy, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

halogen is selected from chlorine, bromine, iodine or fluorine;

or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

In an embodiment, the compound of Formula (I) encompasses the compound of Formula (Ia),

wherein,
R₁ is hydrogen or (C₁-C₆) alkyl;
R₂ and R₃ together form a saturated or a partially unsaturated 3 to 9-membered heterocyclyl ring containing one or two heteroatoms independently selected from O, N and S;
R₄ at each occurrence is independently selected from hydrogen, (C₁-C₆) alkyl, halogen, halo(C₁-C₆) alkyl, hydroxy, —O(C₁-C₆)alkyl, amino, cyano, —C(O)R₉ and —S(O)ₚR₆;

### R_{y} is R₅;

R₅ is selected from hydrogen, (C₁-C₆)alkyl, halogen, halo(C₁-C₆) alkyl, hydroxy, —O(C₁-C₆)alkyl, amino, cyano, —C(O)R₉ or —S(O)ₚR₆;
R₆ is selected from hydrogen, (C₁-C₆) alkyl or amino;
R₇ is hydrogen or (C₁-C₆)alkyl;
X is selected from O, NR₈ or S;
R₈ is selected from hydrogen, (C₁-C₆) alkyl, —C(O)(C₁-C₆)alkyl, —C(O)O(C₁-C₆)alkyl, —C(O)NH₂ or —S(O)ₚR₆, wherein R₆ is as defined above;
R₉ is selected from (C₁-C₆) alkyl, —O(C₁-C₆)alkyl, hydroxy or amino;
A is selected from (C₆-C₁₀)aryl, heteroaryl,

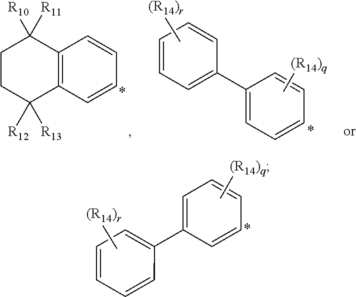

R₁₀, R₁₁, R₁₂ and R₁₃ are independently selected from hydrogen and (C₁-C₆) alkyl; or R₁₀ and R₁₁ can together form a (C₃-C₈)cycloalkyl ring and R₁₂ and R₁₃ are hydrogen; or R₁₂ and R₁₃ can together form a (C₃-C₈)cycloalkyl ring and R₁₂ and R₁₃ are hydrogen;
R₁₄ at each occurrence is independently selected from hydrogen, (C₁-C₆) alkyl, halogen, halo(C₁-C₆) alkyl, hydroxy, —O(C₁-C₆)alkyl, —O(C₃-C₈)cycloalkyl, halo(C₁-C₆)alkoxy, —O(C₁-C₆)alkyl-S(O)ₚR₆, —O(C₁-C₆)alkyl-heterocyclyl, —O-heterocyclyl, (C₆-C₁₀)aryl, amino, cyano, nitro, —C(O)R₉, —S(O)ₚR₆, —(CH₂)ₛNR₁₅R₁₆ and —X(CH₂)ₛNR₁₅R₁₆; wherein X, R₆ and R₉ are as defined above;
R₁₅ and R₁₆ are independently selected from hydrogen, (C₁-C₆)alkyl and —(CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4;
t is an integer from 1 to 4;
* indicates the point of attachment to —CH of CH(R₇)—X;
wherein,

(C₁-C₆)alkyl is unsubstituted or substituted with one or more groups independently selected from (C₁-C₆)alkyl, halogen, halo(C₁-C₆) alkyl, hydroxy, —O(C₁-C₆)alkyl, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

—O(C₁-C₆)alkyl is unsubstituted or substituted with one or more groups independently selected from (C₁-C₆)alkyl, (C₃-C₈)cycloalkyl, heterocyclyl, hydroxy, halogen, amino, cyano, —(C₁-C₆)alkyl-S(O)ₚR₆, —S(O)ₚR₆, —NR₁₅R₁₆ and —(CH₂)ₛNR₁₅R₁₆; wherein R₆, R₁₅, R₁₆, p and s are as defined above;

(C₆-C₁₀)aryl is unsubstituted or substituted with one or more groups independently selected from (C₁-C₆)alkyl, halogen, halo(C₁-C₆) alkyl, hydroxy, —O(C₁-C₆) alkyl, halo(C₁-C₆)alkoxy, (C₆-C₁₀)aryl, heteroaryl, amino, cyano, nitro, —C(O)R₉ and —O(C₁-C₆)alkyl-S(O)ₚR₆; wherein R₆, R₉, and p are as defined above;

heterocyclyl is a 3- to 9-membered ring, which is unsubstituted or substituted with one or more groups independently selected from (C₁-C₆)alkyl, halogen, halo(C₁-C₆) alkyl, hydroxy, —O(C₁-C₆) alkyl, halo(C₁-C₆)alkoxy, (C₆-C₁₀)aryl, amino, cyano, nitro, —(C₁-C₆)alkyl-OH, (C₁-C₆)alkyl-O—(C₁-C₆)alkyl and —O(C₁-C₆)alkyl-S(O)ₚR₆; wherein R₆ and p are as defined above;

heteroaryl is a 3- to 10-membered ring, which is unsubstituted or substituted with one or more groups independently selected from (C₁-C₆)alkyl, halogen, halo(C₁-C₆) alkyl, hydroxy, —O(C₁-C₆) alkyl, halo(C₁-C₆)alkoxy, (C₆-C₁₀)aryl, heteroaryl, heterocyclyl, amino, cyano, nitro, —C(O)R₉ and —O(C₁-C₆)alkyl-S(O)ₚR₆; wherein R₆, R₉, and p are as defined above;

halogen is selected from chlorine, bromine, iodine or fluorine;

or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

In an embodiment, the compound of Formula (I) encompasses the compound of Formula (Ia),

wherein,
R₁ is hydrogen or (C₁-C₆) alkyl;
R₂ and R₃ together form a saturated or a partially unsaturated 3- to 9-membered heterocyclyl ring containing one or two oxygen atoms;
R₄ at each occurrence is independently selected from hydrogen, (C₁-C₆) alkyl, halogen, halo(C₁-C₆) alkyl, hydroxy, —O(C₁-C₆)alkyl, amino, cyano, —C(O)R₉ and —S(O)ₚR₆;

### R_{y} is R₅;

R₅ is selected from hydrogen, (C₁-C₆) alkyl, halogen, halo(C₁-C₆)alkyl, hydroxy, —O(C₁-C₆)alkyl, amino, cyano, —C(O)R₉ or —S(O)ₚR₆;
R₆ is selected from hydrogen, (C₁-C₆) alkyl or amino;
R₇ is hydrogen or (C₁-C₆)alkyl;

### X is O;

R₉ is selected from (C₁-C₄) alkyl, —O(C₁-C₆)alkyl, hydroxy or amino;
A is selected from

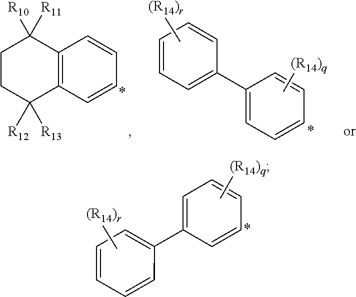

R₁₀, R₁₁, R₁₂ and R₁₃ are independently selected from hydrogen and (C₁-C₆) alkyl; or R₁₀ and R₁₁ can together form a (C₃-C₈) cycloalkyl ring and R₁₂ and R₁₃ are hydrogen; or, R₁₂ and R₁₃ can together form a (C₃-C₈) cycloalkyl ring and R₁₂ and R₁₃ are hydrogen;
R₁₄ at each occurrence is independently selected from hydrogen, (C₁-C₆) alkyl, halogen, halo(C₁-C₆) alkyl, hydroxy, —O(C₁-C₆)alkyl, —O(C₃-C₈)cycloalkyl, halo(C₁-C₆)alkoxy, —O(C₁-C₆)alkyl-S(O)ₚR₆, —O(C₁-C₆)alkyl-heterocyclyl, —O-heterocyclyl, (C₆-C₁₀)aryl, amino, cyano, nitro, —C(O)R₉, —S(O)ₚR₆, —(CH₂)ₛNR₁₅R₁₆ and —X(CH₂)ₛNR₁₅R₁₆; wherein X, R₆ and R₉ are as defined above;
R₁₅ and R₁₆ are independently selected from hydrogen, (C₁-C₆)alkyl and (CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4;
t is an integer from 1 to 4;
* indicates the point of attachment to —CH of CH(R₇)—X;
wherein,

(C₁-C₆)alkyl is unsubstituted or substituted with one or more groups independently selected from (C₁-C₆)alkyl, halogen, halo(C₁-C₆) alkyl, hydroxy, O(C₁-C₆)alkyl, (C₃-C₈)cycloalkyl, (C₆-C₁₀)aryl, heterocyclyl, heteroaryl, amino, cyano, nitro, C(O)R₉ and O(C₁-C₆)alkyl-S(O)ₚR₆; wherein R₆, R₉, and p are as defined above;

—O(C₁-C₆)alkyl is unsubstituted or substituted with one or more groups independently selected from (C₁-C₆)alkyl, (C₃-C₈)cycloalkyl, heterocyclyl, hydroxy, halogen, amino, cyano, (C₁-C₆)alkyl-S(O)ₚR₆, —S(O)ₚR₆, —NR₁₅R₁₆ and —(CH₂)ₛNR₁₅R₁₆; wherein R₆, R₁₅, R₁₆, p and s are as defined above;

(C₆-C₁₀)aryl is unsubstituted or substituted with one or more groups independently selected from (C₁-C₆)alkyl, halogen, halo(C₁-C₆) alkyl, hydroxy, O(C₁-C₆) alkyl, halo(C₁-C₆)alkoxy, (C₆-C₁₀)aryl, heteroaryl, amino, cyano, nitro, C(O)R₉ and O(C₁-C₆)alkyl-S(O)ₚR₆; wherein R₆, R₉, and p are as defined above;

heterocyclyl is a 3- to 9-membered ring, which is unsubstituted or substituted with one or more groups independently selected from (C₁-C₆)alkyl, halogen, halo(C₁-C₆) alkyl, hydroxy, O(C₁-C₆) alkyl, halo(C₁-C₆)alkoxy, (C₆-C₁₀)aryl, amino, cyano, nitro, (C₁-C₆)alkyl-OH, (C₁-C₆)alkyl-O—(C₁-C₆)alkyl and O(C₁-C₆)alkyl-S(O)ₚR₆; wherein R₆ and p are as defined above;

heteroaryl is a 3- to 10-membered ring, which is unsubstituted or substituted with one or more groups independently selected from (C₁-C₆)alkyl, halogen, halo(C₁-C₆) alkyl, hydroxy, O(C₁-C₆) alkyl, halo(C₁-C₆)alkoxy, (C₆-C₁₀)aryl, heteroaryl, amino, cyano, nitro, C(O)R₉ and O(C₁-C₆)alkyl-S(O)ₚR₆; wherein R₆, R₉, and p are as defined above;

halogen is selected from chlorine, bromine, iodine or fluorine; or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

In an embodiment, the compound of Formula (I) encompasses the compound of Formula (Ia),

wherein,
R₁ is hydrogen or (C₁-C₆) alkyl;
R₂ and R₃ together form a saturated or a partially unsaturated 3- to 9-membered heterocyclyl ring containing one or two oxygen atoms;
R₄ at each occurrence is independently selected from hydrogen, (C₁-C₆) alkyl, halogen, halo(C₁-C₆) alkyl, hydroxy, —O(C₁-C₆)alkyl, amino, cyano, —C(O)R₉ and —S(O)ₚR₆;

### R_{y} is R₅;

R₅ is selected from hydrogen, (C₁-C₆) alkyl, halogen, halo(C₁-C₆) alkyl, hydroxy, —O(C₁-C₆)alkyl, amino, cyano, —C(O)R₉ or —S(O)ₚR₆;
R₆ is selected from hydrogen, (C₁-C₆) alkyl or amino;
R₇ is hydrogen or (C₁-C₆)alkyl;

### X is O;

R₉ is selected from (C₁-C₄) alkyl, —O(C₁-C₆)alkyl, hydroxy or amino;

### A is

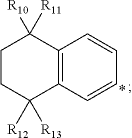

R₁₀, R₁₁, R₁₂ and R₁₃ represent (C₁-C₆) alkyl;
R₁₅ and R₁₆ are independently selected from hydrogen, (C₁-C₆)alkyl and —(CH₂)ₜOH;
n is an integer from 1 to 3;
m is an integer from 0 to 4;
p is an integer from 0 to 2;
s is an integer from 1 to 4;
t is an integer from 1 to 4;
* indicates the point of attachment to —CH of CH(R₇)—X;
wherein,

(C₁-C₆)alkyl is unsubstituted or substituted with one or more groups independently selected from (C₁-C₆)alkyl, halogen, halo(C₁-C₆) alkyl, hydroxy, —O(C₁-C₆) alkyl, amino, cyano, nitro, —C(O)R₉ and —O(C₁-C₆)alkyl-S(O)ₚR₆; wherein R₆, R₉, and p are as defined above;

—O(C₁-C₆)alkyl is unsubstituted or substituted with one or more groups independently selected from hydroxy, halogen, amino, —(C₁-C₆)alkyl-S(O)ₚR₆, —S(O)ₚR₆, —NR₁₅R₁₆ and —(CH₂)ₛNR₁₅R₁₆; wherein R₆, R₁₅, R₁₆, p and s are as defined above;

halogen is selected from chlorine, bromine, iodine or fluorine;

or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

In an embodiment, the compounds of Formula (I) encompasses a compound of Formula (Ia),

wherein,
R₁ is hydrogen or (C₁-C₆) alkyl;
R₂ and R₃ together form a saturated or a partially unsaturated 3- to 9-membered heterocyclyl ring containing one or two oxygen atoms;
R₄ at each occurrence is independently selected from hydrogen, (C₁-C₆) alkyl, halogen, halo(C₁-C₆) alkyl, hydroxy, —O(C₁-C₆)alkyl, amino, cyano, —C(O)R₉ and —S(O)ₚR₆;

### R_{y} is R₅;

R₅ is selected from hydrogen, (C₁-C₆) alkyl, halogen, halo(C₁-C₆) alkyl, hydroxy, —O(C₁-C₆)alkyl, amino, cyano, —C(O)R₉ or —S(O)ₚR₆;
R₆ is selected from hydrogen, (C₁-C₆) alkyl or amino;
R₇ is hydrogen or (C₁-C₆)alkyl;

### X is O;

R₉ is selected from (C₁-C₆) alkyl, O(C₁-C₆)alkyl, hydroxy or amino;
A is selected from

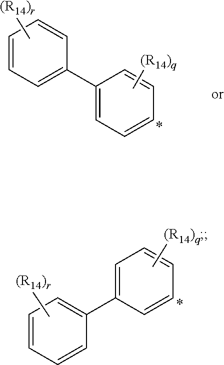

R₁₄ at each occurrence is independently selected from hydrogen, (C₁-C₆)alkyl, halogen, halo(C₁-C₆) alkyl, —O(C₁-C₆)alkyl, halo(C₁-C₆)alkoxy, —O(C₁-C₆)alkyl-S(O)ₚR₆, —O(C₃-C₈)cycloalkyl, —O(C₁-C₆)alkyl-heterocyclyl, —O-heterocyclyl, cyano, —S(O)ₚR₆, —(CH₂)ₛNR₁₅R₁₆ and —X(CH₂)ₛNR₁₅R₁₆; wherein X and R₆ are as defined above;
R₁₅ and R₁₆ are independently selected from hydrogen, (C₁-C₆)alkyl and —(CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4;
t is an integer from 1 to 4;
* indicates the point of attachment to —CH of CH(R₇)—X;
wherein,

(C₁-C₆)alkyl is unsubstituted or substituted with one or more groups independently selected from (C₁-C₆)alkyl, halogen, halo(C₁-C₆) alkyl, hydroxy, —O(C₁-C₆) alkyl, (C₆-C₁₀)aryl, amino, cyano, nitro, —C(O)R₉ and —O(C₁-C₆)alkyl-S(O)ₚR₆; wherein R₆, R₉, and p are as defined above;

—O(C₁-C₆)alkyl is unsubstituted or substituted with one or more groups independently selected from (C₁-C₆)alkyl, (C₃-C₈)cycloalkyl, heterocyclyl, hydroxy, halogen, cyano, —(C₁-C₆)alkyl-S(O)ₚR₆, —S(O)ₚR₆, —NR₁₅R₁₆ and —(CH₂)ₛNR₁₅R₁₆; wherein R₆, R₁₅, R₁₆, p and s are as defined above;

heterocyclyl is 3- to 9-membered ring, which is unsubstituted or substituted with one or more groups independently selected from (C₁-C₆)alkyl, halogen, halo(C₁-C₆) alkyl, hydroxy, —O(C₁-C₆) alkyl, halo(C₁-C₆)alkoxy, (C₆-C₁₀)aryl, amino, cyano, nitro, —(C₁-C₆)alkyl-OH, (C₁-C₆)alkyl-O—(C₁-C₆)alkyl and —O(C₁-C₆)alkyl-S(O)ₚR₆; wherein R₆ and p are as defined above;

halogen is selected from chlorine, bromine, iodine or fluorine;

or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

In an embodiment, the compounds of Formula (I) encompasses a compound of Formula (Ia),

wherein,
R₁ is hydrogen or (C₁-C₆) alkyl;
R₂ and R₃ together form a saturated or a partially unsaturated 3- to 9-membered heterocyclyl ring containing one or two oxygen atoms;
R₄ at each occurrence is independently selected from hydrogen, (C₁-C₆) alkyl, halogen, halo(C₁-C₆) alkyl, hydroxy, —O(C₁-C₆)alkyl, amino, cyano, —C(O)R₉ or —S(O)ₚR₆;

### R_{y} is R₅;

R₅ is selected from hydrogen, (C₁-C₆) alkyl, halogen, halo(C₁-C₆) alkyl, hydroxy, —O(C₁-C₆)alkyl, amino, cyano, —C(O)R₉ or —S(O)ₚR₆;
R₆ is selected from hydrogen, (C₁-C₆) alkyl or amino;
R₇ is hydrogen or (C₁-C₆)alkyl;

### X is O;

R₉ is selected from (C₁-C₆) alkyl, O(C₁-C₆)alkyl, hydroxy or amino;

### A is

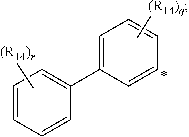

R₁₄ at each occurrence is independently selected from hydrogen, (C₁-C₆) alkyl, halogen, halo(C₁-C₆) alkyl, —O(C₁-C₆)alkyl, halo(C₁-C₆)alkoxy, —O(C₁-C₆)alkyl-S(O)ₚR₆, —O(C₃-C₈)cycloalkyl, —O(C₁-C₆)alkyl-heterocyclyl, —O-heterocyclyl, cyano, —S(O)ₚR₆, —(CH₂)ₛNR₁₅R₁₆ and —X(CH₂)ₛNR₁₅R₁₆; wherein X and R₆ are as defined above;
R₁₅ and R₁₆ are independently selected from hydrogen, (C₁-C₆)alkyl and —(CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4;
t is an integer from 1 to 4;
* indicates the point of attachment to —CH of CH(R₇)—X;
wherein,

(C₁-C₆)alkyl is unsubstituted or substituted with one or more groups independently selected from (C₁-C₆)alkyl, halogen, halo(C₁-C₆) alkyl, hydroxy, amino, cyano, nitro, —C(O)R₉ and —O(C₁-C₆)alkyl-S(O)ₚR₆; wherein R₆, R₉, and p are as defined above;

—O(C₁-C₆)alkyl is unsubstituted or substituted with one or more groups independently selected from (C₁-C₆)alkyl, (C₃-C₈)cycloalkyl, heterocyclyl, hydroxy, halogen, amino, cyano, —(C₁-C₆)alkyl-S(O)ₚR₆, —S(O)ₚR₆, —NR₁₅R₁₆ and —(CH₂)ₛNR₁₅R₁₆; wherein R₆, R₁₅, R₁₆, p and s are as defined above;

heterocyclyl is a 3- to 9-membered ring, which is unsubstituted or substituted with one or more groups independently selected from (C₁-C₆)alkyl, halogen, halo(C₁-C₆) alkyl, hydroxy, —O(C₁-C₆) alkyl, halo(C₁-C₆)alkoxy, (C₆-C₁₀)aryl, amino, —(C₁-C₆)alkyl-OH, —(C₁-C₆)alkyl-O—(C₁-C₆)alkyl and —O(C₁-C₆)alkyl-S(O)ₚR₆; wherein R₆ and p are as defined above;

halogen is selected from chlorine, bromine, iodine or fluorine;

or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

In an embodiment, the compounds of Formula (I) encompasses a compound of Formula (Ia),

wherein,
R₁ is hydrogen or (C₁-C₆) alkyl;
R₂ and R₃ together form a saturated or a partially unsaturated 3- to 9-membered heterocyclyl ring containing one or two oxygen atoms;
R₄ at each occurrence is independently selected from hydrogen, (C₁-C₆) alkyl, halogen, halo(C₁-C₆) alkyl, hydroxy, —O(C₁-C₆)alkyl, amino, cyano, —C(O)R₉ and —S(O)ₚR₆;

### R_{y} is R₅;

R₅ is selected from hydrogen, (C₁-C₆) alkyl, halogen, halo(C₁-C₆) alkyl, hydroxy, —O(C₁-C₆)alkyl, amino, cyano, —C(O)R₉ or —S(O)ₚR₆;
R₆ is selected from hydrogen, (C₁-C₆) alkyl or amino;
R₇ is hydrogen or (C₁-C₆)alkyl;

### X is O;

R₉ is selected from (C₁-C₄) alkyl, —O(C₁-C₆)alkyl, hydroxy or amino;
A is (C₆-C₁₀)aryl or heteroaryl;
n is an integer from 1 to 3;
m is an integer from 0 to 4;
p is an integer from 0 to 2;
wherein,

(C₁-C₆)alkyl is unsubstituted or substituted with one or more groups independently selected from (C₁-C₆)alkyl, halogen, halo(C₁-C₆) alkyl, hydroxy, amino, cyano, nitro, —C(O)R₉ and —O(C₁-C₆)alkyl-S(O)ₚR₆; wherein R₆, R₉, and p are as defined above;

—O(C₁-C₆)alkyl is unsubstituted or substituted with one or more groups independently selected from (C₁-C₆)alkyl, (C₃-C₈)cycloalkyl, heterocyclyl, hydroxy, halogen, amino, cyano, —(C₁-C₆)alkyl-S(O)ₚR₆, —S(O)ₚR₆, —NR₁₅R₁₆ and —(CH₂)ₛNR₁₅R₁₆; wherein R₆, R₁₅, R₁₆, p and s are as defined above;

(C₆-C₁₀)aryl is unsubstituted or substituted with one or more groups independently selected from (C₁-C₆)alkyl, halogen, halo(C₁-C₆) alkyl, hydroxy, —O(C₁-C₆) alkyl, halo(C₁-C₆)alkoxy, heteroaryl, amino, cyano, nitro, —C(O)R₉ and —O(C₁-C₆)alkyl-S(O)ₚR₆; wherein R₆, R₉, and p are as defined above;

heterocyclyl is a 3- to 9-membered ring, which is unsubstituted or substituted with one or more groups independently selected from (C₁-C₆)alkyl, halogen, halo(C₁-C₆) alkyl, hydroxy, —O(C₁-C₆) alkyl, halo(C₁-C₆)alkoxy, (C₆-C₁₀)aryl, amino, —(C₁-C₆)alkyl-OH, (C₁-C₆)alkyl-O—(C₁-C₆)alkyl and —O(C₁-C₆)alkyl-S(O)ₚR₆; wherein R₆ and p are as defined above;

heteroaryl is a 3- to 10-membered ring, which is unsubstituted or substituted with one or more groups independently selected from (C₁-C₆)alkyl, halogen, halo(C₁-C₆) alkyl, hydroxy, —O(C₁-C₆) alkyl, halo(C₁-C₆)alkoxy, amino, cyano, nitro, (C₆-C₁₀)aryl, heterocyclyl, —C(O)R₉ and —O(C₁-C₆)alkyl-S(O)ₚR₆; wherein R₆, R₉, and p are as defined above;

halogen is selected from chlorine, bromine, iodine or fluorine;

or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

In an embodiment, the compounds of Formula (I) encompasses a compound of Formula (Ia),

wherein,
R₁ is hydrogen or (C₁-C₆) alkyl;
R₂ and R₃ together form a saturated or a partially unsaturated 3- to 9-membered heterocyclyl ring containing one or two oxygen atoms;
R₄ at each occurrence is independently selected from hydrogen, (C₁-C₆) alkyl, halogen, halo(C₁-C₆) alkyl, hydroxy, —O(C₁-C₆)alkyl, amino, cyano, —C(O)R₉ or —S(O)ₚR₆;

### R_{y} is R₅;

R₅ is selected from hydrogen, (C₁-C₆) alkyl, halogen, halo(C₁-C₆) alkyl, hydroxy, —O(C₁-C₆)alkyl, amino, cyano, —C(O)R₉ or —S(O)ₚR₆;
R₆ is selected from hydrogen, (C₁-C₆) alkyl or amino;
R₇ is hydrogen or (C₁-C₆)alkyl;

### X is NR₈;

R₈ is hydrogen or (C₁-C₆)alkyl;
R₉ is selected from (C₁-C₄) alkyl, —O(C₁-C₆)alkyl, hydroxy or amino;
A is selected from

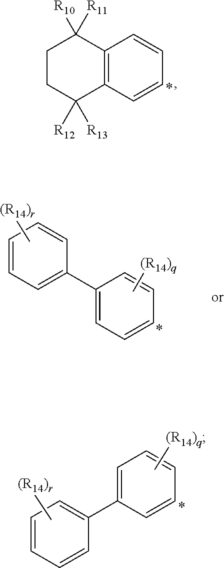

R₁₀, R₁₁, R₁₂ and R₁₃ are independently selected from hydrogen or (C₁-C₆) alkyl; or R₁₀ and R₁₁ can together form a (C₃-C₈) cycloalkyl ring and R₁₂ and R₁₃ are hydrogen; or, R₁₂ and R₁₃ can together form a (C₃-C₈) cycloalkyl ring and R₁₂ and R₁₃ are hydrogen;
R₁₄ at each occurrence is independently selected from hydrogen, (C₁-C₆) alkyl, halogen, halo(C₁-C₆) alkyl, hydroxy, —O(C₁-C₆)alkyl, halo(C₁-C₆)alkoxy, —O(C₁-C₆)alkyl-S(O)ₚR₆, —O(C₃-C₈)cycloalkyl, —O(C₁-C₆)alkylheterocyclyl, —O-heterocyclyl, (C₆-C₁₀)aryl, amino, cyano, nitro, —C(O)R₉, —S(O)ₚR₆, —(CH₂)ₛNR₁₅R₁₆ and —X(CH₂)ₛNR₁₅R₁₆; wherein X, R₆ and R₉ are as defined above;
R₁₅ and R₁₆ are independently selected from hydrogen, (C₁-C₆)alkyl and —(CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4;
t is an integer from 1 to 4;
* indicates the point of attachment to —CH of CH(R₇)—X;
wherein,

(C₁-C₆)alkyl is unsubstituted or substituted with one or more groups independently selected from (C₁-C₆)alkyl, halogen, halo(C₁-C₆) alkyl, hydroxy, O(C₁-C₆)alkyl, (C₃-C₈)cycloalkyl, (C₆-C₁₀)aryl, heterocyclyl, heteroaryl, amino, cyano, nitro, C(O)R₉ and O(C₁-C₆)alkyl-S(O)ₚR₆; wherein R₆, R₉, and p are as defined above;

—O(C₁-C₆)alkyl is unsubstituted or substituted with one or more groups independently selected from (C₁-C₆)alkyl, (C₃-C₈)cycloalkyl, heterocyclyl, hydroxy, halogen, amino, cyano, —(C₁-C₆)alkyl-S(O)ₚR₆, —S(O)ₚR₆, —NR₁₅R₁₆ and —(CH₂)ₛNR₁₅R₁₆; wherein R₆, R₁₅, R₁₆, p and s are as defined above;

(C₆-C₁₀)aryl is unsubstituted or substituted with one or more groups independently selected from (C₁-C₆)alkyl, halogen, halo(C₁-C₆) alkyl, hydroxy, —O(C₁-C₆) alkyl, halo(C₁-C₆)alkoxy, (C₆-C₁₀)aryl, heteroaryl, amino, cyano, nitro, —C(O)R₉ and —O(C₁-C₆)alkyl-S(O)ₚR₆; wherein R₆, R₉, and p are as defined above;

heterocyclyl is a 3- to 9-membered ring, which is unsubstituted or substituted with one or more groups independently selected from (C₁-C₆)alkyl, halogen, halo(C₁-C₆)alkyl, hydroxy, —O(C₁-C₆) alkyl, halo(C₁-C₆)alkoxy, (C₆-C₁₀)aryl, amino, cyano, nitro, —(C₁-C₆)alkyl-OH, (C₁-C₆)alkyl-O—(C₁-C₆)alkyl and —O(C₁-C₆)alkyl-S(O)ₚR₆; wherein R₆ and p are as defined above;

heteroaryl is a 3- to 10-membered ring, which is unsubstituted or substituted with one or more groups independently selected from (C₁-C₆)alkyl, halogen, halo(C₁-C₆) alkyl, hydroxy, —O(C₁-C₆) alkyl, halo(C₁-C₆)alkoxy, (C₆-C₁₀)aryl, heteroaryl, amino, cyano, nitro, —C(O)R₉ and —O(C₁-C₆)alkyl-S(O)ₚR₆; wherein R₆, R₉, and p are as defined above;

halogen is selected from chlorine, bromine, iodine or fluorine;

or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

In an embodiment, the compounds of Formula (I) encompasses compounds of Formula (Ia),

wherein,
R₁ is hydrogen or (C₁-C₆) alkyl;
R₂ and R₃ together form a saturated or a partially unsaturated 3- to 9-membered heterocyclyl ring containing one or two oxygen atoms;
R₄ at each occurrence is independently selected from hydrogen, (C₁-C₆) alkyl, halogen, halo(C₁-C₆) alkyl, hydroxy, —O(C₁-C₆)alkyl, amino, cyano, —C(O)R₉ and —S(O)ₚR₆;

### R_{y} is R₅;

R₅ is selected from hydrogen, (C₁-C₆) alkyl, halogen, halo(C₁-C₆) alkyl, hydroxy, —O(C₁-C₆)alkyl, amino, cyano, —C(O)R₉ or —S(O)ₚR₆;
R₆ is selected from hydrogen, (C₁-C₆) alkyl or amino;
R₇ is hydrogen or (C₁-C₆)alkyl;

### X is NR₈;

R₈ is hydrogen or (C₁-C₆) alkyl;
R₉ is selected from (C₁-C₆) alkyl, —O(C₁-C₆)alkyl, hydroxy or amino;

### A is

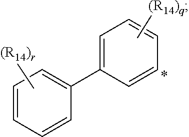

R₁₄ at each occurrence is independently selected from hydrogen, (C₁-C₆) alkyl, halogen, halo(C₁-C₆) alkyl, —O(C₁-C₆)alkyl, halo(C₁-C₆)alkoxy, —O(C₁-C₆)alkyl-S(O)ₚR₆, —O(C₃-C₈)cycloalkyl, —O(C₁-C₆)alkyl-heterocyclyl, —O-heterocyclyl, cyano, —S(O)ₚR₆, —(CH₂)ₛNR₁₅R₁₆ and —X(CH₂)ₛNR₁₅R₁₆; wherein X and R₆ are as defined above;
R₁₅ and R₁₆ are independently selected from hydrogen, (C₁-C₆)alkyl and —(CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4;
t is an integer from 1 to 4;
* indicates the point of attachment to —CH of CH(R₇)—X;
wherein,

(C₁-C₆)alkyl is unsubstituted or substituted with one or more groups independently selected from (C₁-C₆)alkyl, halogen, halo(C₁-C₆) alkyl, hydroxy, amino, cyano, nitro, —C(O)R₉ and —O(C₁-C₆)alkyl-S(O)ₚR₆; wherein R₆, R₉, and p are as defined above;

—O(C₁-C₆)alkyl is unsubstituted or substituted with one or more groups independently selected from (C₁-C₆)alkyl, (C₃-C₈)cycloalkyl, heterocyclyl, hydroxy, halogen, amino, cyano, —(C₁-C₆)alkyl-S(O)ₚR₆, —S(O)ₚR₆, —NR₁₅R₁₆ and —(CH₂)ₛNR₁₅R₁₆; wherein R₆, R₁₅, R₁₆, p and s are as defined above;

heterocyclyl is a 3- to 9-membered ring, which is unsubstituted or substituted with one or more groups independently selected from (C₁-C₆)alkyl, halogen, halo(C₁-C₆) alkyl, hydroxy, —O(C₁-C₆)alkyl, halo(C₁-C₆)alkoxy, (C₆-C₁₀)aryl, amino, —(C₁-C₆)alkyl-OH, (C₁-C₆)alkyl-O—(C₁-C₆)alkyl and —O(C₁-C₆)alkyl-S(O)ₚR₆; wherein R₆ and p are as defined above;

halogen is selected from chlorine, bromine, iodine or fluorine;

or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

In an embodiment, the compounds of Formula (I) encompasses a compound of Formula (Ia),

wherein,
R₁ is hydrogen or (C₁-C₆) alkyl;
R₂ and R₃ together form a saturated or a partially unsaturated 3- to 9-membered heterocyclyl ring containing one or two heteroatoms selected independently from N or S atoms, when the heteroatom is N, it is substituted with hydrogen, (C₁-C₆)alkyl, —C(O) (C₁-C₆)alkyl or —S(O)₂(C₁-C₆)alkyl;
R₄ at each occurrence is independently selected from hydrogen, (C₁-C₆) alkyl, halogen, halo(C₁-C₆) alkyl, hydroxy, —O(C₁-C₆)alkyl, amino, cyano, —C(O)R₉ or —S(O)ₚR₆;

### R_{y} is R₅;

R₅ is selected from hydrogen, (C₁-C₆) alkyl, halogen, halo(C₁-C₆) alkyl, hydroxy, —O(C₁-C₆)alkyl, amino, cyano, —C(O)R₉ or —S(O)ₚR₆;
R₆ is selected from hydrogen, (C₁-C₄) alkyl or amino;
R₇ is hydrogen or (C₁-C₆)alkyl;

### X is O;

R₉ is selected from (C₁-C₄) alkyl, —O(C₁-C₆)alkyl, hydroxy or amino;
A is selected from (C₆-C₁₀)aryl, heteroaryl,

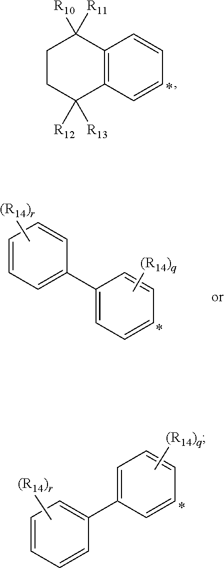

R₁₀, R₁₁, R₁₂ and R₁₃ are independently selected from hydrogen and (C₁-C₆) alkyl; or R₁₀ and R₁₁ can together form a (C₃-C₈) cycloalkyl ring and R₁₂ and R₁₃ are hydrogen; or R₁₂ and R₁₃ can together form a (C₃-C₈) cycloalkyl ring and R₁₂ and R₁₃ are hydrogen;
R₁₄ at each occurrence is independently selected from hydrogen, (C₁-C₆) alkyl, halogen, halo(C₁-C₆) alkyl, hydroxy, —O(C₁-C₆)alkyl, halo(C₁-C₆)alkoxy, —O(C₁-C₆)alkyl-S(O)ₚR₆, —O(C₃-C₈)cycloalkyl, —O(C₁-C₆)alkyl-heterocyclyl, —O-heterocyclyl, (C₆-C₁₀)aryl, amino, cyano, nitro, —C(O)R₉, —S(O)ₚR₆, —(CH₂)ₛNR₁₅R₁₆ and —X(CH₂)ₛNR₁₅R₁₆; wherein X, R₆ and R₉ are as defined above;
R₁₅ and R₁₆ are independently selected from hydrogen, (C₁-C₆)alkyl and —(CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4;
is an integer from 1 to 4;
* indicates the point of attachment to —CH of CH(R₇)—X;
wherein,

(C₁-C₆)alkyl is unsubstituted or substituted with one or more groups independently selected from (C₁-C₆)alkyl, halogen, halo(C₁-C₆) alkyl, hydroxy, —O(C₁-C₆)alkyl, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

—O(C₁-C₆)alkyl is unsubstituted or substituted with one or more groups independently selected from (C₁-C₆)alkyl, (C₃-C₈)cycloalkyl, heterocyclyl, hydroxy, halogen, amino, cyano, —(C₁-C₆)alkyl-S(O)ₚR₆, —S(O)ₚR₆, —NR₁₅R₁₆ and —(CH₂)ₛNR₁₅R₁₆; wherein R₆, R₁₅, R₁₆, p and s are as defined above;

(C₆-C₁₀)aryl is unsubstituted or substituted with one or more groups independently selected from (C₁-C₆)alkyl, halogen, halo(C₁-C₆)alkyl, hydroxy, —O(C₁-C₆) alkyl, halo(C₁-C₆)alkoxy, (C₆-C₁₀)aryl, heteroaryl, amino, cyano, nitro, —C(O)R₉ and —O(C₁-C₆)alkyl-S(O)ₚR₆; wherein R₆, R₉, and p are as defined above;

heterocyclyl is a 3- to 9-membered ring, which is unsubstituted or substituted with one or more groups independently selected from (C₁-C₆)alkyl, halogen, halo(C₁-C₆) alkyl, hydroxy, —O(C₁-C₆) alkyl, halo(C₁-C₆)alkoxy, (C₆-C₁₀)aryl, amino, cyano, nitro, —C(O)R₉, —(C₁-C₆)alkyl-OH, (C₁-C₆)alkyl-O—(C₁-C₆)alkyl and —O(C₁-C₆)alkyl-S(O)ₚR₆; wherein R₆, R₉ and p are as defined above;

heteroaryl is a 3- to 10-membered ring, which is unsubstituted or substituted with one or more groups independently selected from (C₁-C₆)alkyl, halogen, halo(C₁-C₆) alkyl, hydroxy, —O(C₁-C₆) alkyl, halo(C₁-C₆)alkoxy, (C₆-C₁₀)aryl, heteroaryl, amino, cyano, nitro, —C(O)R₉ and —O(C₁-C₆)alkyl-S(O)ₚR₆; wherein R₆, R₉, and p are as defined above;

halogen is selected from chlorine, bromine, iodine or fluorine;

or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

In an embodiment, the compounds of Formula (I) encompasses a compound of Formula (Ia),

wherein,
R₁ is hydrogen or (C₁-C₆) alkyl;
R₂ and R₃ together form a saturated or a partially unsaturated (C₄-C₈)cycloalkyl ring;
R₄ at each occurrence is independently selected from hydrogen, (C₁-C₆)alkyl, halogen, halo(C₁-C₆)alkyl, hydroxy, —O(C₁-C₆)alkyl, amino, cyano, —C(O)R₉ and —S(O)ₚR₆;

### R_{y} is R₅;

R₅ is selected from hydrogen, (C₁-C₆) alkyl, halogen, halo(C₁-C₆)alkyl, hydroxy, —O(C₁-C₆)alkyl, amino, cyano, —C(O)R₉ or —S(O)ₚR₆;
R₆ is selected from hydrogen, (C₁-C₆) alkyl or amino;
R₇ is hydrogen or (C₁-C₆)alkyl;
X is selected from O, NR₈ or S;
R₈ is selected from hydrogen, (C₁-C₆) alkyl, —C(O)(C₁-C₆)alkyl, —C(O)O(C₁-C₆)alkyl, —C(O)NH₂ or —S(O)ₚR₆; wherein R₆ is as defined above;
R₉ is selected from (C₁-C₄) alkyl, —O(C₁-C₆)alkyl, hydroxy or amino;
A is selected from (C₆-C₁₀)aryl, heteroaryl,

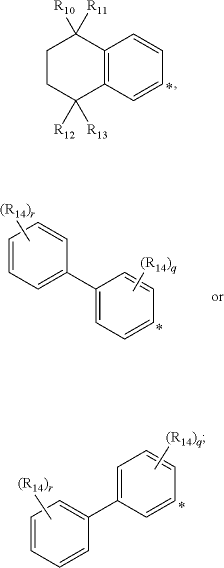

R₁₀, R₁₁, R₁₂ and R₁₃ are independently selected from hydrogen and (C₁-C₆) alkyl; or R₁₀ and R₁₁ can together form a (C₃-C₈) cycloalkyl ring and R₁₂ and R₁₃ are hydrogen; or R₁₂ and R₁₃ can together form a (C₃-C₈) cycloalkyl ring and R₁₂ and R₁₃ are hydrogen;
R₁₄ at each occurrence is independently selected from hydrogen, (C₁-C₆) alkyl, halogen, halo(C₁-C₆) alkyl, hydroxy, —O(C₁-C₆)alkyl, halo(C₁-C₆)alkoxy, —O(C₁-C₆)alkyl-S(O)ₚR₆, —O(C₃-C₈)cycloalkyl, —O(C₁-C₆)alkyl-heterocyclyl, —O-heterocyclyl, (C₆-C₁₀)aryl, amino, cyano, nitro, —C(O)R₉, —S(O)ₚR₆, —(CH₂)ₛNR₁₅R₁₆ and —X(CH₂)ₛNR₁₅R₁₆; wherein X, R₆ and R₉ are as defined above;
R₁₅ and R₁₆ are independently selected from hydrogen, (C₁-C₆)alkyl and —(CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4;
t is an integer from 1 to 4;
* indicates the point of attachment to —CH of CH(R₇)—X;
wherein,

(C₁-C₆)alkyl is unsubstituted or substituted with one or more groups independently selected from (C₁-C₆)alkyl, halogen, halo(C₁-C₆) alkyl, hydroxy, —O(C₁-C₆)alkyl, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

—O(C₁-C₆)alkyl is unsubstituted or substituted with one or more groups independently selected from (C₁-C₆)alkyl, hydroxy, halogen, amino, cyano, —(C₁-C₆)alkyl-S(O)ₚR₆, —S(O)ₚR₆, —NR₁₅R₁₆ and —(CH₂)ₛNR₁₅R₁₆; wherein R₆, R₁₅, R₁₆, p and s are as defined above;

(C₆-C₁₀)aryl is unsubstituted or substituted with one or more groups independently selected from (C₁-C₆)alkyl, halogen, halo(C₁-C₆) alkyl, hydroxy, —O(C₁-C₆) alkyl, halo(C₁-C₆)alkoxy, (C₆-C₁₀)aryl, heteroaryl, amino, cyano, nitro, —C(O)R₉ and —O(C₁-C₆)alkyl-S(O)ₚR₆; wherein R₆, R₉, and p are as defined above;

heterocyclyl is a 3- to 9-membered ring, which is unsubstituted or substituted with one or more groups independently selected from (C₁-C₆)alkyl, halogen, halo(C₁-C₆) alkyl, hydroxy, —O(C₁-C₆) alkyl, halo(C₁-C₆)alkoxy, (C₆-C₁₀)aryl, amino, cyano, nitro, —C(O)R₉, —(C₁-C₆)alkyl-OH, (C₁-C₆)alkyl-O—(C₁-C₆)alkyl and —O(C₁-C₆)alkyl-S(O)ₚR₆; wherein R₆, R₉ and p are as defined above;

heteroaryl is a 3- to 10-membered ring, which is unsubstituted or substituted with one or more groups independently selected from (C₁-C₆)alkyl, halogen, halo(C₁-C₆) alkyl, hydroxy, —O(C₁-C₆) alkyl, halo(C₁-C₆)alkoxy, (C₆-C₁₀)aryl, heteroaryl, amino, cyano, nitro, —C(O)R₉ and —O(C₁-C₆)alkyl-S(O)ₚR₆; wherein R₆, R₉, and p are as defined above;

halogen is selected from chlorine, bromine, iodine or fluorine;

or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

In an embodiment, the compounds of Formula (I) encompasses a compound of Formula (Ib),

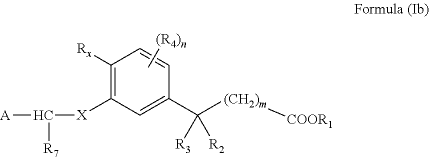

wherein,
R₁ is hydrogen or (C₁-C₆) alkyl;
R₂ and R₃ together form a saturated or a partially unsaturated 3- to 9-membered heterocyclyl ring containing one or two heteroatoms selected from O, N or S; or R₂ and R₃ together form a saturated or a partially unsaturated (C₄-C₈) cycloalkyl ring;
R₄ at each occurrence is independently selected from hydrogen, (C₁-C₆)alkyl, halogen, halo(C₁-C₆) alkyl, hydroxy, —O(C₁-C₆)alkyl, (C₆-C₁₀)aryl, amino, cyano, nitro, —C(O)R₉ and —S(O)ₚR₆;

### Rₓ is A-CH(R₇)—X or R₅;

R₅ is selected from hydrogen, (C₁-C₆) alkyl, halogen, halo(C₁-C₆) alkyl, hydroxy, —O(C₁-C₆)alkyl, (C₆-C₁₀)aryl, amino, cyano, nitro, —C(O)R₉ or —S(O)ₚR₆;
R₆ is selected from hydrogen, (C₁-C₆) alkyl or amino;
R₇ is hydrogen or (C₁-C₆)alkyl;
X is selected from O, NR₈ or S;
R₈ is selected from hydrogen, (C₁-C₆) alkyl, (C₃-C₈)cycloalkyl, (C₆-C₁₀)aryl, heterocyclyl, heteroaryl, cyano, —C(O)(C₁-C₆)alkyl, —C(O)O(C₁-C₆)alkyl, —C(O)NH₂ or —S(O)ₚR₆; wherein R₆ is as defined above;
R₉ is selected from (C₁-C₆) alkyl, —O(C₁-C₆)alkyl, hydroxy or amino;
A is selected from (C₃-C₈)cycloalkyl, (C₆-C₁₀)aryl, heterocyclyl, heteroaryl,

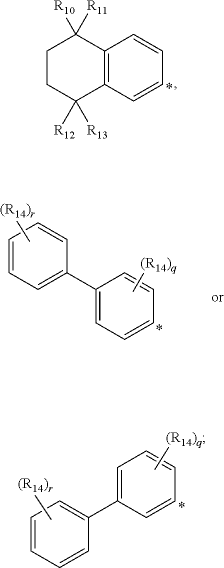

R₁₀, R₁₁, R₁₂ and R₁₃ are independently selected from hydrogen or (C₁-C₆) alkyl; or R₁₀ and R₁₁ can together form a (C₃-C₈)cycloalkyl ring and R₁₂ and R₁₃ are hydrogen; or R₁₂ and R₁₃ can together form a (C₃-C₈) cycloalkyl ring and R₁₀ and R₁₁ are hydrogen;
R₁₄ at each occurrence is independently selected from hydrogen, (C₁-C₆) alkyl, halogen, halo(C₁-C₆) alkyl, hydroxy, —O(C₁-C₆)alkyl, halo(C₁-C₆)alkoxy, —O(C₁-C₆)alkyl-S(O)ₚR₆, —O(C₃-C₈)cycloalkyl, —O(C₁-C₆)alkyl-heterocyclyl, —O-heterocyclyl, (C₆-C₁₀)aryl, amino, cyano, nitro, —C(O)R₉, —S(O)ₚR₆, —(CH₂)ₛNR₁₅R₁₆ and —X(CH₂)ₛNR₁₅R₁₆; wherein X, R₆ and R₉ are as defined above;
R₁₅ and R₁₆ are independently selected from hydrogen, (C₁-C₆)alkyl and —(CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4;
t is an integer from 1 to 4;
* indicates the point of attachment to —CH of CH(R₇)—X;
wherein,

(C₁-C₆)alkyl is unsubstituted or substituted with one or more groups independently selected from (C₁-C₆)alkyl, (C₂-C₈)alkenyl, (C₂-C₈)alkynyl, halogen, halo(C₁-C₆) alkyl, hydroxy, —O(C₁-C₆)alkyl, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

—O(C₁-C₆)alkyl is unsubstituted or substituted with one or more groups independently selected from (C₁-C₆)alkyl, (C₃-C₈)cycloalkyl, heterocyclyl, hydroxy, halogen, amino, cyano, —(C₁-C₆)alkyl-S(O)ₚR₆, —S(O)ₚR₆, —NR₁₅R₁₆ and —(CH₂)ₛNR₁₅R₁₆; wherein R₆, R₁₅, R₁₆, p and s are as defined above;

(C₆-C₁₀)aryl is unsubstituted or substituted with one or more groups independently selected from (C₁-C₆)alkyl, (C₂-C₈) alkenyl, (C₂-C₈) alkynyl, halogen, halo(C₁-C₆) alkyl, hydroxy, —O(C₁-C₆) alkyl, halo(C₁-C₆)alkoxy, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

heterocyclyl is a 3- to 9-membered ring, which is unsubstituted or substituted with one or more groups independently selected from (C₁-C₆)alkyl, (C₂-C₈) alkenyl, (C₂-C₈) alkynyl, halogen, halo(C₁-C₆) alkyl, hydroxy, —O(C₁-C₆) alkyl, halo(C₁-C₆)alkoxy, (C₃-C₈)cycloalkyl, (C₆-C₁₀)aryl, heterocyclyl, heteroaryl, amino, cyano, nitro, —C(O)R₉, —(C₁-C₆)alkyl-OH, (C₁-C₆)alkyl-O—(C₁-C₆)alkyl and —O(C₁-C₆)alkyl-S(O)ₚR₆; wherein R₆, R₉, and p are as defined above;

heteroaryl is a 3- to 10-membered ring, which is unsubstituted or substituted with one or more groups independently selected from (C₁-C₆)alkyl, (C₂-C₈) alkenyl, (C₂-C₈) alkynyl, halogen, halo(C₁-C₆) alkyl, hydroxy, —O(C₁-C₆) alkyl, halo(C₁-C₆)alkoxy, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

halogen is selected from chlorine, bromine, iodine or fluorine;

or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

In one embodiment, the present invention encompasses a compound of Formula (I),

R₁ is hydrogen or (C₁-C₆)alkyl;
R₂ and R₃ together form an oxetane ring;
R₄ at each occurrence is independently selected from hydrogen, (C₁-C₆) alkyl, halogen, halo(C₁-C₆) alkyl, hydroxy, —O(C₁-C₆)alkyl, amino, cyano, nitro, —C(O)R₉ and —S(O)ₚR₆;

### Rₓ is A-CH(R₇)—X;

### R_{y} is R₅;

R₅ is selected from hydrogen, (C₁-C₆) alkyl, halogen, halo(C₁-C₆) alkyl, hydroxy, —O(C₁-C₆)alkyl, amino, cyano, nitro, —C(O)R₉ or —S(O)ₚR₆;

### X is O;

A is selected from (C₆-C₁₀)aryl, heteroaryl,

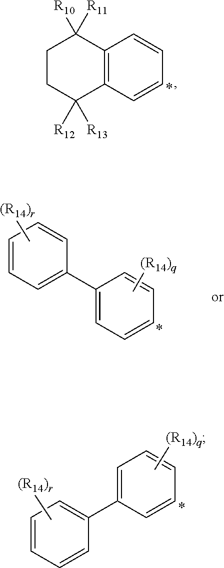

m is 1;
R₆, R₇, R₉, R₁₀, R₁₁, R₁₂, R₁₃, R₁₄, n, p, q and r are as defined above;
wherein,

(C₁-C₆)alkyl is unsubstituted or substituted with one or more groups independently selected from (C₁-C₆)alkyl, (C₂-C₈) alkenyl, (C₂-C₈) alkynyl, halogen, halo(C₁-C₆) alkyl, hydroxy, —O(C₁-C₆)alkyl, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

—O(C₁-C₆)alkyl is unsubstituted or substituted with one or more groups independently selected from (C₁-C₆)alkyl, (C₃-C₈)cycloalkyl, heterocyclyl, hydroxy, halogen, amino, cyano, —(C₁-C₆)alkyl-S(O)ₚR₆, —S(O)ₚR₆, —NR₁₅R₁₆ and —(CH₂)ₛNR₁₅R₁₆; wherein R₆, R₁₅, R₁₆, p and s are as defined above;

(C₆-C₁₀)aryl is unsubstituted or substituted with one or more groups independently selected from (C₁-C₆)alkyl, (C₂-C₈) alkenyl, (C₂-C₈) alkynyl, halogen, halo(C₁-C₆) alkyl, hydroxy, O(C₁-C₆) alkyl, halo(C₁-C₆)alkoxy, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

heterocyclyl is a 3- to 9-membered ring, which is unsubstituted or substituted with one or more groups independently selected from (C₁-C₆)alkyl, halogen, halo(C₁-C₆) alkyl, hydroxy, —O(C₁-C₆) alkyl, halo(C₁-C₆)alkoxy, (C₆-C₁₀)aryl, amino, cyano, nitro, —(C₁-C₆)alkyl-OH, (C₁-C₆)alkyl-O—(C₁-C₆)alkyl and —O(C₁-C₆)alkyl-S(O)ₚR₆; wherein R₆, and p are as defined above;

heteroaryl is a 3- to 10-membered ring, which is unsubstituted or substituted with one or more groups selected from (C₁-C₆)alkyl, (C₂-C₈) alkenyl, (C₂-C₈) alkynyl, halogen, halo(C₁-C₆) alkyl, hydroxy, —O(C₁-C₆) alkyl, halo(C₁-C₆)alkoxy, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

halogen is selected from chlorine, bromine, iodine or fluorine;

or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

In another embodiment, the present invention encompasses a compound of Formula (I),

wherein
R₁ is hydrogen or (C₁-C₆)alkyl;
R₂ and R₃ together form an oxetane ring;
R₄ at each occurrence is independently selected from hydrogen, (C₁-C₆) alkyl, halogen, halo(C₁-C₆) alkyl, hydroxy, —O(C₁-C₆)alkyl, amino, cyano, nitro, —C(O)R₉ and —S(O)ₚR₆;

### Rₓ is A-CH(R₇)—X;

### R_{y} is R₅;

R₅ is selected from hydrogen, (C₁-C₆) alkyl, halogen, halo(C₁-C₆) alkyl, hydroxy, —O(C₁-C₆)alkyl, amino, cyano, nitro, —C(O)R₉ or —S(O)ₚR₆;

### X is O;

A is selected from

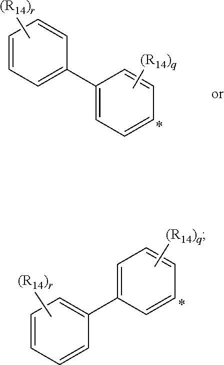

m is 1;
R₆, R₇, R₉, R₁₄, n, p, q and r are as defined above in Formula (I);
wherein

(C₁-C₆)alkyl is unsubstituted or substituted with one or more groups independently selected from (C₁-C₆)alkyl, (C₂-C₈) alkenyl, (C₂-C₈) alkynyl, halogen, halo(C1-C6) alkyl, hydroxy, —O(C₁-C₆)alkyl, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

—O(C₁-C₆)alkyl is unsubstituted or substituted with one or more groups independently selected from (C₁-C₆)alkyl, (C₃-C₈)cycloalkyl, heterocyclyl, hydroxy, halogen, amino, cyano, —(C₁-C₆)alkyl-S(O)ₚR₆, —S(O)ₚR₆, —NR₁₅R₁₆ and —(CH₂)ₛNR₁₅R₁₆; wherein R₆, R₁₅, R₁₆, p and s are as defined above;

(C₆-C₁₀)aryl is unsubstituted or substituted with one or more groups independently selected from (C₁-C₆)alkyl, (C₂-C₈) alkenyl, (C₂-C₈) alkynyl, halogen, halo(C₁-C₆) alkyl, hydroxy, —O(C₁-C₆) alkyl, halo(C₁-C₆)alkoxy, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

heterocyclyl is a 3- to 9-membered ring, which is unsubstituted or substituted with one or more groups independently selected from (C₁-C₆)alkyl, halogen, halo(C₁-C₆) alkyl, hydroxy, —O(C₁-C₆) alkyl, halo(C₁-C₆)alkoxy, (C₆-C₁₀)aryl, amino, cyano, nitro, —(C₁-C₆)alkyl-OH, (C₁-C₆)alkyl-O—(C₁-C₆)alkyl and —O(C₁-C₆)alkyl-S(O)ₚR₆; wherein R₆, and p are as defined above;

heteroaryl is a 3- to 9-membered ring, which is unsubstituted or substituted with one or more groups independently selected from (C₁-C₆)alkyl, (C₂-C₈) alkenyl, (C₂-C₈) alkynyl, halogen, halo(C₁-C₆) alkyl, hydroxy, —O(C₁-C₆) alkyl, halo(C₁-C₆)alkoxy, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

halogen is selected from chlorine, bromine, iodine or fluorine;

or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

Representative compounds of Formula (I) encompassed in accordance with the present invention include:
Ethyl 2-(3-(4-((4′-(trifluoromethyl)biphenyl-3-yl)methoxy)phenyl)oxetan-3-yl)acetate;
2-(3-(4-((4′-(Trifluoromethyl)biphenyl-3-yl)methoxy)phenyl)oxetan-3-yl)acetic acid;
Ethyl 2-(3-(4-([1,1′-biphenyl]-3-ylmethoxy)phenyl)oxetan-3-yl)acetate;
2-(3-(4-([1,1′-Biphenyl]-3-ylmethoxy)phenyl)oxetan-3-yl)acetic acid;
Ethyl 2-(3-(4-((2′-cyano-[1,1′-biphenyl]-4-yl)meth oxy)phenyl)oxetan-3-yl)acetate;
2-(3-(4-((2′-Cyano-[1,1′-biphenyl]-4-yl)methoxy)phenyl)oxetan-3-yl)acetic acid;
Ethyl 2-(3-(4-([1,1′-biphenyl]-4-ylmethoxy)phenyl)oxetan-3-yl)acetate;
2-(3-(4-([1,1′-Biphenyl]-4-ylmethoxy)phenyl)oxetan-3-yl)acetic acid;
Ethyl 2-(3-(4-((2′,6′-dimethyl-4′-(3-(methylsulfonyl)propoxy)-[1,1′-biphenyl]-3-yl)methoxy)phenyl)oxetan-3-yl)acetate;
2-(3-(4-((2′,6′-Dimethyl-4′-(3-(methylsulfonyl)propoxy)-[1,1′-biphenyl]-3-yl)methoxy)phenyl)oxetan-3-yl)acetic acid;
Ethyl 2-(3-(4-([1,1′-biphenyl]-3-ylmethoxy)-3-fluorophenyl)oxetan-3-yl)acetate;
2-(3-(4-([1,1′-Biphenyl]-3-ylmethoxy)-3-fluorophenyl)oxetan-3-yl)acetic acid;
Ethyl 2-(3-(4-([1,1′-biphenyl]-4-ylmethoxy)-3-fluorophenyl)oxetan-3-yl)acetate;
2-(3-(4-([1,1′-Biphenyl]-4-ylmethoxy)-3-fluorophenyl)oxetan-3-yl)acetic acid;
Ethyl 2-(3-(4-((2′,6′-dimethyl-4′-(3-(methylsulfonyl)propoxy)-[1,1′-biphenyl]-3-yl)methoxy)-3-fluorophenyl)oxetan-3-yl)acetate;
2-(3-(4-((2′,6′-Dimethyl-4′-(3-(methylsulfonyl)propoxy)-[1,1′-biphenyl]-3-yl)methoxy)-3-fluorophenyl)oxetan-3-yl)acetic acid;
Ethyl 2-(3-(4-((5,5,8,8-tetramethyl-5,6,7,8-tetrahydronaphthalen-2-yl)methoxy)phenyl)oxetan-3-yl)acetate;
2-(3-(4-((5,5,8,8-Tetramethyl-5,6,7,8-tetrahydronaphthalen-2-yl)methoxy)phenyl)oxetan-3-yl)acetic acid;
Ethyl 2-(3-(3-fluoro-4-((5,5,8,8-tetramethyl-5,6,7,8-tetrahydronaphthalen-2-yl)methoxy)phenyl)oxetan-3-yl)acetate;
2-(3-(3-Fluoro-4-((5,5,8,8-tetramethyl-5,6,7,8-tetrahydronaphthalen-2-yl)methoxy)phenyl)oxetan-3-yl)acetic acid;
Ethyl 2-(3-(4-((4-methoxy-3-(trifluoromethyl)benzyl)oxy)phenyl)oxetan-3-yl)acetate;
2-(3-(4-(4-Methoxy-3-(trifluoromethyl)benzyl)oxy)phenyl)oxetan-3-yl)acetic acid;
Ethyl 2-(3-(4-((2-methyl-5-(trifluoromethyl)benzyl)oxy)phenyl)oxetan-3-yl)acetate;
2-(3-(4-(2-Methyl-5-(trifluoromethyl)benzyl)oxy)phenyl)oxetan-3-yl)acetic acid;
Ethyl 2-(3-(4-((2-methoxy-5-(trifluoromethyl)benzyl)oxy)phenyl)oxetan-3-yl)acetate;
2-(3-(4-(2-Methoxy-5-(trifluoromethyl)benzyl)oxy)phenyl)oxetan-3-yl)acetic acid;
Ethyl 2-(3-(4-((4-methyl-3-(trifluoromethyl)benzyl)oxy)phenyl)oxetan-3-yl)acetate;
2-(3-(4-((4-Methyl-3-(trifluoromethyl)benzyl)oxy)phenyl)oxetan-3-yl)acetic acid;
Ethyl 2-(3-(4-(3-methoxy-4-(trifluoromethyl)benzyl)oxy)phenyl)oxetan-3-yl)acetate;
2-(3-(4-((3-Methoxy-4-(trifluoromethyl)benzyl)oxy)phenyl)oxetan-3-yl)acetic acid;
Ethyl 2-(3-(4-(3-fluoro-4-(trifluoromethyl)benzyl)oxy)phenyl)oxetan-3-yl)acetate;
2-(3-(4-((3-Fluoro-4-(trifluoromethyl)benzyl)oxy)phenyl)oxetan-3-yl)acetic acid;
Ethyl 2-(3-(4-((3-fluoro-5-(trifluoromethoxy)benzyl)oxy)phenyl)oxetan-3-yl)acetate;
2-(3-(4-((3-Fluoro-5-(trifluoromethoxy)benzyl)oxy)phenyl)oxetan-3-yl)acetic acid;
Ethyl 2-(3-(4-((3-fluoro-4-(trifluoromethoxy)benzyl)oxy)phenyl)oxetan-3-yl)acetate;
2-(3-(4-((3-Fluoro-4-(trifluoromethoxy)benzyl)oxy)phenyl)oxetan-3-yl)acetic acid;
Ethyl 2-(3-(4-((2-fluoro-3-(trifluoromethyl)benzyl)oxy)phenyl)oxetan-3-yl)acetate;
2-(3-(4-((2-Fluoro-3-(trifluoromethyl)benzyl)oxy)phenyl)oxetan-3-yl)acetic acid;
Ethyl 2-(3-(4-((4′-hydroxy-2′,6′-dimethyl-[1,1′-biphenyl]-3-yl)methoxy)phenyl)oxetan-3-yl)acetate;
Ethyl 2-(3-(4-((2′,6′-dimethyl-4′-((tetrahydrofuran-3-yl)methoxy)-[1,1′-biphenyl]-3-yl)methoxy)phenyl)oxetan-3-yl)acetate;
2-(3-(4-((2′,6′-Dimethyl-4′-((tetrahydrofuran-3-yl)methoxy)-[1,1′-biphenyl]-3-yl)methoxy)phenyl)oxetan-3-yl)acetic acid;
Ethyl 2-(3-(4-((2′,6′-dimethyl-4′-((tetrahydro-2H-pyran-4-yl)methoxy)-[1,1′-biphenyl]-3-yl)methoxy)phenyl)oxetan-3-yl)acetate;
2-(3-(4-((2′,6′-Dimethyl-4′-((tetrahydro-2H-pyran-4-yl)methoxy)-[1,1′-biphenyl]-3-yl)methoxy)phenyl)oxetan-3-yl)acetic acid;
Ethyl 2-(3-(4-((4′-((1,1-dioxidotetrahydro-2H-thiopyran-4-yl)methoxy)-2′,6′-dimethyl-[1,1′-biphenyl]-3-yl)methoxy)phenyl)oxetan-3-yl)acetate;
2-(3-(4-((4-(1,1-Dioxidotetrahydro-2H-thiopyran-4-yl)meth oxy)-2′,6′-dimethyl-[1,1′-biphenyl]-3-yl)methoxy)phenyl)oxetan-3-yl)acetic acid;
Ethyl 2-(3-(4-((2′,6′-dimethyl-4′-((tetrahydrofuran-2-yl)methoxy)-[1,1′-biphenyl]-3-yl)methoxy)phenyl)oxetan-3-yl)acetate;
2-(3-(4-((2′,6′-Dimethyl-4′-((tetrahydrofuran-2-yl)methoxy)-[1,1′-biphenyl]-3-yl)methoxy)phenyl)oxetan-3-yl)acetic acid;
(R)-ethyl 2-(3-(4-((2′,6′-dimethyl-4′-((tetrahydrofuran-3-yl)methoxy)-[1,1′-biphenyl]-3-yl)methoxy)phenyl)oxetan-3-yl)acetate;
(R)-2-(3-(4-((2′,6′-dimethyl-4′-((tetrahydrofuran-3-yl)meth oxy)-[1,1′-biphenyl]-3-yl)methoxy)phenyl)oxetan-3-yl)acetic acid;
Ethyl 2-(3-(4-((2′,6′-dimethyl-4′-((3-methyloxetan-3-yl)methoxy)-[1,1′-biphenyl]-3-yl)methoxy)phenyl)oxetan-3-yl)acetate;
2-(3-(4-((2′,6′-Dimethyl-4′-((3-methyloxetan-3-yl)methoxy)-[1,1′-biphenyl]-3-yl)methoxy)phenyl)oxetan-3-yl)acetic acid;
Ethyl 2-(3-(4-((4′-((1,1-dioxidotetrahydrothiophen-3-yl)methoxy)-2′,6′-dimethyl-[1,1′-biphenyl]-3-yl)methoxy)phenyl)oxetan-3-yl)acetate;
2-(3-(4-((4′-((1,1-Dioxidotetrahydrothiophen-3-yl)methoxy)-2′,6′-dimethyl-[1,1′-biphenyl]-3-yl)methoxy)phenyl)oxetan-3-yl)acetic acid;
Ethyl 2-(3-(4-((4′-((3-(hydroxymethyl)oxetan-3-yl)methoxy)-2′,6′-dimethyl-[1,1′-biphenyl]-3-yl)methoxy)phenyl)oxetan-3-yl)acetate;
2-(3-(4-((4′-((3-(Hydroxymethyl)oxetan-3-yl)methoxy)-2′,6′-dimethyl-[1,1′-biphenyl]-3-yl)methoxy)phenyl)oxetan-3-yl)acetic acid;
Ethyl 2-(3-(4-((4′-((1,1-dioxidotetrahydro-2H-thiopyran-4-yl)oxy)-2′,6′-dimethyl-[1,1′-biphenyl]-3-yl)methoxy)phenyl)oxetan-3-yl)acetate;
2-(3-(4((4-(1,1-Dioxidotetrahydro-2H-thiopyran-4-yl)oxy)-2′,6′-dimethyl-[1,1′-biphenyl]-3-yl)methoxy)phenyl)oxetan-3-yl)acetic acid;
Ethyl 2-(3-(4-((4′-(cyclopentyloxy)-2′,6′-dimethyl-[1,1′-biphenyl]-3-yl)meth oxy)phenyl)oxetan-3-yl)acetate;
2-(3-(4-((4′-(Cyclopentyloxy)-2′,6′-dimethyl-[1,1′-biphenyl]-3-yl)methoxy)phenyl)oxetan-3-yl)acetic acid;
Ethyl 2-(3-(4-((2′-chloro-4′-hydroxy-[1,1′-biphenyl]-3-yl)methoxy)phenyl)oxetan-3-yl)acetate;
Ethyl 2-(3-(4-((2′-chloro-4′-(3-(methylsulfonyl)propoxy)-[1,1′-biphenyl]-3-yl)methoxy)phenyl)oxetan-3-yl)acetate;
2-(3-(4-((2′-Chloro-4′-(3-(methylsulfonyl)propoxy)-[1,1′-biphenyl]-3-yl)methoxy)phenyl)oxetan-3-yl)acetic acid;
Ethyl 2-(3-(4-((2′-chloro-4′-((3-methyloxetan-3-yl)methoxy)-[1,1′-biphenyl]-3-yl)methoxy)phenyl)oxetan-3-yl)acetate;
2-(3-(4-((2′-Chloro-4′-((3-methyloxetan-3-yl)methoxy)-[1,1′-biphenyl]-3-yl)methoxy)phenyl)oxetan-3-yl)acetic acid;
Ethyl 2-(3-(4-((2′-chloro-4′-((3-(hydroxymethyl)oxetan-3-yl)methoxy)-[1,1′-biphenyl]-3-yl)methoxy)phenyl)oxetan-3-yl)acetate;
2-(3-(4-((2′-Chloro-4′-((3-(hydroxymethyl)oxetan-3-yl)methoxy)-[1,1′-biphenyl]-3-yl)methoxy)phenyl)oxetan-3-yl)acetic acid;
Ethyl 2-(3-(4-((2′-chloro-4′-((1,1-dioxidotetrahydrothiophen-3-yl)methoxy)-[1,1′-biphenyl]-3-yl)methoxy)phenyl)oxetan-3-yl)acetate;
2-(3-(4-((2′-Chloro-4′-((1,1-dioxidotetrahydrothiophen-3-yl)methoxy)-[1,1′-biphenyl]-3-yl)methoxy)phenyl)oxetan-3-yl)acetic acid;
Ethyl 2-(3-(4-((2′-chloro-4′-((1,1-dioxidotetrahydro-2H-thiopyran-4-yl)methoxy)-[1,1′-biphenyl]-3-yl)methoxy)phenyl)oxetan-3-yl)acetate;
2-(3-(4-((2′-Chloro-4′-((1,1-dioxidotetrahydro-2H-thiopyran-4-yl)methoxy)-[1,1′-biphenyl]-3-yl)methoxy)phenyl)oxetan-3-yl)acetic acid;
Ethyl 2-(3-(4-((2′-chloro-4′-((tetrahydro-2H-pyran-4-yl)methoxy)-[1,1′-biphenyl]-3-yl)methoxy)phenyl)oxetan-3-yl)acetate;
2-(3-(4-((2′-Chloro-4′-((tetrahydro-2H-pyran-4-yl)methoxy)-[1,1′-biphenyl]-3-yl)methoxy)phenyl)oxetan-3-yl)acetic acid;
Ethyl 2-(3-(4-((4′-hydroxy-[1,1′-biphenyl]-3-yl) methoxy)phenyl)oxetan-3-yl)acetate;
Ethyl 2-(3-(4-((4′-(cyclobutylmethoxy)-[1,1′-biphenyl]-3-yl)methoxy)phenyl)oxetan-3-yl)acetate;
2-(3-(4-((4′-(Cyclobutylmethoxy)-[1,1′-biphenyl]-3-yl)methoxy)phenyl)oxetan-3-yl) acetic acid;
Ethyl 2-(3-(4-((2′-methyl-4′-(3-(methylsulfonyl)propoxy)-[1,1′-biphenyl]-3-yl)methoxy)phenyl)oxetan-3-yl)acetate;
2-(3-(4-((2′-Methyl-4′-(3-(methylsulfonyl)propoxy)-[1,1′-biphenyl]-3-yl)methoxy)phenyl)oxetan-3-yl)acetic acid;
Ethyl 2-(3-(4-((3′,5′-dimethyl-4′-(3-(methylsulfonyl)propoxy)-[1,1′-biphenyl]-3-yl)methoxy)phenyl)oxetan-3-yl)acetate;
2-(3-(4-((3′,5′-Dimethyl-4′-(3-(methylsulfonyl)propoxy)-[1,1′-biphenyl]-3-yl)methoxy)phenyl)oxetan-3-yl)acetic acid;
Ethyl 2-(3-(4-((3′-methoxy-4′-(3-(methylsulfonyl)propoxy)-[1,1′-biphenyl]-3-yl)methoxy)phenyl)oxetan-3-yl)acetate;
2-(3-(4-((3′-Methoxy-4′-(3-(methylsulfonyl)propoxy)-[1,1′-biphenyl]-3-yl)methoxy)phenyl)oxetan-3-yl)acetic acid;
Ethyl 2-(3-(4-((4′-(methylthio)-[1,1′-biphenyl]-3-yl)methoxy)phenyl)oxetan-3-yl)acetate;
2-(3-(4-((4′-(Methylthio)-[1,1′-biphenyl]-3-yl)methoxy)phenyl)oxetan-3-yl)acetic acid;
Ethyl 2-(3-(4-((4′-(butylthio)-[1,1′-biphenyl]-3-yl)methoxy)phenyl)oxetan-3-yl)acetate;
2-(3-(4-((4′-(Butylthio)-[1,1′-biphenyl]-3-yl)methoxy)phenyl)oxetan-3-yl)acetic acid;
Ethyl 2-(3-(4-((4′-(3-(methylsulfonyl)propoxy)-3′-(trifluoromethyl)-[1,1′-biphenyl]-3-yl)methoxy)phenyl)oxetan-3-yl)acetate;
2-(3-(4-((4′-(3-(Methylsulfonyl)propoxy)-3′-(trifluoromethyl)-[1,1′-biphenyl]-3-yl)methoxy)phenyl)oxetan-3-yl)acetic acid;
Ethyl 2-(3-(4-((4′-(isopropylthio)-[1,1′-biphenyl]-3-yl)methoxy)phenyl)oxetan-3-yl)acetate;
2-(3-(4-((4′-(Isopropylthio)-[1,1′-biphenyl]-3-yl)methoxy)phenyl)oxetan-3-yl)acetic acid;
Ethyl 2-(3-(4-((5-methyl-2-phenyloxazol-4-yl)methoxy)phenyl)oxetan-3-yl)acetate;
2-(3-(4-((5-Methyl-2-phenyloxazol-4-yl)methoxy)phenyl)oxetan-3-yl)acetic acid;
Ethyl 2-(3-(4-((2′,6′-dimethyl-4′-(3-(methylsulfonyl)propoxy)-[1,1′-biphenyl]-3-yl)methoxy)phenyl)azetidin-3-yl)acetate;
Ethyl 2-(3-(4-((2′,6′-dimethyl-4′-(3-(methylsulfonyl)propoxy)-[1,1′-biphenyl]-3-yl)methoxy)phenyl)-1-(methylsulfonyl)azetidin-3-yl)acetate;
2-(3-(4-((2′,6′-Dimethyl-4′-(3-(methylsulfonyl)propoxy)-[1,1′-biphenyl]-3-yl)methoxy)phenyl)-1-(methylsulfonyl)azetidin-3-yl)acetic acid;
Ethyl 2-(1-acetyl-3-(4-((2′,6′-dimethyl-4′-(3-(methylsulfonyl)propoxy)-[1,1′-biphenyl]-3-yl)methoxy)phenyl)azetidin-3-yl)acetate;
2-(1-Acetyl-3-(4-((2′,6′-dimethyl-4′-(3-(methylsulfonyl)propoxy)-[1,1′-biphenyl]-3-yl)methoxy)phenyl)azetidin-3-yl)acetic acid;
Ethyl 2-(3-(3-fluoro-4-((4′-(trifluoromethyl)-[1,1′-biphenyl]-3-yl)methoxy)phenyl)oxetan-3-yl)acetate:
2-(3-(3-Fluoro-4-((4′-(trifluoromethyl)-[1,1′-biphenyl]-3-yl)methoxy)phenyl)oxetan-3-yl)acetic acid;
Ethyl 2-(3-(4-((4-fluoro-3-(trifluoromethoxy)benzyl)oxy)phenyl)oxetan-3-yl)acetate;
2-(3-(4-((4-Fluoro-3-(trifluoromethoxy)benzyl)oxy)phenyl)oxetan-3-yl)acetic acid;
Ethyl 2-(3-(4-((3-fluorobenzyl)oxy)phenyl)oxetan-3-yl)acetate;
2-(3-(4-((3-Fluorobenzyl)oxy)phenyl)oxetan-3-yl)acetic acid;
Ethyl 2-(3-(4-((2-fluoro-5-(trifluoromethoxy)benzyl)oxy)phenyl)oxetan-3-yl)acetate;
2-(3-(4-((2-Fluoro-5-(trifluoromethoxy)benzyl)oxy)phenyl)oxetan-3-yl)acetic acid;
Ethyl 2-(3-(4-((3-(5-methoxypyridin-3-yl)benzyl)oxy)phenyl)oxetan-3-yl)acetate;
2-(3-(4-((3-(5-Methoxypyridin-3-yl)benzyl)oxy)phenyl)oxetan-3-yl)acetic acid;
Ethyl 2-(3-(4-((3-(2-morpholinopyrimidin-5-yl)benzyl)oxy)phenyl)oxetan-3-yl)acetate;
2-(3-(4-((3-(2-Morpholinopyrimidin-5-yl)benzyl)oxy)phenyl)oxetan-3-yl)acetic acid;
Ethyl 2-(3-(4-((3-(6-(3-(methylsulfonyl)propoxy)pyridin-3-yl)benzyl)oxy)phenyl)oxetan-3-yl)acetate;
2-(3-(4-((3-(6-(3-(Methylsulfonyl)propoxy)pyridin-3-yl)benzyl)oxy)phenyl)oxetan-3-yl)acetic acid;
Ethyl 2-(3-(4-((4′-(isopentyloxy)-2′,6′-dimethyl-[1,1′-biphenyl]-3-yl)methoxy)phenyl)oxetan-3-yl)acetate;
2-(3-(4-((4′-(Isopentyloxy)-2′,6′-dimethyl-[1,1′-biphenyl]-3-yl)methoxy)phenyl)oxetan-3-yl)acetic acid;
Ethyl 2-(3-(4-((4′-((1,3-difluoropropan-2-yl)oxy)-2′,6′-dimethyl-[1,1′-biphenyl]-3-yl)methoxy)phenyl)oxetan-3-yl)acetate;
2-(3-(4-((4′-((1,3-Difluoropropan-2-yl)oxy)-2′,6′-dimethyl-[1,1′-biphenyl]-3-yl)methoxy)phenyl)oxetan-3-yl)acetic acid;
Ethyl 2-(3-(4-((2′,6′-dimethyl-4′-(neopentyloxy)-[1,1′-biphenyl]-3-yl)methoxy)phenyl)oxetan-3-yl)acetate;
2-(3-(4-((2′,6′-Dimethyl-4′-(neopentyloxy)-[1,1′-biphenyl]-3-yl)methoxy)phenyl)oxetan-3-yl)acetic acid;
Ethyl 2-(3-(4-((4′-(2-methoxyethoxy)-2′,6′-dimethyl-[1,1′-biphenyl]-3-yl)methoxy)phenyl)oxetan-3-yl)acetate;
2-(3-(4-((4′-(2-Methoxyethoxy)-2′,6′-dimethyl-[1,1′-biphenyl]-3-yl)methoxy)phenyl)oxetan-3-yl)acetic acid;
Ethyl 2-(3-(4-((4′-((3-(methoxymethyl)oxetan-3-yl)methoxy)-2′,6′-dimethyl-[1,1′-biphenyl]-3-yl)methoxy)phenyl)oxetan-3-yl)acetate;
2-(3-(4-((4′-((3-(Methoxymethyl)oxetan-3-yl)methoxy)-2′,6′-dimethyl-[1,1′-biphenyl]-3-yl)methoxy)phenyl)oxetan-3-yl)acetic acid;
Ethyl 2-(3-(4-(((4′-((1,1-dioxidotetrahydrothiophen-3-yl)methoxy)-2′,6′-dimethyl-[1,1′-biphenyl]-3-yl)methyl)amino)phenyl)oxetan-3-yl)acetate; and
2-(3-(4-(((4′-((1,1-Dioxidotetrahydrothiophen-3-yl)methoxy)-2′,6′-dimethyl-[1,1′-biphenyl]-3-yl)methyl)amino)phenyl)oxetan-3-yl)acetic acid;
   or an isotopic form or a stereoisomer or a tautomer or a pharmaceutically acceptable salt, a solvate, a prodrug, a polymorph, a carboxylic acid isostere, an N-oxide or a S-oxide thereof.

The present invention also relates to processes for the preparation of the compounds of Formula (I) or pharmaceutically acceptable salts thereof. The compounds of Formula (I) may be prepared by the schemes depicted herein below but not limited thereto. The starting materials and reagents employed in the processes for preparation of the compounds of Formula (I) may be commercially available or may be prepared by processes known in the art.

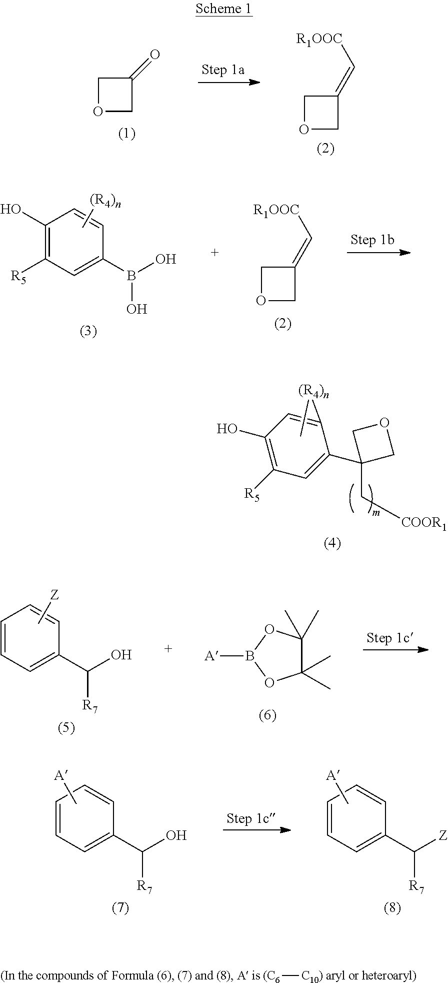

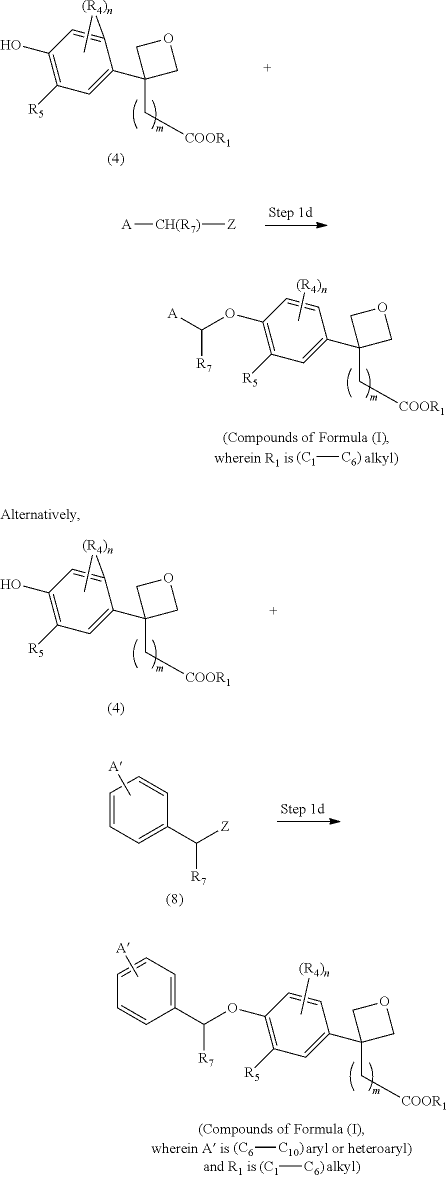

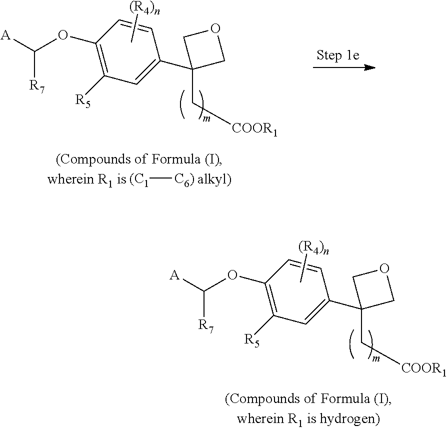

### Reaction Conditions:

Step 1a: Ethyl 2-(triphenylphosphoranylidene)acetate (PPh₃CHCOOC₂H₅), dichloromethane (DCM), Room Temperature (RT) (20° C.-25° C.);

Step 1b: Cyclooctadiene rhodium chloride dimer (Rh(COD)₂Cl₂), KOH, dioxane;
Step 1c′: Palladium catalyst, N, N-dimethylformamide (DMF), Na₂CO₃;
Step 1c″: Carbon tetrabromide, triphenyl phosphine catalyst;
Step 1d: Cesium carbonate (Cs₂CO₃), DMF, RT;
Step 1e: LiOH.H₂O, tetrahydrofuran (THF), Methanol (MeOH), Hydrochloric acid (HCl), RT;

In one embodiment, there are provided processes for the preparation of the compound of Formula (I), wherein

R₁ is hydrogen or (C₁-C₆)alkyl;
R₂ and R₃ together form an oxetane ring;
R₄ at each occurrence is independently selected from hydrogen, (C₁-C₆)alkyl, halogen, halo(C₁-C₆)alkyl, hydroxy, —O(C₁-C₆)alkyl, amino, cyano, nitro, —C(O)R₉ and —S(O)ₚR₆;

### Rₓ is A-CH(R₇)—X;

### R_{y} is R₅;

R₅ is selected from hydrogen, (C₁-C₆) alkyl, halogen, halo(C₁-C₆)alkyl, hydroxy, —O(C₁-C₆)alkyl, amino, cyano, nitro, —C(O)R₉ or —S(O)ₚR₆;

### X is O;

A is selected from (C₆-C₁₀)aryl, heteroaryl,

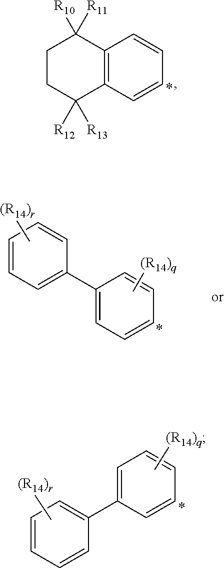

m is 1;
R₆, R₇, R₉, R₁₀, R₁₁, R₁₂, R₁₃, R₁₄, n, p, q and r are as defined above in Formula (I); wherein

(C₁-C₆)alkyl is unsubstituted or substituted with one or more groups independently selected from (C₁-C₆)alkyl, (C₂-C₈) alkenyl, (C₂-C₈) alkynyl, halogen, halo(C₁-C₆) alkyl, hydroxy, —O(C₁-C₆)alkyl, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

—O(C₁-C₆)alkyl is unsubstituted or substituted with one or more groups independently selected from (C₁-C₆)alkyl, (C₃-C₈)cycloalkyl, heterocyclyl, hydroxy, halogen, amino, cyano, —(C₁-C₆)alkyl-S(O)ₚR₆, —S(O)ₚR₆, —NR₁₅R₁₆ and —(CH₂)ₛNR₁₅R₁₆; wherein R₆, R₁₅, R₁₆, p and s are as defined above;

(C₆-C₁₀)aryl is unsubstituted or substituted with one or more groups independently selected from (C₁-C₆)alkyl, (C₂-C₈) alkenyl, (C₂-C₈) alkynyl, halogen, halo(C₁-C₆) alkyl, hydroxy, O(C₁-C₆) alkyl, halo(C₁-C₆)alkoxy, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

heterocyclyl is a 3- to 9-membered ring, which is unsubstituted or substituted with one or more groups independently selected from (C₁-C₆)alkyl, halogen, halo(C₁-C₆) alkyl, hydroxy, —O(C₁-C₆) alkyl, halo(C₁-C₆)alkoxy, (C₆-C₁₀)aryl, amino, cyano, nitro, —(C₁-C₆)alkyl-OH, (C₁-C₆)alkyl-O—(C₁-C₆)alkyl and —O(C₁-C₆)alkyl-S(O)ₚR₆; wherein R₆, and p are as defined above;

heteroaryl is a 3- to 10-membered ring, which is unsubstituted or substituted with one or more groups selected from (C₁-C₆)alkyl, (C₂-C₈) alkenyl, (C₂-C₈) alkynyl, halogen, halo(C₁-C₆) alkyl, hydroxy, —O(C₁-C₆) alkyl, halo(C₁-C₆)alkoxy, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

halogen is selected from chlorine, bromine, iodine or fluorine;

consists of the reaction steps as outlined in the above Scheme 1 described herein below:

### Step 1a:

This process step involves reacting commercially available oxetone (compound (1)) in a solvent such as dichloromethane with a reagent such as ethyl 2-(triphenylphosphoranylidene)acetate at room temperature, according to the method described in Angew Chem. Intl. Ed. 45:7736-39, to obtain the intermediate, compound (2), wherein R₁ is (C₁-C₆)alkyl.

### Step 1b:

Compound (3) is reacted with the compound (2) (obtained in Step 1a) in the presence of a suspension comprising a catalyst selected from cyclooctadiene rhodium chloride dimer, trimethylsilylchloride or nBuLi—CuI in a solvent selected from dioxane, THF, toluene, acetonitrile or dimethoxyethane and a base selected from potassium hydroxide (KOH), sodium hydroxide (NaOH), potassium bicarbonate (KHCO₃), sodium bicarbonate (NaHCO₃), 1,8-diazabicyclo[5.4.0]undec-7-ene (DBU), pyrrolidine or triethylamine, according to the method described in Angew Chem. Intl. Ed. 45:7736-39 and J. Med. Chem., 2010, 53(8):3227-3246, to obtain the compound (4).

Step 1c′:

In this step, the compound (5) (wherein Z is halogen) is reacted with the compound (6) (wherein A′ is (C₆-C₁₀) aryl or heteroaryl) in N, N-dimethylformamide as the solvent in the presence of sodium carbonate (Na₂CO₃) as the base, and a Palladium catalyst, according to the method described in PCT published application number WO2004000315 A1 and J. Med. Chem., 2004, 47(21):4998-5008, to obtain compound (7) (wherein A′ is (C₆-C₁₀) aryl or heteroaryl).

Step 1c″:

In this step, compound (7) (wherein A′ is (C₆-C₁₀) aryl or heteroaryl) is treated with an halogenating reagent such as carbon tetrabromide, in the presence of a catalyst selected from triphenyl phosphine; phosphorous tribromide (PBr₃) or thionyl chloride (SOC₂), in a solvent such as dichloromethane, as per the method described in Tetrahedron Let., 1996, 37(29):5171-5174, to obtain the compound (8) (wherein Z is halogen, A′ is (C₆-C₁₀) aryl and heteroaryl).

### Step 1d:

The compound (4) (obtained in Step 1b) is reacted with a compound of formula: A-CH(R₇)—Z or alternatively with the compound (8) (obtained in Step 1c″) in the presence of a solvent selected from DMF, acetone, dimethylether, acetonitrile, dioxane or THF, and a base selected from cesium carbonate (Cs₂CO₃) or potassium carbonate (K₂CP₃), according to the method described in PCT published application WO2005117909 and Bioorg. Med. Chem. Lett., 2008, 18 (14):3887-3890, to obtain the compound of Formula (I), wherein R₁ is (C₁-C₆)alkyl.

### Step 1e:

The compound of Formula (I) (obtained in Step 1d, wherein R₁ is (C₁-C₆)alkyl) was taken in a solvent selected from THF, ethanol, MeOH, water or a mixture thereof, and was hydrolysed using a base selected from NaOH, KOH, Lithium hydroxide (LiOH) or barium hydroxide (Ba(OH)₂), followed by neutralization with HCl, according to the method described in J. Med. Chem., 1995, 38(3):1386-96, to obtain compound of Formula (I), wherein R₁ is hydrogen.

The process for the preparation of the compounds of Formula (I) as illustrated in Scheme 1 can be modified to prepare the compounds of Formula (I), wherein R₂ and R₃ together form an azetidine ring, wherein N of the azetidine ring is substituted with a group selected from H, (C₁-C₆) alkyl, C(O)(C₁-C₆)alkyl or —S(O)₂(C₁-C₆)alkyl. For instance, the process as illustrated in Scheme 1 can be modified such that in Step 1a, commercially available compound namely tert-butyl 3-oxoazetidine-1-carboxylate can be used as the starting material in place of the commercially available oxetone (denoted as compound (1) in Scheme 1). Whereas, all the other reagents and the reaction conditions that can be used in the process will remain the same.

Alternatively, the compounds of Formula (I) can be prepared in accordance with a process involving the reaction steps depicted in the following Scheme 2:

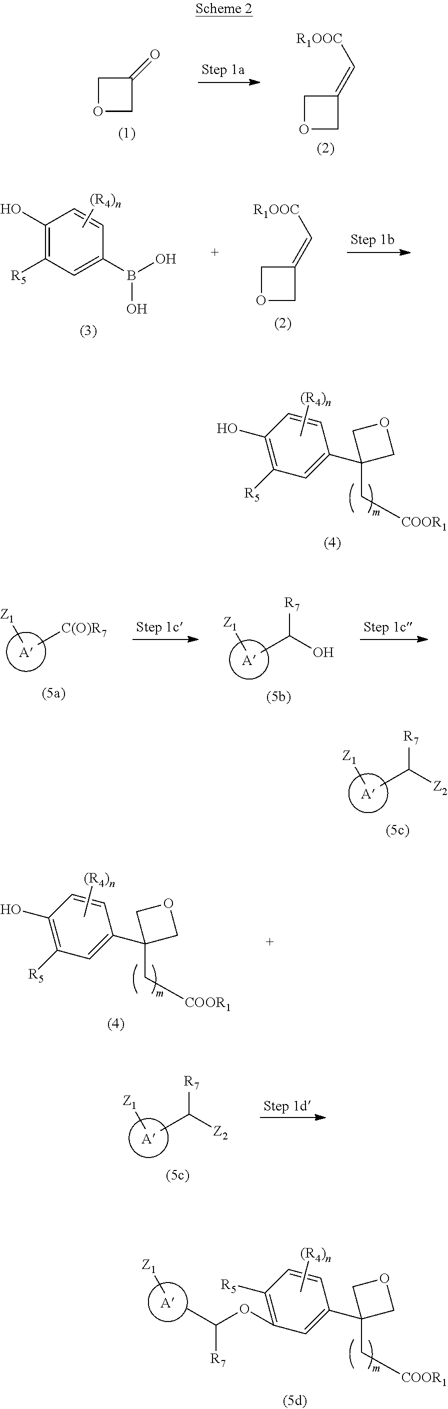

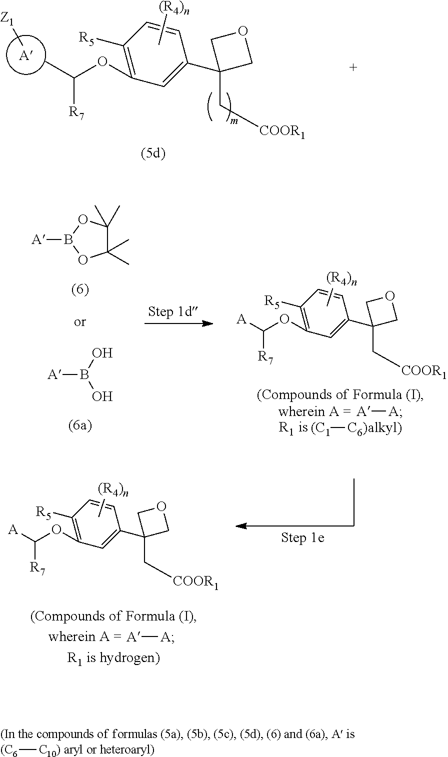

### Reaction Conditions:

Step 1a: Ethyl 2-(triphenylphosphoranylidene)acetate (PPh₃CHCOOC₂H₅), dichloromethane (DCM), Room Temperature (RT) (20° C.-25° C.);
Step 1b: Cyclooctadiene rhodium chloride dimer (Rh(COD)₂Cl₂), KOH, dioxane;
Step 1c′: NaBH₄ or LiAlH₄ or Mg, methanol or THF;
Step 1c″: Carbon tetrabromide, triphenyl phosphine catalyst;
Step 1d′: Cesium carbonate (Cs₂CO₃), DMF, RT;
Step 1d″: Palladium catalyst, N, N-dimethylformamide (DMF), Na₂CO₃;
Step 1e: LiOH.H₂O, tetrahydrofuran (THF), Methanol (MeOH), Hydrochloric acid (HCl), RT;

In another embodiment, the processes for the preparation of the compound of Formula (I), wherein R₁ is hydrogen or (C₁-C₆)alkyl;

R₂ and R₃ together form an oxetane ring;
R₄ at each occurrence is independently selected from hydrogen, (C₁-C₆) alkyl, halogen, halo(C₁-C₆) alkyl, hydroxy, —O(C₁-C₆)alkyl, amino, cyano, nitro, —C(O)R₉ and —S(O)ₚR₆;

### Rₓ is A-CH(R₇)—X;

### R_{y} is R₅;

R₅ is selected from hydrogen, (C₁-C₆) alkyl, halogen, halo(C₁-C₆) alkyl, hydroxy, —O(C₁-C₆)alkyl, amino, cyano, nitro, —C(O)R₉ or —S(O)ₚR₆;

### X is O;

A is selected from (C₆-C₁₀)aryl, heteroaryl,

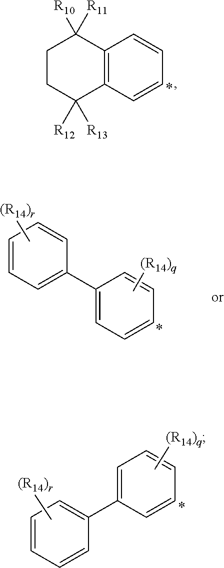

m is 1;
R₆, R₇, R₉, R₁₀, R₁₁, R₁₂, R₁₃, R₁₄, n, p, q and r are as defined above in Formula (I); wherein

(C₁-C₆)alkyl is unsubstituted or substituted with one or more groups independently selected from (C₁-C₆)alkyl, (C₂-C₈) alkenyl, (C₂-C₈) alkynyl, halogen, halo(C₁-C₆) alkyl, hydroxy, —O(C₁-C₆)alkyl, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

—O(C₁-C₆)alkyl is unsubstituted or substituted with one or more groups independently selected from (C₁-C₆)alkyl, (C₃-C₈)cycloalkyl, heterocyclyl, hydroxy, halogen, amino, cyano, —(C₁-C₆)alkyl-S(O)ₚR₆, —S(O)ₚR₆, —NR₁₅R₁₆ and —(CH₂)ₛNR₁₅R₁₆; wherein R₆, R₁₅, R₁₆, p and s are as defined above;

(C₆-C₁₀)aryl is unsubstituted or substituted with one or more groups independently selected from (C₁-C₆)alkyl, (C₂-C₈) alkenyl, (C₂-C₈) alkynyl, halogen, halo(C₁-C₆) alkyl, hydroxy, —O(C₁-C₆) alkyl, halo(C₁-C₆)alkoxy, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

heterocyclyl is 3- to 9-membered ring, which is unsubstituted or substituted with one or more groups independently selected from (C₁-C₆)alkyl, halogen, halo(C₁-C₆) alkyl, hydroxy, —O(C₁-C₆) alkyl, halo(C₁-C₆)alkoxy, (C₆-C₁₀)aryl, amino, cyano, nitro, —(C₁-C₆)alkyl-OH, (C₁-C₆)alkyl-O—(C₁-C₆)alkyl and —O(C₁-C₆)alkyl-S(O)ₚR₆; wherein R₆, and p are as defined above;

heteroaryl is 3- to 10-membered ring, which is unsubstituted or substituted with one or more groups independently selected from (C₁-C₆)alkyl, (C₂-C₈) alkenyl, (C₂-C₈) alkynyl, halogen, halo(C₁-C₆) alkyl, hydroxy, —O(C₁-C₆) alkyl, halo(C₁-C₆)alkoxy, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

halogen is selected from chlorine, bromine, iodine or fluorine;

consists of the reaction steps as outlined in the above Scheme 2 described herein below:

### Step 1a:

In this process step, compound (2), wherein R₁ is (C₁-C₆)alkyl, is prepared from compound (1) in accordance with the method described in the reaction Step 1a of Scheme 1.

### Step 1b:

In this process step, compound (4) is obtained by reaction of the compound (3) with the compound (2) in accordance with the method described in the reaction Step 1b of Scheme 1.

Step 1c′:

In this step, compound (5a) (wherein ring A′ is (C₆-C₁₀) aryl or heteroaryl and Z₁ is halogen) is subjected to reduction in the presence of a reducing agent selected from sodium borohydride, lithium aluminium hydride or magnesium and a solvent selected from methanol or THF (tetrahydrofuran) to obtain the compound (5b).

Step 1c″:

In this step, compound (5b) (as obtained in Step 1c′) is treated with carbon tetrabromide as the halogenating agent, in the presence of triphenylphosphine as the catalyst in accordance with Step 1c″ of Scheme 1. Alternatively, compound (5b) is treated with a halogenating reagent selected from phosphorous tribromide and phosphorous pentachloride, or a protecting sulphonating reagent selected from p-toluene sulfonyl chloride (tosyl chloride) and methane sulfonyl chloride (mesyl chloride/anhydride), in a solvent selected from dichloromethane or dioxane to obtain the compound (5c), wherein Z₂ is halogen,

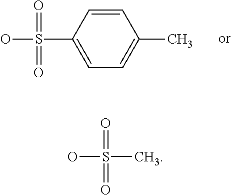

Step 1d′:

In this step, the compound (4) is reacted with compound (5c) (wherein Z₂ is halogen,

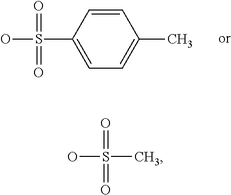

the compound obtained in Step 1c″) in accordance with the method described in reaction Step 1d of Scheme 1, to obtain the compound (5d), wherein Z, is halogen and R₁ is (C₁-C₆)alkyl [which corresponds to the compound of Formula (I), wherein A is (C₆-C₁₀) aryl or heteroaryl substituted with halogen and R₁ is (C₁-C₆)alkyl].
Step 1d″:

In this step, compound (5d) wherein Z₁ is halogen and R₁ is (C₁-C₆)alkyl; is reacted with the compound (6) or the compound (6a) (wherein A′ is (C₆-C₁₀) aryl or heteroaryl) in accordance with the method described in reaction Step 1c′ of Scheme 1, to obtain the compound of Formula (I), wherein A is A′-A′ and R₁ is (C₁-C₆)alkyl.

### Step 1e:

The compound of Formula (I) (obtained in Step 1d″, wherein R₁ is (C₁-C₆)alkyl) is hydrolysed followed by neutralization according to the method described in reaction Step 1e of Scheme 1, to obtain compound of Formula (I), wherein R₁ is H.

Alternatively, the compounds of Formula (I) can be prepared in accordance with a process involving the reaction steps depicted in the following Scheme 3. In respect of Scheme 3, it would be understood by a skilled artisan that in the compound (9) and the compounds of Formula (I) presented in the said scheme, the variable point of attachment of the phenyl ring on another phenyl ring corresponds to the biphenyl rings presented in the definition of group A in the compounds of Formula (I) as described in one or more of the embodiments discussed herein.

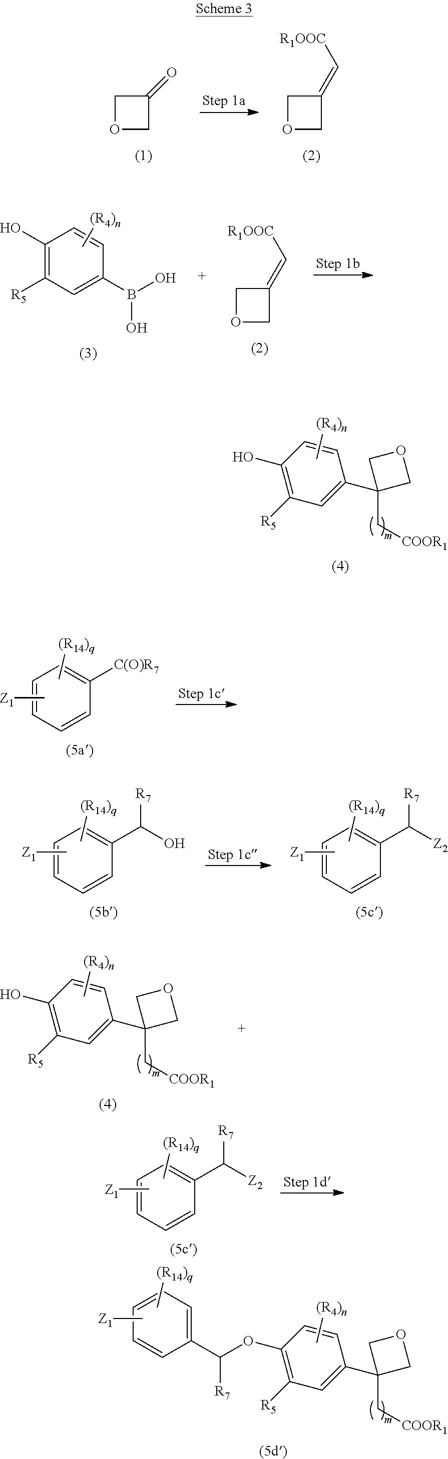

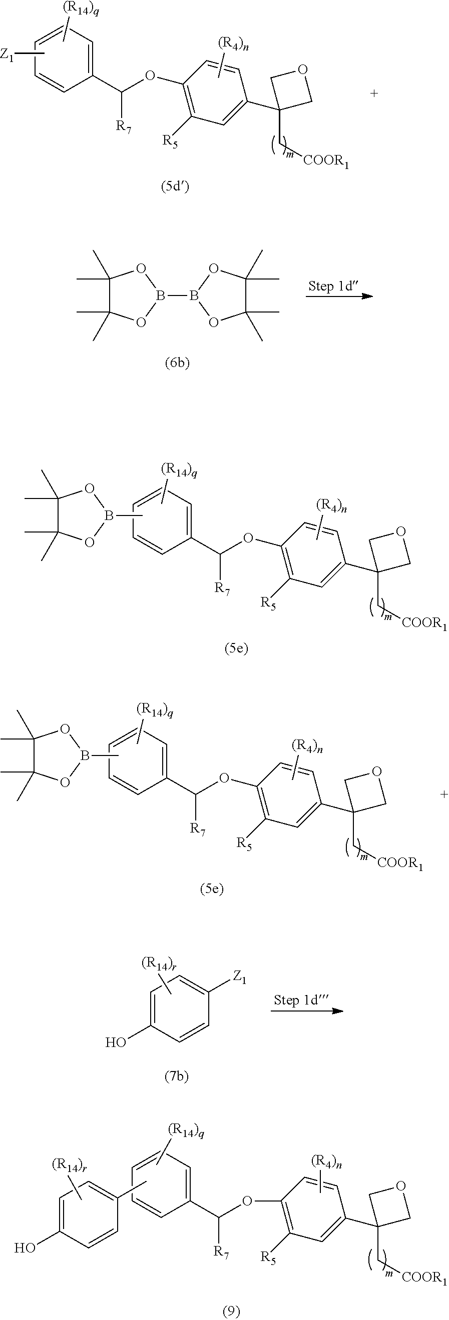

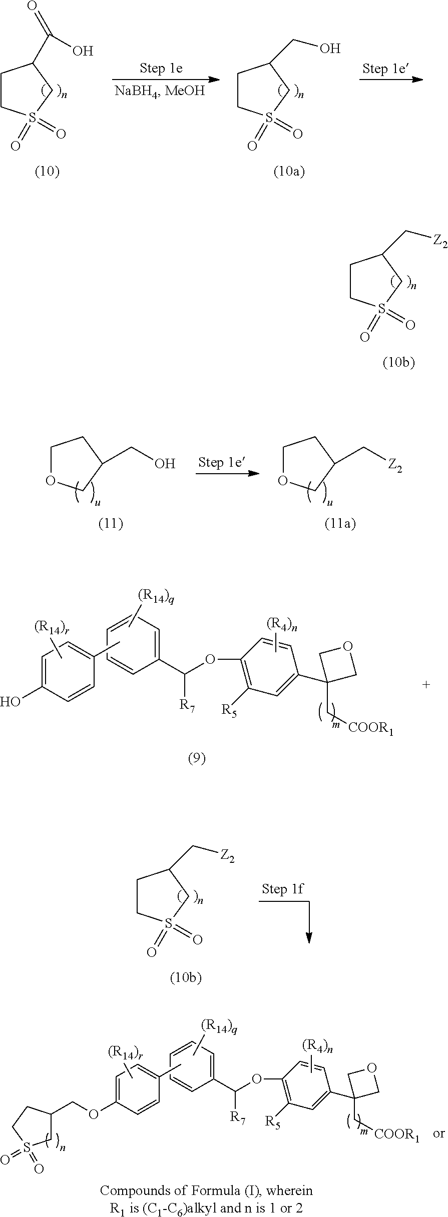

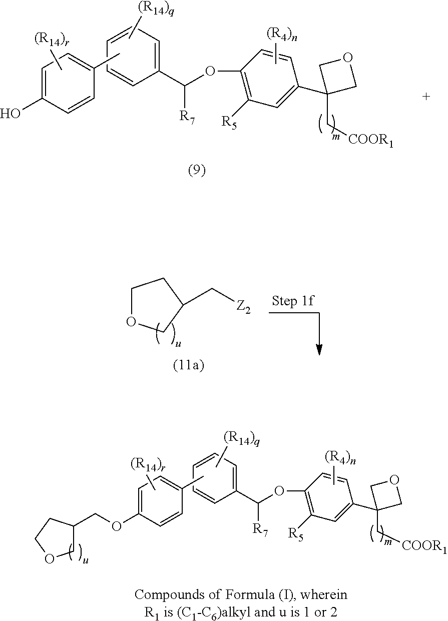

### Reaction Conditions:

Step 1a: Ethyl 2-(triphenylphosphoranylidene)acetate (PPh₃CHCOOC₂H₅), dichloromethane (DCM), Room Temperature (RT) (20° C.-25° C.);
Step 1b: Cyclooctadiene rhodium chloride dimer (Rh(COD)₂Cl₂), KOH, dioxane;
Step 1c′: NaBH₄ or LiAlH₄ or Mg, methanol or THF;
Step 1c″: Carbon tetrabromide, triphenyl phosphine catalyst;
Step 1d′: Cesium carbonate (Cs₂CO₃), DMF, RT;
Step 1d″: (Pd(dppf)Cl₂.DCM, potassium acetate, dioxane;
Step 1d′″: (PPh₃)₄Pd, dioxane;
Step 1e: Ethyl chloroformate, N-methyl morpholine, THF; NaBH₄ or LiAlH₄; THF, dioxane;
Step 1e′: para-toluene sulfonyl chloride, triethylamine, DCM;
Step 1f: Cesium carbonate (Cs₂CO₃), DMF, RT;

In another embodiment, the processes for the preparation of the compounds of Formula (I), wherein

R₁ is hydrogen or (C₁-C₆)alkyl;
R₂ and R₃ together form an oxetane ring;
R₄ at each occurrence is independently selected from hydrogen, (C₁-C₆) alkyl, halogen, halo(C₁-C₆) alkyl, hydroxy, —O(C₁-C₆)alkyl, amino, cyano, nitro, —C(O)R₉ and —S(O)ₚR₆;

### Rₓ is A-CH(R₇)—X;

### R_{y} is R₅;

R₅ is selected from hydrogen, (C₁-C₆) alkyl, halogen, halo(C₁-C₆) alkyl, hydroxy, —O(C₁-C₆)alkyl, amino, cyano, nitro, —C(O)R₉ or —S(O)ₚR₆;

### X is O;

A is selected from

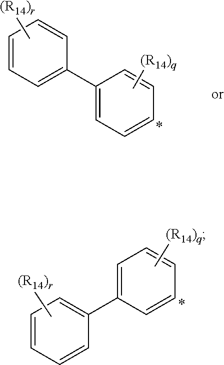

m is 1;
R₆, R₇, R₉, R₁₄, n, p, q and r are as defined above in Formula (I);
wherein

(C₁-C₆)alkyl is unsubstituted or substituted with one or more groups independently selected from (C₁-C₆)alkyl, (C₂-C₈) alkenyl, (C₂-C₈) alkynyl, halogen, halo(C₁-C₆) alkyl, hydroxy, —O(C₁-C₆)alkyl, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

—O(C₁-C₆)alkyl is unsubstituted or substituted with one or more groups independently selected from (C₁-C₆)alkyl, (C₃-C₈)cycloalkyl, heterocyclyl, hydroxy, halogen, amino, cyano, —(C₁-C₆)alkyl-S(O)ₚR₆, —S(O)ₚR₆, —NR₁₅R₁₆ and —(CH₂)ₛNR₁₅R₁₆; wherein R₆, R₁₅, R₁₆, p and s are as defined above;

(C₆-C₁₀)aryl is unsubstituted or substituted with one or more groups independently selected from (C₁-C₆)alkyl, (C₂-C₈) alkenyl, (C₂-C₈) alkynyl, halogen, halo(C₁-C₆) alkyl, hydroxy, —O(C₁-C₆) alkyl, halo(C₁-C₆)alkoxy, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

heterocyclyl is a 3- to 9-membered ring, which is unsubstituted or substituted with one or more groups independently selected from (C₁-C₆)alkyl, halogen, halo(C₁-C₆) alkyl, hydroxy, —O(C₁-C₆) alkyl, halo(C₁-C₆)alkoxy, (C₆-C₁₀)aryl, amino, cyano, nitro, —(C₁-C₆)alkyl-OH, (C₁-C₆)alkyl-O—(C₁-C₆)alkyl and —O(C₁-C₆)alkyl-S(O)ₚR₆; wherein R₆, and p are as defined above;

heteroaryl is a 3- to 10-membered ring, which is unsubstituted or substituted with one or more groups independently selected from (C₁-C₆)alkyl, (C₂-C₈) alkenyl, (C₂-C₈) alkynyl, halogen, halo(C₁-C₆) alkyl, hydroxy, —O(C₁-C₆) alkyl, halo(C₁-C₆)alkoxy, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;

halogen is selected from chlorine, bromine, iodine or fluorine;

consists of the reaction steps as outlined in Scheme 3 described herein below:

### Step 1a:

In this process step, compound (2) wherein R₁ is (C₁-C₆)alkyl, is prepared from the compound (1) in accordance with the method described in reaction Step 1a of Scheme 1.

### Step 1b:

In this process step, compound (4) is obtained by reaction of the compound (3) with the compound (2) in accordance with the method described in reaction Step 1b of Scheme 1.

Step 1c′:

In this step, the compound (5a′) (wherein Z₁ is halogen) is subjected to reduction using a reducing agent selected from sodium borohydride, lithium aluminium hydride or magnesium in a solvent selected from methanol or THF (tetrahydrofuran) or like solvents to obtain the compound (5b′).

Step 1c″:

In this step, the compound (5b′) is treated with a halogenating reagent such as carbon tetrabromide, in the presence of a catalyst such as triphenylphosphine in accordance with Step 1c″ in Scheme (1). Alternatively, the compound (5b′) is further treated with a halogenating reagent such as phosphorous tribromide (PBr₃) or phosphorous pentachloride; or a sulphonating reagent such as p-toluene sulfonyl chloride (tosyl chloride) or methane sulfonyl chloride (mesyl chloride), in an appropriate solvent, for example, dichloromethane or dioxane to obtain the compound (5c′) (wherein Z₂ is halogen,

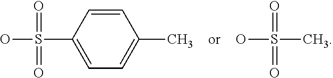

Step 1d′:

In this step, the compound (4) (as obtained in Step (b) above) is reacted with the compound (5c′) (compound obtained in Step 1c″) in accordance with the method described in reaction Step 1d of Scheme 1, to obtain the compound (5d′) (wherein Z₁ is halogen and R₁ is (C₁-C₆)alkyl).

Step 1d″:

In this step, the compound (5d′) is reacted with the compound (6b) in a solvent selected from dichloromethane (DCM), acetonitrile, dioxane or toluene, in the presence of a base such as potassium acetate and a Palladium catalyst, such as [1,1′-bis(diphenylphosphino)ferrocene]dichloropalladium(II), complex with dichloro methane (Pd(dppf)Cl₂.DCM), Pd(dppf)C₂ or palladium tetrakistriphenylphosphine (Pd(PPh₃)₄), at a temperature ranging from 25 to 100° C. for a reaction time ranging from 8 h to 24 h, to obtain the compound (5e).

Step 1d′″:

In this step, the compound (5e) is reacted with the compound (7b) in a solvent selected from dioxane, DMF, toluene, THF or acetonitrile in the presence of a Palladium catalyst such [1,1′-bis(diphenyl phosphino)ferrocene]dichloro palladium (11), complex with dichloromethane (Pd(dppf)Cl₂.DCM), Pd(dppf)C₂ or palladium tetrakistriphenylphosphine (Pd(PPh₃)₄) to obtain compound (9) (which corresponds to the compound of Formula (I) wherein R₁ is (C₁-C₆)alkyl).

### Step 1e:

In this step, the compound (10) (wherein n is 1 or 2) is esterified in the presence of an esterifying agent such as ethyl chloroformate in the presence of a base such as N-methyl morpholine and a solvent such as THF and the resulting compound (an ester) is further subjected to reduction using a reducing agent selected from NaBH₄ or LiAlH₄, in a solvent selected from THF, dioxane or water or a mixture thereof at 0° C. to 50° C. for 1 h to 5 h to obtain the compound (10a).

Step 1e′:

In this step, the compound (10a) wherein n is 1 or 2, is reacted with a sulphonating reagent, such as p-toluene sulfonyl chloride (tosyl chloride), benzene sulfonyl chloride or methane sulfonyl chloride (mesyl chloride) in a solvent selected from DCM, chloroform or THF and in the presence of a base selected from triethyl amine, diisopropyl amine or pyridine to obtain the compound (10b) wherein Z₂ is a protecting group such as p-toluene sulfonyl, benzene sulfonyl or methane sulfonyl group.

Similarly, compound (11) (wherein u is 1 or 2), is reacted with a sulphonating reagent, such as p-toluene sulfonyl chloride (tosyl chloride), benzene sulfonyl chloride or methane sulfonyl chloride (mesyl chloride) in a solvent selected from DCM, chloroform or THF and in the presence of a base selected from triethyl amine, diisopropyl amine or pyridine to obtain the compound (11a) wherein Z₂ is a protecting group such as p-toluene sulfonyl, benzene sulfonyl or methane sulfonyl group.

### Step 1f:

In this step, the compound (9) is reacted with the compound (10b) in accordance with the procedure in Step 1d of Scheme 1, to obtain the compound of Formula (I) wherein R₁ is (C₁-C₆)alkyl and n is 1 or 2.

Similarly, compound (9) is reacted with compound (11a) in accordance with the procedure in Step 1d of Scheme 1, to obtain compound of Formula (I) (wherein R₁ is (C₁-C₆)alkyl and u is 1 or 2).

The compounds of Formula (I) (wherein R₁ is (C₁-C₆)alkyl and n is 1 or 2) can be further hydrolysed by the procedure in Step 1e of Scheme 1, to obtain the corresponding acid i.e. the compounds of Formula (I) (wherein R₁ is hydrogen and n is 1 or 2).

Similarly, the compounds of Formula (I) (wherein R₁ is (C₁-C₆)alkyl and u is 1 or 2) can be further hydrolysed by the procedure in Step 1e of Scheme 1, to obtain the corresponding acid i.e. the compound of Formula (I) (wherein R₁ is hydrogen and u is 1 or 2).

Those skilled in the art will recognize that the compounds of Formula (I) of the present invention contain asymmetric or chiral centers, and therefore exist in different stereoisomeric forms, as racemic mixtures of enantiomers, mixtures of diastereomers or enantiomerically or optically pure compounds. The term “chiral” refers to molecules which have the property of non-superimposability of the mirror image cohort, while the term “achiral” refers to molecules which are superimposable on their mirror image partner. It is intended that all stereoisomeric forms of the compounds of the invention, including but not limited to, diastereomers and enantiomers, as well as mixtures thereof such as racemic mixtures, geometric isomers form part of the present invention.

When the compounds of Formula (I) of the present invention contain one chiral center, the compounds exist in two enantiomeric forms and the present invention includes both enantiomers and mixtures of enantiomers, such as the specific 50:50 mixture referred to as a racemic mixtures. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation. Designation of a specific absolute configuration at a chiral carbon of the compounds of the invention is understood to mean that the designated enantiomeric form of the compounds is in enantiomeric excess (ee) or in other words is substantially free from the other enantiomer. For example, the “R” forms of the compounds are substantially free from the “S” forms of the compounds and are, thus, in enantiomeric excess of the “S” forms. Conversely, “S” forms of the compounds are substantially free of “R” forms of the compounds and are, thus, in enantiomeric excess of the “R” forms. Enantiomeric excess, as used herein, is the presence of a particular enantiomer at greater than 50%. In a particular embodiment when a specific absolute configuration is designated, the enantiomeric excess of depicted compounds is at least about 90%. When a compound of Formula (I) of the present invention has two or more chiral carbons it can have more than two optical isomers and can exist in diastereoisomeric forms. For example, when there are two chiral carbons, the compound can have up to 4 optical isomers and 2 pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs may be separated by methods known to those skilled in the art, for example chromatography or crystallization and the individual enantiomers within each pair may be separated as described above. The present invention includes each diastereoisomer of such compounds and mixtures thereof.

The isotopically labeled forms of compounds of Formula (I), can be prepared by conventional techniques known to those skilled in the art or by processes analogous to those described above or in the subsequent section on examples by using a corresponding isotopically labeled reagent in place of the non-labeled reagent.

In one embodiment, the compounds of Formula (I) exists as tautomers, and it is intended to encompass all the tautomeric forms of the compounds within the scope of the present invention.

In an embodiment, the compounds of Formula (I) in their free base form are converted to their corresponding pharmaceutically acceptable salts. The pharmaceutically acceptable salt of the compounds of Formula (I) are prepared with relatively non-toxic acids or bases, depending on the particular substituents found on the compound described herein. When the compounds of Formula (I) of the present invention contain an acidic group they can form an addition salt with a suitable base. For example, pharmaceutically acceptable base addition salts of the compounds of the present invention may include their alkali metal salts such as sodium, potassium, calcium, magnesium, ammonium or an organic base addition salt. Examples of pharmaceutically acceptable organic base addition salts of the compounds of the present invention include those derived from organic bases like lysine, arginine, guanidine, diethanolamine, metformin or other organic bases known to the person skilled in the art.

When the compounds of Formula (I) of the present invention contain one or more basic groups, they can form an addition salt with an inorganic or an organic acid. Examples of pharmaceutically acceptable acid addition salts include those derived from inorganic acids like boric acid, perchloric acid, hydrochloric acid, hydrobromic acid, hydrofluoric acid, hydriodic acid, nitric acid, carbonic acid, monohydrogencarbonic acid, phosphoric acid, monohydrogenphosphoric acid, dihydrogenphosphoric acid, sulfuric acid, monohydrogensulfuric acid, phosphorous acids or other inorganic acids known to the person skilled in the art. Furthermore, examples of pharmaceutically acceptable acid addition salts include the salts derived from organic acids such as acetic acid, propionic acid, isobutyric acid, oxalic acid, malic acid, tartaric acid, citric acid, ascorbic, maleic acid, malonic acid, benzoic acid, succinic acid, suberic acid, fumaric acid, mandelic acid, phthalic acid, benzenesulfonic acid, toluenesulfonic acid, methanesulfonic acid, glucuronic acid, galacturonic acid, naphthoic acid, camphoric acid or other organic acids known to the person skilled in the art. Certain specific compounds of the present invention contain both basic and acidic functionalities that allow the compounds to be converted into either base or acid addition salts.

The pharmaceutically acceptable salts of the present invention can be synthesized from the subject compound i.e. the compound of Formula (I) which contains a basic or acidic moiety by conventional chemical methods. Generally the salts are prepared by contacting the free base or acid with desired salt-forming inorganic or organic acid or a base in a suitable solvent or dispersant or by anion exchange or cation exchange with other salts. Suitable solvents are, for example, ethyl acetate, ethers, alcohols, acetone, or mixtures of these solvents.

The present invention furthermore includes all the solvates of the compounds of Formula (I), for example, hydrates and the solvates formed with other solvents of crystallisation, selected from alcohols such as methanol, ethanol, 1-propanol or 2-propanol, ethers such as diethyl ether, isopropyl ether or tetrahydrofuran, esters such as methyl acetate or ethyl acetate, ketone such as acetone or their mixtures thereof. Certain compounds of the present invention can exist in unsolvated forms as well as solvated forms, including hydrated forms.

It is further intended to encompass various polymorphs of compounds of Formula (I) within the scope of the present invention. Various polymorphs of compounds of the present invention can be prepared by standard crystallisation procedures known in the art. The crystallisation technique employed can utilize various solvents or their mixtures, temperature conditions and various modes of cooling, ranging from very fast to very slow cooling. The presence of polymorphs can be determined by IR (Infra-red) spectroscopy, solid probe NMR (Nuclear Magnetic Resonance) spectroscopy, differential scanning calorimetry, powder X-ray diffraction or such other standard techniques.

Furthermore, the present invention also includes prodrugs of the compounds of Formula (I). The prodrugs of the compounds of the present invention are derivatives of the aforesaid compounds of the invention which upon administration to a subject in need thereof undergoes chemical conversion by metabolic or chemical processes to release the parent drug in vivo from which the prodrug is derived. The preferred prodrugs are pharmaceutically acceptable ester derivatives e.g., alkyl esters, cycloalkyl esters, alkenyl esters, benzyl esters, mono- or di-substituted alkyl esters convertible by solvolysis under physiological conditions to the parent carboxylic acid, and those conventionally used in the art.

The present invention further relates to carboxylic acid isosteres of the compounds of Formula (I).

The present invention also relates to N-oxide derivatives of the compounds of Formula (I).

The present invention also relates to S-oxide derivatives of the compounds of Formula (I).

In one aspect of the present invention, i.e. the compounds of Formula (I) are GPR40 agonists.

In an embodiment of the present invention, the compounds of Formula (I) find use in the treatment of a disease or a condition mediated by GPR40.

In another aspect, the present invention relates to a method for the treatment of a disease or a condition mediated by GPR40, comprising administering to a subject in need thereof a therapeutically effective amount of a compound of Formula (I) or a stereoisomer or a tautomer or a pharmaceutically acceptable salt, a solvate, a prodrug, a polymorph, a carboxylic acid isostere, an N-oxide or a S-oxide thereof.

In an embodiment, the present invention relates to a method for the treatment of a disease or a condition mediated by GPR40, comprising administering to a subject in need thereof a therapeutically amount of a compound of Formula (I) or a stereoisomer or a tautomer or a pharmaceutically acceptable salt thereof.

In yet another aspect, the present invention provides use of the compound of Formula (I) or a stereoisomer or a tautomer or a pharmaceutically acceptable salt, a solvate, a prodrug, a polymorph, a carboxylic acid isostere, an N-oxide or a S-oxide thereof for the treatment of a disease or a condition mediated by GPR40.

In an embodiment, the present invention relates to use of the compound of Formula (I) or a stereoisomer or a tautomer or a pharmaceutically acceptable salt thereof for the treatment of a disease or a condition mediated by GPR40.

According to an aspect, the present invention relates to use of the compounds of Formula (I) or a stereoisomer or a tautomer or a pharmaceutically acceptable salt, a solvate, a prodrug, a polymorph, a carboxylic acid isostere, an N-oxide or a S-oxide thereof in the manufacture of a medicament, for the treatment of a disease or a condition mediated by GPR40.

According to one embodiment, the present invention relates to use of the compounds of Formula (I) or a stereoisomer or a tautomer or a pharmaceutically acceptable salt thereof; in the manufacture of a medicament for the treatment of a disease or a condition mediated by GPR40.

As used herein, the term “a disease or a condition mediated by GPR40” or “GPR40 mediated disease(s) or condition(s)” refers to a disease or a disorder or a condition characterized by inappropriate, for example, less than or greater than normal, GPR40 activity. A GPR40-mediated disease or disorder may be completely or partially mediated by inappropriate GPR40 activity.

In an embodiment of the invention the disease or condition mediated by GPR40 is selected from: diabetes, obesity, hyperglycemia, glucose intolerance, insulin resistance, hyperinsulinemia, hypercholesterolemia, hypertension, hyperlipoproteinemia, hyperlipidemia, hypertriglyceridemia, dyslipidemia, metabolic syndrome, syndrome X, cardiovascular disease, atherosclerosis, kidney disease, polycystic ovary syndrome, ketoacidosis, thrombotic disorders, nephropathy, diabetic neuropathy, diabetic retinopathy, sexual dysfunction, fatty liver development, dermatopathy, dyspepsia, hypoglycemia, cancer, edema or a disorder related to glucose levels such as pancreatic beta cell regeneration.

In an embodiment of the invention the disease or condition mediated by GPR40 is selected from: diabetes, obesity, insulin resistance, hyperglycemia, glucose intolerance, hypercholesterolemia, hypertriglyceridemia, dyslipidemia, hyperlipoproteinemia, hyperinsulinemia, atherosclerosis, diabetic neuropathy, diabetic retinopathy, metabolic syndrome, syndrome X, hypertension or pancreatic beta cell degeneration.

In an embodiment of the invention the disease or condition mediated by GPR40 is selected from: diabetes, obesity, insulin resistance, hyperglycemia, glucose intolerance, metabolic syndrome, syndrome X or pancreatic beta cell degeneration.

In an embodiment of the invention, diabetes is Type 2 diabetes.

In an embodiment the disease or condition mediated by GPR40 is a metabolic disorder which refers to one or more diseases or conditions as identified above.

Accordingly, the present invention relates to a method for the treatment of a metabolic disorder, comprising administering to a subject in need thereof a therapeutically amount of a compound of Formula (I) or a stereoisomer or a tautomer or a pharmaceutically acceptable salt thereof.

In an embodiment, the present invention provides use of the compound of Formula (I) or a stereoisomer or a tautomer or a pharmaceutically acceptable salt thereof for the treatment of a metabolic disorder.

According to one embodiment, the present invention relates to use of the compounds of Formula (I) or pharmaceutically acceptable salts thereof in the manufacture of a medicament, for the treatment of a metabolic disorder.

The term “metabolic disorder” as used herein refers a disorder relating to abnormality of metabolism. Accordingly, in the context of the present invention all the disorders relating to abnormality of metabolism are encompassed in the term “metabolic disorders”.

In one embodiment, the metabolic disorders are selected from diabetes, obesity, cardiovascular disease, hypertension, ketoacidosis, insulin resistance, glucose intolerance, hyperglycemia, hypertriglyceridemia, polycystic ovary syndrome, hypercholesterolemia, hyperlipoproteinemia, dyslipidemia, metabolic syndrome, syndrome X, hyperlipidemia, diabetic neuropathy, diabetic retinopathy, edema and related disorders associated with abnormal plasma lipoprotein, triglycerides or pancreatic beta cell degeneration.

The term “diabetes mellitus” or “diabetes” refers to a chronic disease or condition, which occurs when the pancreas does not produce enough insulin, or when the body cannot effectively use the insulin it produces. This leads to an increased concentration of glucose in the blood (hyperglycaemia). Two major forms of diabetes are Type 1 diabetes (Insulin-dependent diabetes mellitus) and Type 2 diabetes (Non-insulin dependent diabetes mellitus (NIDDM)). Type 1 diabetes is an autoimmune condition in which the insulin-producing β-cells of the pancreas are destroyed which generally results in an absolute deficiency of insulin, the hormone that regulates glucose utilization. Type 2 diabetes often occurs in the face of normal, or even elevated levels of insulin and can result from the inability of tissues to respond appropriately to insulin. Other categories of diabetes include gestational diabetes (a state of hyperglycemia which develops during pregnancy) and “other” rarer causes (genetic syndromes, acquired processes such as pancreatitis, diseases such as cystic fibrosis, exposure to certain drugs, viruses, and unknown causes). In an embodiment of the invention, diabetes refers to Type 2 diabetes.

The term “metabolic syndrome” refers to a cluster of metabolic abnormalities including abdominal obesity, insulin resistance, glucose intolerance, diabetes, hypertension and dyslipidemia. These abnormalities are known to be associated with an increased risk of vascular events.

The term “cardiovascular disease” as used herein refers to any disease of the heart or blood vessels. One or more diseases of heart encompassed in the term “cardiovascular disease” is selected from, but not limited to, angina, arrhythmia, coronary artery disease (CAD), cardiomyopathy, myocardial infarction, heart failure, hypertrophic cardiomyopathy, mitral regurgitation, mitral valve prolapse, pulmonary stenosis, etc. The blood vessel disease encompassed in the term “cardiovascular diseases”, is selected from, but not limited to, for example, peripheral vascular disease, artery disease, carotid artery disease, deep vein thrombosis, venous diseases, atherosclerosis and the like.

In an embodiment, the metabolic disorder is selected from: diabetes, obesity, insulin resistance, hyperglycemia, glucose intolerance, hypercholesterolemia, hypertriglyceridemia, dyslipidemia, hyperlipoproteinemia, hyperinsulinemia, atherosclerosis, diabetic neuropathy, diabetic retinopathy, metabolic syndrome, syndrome X, hypertension or pancreatic beta cell degeneration.

In an embodiment, the metabolic disorder is selected from diabetes, obesity, insulin resistance, glucose intolerance, dyslipidemia, hyperinsulinemia, syndrome X, metabolic syndrome or pancreatic beta cell degeneration.

In an embodiment, the metabolic disorder is Type 2 diabetes.

### Pharmaceutical Compositions

The present invention furthermore relates to pharmaceutical compositions that contain a therapeutically effective amount of at least one compound of Formula (I) or its physiologically tolerable salt in addition to a customary pharmaceutically acceptable carrier, and to a process for the production of a pharmaceutical composition, which includes bringing at least one compound of Formula (I), into a suitable administration form using a pharmaceutically suitable and physiologically tolerable excipient and, if appropriate, further suitable active compounds, additives or auxiliaries.

According to one embodiment, the present invention relates to a pharmaceutical composition comprising phenyl alkanoic acid derivatives, the compounds of Formula (I) or pharmaceutically acceptable salts thereof and a pharmaceutically acceptable excipient for use as GPR40 agonists and in the treatment of a disease or a condition mediated by GPR40.

The term “pharmaceutically acceptable” as used herein in the present invention means that the carrier, diluents, excipients, and/or salt must be compatible with the other ingredients of the formulation, and not deleterious to the recipient thereof.

The term “pharmaceutically acceptable carrier” as used herein means a non-toxic, inert, solid, semi-solid, diluent, encapsulating material or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose and cellulose acetate; malt; gelatin; talc; as well as other non-toxic compatible lubricants such as sodium lauryl sulfate and magnesium stearate, as well as coloring agents, releasing agents, coating agents, sweetening, flavoring and perfuming agents; preservatives and antioxidants can also be present in the composition, according to the judgment of the formulator.

It is further intended to include within the scope of the present invention the use of the compounds of Formula (I) or its pharmaceutically acceptable salts thereof in combination with at least one pharmacologically active compound as GPR40 agonists.

According to one embodiment, the present invention provides a pharmaceutical composition, comprising a therapeutically effective amount of a compound of Formula (I) or a pharmaceutically acceptable salt thereof and at least one further therapeutically active agent, together with a pharmaceutically acceptable carrier.

In an embodiment, the present invention relates to use of the compound of Formula (I) or a pharmaceutically acceptable salt thereof; in combination with a further therapeutically active compound, in the treatment of a disease or a condition mediated by GPR40.

The therapeutically active agent used in combination with one or more of the compounds of Formula (I) can be selected from the compounds or active substances known to be used in the treatment of diabetes and other conditions such as obesity, insulin resistance, hyperglycemia, glucose intolerance, hypercholesterolemia, hypertriglyceridemia, dyslipidemia, hyperlipoproteinemia, hyperinsulinemia or atherosclerosis. According to the present invention, the therapeutically active agent, used in combination with the compounds of Formula (I) of the present invention can be selected from, but not limited to, insulin, sulfonylureas, biguanidines, meglitinides, oxadiazolidinediones, thiazolidinediones, glucosidase inhibitors, inhibitors of glycogen phosphorylase, glucagon antagonists, HMGCoA reductase inhibitor, GLP-1 (Glucogen-like peptide-1) agonists, potassium channel openers, inhibitors of dipeptidylpeptidase IV (DPP-IV), insulin sensitizers, modulators of glucose uptake, of glucose transport and of glucose reabsorption, modulators of the sodium-dependent glucose transporter 1 or 2 (SGLT1, SGLT2), compounds which alter lipid metabolism such as antihyperlipidemic active ingredients and antilipidemic active ingredients, PPARgamma agonists and agents with combined PPARalpha and gamma activity and active ingredients which act on the ATP-dependent potassium channel of the beta cells.

In an embodiment, the compound of Formula (I) can be used in combination with a PPAR gamma agonist selected from rosiglitazone, pioglitazone, rivoglitazone and the like.

In an embodiment, the compound of Formula (I) can be used in combination with a HMGCoA reductase inhibitor selected from simvastatin, fluvastatin, pravastatin, lovastatin, atorvastatin, cerivastatin, rosuvastatin and the like.

In an embodiment, the compound of Formula (I) can be used in combination with a sulfonylurea selected from tolbutamide, glibenclamide, glipizide, glimepiride and the like.

In another embodiment, the compound of the Formula (I) can be used in combination with a meglitinide selected from repaglinide, nateglinide, mitiglinide and the like.

In another embodiment, the compound of the Formula (I) can be used in combination with GLP-1 agonist selected from exenatide, liraglutide, taspoglutide albiglutide, lixisenatide and the like.

In another embodiment, the compound of the Formula (I) can be used in combination with DPP-IV inhibitor selected from alogliptin, gemigliptin, linagliptin, saxagliptin, sitagliptin, vildagliptin and the like.

Accordingly, in an embodiment the further therapeutically active agent that can be used in combination with one or more compounds of Formula (I) encompassed in the present invention, can be selected from one or more of the agents including, but not limited to, insulin, rosiglitazone, pioglitazone, rivoglitazone, simvastatin, fluvastatin, pravastatin, lovastatin, atorvastatin, cerivastatin, rosuvastatin, tolbutamide, glibenclamide, glipizide, glimepiride, repaglinide, nateglinide, mitiglinide, exenatide, liraglutide, taspoglutide albiglutide, lixisenatide, alogliptin, gemigliptin, linagliptin, saxagliptin, sitagliptin, vildagliptin and the like.

The pharmaceutical compositions according to the present invention are prepared in a manner known and familiar to one skilled in the art. Pharmaceutically acceptable inert inorganic and/or organic carriers and/or additives can be used in addition to the compounds of Formula (I) and/or its pharmaceutically acceptable salts. For the production of pills, tablets, coated tablets and hard gelatin capsules it is possible to use, for example, lactose, corn starch or derivatives thereof, gum arabic, magnesia or glucose, etc. Carriers for soft gelatin capsules and suppositories are, for example, fats, waxes, natural or hardened oils, etc. Suitable carriers for the production of solutions, for example injection solutions, or of emulsions or syrups are, for example, water, physiological sodium chloride solution or alcohols, for example, ethanol, propanol or glycerol, sugar solutions, such as glucose solutions or mannitol solutions, or a mixture of the various solvents which have been mentioned.

Further, the pharmaceutical composition of the present invention also contains additives such as, for example, fillers, antioxidants, emulsifiers, preservatives, flavours, solubilisers or colourants. The pharmaceutical composition of the present invention may also contain two or more phenyl alkanoic acid derivatives i.e. compounds of Formula (I) and/or its physiologically tolerable salts, the pharmaceutical compositions can also contain one or more other therapeutically or prophylactically active ingredients.

The pharmaceutical compositions normally contain about 1 to 99%, for example, about 10 to 80%, by weight of the compounds of Formula (I) or their pharmaceutically acceptable salts.

The amount of the active ingredient, phenyl alkanoic acid derivative i.e. the compound of Formula (I) or its pharmaceutically acceptable salt in the pharmaceutical compositions can, for example, vary from about 1 to 500 mg. In case of higher body weight of the mammal in need of the treatment, the pharmaceutical composition may contain the compound of Formula (I) in an amount ranging from 5 mg to 1000 mg. The desirable dosage of the phenyl alkanoic acid derivatives i.e. the compounds of Formula (I) can be selected over a wide range. The daily dosage to be administered is selected to achieve the desired therapeutic effect in subjects being treated for metabolic disorders. A dosage of about 0.05 to 50 mg/kg/day of the phenyl alkanoic acid derivatives i.e. the compounds of Formula (I) or its pharmaceutically acceptable salt may be administered. In case of higher body weight of the mammal in need of the treatment, a dosage of about 0.1 to 100 mg/kg/day of the compound of Formula (I) or its pharmaceutically acceptable salt may be administered. If required, higher or lower daily dosages can also be administered. Actual dosage levels of the active ingredients in the pharmaceutical composition of this present invention can be varied so as to obtain an amount of the active ingredient, which is effective to achieve the desired therapeutic response for a particular patient, composition, and mode of administration without being toxic to the patient. The selected dosage level can be readily determined by a skilled medical practitioner in the light of the relevant circumstances, including the condition (diseases or disorder) to be treated, the chosen route of administration depending on a number of factors, such as age, weight and physical health and response of the individual patient, pharmacokinetics, severity of the disease and the like, factors known in the medical art.

The pharmaceutical compositions according to the present invention can be administered orally, for example in the form of pills, tablets, coated tablets, capsules, granules or elixirs. Administration, however, can also be carried out rectally, for example in the form of suppositories, or parenterally, for example intravenously, intramuscularly or subcutaneously, in the form of injectable sterile solutions or suspensions, or topically, for example in the form of solutions or transdermal patches, or in other ways, for example in the form of aerosols or nasal sprays.

It is understood that modifications that do not substantially affect the activity of the various embodiments of this invention are included within scope of the invention disclosed herein. Accordingly, the following examples are intended to illustrate but not to limit scope of the present invention.

### EXPERIMENTAL

The abbreviations and terms that are used herein:

LIST OF ABBREVIATIONS

| | | | |
|---|---|---|---|
| Cs₂CO₃ | Cesium carbonate | mm | Millimeter |
| DCM | Dichloromethane | μM | Micromolar |
| DMSO | Dimethyl sulfoxide | MeOH | Methanol |
| FBS | Fetal Bovine Serum | NaOH | Sodium hydroxide |
| h | Hour(s) | NaBH₄ | Sodium borohydride |
| HCl | Hydrochloric acid | NaHCO₃ | Sodium bicarbonate |
| LiOH | Lithium hydroxide | Na₂CO₃ | Sodium carbonate |
| KOH | Potassium hydroxide | Na₂SO₄ | Sodium sulfate |
| KHCO₃ | Potassium bicarbonate | NaCl | Sodium chloride |
| K₂CO₃ | Potassium carbonate | NH₄Cl | Ammonium chloride |
| KCl | Potassium chloride | μl | Microlitre |
| MgCl₂ | Magnesium chloride | ml | Millilitre |
| mM | Millimolar | LiCl | Lithium chloride |
| min | Minute | CaCl₂ | Calcium chloride |
| nM | Nanomolar | DMF | Dimethyl formamide |
| pM | Picomolar | THF | Tetrahydrofuran |
| μg | Microgram | TEA | Triethanolamine |
| mg | Milligram | DMAP | 4-Dimethylaminopyridine |
| g | Gram | EC₅₀ | Effective concentration (50%) |

| | |
|---|---|
| RT | Room temperature (20° C.-25° C.) |
| Ar | Argon |
| DIAD | Diisopropyl azodicarboxylate |
| DBU | 1,8-Diazabicyclo[5.4.0]undec-7-ene |
| HEPES | N-2-Hydroxyethylpiperazine-N′-2-ethanesulfonic acid |
| Oxone | 2KHSO₅•KHSO₄•K₂SO₄ (Potassium peroxymonosulfate) |
| PdCl₂(PPh₃)₂ | Bis(triphenylphosphine)palladium(II) dichloride |
| PPh₃ | Triphenyl phosphene |
| PPh₃CHCOOC₂H₅ | Ethyl 2-(triphenylphosphoranylidene) acetate |
| Rh(COD)₂Cl₂ | Cyclooctadiene rhodium chloride dimer |
| Pd(dppf)Cl₂•DCM | [1,1′- Bis(diphenylphosphino)ferrocene]dichloro |
| | palladium(II), complex with dichloromethane |
| Pd(PPh₃)₄ | Palladium tetrakistriphenylphosphine |

### Example 1

### Ethyl 2-(3-(4-((4′-(trifluoromethyl)biphenyl-3-yl)methoxy)phenyl)oxetan-3-yl)acetate (Compound 1)

### Step 1a

### Synthesis of Ethyl 2-(oxetan-3-ylidene)acetate

An ice cold solution of oxetone (5 g, 69.4 mM) in anhydrous DCM (70 ml) was treated with the reagent, PPh₃CHCOOC₂H₅ (26.6 g, 76 mM). The reaction mixture obtained was allowed to warm to RT and stirred for 1 h. The reaction mixture was concentrated to obtain a crude product, which was purified by column chromatography (silica gel, 100-200 mesh, eluted with 3% ethyl acetate in petroleum ether) to afford the title compound (5.99 g) as colorless oil. Yield: 60.7%; ¹H NMR (CDCl₃, 300 MHz): δ 5.64 (bs, 1H), 5.53-5.51 (m, 2H), 5.32-5.31 (m, 2H), 4.18 (q, J=6.89, 2H), 1.28 (t, J=6.89, 3H); MS: m/z 143 (M+1).

### Step 1b

### Synthesis of Ethyl 2-(3-(4-hydroxyphenyl)oxetan-3-yl)acetate

Aqueous KOH (46.9 ml, 70.3 mM) was added to a suspension of Rh(COD)₂Cl₂ in dioxane (15 ml) and the mixture was stirred for 10 min. (4-hydroxyphenyl)boronic acid (9.70 g, 70.3 mM) and successively ethyl 2-(oxetan-3-ylidene)acetate (compound of Step 1a, 5 g, 35.2 mM) in dioxane were added and the reaction mixture was stirred for 6 h. The reaction mixture was extracted using ethyl acetate (30×3 ml). The organic layer was washed with brine, dried over Na₂SO₄ and concentrated to obtain a crude product, which was purified by column chromatography (silica gel, 100-200 mesh, eluted with 10% ethyl acetate in hexane) to afford ethyl 2-(3-(4-hydroxyphenyl)oxetan-3-yl)acetate. Yield: 4.2 g (50.5%); ¹H NMR (CDCl₃, 300 MHz): δ 7.07 (d, J=8.4 Hz, 2H), 6.81 (d, J=8.4 Hz, 2H), 5.18 (bs, 1H), 5.01 (d, J=6.0 Hz, 2H), 4.88 (d, J=6.0 Hz, 2H), 4.07 (q, J=6.90 Hz, 2H), 3.11 (s, 2H), 1.15 (t, J=6.90 Hz, 3H); MS: m/z 259 (M+Na).

### Step 1c

### Synthesis of 3-(Bromomethyl)-4′-(trifluoromethyl)-1,1′-biphenyl

### Step 1c′: Synthesis of (4′-(Trifluoromethyl)-[1,1′-biphenyl]-3-yl)methanol

To a solution of 3-bromo benzyl alcohol (0.1 g, 0.53 mM) and 4-(trifluoromethyl)phenyl)boronic acid (0.121 g, 0.64 mM), DMF/water (8:1), Na₂CO₃ (0.142 g, 1.33 mM) and PdCl₂(PPh₃)₂ (0.010 mM) were added. The reaction mixture was heated in a microwave at 110° C. for 6 min. The reaction mixture was quenched with water and extracted with ethyl acetate (3×10 ml). The organic layer was washed with brine, dried over Na₂SO₄ and concentrated to obtain a crude product, which was purified by column chromatography (silica gel, 100-200 mesh, eluted with 15% ethyl acetate in petroleum ether) to afford the title compound (4′-(trifluoromethyl)-[1,1′-biphenyl]-3-yl)methanol as a colorless oil. Yield: 0.108 g (80%); ¹H NMR (DMSO-d₆, 300 MHz): δ 7.91 (d, J=8.1 Hz, 2H), 7.84-7.82 (m, 3H), 7.70 (m, 1H), 7.50-7.48 (m, 2H), 5.30 (t, J=5.7 Hz, 1H, OH), 4.60 (d, J=5.7 Hz, 2H); MS: m/z 275 (M+Na).

### Step 1c″

### Synthesis of 3-(Bromomethyl)-4′-(trifluoromethyl)-1,1′-biphenyl

Carbon tetrabromide (263 mg, 0.793 mM) was added to a solution of (4′-(trifluoromethyl)-[1,1′-biphenyl]-3-yl)methanol (compound of Step 1c′, 100 mg, 0.396 mM) and triphenyl phosphene (260 mg, 0.991 mM) in DCM (4 ml) at 0° C. The reaction mixture was stirred at 0° C. for 15 min, allowed to warm to RT and stirred for 1 h. The solvent was evaporated, and the crude product obtained was purified by column chromatography (silica gel, 100-200 mesh, eluted with 5% ethyl acetate in petroleum ether) to afford 3-(bromomethyl)-4′-(trifluoromethyl)-1,1′-biphenyl as a white solid. Yield: 94 mg (75%); ¹H NMR (DMSO-d₆, 300 MHz): δ 7.91 (d, J=8.1 Hz, 2H), 7.84-7.82 (m, 3H), 7.70-7.69 (m, 1H), 7.5-7.48 (m, 2H), 4.80 (s, 2H); MS: m/z 315 (M+).

### Step 1d

### Ethyl 2-(3-(4-((4′-(trifluoromethyl)biphenyl-3-yl)methoxy)phenyl)oxetan-3-yl)acetate (Compound 1)

To a solution of ethyl 2-(3-(4-hydroxyphenyl)oxetan-3-yl)acetate (compound of Step 1b, 60 mg, 0.254 mM) and 3-(bromomethyl)-4′-(trifluoromethyl)-1,1′-biphenyl (compound of Step 1c″, 80 mg, 0.254 mM) in anhydrous DMF (2 ml) was added Cs₂CO₃ (165 mg, 0.508 mM) at RT. The reaction mixture was stirred at RT for 2 h, quenched with water (5 ml), further stirred for 10 min and extracted with ethyl acetate. The organic layer was washed with brine, dried with Na₂SO₄ and concentrated under reduced pressure. The crude product was purified by flash column chromatography (silica gel, 100-200 mesh, eluted with 10% ethyl acetate in petroleum ether) to afford the title compound. Yield: 95%; ¹H NMR (DMSO-d₆, 300 MHz): δ 7.92 (d, J=8.1 Hz, 2H), 7.84 (d, J=8.4 Hz, 3H), 7.71 (m, 1H), 7.54 (d, J=5.7 Hz, 2H), 7.19 (d, J=8.4 Hz, 2H), 7.02 (d, J=8.7 Hz, 2H), 5.19 (s, 2H), 4.75 (s, 4H), 3.92 (q, J=6.9 Hz, 2H), 3.08 (s, 2H), 1.03 (t, J=6.9 Hz, 3H); MS: m/z 494 (M+Na).

### Example 2

### 2-(3-(4-((4′-(Trifluoromethyl)biphenyl-3-yl)methoxy)phenyl)oxetan-3-yl)acetic acid (Compound 2)

To a solution of 2-(3-(4-((4′-(trifluoromethyl)biphenyl-3-yl)methoxy)phenyl)oxetan-3-yl)acetate (compound of Example 1, 50 mg, 0.110 mM) in THF (2 ml) and MeOH (0.5 ml) was added LiOH (0.222 μl, 0.333 mM) and the reaction mixture was stirred at RT for 2 to 3 h. The solvent was removed, the reaction mixture was neutralized with 1M HCl and extracted with ethyl acetate. The organic layer was washed with brine, dried with Na₂SO₄ and concentrated to obtain a crude product, which was purified by washing with acetonitrile or by flash column chromatography (silica gel, eluted with 5% MeOH in chloroform) to afford the title compound. Yield: 31%; ¹H NMR (DMSO-d₆, 300 MHz): δ 12.13 (s, 1H), 7.93 (d, J=8.1 Hz, 2H), 7.84 (d, J=7.2 Hz, 2H), 7.70 (s, 2H), 7.53 (s, 2H), 7.23 (d, J=8.4 Hz, 2H), 7.03 (d, J=8.4 Hz, 2H), 5.18 (s, 2H), 4.74 (s, 4H), 3.01 (s, 2H); MS: m/z 442 (M+1).

The compounds of examples 3, 5, 7, 9, 11, 13, 15, 17, 19, 21, 23, 25, 27, 29, 31, 33, 35, 37 and 90 were prepared by following the procedure exemplified in Example 1. The compounds of examples 4, 6, 8, 10, 12, 14, 16, 18, 20, 22, 24, 26, 28, 30, 32, 34, 36, 38 and 91 were prepared by following the procedure exemplified in Example 2. The characterisation data for the compounds of examples 3 to 38 is described below.

### Example 3

### Ethyl 2-(3-(4-([1,1′-biphenyl]-3-ylmethoxy)phenyl)oxetan-3-yl)acetate (Compound 3)

The title compound was prepared in an analogous manner as the Compound 1 of Example 1 involving reaction of ethyl 2-(3-(4-hydroxyphenyl)oxetan-3-yl)acetate (compound of Step 1b of Example 1) with 3-phenyl benzylbromide. Yield: 71%; ¹H NMR (CDCl₃, 300 MHz): δ 7.67 (d, J=8.9 Hz, 1H), 7.61 (d, J=8.7 Hz, 2H), 7.48-7.38 (m, 6H), 7.14 (d, J=8.4 Hz, 2H), 7.00 (d, J=8.4 Hz, 2H), 5.13 (s, 2H), 5.01 (d, J=6.0 Hz, 2H), 4.88 (d, J=6.0 Hz, 2H) 4.06 (q, J=7.2 Hz, 2H), 3.11 (s, 2H), 1.13 (t, J=7.2 Hz, 3H); MS: m/z 403 (M+1) and 425 (M+Na).

### Example 4

### 2-(3-(4-([1,1′-Biphenyl]-3-ylmethoxy)phenyl)oxetan-3-yl)acetic acid (Compound 4)

The title compound was prepared in an analogous manner as the compound 2 of Example 2. Compound 4 was obtained by hydrolyzing the compound of Example 3. Yield: 43%; ¹H NMR (CDCl₃, 300 MHz): δ 7.66-7.56 (m, 4H), 7.46-7.37 (d, J=8.7 Hz, 5H), 7.15 (d, J=8.1 Hz, 2H), 7.00 (d, J=8.4 Hz, 2H), 5.12 (m, 2H), 5.01 (d, J=6.0 Hz, 2H), 4.88 (d, J=6.0 Hz, 2H) 3.17 (s, 2H); MS: m/z 375 (M+1).

### Example 5

### Ethyl 2-(3-(4-((2′-cyano-[1,1′-biphenyl]-4-yl)methoxy)phenyl)oxetan-3-yl)acetate (Compound 5)

The title compound was prepared in an analogous manner as the compound 1 of Example 1 involving reaction of ethyl 2-(3-(4-hydroxyphenyl)oxetan-3-yl)acetate (compound of Step 1b of Example 1) with 4′-(bromomethyl)-[1,1′-biphenyl]-2-carbonitrile. Yield: 72%; ¹H NMR (DMSO-d₆, 300 MHz): δ 7.98 (d, J=7.8 Hz, 1H), 7.83 (t, J=7.5 Hz, 1H), 7.66-7.57 (m, 6H), 7.21 (d, J=8.4 Hz, 2H), 7.04 (d, J=8.7 Hz, 2H), 5.19 (s, 2H), 4.76 (s, 4H), 3.94 (q, J=6.9 Hz, 2H), 3.09 (s, 2H), 1.06 (t, J=7.2 Hz, 3H); MS: m/z 428 (M+1).

### Example 6

### 2-(3-(4-((2′-Cyano-[1,1′-biphenyl]-4-yl)methoxy)phenyl)oxetan-3-yl)acetic acid (Compound 6)

The title compound was prepared in an analogous manner as the compound 2 of Example 2. Compound 6 was obtained by hydrolyzing compound of Example 5. Yield: 62%; ¹H NMR (DMSO-d₆, 300 MHz): δ 12.14 (s, 1H), 7.98 (d, J=7.8 Hz, 1H), 7.83 (t, J=7.8 Hz, 1H), 7.66 (m, 6H), 7.25 (d, J=8.4 Hz, 2H), 7.04 (d, J=8.4 Hz, 2H), 5.18 (s, 2H), 4.75 (s, 4H), 3.02 (s, 2H); MS: m/z 400 (M+1).

### Example 7

### Ethyl 2-(3-(4-([1,1′-biphenyl]-4-ylmethoxy)phenyl)oxetan-3-yl)acetate (Compound 7)

The title compound was prepared in an analogous manner as the compound 1 of Example 1 involving reaction of ethyl 2-(3-(4-hydroxyphenyl)oxetan-3-yl)acetate (compound of Step 1b of Example 1) with 4-phenylbenzylbromide. Yield: 99%; ¹H NMR (DMSO-d₆, 300 MHz): δ 7.70-7.67 (m, 4H), 7.54-7.45 (m, 4H), 7.39 (d, J=6.9 Hz, 1H), 7.19 (d, J=8.4 Hz, 2H), 7.04 (d, J=8.4 Hz, 2H), 5.14 (s, 2H), 4.76 (s, 4H), 3.94 (q, J=7.2 Hz, 2H), 3.08 (s, 2H), 1.05 (t, J=6.9 Hz, 3H); MS: m/z 402 (M+1).

### Example 8

### 2-(3-(4-([1,1′-Biphenyl]-4-ylmethoxy)phenyl)oxetan-3-yl)acetic acid (Compound 8)

The title compound was prepared in an analogous manner as the compound 2 of Example 2. Compound 8 was obtained by hydrolyzing the compound of Example 7. Yield: 74%; ¹H NMR (DMSO-d₆, 300 MHz): δ 12.13 (s, 1H), 7.70 (m, 4H), 7.55 (d, J=8.1 Hz, 2H), 7.50 (t, J=7.5 Hz, 2H), 7.39 (m, 1H), 7.23 (d, J=8.4 Hz, 2H), 7.02 (d, J=8.7 Hz, 2H), 5.14 (s, 2H), 4.75 (s, 4H), 3.02 (s, 2H); MS: m/z 375 (M+1).

### Example 9

### Ethyl 2-(3-(4-((2′,6′-dimethyl-4′-(3-(methylsulfonyl)propoxy)-[1,1′-biphenyl]-3-yl) methoxy)phenyl)oxetan-3-yl)acetate (Compound 9)

The title compound was prepared in an analogous manner as the compound 1 of Example 1 involving reaction of ethyl 2-(3-(4-hydroxyphenyl)oxetan-3-yl)acetate (compound of Step 1b of Example 1) with 3′-(bromomethyl)-2,6-dimethyl-4-(3-(methylsulfonyl)propoxy)-1,1′-biphenyl. The compound 3′-(bromomethyl)-2,6-dimethyl-4-(3-(methylsulfonyl)propoxy)-1,1′-biphenyl was prepared in accordance with Step 1c″ of Example 1 by reacting (2′,6′-dimethyl-4′-(3-(methylsulfonyl)propoxy)-[1,1′-biphenyl]-3-yl)methanol with carbon tetrabromide. (2′,6′-dimethyl-4′-(3-(methylsulfonyl)propoxy)-[1,1′-biphenyl]-3-yl)methanol was prepared in accordance with the method described in PCT published application No. WO2008001931 A2. Yield: 54%; ¹H NMR (DMSO-d₆, 300 MHz): δ 7.45-7.38 (m, 3H), 7.16 (d, J=6.3 Hz, 2H), 7.07 (d, J=6.9 Hz, 1H), 6.99 (d, J=8.1 Hz, 2H), 6.71 (s, 2H), 5.14 (s, 2H), 4.75 (s, 4H), 4.09 (s, 2H), 3.90 (q, J=6.9 Hz, 2H), 3.33 (m, 2H), 3.07 (m, 2H), 3.03 (s, 3H), 2.14 (s, 2H), 1.91 (s, 6H), 1.04 (t, J=6.9 Hz, 3H); MS: m/z 567 (M+1).

### Example 10

### 2-(3-(4-((2′,6′-Dimethyl-4′-(3-(methylsulfonyl)propoxy)-[1,1′-biphenyl]-3-yl)methoxy)phenyl)oxetan-3-yl)acetic acid (Compound 10)

The title compound was prepared in an analogous manner as the compound 2 of Example 2. The title compound was obtained by hydrolyzing the compound of Example 9. Yield: 53%; ¹H NMR (CDCl₃, 300 MHz): δ 7.47 (m, 2H), 7.17-7.08 (m, 4H), 6.96 (d, J=8.4 Hz, 2H), 6.65 (s, 2H), 5.11 (s, 2H), 4.99 (d, J=6 Hz, 2H), 4.85 (d, J=5.7 Hz, 2H), 4.14 (t, J=5.4 Hz, 2H), 3.31 (t, J=7.2 Hz, 2H), 3.16 (s, 2H), 2.98 (s, 3H), 2.36 (s, 2H), 1.99 (s, 6H); MS: m/z 539.3 (M+1).

### Example 11

### Ethyl 2-(3-(4-([1,1′-biphenyl]-3-ylmethoxy)-3-fluorophenyl)oxetan-3-yl)acetate (Compound 11)

The title compound was prepared in an analogous manner as the compound 1 of Example 1 by involving reaction of 2-(3-(3-fluoro-4-hydroxyphenyl)oxetan-3-yl)acetate and 3-phenyl benzyl bromide. The compound, 2-(3-(3-fluoro-4-hydroxyphenyl)oxetan-3-yl)acetate was prepared by following the procedure depicted in Step 1b of Example 1 involving reaction of (3-fluoro-4-hydroxyphenyl)boronic acid with ethyl 2-(oxetan-3-ylidene)acetate. Yield: 86%; ¹H NMR (CDCl₃, 300 MHz): δ 7.67-7.57 (m, 4H), 7.47-7.37 (m, 5H), 7.04-6.95 (m, 2H), 6.87 (d, J=8.1 Hz, 1H), 5.21 (s, 2H), 4.95 (d, J=6.0 Hz, 2H), 4.84 (d, J=6.0 Hz, 2H), 4.03 (q, J=7.2 Hz, 2H), 3.10 (s, 2H), 1.14 (t, J=6.9 Hz, 3H); MS: m/z 421.2 (M+1), 443.2 (M+Na).

### Example 12

### 2-(3-(4-([1,1′-Biphenyl]-3-ylmethoxy)-3-fluorophenyl)oxetan-3-yl)acetic acid (Compound 12)

The title compound was prepared in an analogous manner as the compound 2 of Example 2. Compound 12 was obtained by hydrolyzing the compound of Example 11. Yield: 87%; ¹H NMR (CDCl₃, DMSO-d₆, 300 MHz): δ 12.18 (bs, 1H), 7.75 (s, 1H), 7.69-7.64 (m, 3H), 7.53-7.36 (m, 5H), 7.25-7.17 (m, 2H), 7.05 (d, J=8.4 Hz, 1H), 5.25 (s, 2H), 4.37 (s, 4H), 3.04 (s, 2H); MS (ESI): m/z 393.2 (M+1), 390.8 (M−1).

### Example 13

### Ethyl 2-(3-(4-([1,1′-biphenyl]-4-ylmethoxy)-3-fluorophenyl)oxetan-3-yl)acetate (Compound 13)

The title compound was prepared in an analogous manner as the compound 1 of Example 1 by involving reaction of 2-(3-(3-fluoro-4-hydroxyphenyl)oxetan-3-yl)acetate (described in Example 11) with 4-phenyl benzyl bromide. Yield: 76%; ¹H NMR (CDCl₃, 300 MHz): δ 7.62 (s, 4H), 7.54-7.37 (m, 5H), 7.04-6.87 (m, 3H), 5.19 (s, 2H), 4.95 (d, J=5.7 Hz, 2H), 4.85 (d, J=5.7 Hz, 2H), 4.03 (q, J=6.9 Hz, 2H), 3.10 (s, 2H), 1.14 (t, J=6.9 Hz, 3H); MS (ESI): m/z 421.2 (M+1), 443.2 (M+Na).

### Example 14

### 2-(3-(4-([1,1′-Biphenyl]-4-ylmethoxy)-3-fluorophenyl)oxetan-3-yl)acetic acid (Compound 14)

The title compound was prepared in an analogous manner as the compound 2 of Example 2. Compound 14 was obtained by hydrolyzing the compound of Example 13. Yield: 87%; ¹H NMR (CDCl₃, DMSO-d₆, 300 MHz): δ 11.62 (bs, 1H), 7.59-7.55 (m, 4H), 7.48 (d, J=7.8 Hz, 2H), 7.41 (t, J=7.2 Hz, 2H), 7.32 (d, J=7.2 Hz, 1H), 7.04-6.87 (m, 3H), 5.13 (s, 2H), 4.88 (d, J=5.7 Hz, 2H), 4.82 (d, J=5.7 Hz, 2H), 3.04 (s, 2H); MS: m/z 393.3 (M+1).

### Example 15

### Ethyl 2-(3-(4-((2′,6′-dimethyl-4′-(3-(methylsulfonyl)propoxy)-[1,1′-biphenyl]-3-yl) methoxy)-3-fluorophenyl)oxetan-3-yl)acetate (Compound 15)

The title compound was prepared in an analogous manner as the compound 1 of Example 1 involving reaction of 2-(3-(3-fluoro-4-hydroxyphenyl)oxetan-3-yl)acetate with 3′-(bromomethyl)-2,6-dimethyl-4-(3-(methylsulfonyl)propoxy)-1,1′-biphenyl. The compound, 3′-(Bromomethyl)-2,6-dimethyl-4-(3-(methylsulfonyl) propoxy)-1,1′-biphenyl was prepared as per the method described in Example 9. Yield: 79%; ¹H NMR (CDCl₃, 300 MHz): δ 7.45-7.42 (m, 2H), 7.18 (s, 1H), 7.10 (d, J=6.3 Hz, 1H), 6.95 (d, J=9.3 Hz, 2H), 6.85 (d, J=8.1 Hz, 1H), 6.66 (s, 2H), 5.18 (s, 2H), 4.94 (d, J=6.0 Hz, 2H), 4.84 (d, J=6.0 Hz, 2H), 4.12 (t, J=5.4 Hz, 2H), 4.03 (q, J=6.9 Hz, 2H), 3.29 (t, J=7.2 Hz, 2H), 3.09 (s, 2H), 2.99 (s, 3H), 2.39-2.37 (m, 2H), 1.99 (s, 6H), 1.14 (t, J=7.2 Hz, 3H); MS (ESI): m/z 585.3 (M+1), 583.3 (M−1).

### Example 16

### 2-(3-(4-((2′,6′-Dimethyl-4′-(3-(methylsulfonyl)propoxy)-[1,1′-biphenyl]-3-yl)methoxy)-3-fluorophenyl)oxetan-3-yl)acetic acid (Compound 16)

The title compound was prepared in an analogous manner as the compound 2 of Example 2. Compound 16 was obtained by hydrolyzing the compound of Example 15. Yield: 79%; ¹H NMR (CDCl₃, 300 MHz): δ 7.14-7.42 (m, 2H), 7.16 (s, 1H), 7.09 (d, J=6.3 Hz, 1H), 6.99-6.93 (m, 2H), 6.84 (d, J=8.1 Hz, 1H), 6.65 (s, 2H), 5.18 (s, 2H), 4.93 (d, J=6.0 Hz, 2H), 4.81 (d, J=6.0 Hz, 2H), 4.14 (t, J=5.3 Hz, 2H), 3.29 (t, J=7.2 Hz, 2H), 3.13 (s, 2H), 2.98 (s, 3H), 2.38-2.35 (m, 2H), 1.97 (s, 6H); MS: m/z 557.3 (M+1), 555.3 (M−1).

### Example 17

### Ethyl 2-(3-(4-((5,5,8,8-tetramethyl-5,6,7,8-tetrahydronaphthalen-2-yl)methoxy)phenyl)oxetan-3-yl)acetate (Compound 17)

The title compound was prepared in an analogous manner as the compound 1 of Example 1 by involving reaction of ethyl 2-(3-(4-hydroxyphenyl)oxetan-3-yl)acetate (compound of Step 1b of Example 1) and commercially available 6-(bromomethyl)-1,1,4,4-tetramethyl-1,2,3,4-tetrahydronaphthalene. The title compound was obtained as a colorless oil. Yield: 64.3%; ¹H NMR (CDCl₃, 300 MHz): δ 7.40-7.38 (m, 1H), 7.36 (d, J=6.9 Hz, 1H), 7.23 (d, J=6.9 Hz, 1H), 7.13 (d, J=8.4 Hz, 2H), 6.99 (d, J=8.4 Hz, 2H), 5.02-4.99 (m, 4H), 4.88 (d, J=6.0 Hz, 2H) 4.06 (q, J=7.2 Hz, 2H), 3.11 (s, 2H), 1.71 (s, 4H), 1.30 (s, 12H), 1.15 (t, J=7.2 Hz, 3H); MS: m/z 437 (M+1).

### Example 18

### 2-(3-(4-((5,5,8,8-Tetramethyl-5,6,7,8-tetrahydronaphthalen-2-yl)methoxy)phenyl)oxetan-3-yl)acetic acid (Compound 18)

The title compound was prepared in an analogous manner as the compound 2 of Example 2. Compound 18 was obtained by hydrolysis of the compound of Example 17. Yield: 62.3%; ¹H NMR (CDCl₃, 300 MHz): δ 7.36-7.30 (m, 2H), 7.23 (d, J=6.9 Hz, 1H), 7.15 (d, J=8.1 Hz, 2H), 7.00 (d, J=8.1 Hz, 2H), 5.01-4.97 (m, 4H), 4.86 (d, J=6.0 Hz, 2H), 3.17 (s, 2H), 1.71 (s, 4H), 1.30 (s, 12H); MS: m/z 408 (M+).

### Example 19

### Ethyl 2-(3-(3-fluoro-4-((5,5,8,8-tetramethyl-5,6,7,8-tetrahydronaphthalen-2-yl) methoxy)phenyl)oxetan-3-yl)acetate (Compound 19)

The title compound was prepared in an analogous manner as the compound 1 of Example 1 by reaction of ethyl 2-(3-(3-fluoro-4-hydroxyphenyl)oxetan-3-yl)acetate (compound of Step 1b of Example 1) and commercially available 6-(bromomethyl)-1,1,4,4-tetramethyl-1,2,3,4-tetrahydronaphthalene. Yield: 95%; ¹H NMR (CDCl₃, 300 MHz): δ 7.34 (d, J=8.7 Hz, 2H), 7.22 (d, J=7.8 Hz, 1H), 7.01-6.86 (m, 3H), 5.07 (s, 2H), 4.95 (d, J=5.7 Hz, 2H), 4.85 (d, J=5.7 Hz, 2H), 4.04 (q, J=6.9 Hz, 2H), 3.10 (s, 2H) 1.70 (s, 4H), 1.29 (s, 12H), 1.15 (t, J=6.9 Hz, 3H); MS (ESI): m/z 455 (M+1).

### Example 20

### 2-(3-(3-Fluoro-4-((5,5,8,8-tetramethyl-5,6,7,8-tetrahydronaphthalen-2-yl)methoxy)phenyl)oxetan-3-yl)acetic acid (Compound 20)

The title compound was prepared in an analogous manner as the compound 2 of Example 2. Compound 20 was obtained by hydrolyzing the compound of Example 19. Yield: 85%; ¹H NMR (DMSO-d₆, 300 MHz): δ 12.18 (bs, 1H), 7.40 (s, 1H), 7.34 (d, J=8.1 Hz, 1H), 7.26-7.15 (m, 3H), 7.05 (d, J=8.1 Hz, 1H), 5.07 (s, 2H), 4.73 (s, 4H), 3.03 (s, 2H), 1.64 (s, 4H), 1.24 (s, 12H); MS (ESI): 449.2 (M+Na).

### Example 21

### Ethyl 2-(3-(4-((4-methoxy-3-(trifluoromethyl)benzyl)oxy)phenyl)oxetan-3-yl)acetate (Compound 21)

The title compound was prepared in an analogous manner as the compound 1 of Example 1 by involving reaction of ethyl 2-(3-(4-hydroxyphenyl)oxetan-3-yl)acetate (compound of Step 1b of Example 1) and 4-methoxy-3-trifluoromethyl benzylbromide. Yield: 77%; ¹H NMR (CDCl₃, 300 MHz): δ 7.65 (s, 1H), 7.59 (d, J=8.4 Hz, 1H), 7.14 (d, J=8.7 Hz, 2H), 7.05 (d, J=8.7 Hz, 2H), 6.96 (d, J=8.4 Hz, 2H), 5.01 (s including d at 4.99, J=6.0 Hz, 3H), 4.87 (d, J=6.0 Hz, 2H), 4.06 (q, J=6.9 Hz, 2H), 3.93 (s, 3H), 3.11 (s, 2H), 1.14 (t, J=7.2 Hz, 3H); MS: m/z 424 (M+).

### Example 22

### 2-(3-(4-(4-Methoxy-3-(trifluoromethyl)benzyl)oxy)phenyl)oxetan-3-yl)acetic acid (Compound 22)

The title compound was prepared in an analogous manner as the compound 2 of Example 2. Compound 22 was obtained by hydrolyzing the compound of Example 21. Yield: 37%; ¹H NMR (DMSO-d₆, 300 MHz): δ 12.14 (s, 1H), 7.74 (bs, 2H), 7.30 (d, J=9 Hz, 1H), 7.22 (d, J=8.4 Hz, 2H), 6.99 (d, J=8.4 Hz, 2H), 5.08 (s, 2H), 4.74 (s, 4H), 3.89 (s, 3H), 3.01 (s, 2H); MS: m/z 419 (M+Na).

### Example 23

### Ethyl 2-(3-(4-((2-methyl-5-(trifluoromethyl)benzyl)oxy)phenyl)oxetan-3-yl)acetate (Compound 23)

The title compound was prepared in an analogous manner as the compound 1 of Example 1 involving reaction of ethyl 2-(3-(4-hydroxyphenyl)oxetan-3-yl)acetate (compound of Step 1b of Example 1) and 2-methyl-5-trifluoromethyl benzylbromide. Yield: 73%; ¹H NMR (CDCl₃, 300 Hz): δ 7.71 (s, 1H), 7.54 (d, J=7.8 Hz, 1H), 7.36 (d, J=7.8 Hz, 1H), 7.16 (d, J=8.4 Hz, H), 7.00 (d, J=8.4 Hz, 2H), 5.06 (s, 2H), 5.02 (d, J=6.0 Hz, 2H), 4.88 (d, J=6.0 Hz, 2H), 4.04 (q, J=6.9 Hz, 2H), 3.12 (s, 2H), 2.44 (s, 3H), 1.14 (t, J=6.0 Hz, 3H); MS: m/z 408 (M+).

### Example 24

### 2-(3-(4-(2-Methyl-5-(trifluoromethyl)benzyl)oxy)phenyl)oxetan-3-yl)acetic acid (Compound 24)

The title compound was prepared in a manner analogous to compound 2 of Example 2. Compound 24 was obtained by hydrolyzing the compound of Example 23. Yield: 100%; ¹H NMR (DMSO-d₆, 300 MHz): δ 12.14 (s, 1H), 7.78 (s, 1H), 7.63 (d, J=7.8 Hz, 1H), 7.49 (d, J=7.8 Hz, 1H), 7.25 (d, J=8.4 Hz, 2H), 7.05 (d, J=8.4 Hz, 2H), 5.16 (s, 2H), 4.75 (s, 4H), 3.03 (s, 2H), 2.41 (s, 3H); MS: m/z 380 (M+1).

### Example 25

### Ethyl 2-(3-(4-((2-methoxy-5-(trifluoromethyl)benzyl)oxy)phenyl)oxetan-3-yl)acetate (Compound 25)

The title compound was prepared in an analogous manner as the compound 1 of Example 1 by involving reaction of ethyl 2-(3-(4-hydroxyphenyl)oxetan-3-yl)acetate (compound of Step 1b of Example 1) and 2-methoxy-5-trifluoromethyl benzylbromide. Yield: 97%; ¹H NMR (DMSO-d₆, 300 MHz): δ 7.73 (s, 2H), 7.27 (d, J=9 Hz, 1H), 7.19 (d, J=8.4 Hz, 2H), 7.01 (d, J=8.4 Hz, 2H), 5.09 (s, 2H), 4.76 (s, 4H), 3.91 (m, 5H), 3.08 (s, 2H), 1.05 (t, J=6.9 Hz, 3H; MS: m/z 448 (M+Na).

### Example 26

### 2-(3-(4-(2-Methoxy-5-(trifluoromethyl)benzyl)oxy)phenyl)oxetan-3-yl)acetic acid (Compound 26)

The title compound was prepared in an analogous manner as the compound 2 of Example 2. Compound 26 was prepared by hydrolyzing the compound of Example 25. Yield: 41%; ¹H NMR (CDCl₃, 300 MHz): δ 7.76 (s, 1H), 7.60 (d, J=8.1 Hz, 1H), 7.14 (s, 2H), 7.01-6.96 (m, 3H), 5.09 (s, 2H), 5.00 (s, 2H), 4.86 (s, 2H), 3.92 (s, 3H), 3.18 (s, 2H); MS: m/z 394 (M−2).

### Example 27

### Ethyl 2-(3-(4-((4-methyl-3-(trifluoromethyl)benzyl)oxy)phenyl)oxetan-3-yl)acetate (Compound 27)

The title compound was prepared in an analogous manner as the compound 1 of Example 1 by involving reaction of ethyl 2-(3-(4-hydroxyphenyl)oxetan-3-yl)acetate (compound of Step 1b of Example 1) and 4-methyl-3-trifluoromethyl benzylbromide. Yield: 90%; ¹H NMR (DMSO-d₆, 300 MHz): δ 7.74 (s, 1H), 7.64 (d, J=7.5 Hz, 1H), 7.47 (d, J=7.8 Hz, 1H), 7.18 (d, J=8.4 Hz, 2H), 6.99 (d, J=8.4 Hz, 2H), 5.15 (s, 2H), 4.75 (s, 4H), 3.93 (q, J=7.2 Hz, 2H), 3.08 (s, 2H), 2.44 (s, 3H), 1.03 (t, J=6.9 Hz, 3H); MS: m/z 432 (M+Na).

### Example 28

### 2-(3-(4-((4-Methyl-3-(trifluoromethyl)benzyl)oxy)phenyl)oxetan-3-yl)acetic acid (Compound 28)

The title compound was prepared in an analogous manner as the compound 2 of Example 2. Compound 28 was obtained by hydrolyzing the compound of Example 27. Yield: 98%; ¹H NMR (CDCl₃, 300 MHz): δ 7.67 (s, 1H), 7.50 (d, J=7.5 Hz, 1H), 7.32 (s, 1H), 7.15 (d, J=8.4 Hz, 2H), 6.97 (d, J=8.4 Hz, 2H), 5.04 (s, 2H), 5.00 (d, J=6 Hz, 2H), 4.86 (d, J=6 Hz, 2H), 3.17 (s, 2H), 2.51 (s, 3H); MS: m/z 403 (M+Na).

### Example 29

### Ethyl 2-(3-(4-(3-methoxy-4-(trifluoromethyl)benzyl)oxy)phenyl)oxetan-3-yl)acetate (Compound 29)

The title compound was prepared in an analogous manner as the compound 1 of Example 1 by involving reaction of ethyl 2-(3-(4-hydroxyphenyl)oxetan-3-yl)acetate (compound of Step 1b of Example 1) and 4-trifluoromethyl-3-methoxy benzylbromide. Yield: 96%; ¹H NMR (DMSO-d₆, 300 MHz): δ 7.63 (d, J=7.8 Hz, 1H), 7.33 (s, 1H), 7.19 (m, 3H), 7.01 (d, J=8.4 Hz, 2H), 5.18 (s, 2H), 4.75 (s, 4H), 3.89 (m, 5H), 3.08 (s, 2H), 1.03 (t, J=6.9 Hz, 3H); MS: m/z 425 (M+1).

### Example 30

### 2-(3-(4-((3-Methoxy-4-(trifluoromethyl)benzyl)oxy)phenyl)oxetan-3-yl)acetic acid (Compound 30)

The title compound was prepared in an analogous manner as the compound 2 of Example 2. Compound 30 was obtained by hydrolyzing compound of Example 29. Yield: 94%; ¹H NMR (CDCl₃, 300 MHz): δ 7.59 (d, J=7.5 Hz, 1H), 7.16 (d, J=8.4 Hz, 2H), 7.08-7.04 (m, 2H), 6.97 (d, J=8.4 Hz, 2H), 5.09 (s, 2H), 5.00 (d, J=6 Hz, 2H), 4.86 (d, J=6 Hz, 2H), 3.92 (s, 3H), 3.17 (s, 2H); MS: m/z 419 (M+Na).

### Example 31

### Ethyl 2-(3-(4-(3-fluoro-4-(trifluoromethyl)benzyl)oxy)phenyl)oxetan-3-yl)acetate (Compound 31)

The title compound was prepared in an analogous manner as the compound 1 of Example 1 by involving reaction of ethyl 2-(3-(4-hydroxyphenyl)oxetan-3-yl)acetate (compound of Step 1b of Example 1) and 3-fluoro-4-trifluoromethyl benzylbromide. Yield: 97%; ¹H NMR (DMSO-d₆, 300 MHz): δ 7.84 (m, 1H), 7.59 (d, J=11.7 Hz, 1H), 7.00 (d, J=7.8 Hz, 1H), 7.20 (d, J=8.7 Hz, 2H), 7.01 (d, J=8.7 Hz, 2H), 5.23 (s, 2H), 4.75 (s, 4H), 3.92 (q, J=7.2 Hz, 2H), 3.08 (s, 2H), 1.03 (t, J=7.2 Hz, 3H); MS: m/z 412 (M+1).

### Example 32

### 2-(3-(4-((3-Fluoro-4-(trifluoromethyl)benzyl)oxy)phenyl)oxetan-3-yl)acetic acid (Compound 32)

The title compound was prepared in an analogous manner as the compound 2 of Example 2. Compound 32 was obtained by hydrolyzing compound of Example 31. Yield: 47%; ¹H NMR (CDCl₃, 300 MHz): δ 7.63 (t, J=7.5 Hz, 1H), 7.33-7.28 (m, 2H), 7.16 (d, J=8.4 Hz, 2H), 6.95 (d, J=8.4 Hz, 2H), 5.11 (s, 2H), 5.00 (d, J=6 Hz, 2H), 4.86 (d, J=6 Hz, 2H), 3.17 (s, 2H); MS (m/z): 385 (M+1).

### Example 33

### Ethyl 2-(3-(4-((3-fluoro-5-(trifluoromethoxy)benzyl)oxy)phenyl)oxetan-3-yl)acetate (Compound 33)

The title compound was prepared in a manner analogous to compound 1 of Example 1 by involving reaction of ethyl 2-(3-(4-hydroxyphenyl)oxetan-3-yl)acetate (compound of Step 1b of Example 1) and 3-fluoro-5-trifluoromethoxy benzyl bromide. Yield: 85%; ¹HNMR (CDCl₃, 300 MHz): δ 7.15-7.12 (m, 4H), 6.95-6.92 (m, 3H), 5.07 (s, 2H), 5.00 (d, J=6.0 Hz, 2H), 4.86 (d, J=5.7 Hz, 2H), 4.02 (q, J=7.2 Hz, 2H), 3.11 (s, 2H), 1.14 (t, J=7.2 Hz, 3H); MS (ESI): 452.1 (M+Na).

### Example 34

### 2-(3-(4-((3-Fluoro-5-(trifluoromethoxy)benzyl)oxy)phenyl)oxetan-3-yl)acetic acid (Compound 34)

The title compound was prepared in an analogous manner as the compound 2 of Example 2. Compound 34 was obtained by hydrolyzing the compound of Example 33. Yield: 92%; ¹H NMR (DMSO-d₆, 300 MHz): δ 12.14 (bs, 1H), 7.37-7.34 (m, 3H), 7.22 (d, J=8.1 Hz, 2H), 7.99 (d, J=7.8 Hz, 2H), 5.17 (s, 2H), 4.74 (bs, 4H), 3.02 (s, 2H); MS: m/z 400.1 (M+1).

### Example 35

### Ethyl 2-(3-(4-((3-fluoro-4-(trifluoromethoxy)benzyl)oxy)phenyl)oxetan-3-yl)acetate (Compound 35)

The title compound was prepared in an analogous manner as the compound 1 of Example 1 by involving reaction of ethyl 2-(3-(4-hydroxyphenyl)oxetan-3-yl)acetate (compound of Step 1b of Example 1) and 3-fluoro-4-trifluoromethoxy benzyl bromide. Yield: 76%; ¹H NMR (CDCl₃, 300 MHz): δ 7.34-7.21 (m, 3H), 7.13 (d, J=8.4 Hz, 2H), 6.93 (d, J=8.4 Hz, 2H), 5.05 (s, 2H), 5.00 (d, J=6.0 Hz, 2H), 4.86 (d, J=6.0 Hz, 2H), 4.02 (q, J=7.2 Hz, 2H), 3.11 (s, 2H), 1.14 (t, J=7.2 Hz, 3H); MS (ESI): 451.8 (M+Na).

### Example 36

### 2-(3-(4-((3-Fluoro-4-(trifluoromethoxy)benzyl)oxy)phenyl)oxetan-3-yl)acetic acid (Compound 36)

The title compound was prepared in an analogous manner as the compound 2 of Example 2 and obtained by hydrolyzing compound of Example 35. Yield: 84%; ¹H NMR (DMSO-d₆, 300 MHz): δ 12.13 (bs, 1H), 7.61-7.58 (m, 2H), 7.41 (d, J=8.4 Hz, 1H), 7.22 (d, J=8.4 Hz, 2H), 6.99 (d, J=8.4 Hz, 2H), 5.14 (s, 2H), 4.74 (s, 4H), 3.02 (s, 2H); MS: m/z 398.8 (M−1).

### Example 37

### Ethyl 2-(3-(4-((2-fluoro-3-(trifluoromethyl)benzyl)oxy)phenyl)oxetan-3-yl)acetate (Compound 37)

The title compound was prepared in an analogous manner as the compound 1 of Example 1 by involving reaction of ethyl 2-(3-(4-hydroxyphenyl)oxetan-3-yl)acetate (compound of Step 1b of Example 1) and 2-fluoro-3-trifluoromethyl benzyl bromide. Yield: 78%; ¹HNMR (CDCl₃, 300 MHz): δ 7.75 (t, J=6.9 Hz, 1H), 7.60 (t, J=6.9 Hz, 1H), 7.32-7.28 (m, 1H), 7.14 (d, J=8.7 Hz, 2H), 6.97 (d, J=8.7 Hz, 2H), 5.18 (s, 2H), 5.00 (d, J=6.0 Hz, 2H), 4.87 (d, J=6.0 Hz, 2H), 4.09 (q, J=7.2 Hz, 2H), 3.12 (s, 2H), 1.14 (t, J=7.2 Hz, 3H); MS (ESI): m/z 436.1 (M+Na).

### Example 38

### 2-(3-(4-((2-fluoro-3-(trifluoromethyl)benzyl)oxy)phenyl)oxetan-3-yl)acetic acid (Compound 38)

The title compound was prepared in an analogous manner as the compound 2 of Example 2. Compound 38 was obtained by hydrolyzing compound of Example 37. Yield: 93%; ¹H NMR (DMSO-d₆, 300 MHz): δ 7.74 (t, J=6.6 Hz, 1H), 7.60 (t, J=6.9 Hz, 1H), 7.31-7.26 (m, 1H), 7.15 (d, J=8.4 Hz, 2H), 6.97 (d, J=8.4 Hz, 2H), 5.17 (s, 2H), 4.99 (d, J=5.7 Hz, 2H), 4.85 (d, J=5.7 Hz, 2H), 3.17 (s, 2H); MS: m/z 385.0 (M+1).

### Example 39

### Ethyl 2-(3-(4-((4′-hydroxy-2′,6′-dimethyl-[1,1′-biphenyl]-3-yl)methoxy)phenyl)oxetan-3-yl)acetate (Compound 39)

### Step 1a

### Synthesis of Ethyl 2-(3-(4-((3-bromobenzyl)oxy)phenyl)oxetan-3-yl)acetate

To a reaction mixture containing ethyl 2-(3-(4-hydroxyphenyl)oxetan-3-yl)acetate (compound of Step 1b of Example 1,200 mg, 0.847 mM), and 1-bromo-3-(bromomethyl)benzene (212 mg, 0.847 mM) in anhydrous THF (5 ml), cesium carbonate (231 mg, 1.693 mM) at 0° C. was added and the reaction mixture was stirred at RT. The residue obtained was purified by column chromatography to obtain ethyl 2-(3-(4-((3-bromobenzyl)oxy)phenyl)oxetan-3-yl)acetate (200 mg). Yield: 58.3%; ¹H NMR (CDCl₃, 300 MHz): δ 7.61 (s, 1H), 7.48 (d, J=8.4 Hz, 1H), 7.37 (d, J=8.4 Hz, 1H), 7.32-7.28 (m, 1H), 7.13 (d, J=8.2 Hz, 2H), 6.59 (d, J=8.2 Hz, 2H), 5.03 (bs, 2H), 5.01 (d, J=6.0 Hz, 2H), 4.88 (d, J=6.0 Hz, 2H), 4.03 (q, J=6.90 Hz, 2H), 3.11 (s, 2H), 1.15 (t, J=6.90 Hz, 3H); MS: m/z 406 (M+1).

### Step 1b

### Synthesis of Ethyl 2-(3-(4-((3-(4,4,5,5-tetramethyl-1,3,2-dioxaborolan-2-yl)benzyl)oxy)phenyl)oxetan-3-yl)acetate

To a degassed solution of ethyl 2-(3-(4-((3-bromobenzyl)oxy)phenyl)oxetan-3-yl)acetate (417 mg, 1.029 mM, compound of Step 1a), bispinacolatodiborane (653 mg, 2.57 mM) and potassium acetate (404 mg, 4.12 mM) in dioxane (10 ml), Pd(dppf)Cl₂.DCM (84 mg, 0.103 mM) was added. The reaction mixture was heated at 80° C. for 8 h. The solvent was removed under reduced pressure. The crude compound was purified by column chromatography to obtain the compound ethyl 2-(3-(4-((3-(4,4,5,5-tetramethyl-1,3,2-dioxaborolan-2-yl)benzyl)oxy)phenyl)oxetan-3-yl) acetate (450 mg) as a white solid. Yield: 97%. ¹H NMR (CDCl₃, 300 MHz): δ 7.87 (s, 1H), 7.80 (d, J=8.4 Hz, 1H), 7.57 (d, J=8.4 Hz, 1H), 7.43 (t, J=7.5 Hz, 1H), 7.11 (d, J=8.2 Hz, 2H), 6.97 (d, J=8.2 Hz, 2H), 5.06 (bs, 2H), 5.01 (d, J=6.0 Hz, 2H), 4.87 (d, J=6.0 Hz, 2H), 4.03 (q, J=6.90 Hz, 2H), 3.10 (s, 2H), 1.37 (s, 12H), 1.15 (t, J=6.90 Hz, 3H); MS: m/z 453 (M+1).

### Step 1c

### Synthesis of Ethyl 2-(3-(4-((4′-hydroxy-2′,6′-dimethyl-[1,1′-biphenyl]-3-yl)methoxy)phenyl)oxetan-3-yl)acetate

To a solution of ethyl 2-(3-(4-((3-(4,4,5,5-tetramethyl-1,3,2-dioxaborolan-2-yl)benzyl)oxy)phenyl)oxetan-3-yl)acetate (12 g, 26.5 mM, compound of Step 1b), 4-bromo-3,5-dimethylphenol (6.40 g, 31.8 mM) in dioxane (40 ml) and water (10 ml), potassium carbonate (11.00 g, 80 mM) was added. The reaction mixture was degassed with Ar for 10 min. Pd(PPh₃)₄ (1.533 g, 1.326 mM) was added to the resulting solution and the mixture was heated at 80° C. for 2 h. The reaction mixture was further diluted with ethyl acetate (200 ml) and water (100 ml) and filtered through celite. The organic layer was washed with brine, dried over Na₂SO₄ and concentrated to obtain the crude product. The crude product was purified by column chromatography to afford the title compound, ethyl 2-(3-(4-((4′-hydroxy-2′,6′-dimethyl-[1,1′-biphenyl]-3-yl)methoxy)phenyl)oxetan-3-yl)acetate (8 g) as a white solid. Yield: 67.5%. ¹H NMR (CDCl₃, 300 MHz): δ 7.44-7.42 (m, 2H), 7.19 (s, 1H), 7.11 (bd, J=8.1 Hz, 3H), 6.97 (d, J=8.1 Hz, 2H), 6.60 (s, 2H), 5.11 (s, 2H), 5.01 (d, J=6.0 Hz, 2H), 4.87 (d, J=6.0 Hz, 2H), 4.77 (s, OH), 4.03 (q, J=6.90 Hz, 2H), 3.10 (s, 2H), 1.98 (s, 6H), 1.13 (t, J=6.90 Hz, 3H); MS: m/z 447 (M+1).

### Example 40

### Ethyl 2-(3-(4-((2′,6′-dimethyl-4′-((tetrahydrofuran-3-yl)methoxy)-[1,1′-biphenyl]-3-yl) methoxy)phenyl)oxetan-3-yl)acetate (Compound 40)

### Step 1a

### Synthesis of (Tetrahydrofuran-3-yl)methyl 4-methylbenzenesulfonate

To a solution of (tetrahydrofuran-3-yl)methanol (500 mg, 4.90 mM) in DCM (10 ml), triethyl amine (991 mg, 9.79 mM) was added. The reaction mixture was stirred for 5 min at 0° C., followed by the addition of 4-methylbenzene-1-sulfonyl chloride (933 mg, 4.90 mM) and DMAP (1 mg). The reaction mixture was further stirred for 2 h, concentrated and purified by column chromatography to afford the title compound (tetrahydrofuran-3-yl)methyl 4-methylbenzenesulfonate (1.07 g) as a white solid; Yield: 86%; ¹H NMR (DMSO-d₆, 300 MHz): δ 7.82 (d, J=8.1 Hz, 2H), 7.39 (d, J=8.1 Hz, 2H), 4.03-3.90 (m, 2H), 3.84-3.66 (m, 4H), 3.53-3.49 (m, 1H), 2.47 (s, 3H), 1.60-1.51 (m, 2H); MS: m/z 279 (M+Na).

### Step 1b

### Ethyl 2-(3-(4-((2′,6′-dimethyl-4′-((tetrahydrofuran-3-yl)methoxy)-[1,1′-biphenyl]-3-yl) methoxy)phenyl)oxetan-3-yl)acetate

To a stirred solution of ethyl 2-(3-(4-((4′-hydroxy-2′,6′-dimethyl-[1,1′-biphenyl]-3-yl)methoxy)phenyl)oxetan-3-yl)acetate (100 mg, 0.224 mM, compound of Step 1c of Example 39), (tetrahydrofuran-3-yl)methyl 4-methylbenzenesulfonate (86 mg, 0.336 mM, compound of Step 1a) in DMF (5 ml) and cesium carbonate (146 mg, 0.448 mM) were added. The reaction mixture was stirred at 60° C. for 2 h. The reaction mixture was quenched with water, extracted with ethyl acetate and purified by column chromatography to afford the title compound ethyl 2-(3-(4-((2′,6′-dimethyl-4′-((tetrahydrofuran-3-yl)methoxy)-[1,1′-biphenyl]-3-yl)methoxy)phenyl)oxetan-3-yl) acetate (95 mg) as a colorless liquid. Yield: 80%; ¹H NMR (CDCl₃, 300 MHz): δ 7.44-7.56 (m, 2H), 7.19 (s, 1H), 7.11 (d, J=8.7 Hz, 2H), 6.96 (d, J=8.7 Hz, 2H), 6.67 (s, 2H), 5.15 (s, 2H), 5.00 (d, J=6.1 Hz, 2H), 4.84 (d, J=6.1 Hz, 2H), 4.05-3.57 (m, 8H), 3.10 (s, 2H), 2.76 (m, 2H), 2.12 (m, 2H), 2.00 (s, 6H), 1.07 (t, J=8.7 Hz, 3H); MS: m/z 530 (M)+.

### Example 41

### 2-(3-(4-((2′,6′-Dimethyl-4′-((tetrahydrofuran-3-yl)methoxy)-[1,1′-biphenyl]-3-yl) methoxy)phenyl)oxetan-3-yl)acetic acid (Compound 41)

Ethyl 2-(3-(4-((2′,6′-dimethyl-4′-((tetrahydrofuran-3-yl)methoxy)-[1,1′-biphenyl]-3-yl)methoxy)phenyl)oxetan-3-yl)acetate (compound of Step 1b of Example 40, 271 mg, 0.511 mM) was dissolved in a mixture containing THF (4 ml) and MeOH (1 ml) and Lithium Hydroxide (2.043 ml, 3.06 mM). The reaction mixture was stirred for 6 h. The reaction was quenched with saturated NH₄Cl and extracted with ethyl acetate. The organic layer was washed with brine, dried over Na₂SO₄ and concentrated to afford the title compound, 2-(3-(4-((2′,6′-dimethyl-4′-((tetra hydro furan-3-yl)methoxy)-[1,1′-biphenyl]-3-yl)methoxy)phenyl)oxetan-3-yl) acetic acid (170 mg) as a white solid. Yield: 66.2%; ¹H NMR (DMSO-d₆, 300 MHz): δ 7.44-7.40 (m, 2H), 7.18 (d, J=8.1 Hz, 1H), 7.13-7.10 (m, 2H), 6.96 (d, J=8.7 Hz, 2H), 6.67 (s, 2H), 5.15 (s, 2H), 4.99 (d, J=6.1 Hz, 2H), 4.85 (d, J=6.1 Hz, 2H), 4.05-3.57 (m, 8H), 3.15 (s, 2H), 2.78-2.67 (m, 1H), 2.13-2.09 (m, 1H), 1.9 (s, 6H), 1.80-1.74 (m, 1H); MS: m/z 525 (M+Na).

### Example 42

### Ethyl 2-(3-(4-((2′,6′-dimethyl-4′-((tetrahydro-2H-pyran-4-yl)methoxy)-[1,1′-biphenyl]-3-yl)methoxy)phenyl)oxetan-3-yl)acetate (Compound 42)

### Step 1a

### Synthesis of Tetrahydro-2H-pyran-4-yl)methyl 4-methylbenzenesulfonate

To a stirred solution of (tetrahydro-2H-pyran-4-yl)methanol (300 mg, 2.58 mM) in DCM (5 ml), triethyl amine (784 mg, 7.75 mM) was added. The reaction mixture was stirred for 5 min at 0° C. followed by the addition of 4-methylbenzene-1-sulfonyl chloride (542 mg, 2.84 mM). The reaction mixture was further stirred for 2 h. RM, concentrated and purified by column chromatography to afford the title compound tetrahydro-2H-pyran-4-yl)methyl 4-methylbenzenesulfonate (634 mg). Yield: 91%; ¹H NMR (CDCl₃, 300 MHz): δ 7.81 (d, J=8.1 Hz, 2H), 7.38 (d, J=8.1 Hz, 2H), 3.97-3.86 (m, 4H), 3.36 (t, J=6.5 Hz, 2H), 2.47 (s, 3H), 1.97-1.94 (m, 1H), 1.62 (d, J=12 Hz, 2H), 1.35-1.23 (m, 2H), MS: m/z 293 (M+Na).

### Step 1b

### Ethyl 2-(3-(4-((2′,6′-dimethyl-4′-((tetrahydro-2H-pyran-4-yl)methoxy)-[1,1′-biphenyl]-3-yl)methoxy)phenyl)oxetan-3-yl)acetate

To a solution of ethyl 2-(3-(4-((4′-hydroxy-2′,6′-dimethyl-[1,1′-biphenyl]-3-yl)methoxy)phenyl)oxetan-3-yl)acetate (compound of Step 1c of Example 39, 150 mg, 0.336 mM) and (tetrahydro-2H-pyran-4-yl)methyl 4-methylbenzenesulfonate (136 mg, 0.504 mM, compound of Step 1a) in anhydrous DMF (2 ml), cesium carbonate (219 mg, 0.672 mM) was added at RT and stirred at 50° C. for 2 h. The reaction mixture was then stirred at RT for 2 h. The reaction was quenched with the addition of water (5 ml), further stirred for 10 min and extracted with ethyl acetate. The organic layer was washed with brine, dried over Na₂SO₄ and concentrated under reduced pressure to obtain a crude product. The crude product was purified by flash column chromatography (eluted with 40% ethyl acetate in n-hexane) to obtain the title compound, ethyl 2-(3-(4-((2′,6′-dimethyl-4′-((tetrahydro-2H-pyran-4-yl)methoxy)-[1,1′-biphenyl]-3-yl)methoxy)phenyl)oxetan-3-yl)acetate (90 mg) as colorless oil. Yield: 49.2%. ¹H NMR (CDCl₃, 300 MHz): δ 7.47-7.42 (m, 2H), 7.19 (s, 1H), 7.11 (d s, J=8.7 Hz, 3H), 6.96 (d, J=8.7 Hz, 2H), 6.67 (s, 2H), 5.11 (s, 2H), 5.00 (d, J=6.1 Hz, 2H), 4.87 (d, J=6.1 Hz, 2H), 4.05-3.57 (m, 4H), 3.84 (d, J=6.1 Hz, 2H), 3.51-3.44 9 (m, 3H), 3.10 (s, 2H), 2.00 (s, 6H), 1.82 (d, J=12.9 Hz, 2H), 1.49-1.46 (m, 2H), 1.13 (t, J=8.7 Hz, 3H); MS: m/z 567 (M+Na).

### Example 43

### 2-(3-(4-((2′,6′-Dimethyl-4′-((tetrahydro-2H-pyran-4-yl)methoxy)-[1,1′-biphenyl]-3-yl) methoxy)phenyl)oxetan-3-yl)acetic acid (Compound 43)

To a solution of ethyl 2-(3-(4-((2′,6′-dimethyl-4′-((tetrahydro-2H-pyran-4-yl)methoxy)-[1,1′-biphenyl]-3-yl)methoxy)phenyl)oxetan-3-yl)acetate (compound of Step 1b of Example 42, 220 mg, 0.404 mM) in THF:MeOH (4:1) (4 ml) aqueous LiOH (1616 μl, 2.423 mM). The reaction mixture was stirred at RT for 4 h. The solvent was removed under reduced pressure and the reaction mixture was neutralized with saturated NH₄Cl. The reaction mixture was further extracted with ethyl acetate, The organic layer was washed with brine, dried over Na₂SO₄ and concentrated to afford the title compound 2-(3-(4-((2′,6′-dimethyl-4′-((tetrahydro-2H-pyran-4-yl)methoxy)-[1,1′-biphenyl]-3-yl)methoxy)phenyl)oxetan-3-yl)acetic acid (110 mg) as a white solid. Yield: 52.7%; ¹H NMR (DMSO-d₆, 300 MHz): δ 12.8 (s, 1H), 7.45-7.42 (m, 2H), 7.21-7.15 (m, 3H), 7.07 (d, J=8.7 Hz, 1H), 6.99 (d, J=8.7 Hz, 2H), 6.69 (s, 2H), 5.14 (s, 2H), 4.74 (s, 4H), 3.90-3.81 (m, 4H), 3.32-3.30 (m, 2H), 3.00 (s, 2H), 1.91 (s, 6H), 1.71 (d, J=12 Hz, 2H), 1.34-1.25 (m, 3H); MS: m/z 516 (M+).

### Example 44

### Ethyl 2-(3-(4-((4′-((1,1-dioxidotetrahydro-2H-thiopyran-4-yl)methoxy)-2′,6′-dimethyl-[1,1′-biphenyl]-3-yl)methoxy)phenyl)oxetan-3-yl)acetate (Compound 44)

### Step 1a

### Synthesis of (1,1-Dioxidotetrahydro-2H-thiopyran-4-yl)methyl4-methylbenzene sulfonate

To a solution of (tetrahydro-2H-thiopyran-4-yl)methanol (400 mg, 3.03 mM) in methanol (10 ml), aqueous solution of oxone (3715 mg, 6.05 mM) in water (10 ml) was added. The reaction mixture was stirred for 6 h and quenched with saturated NaHCO₃ solution. The reaction mixture was then extracted with ethyl acetate. The organic layer was washed with brine and concentrated to afford 4-(hydroxymethyl)tetrahydro-2H-thiopyran 1,1-dioxide (230 mg) used in the subsequent reaction step without purification. Yield: 46.3%.

To a stirred solution of 4-(hydroxymethyl)tetrahydro-2H-thiopyran 1,1-dioxide (230 mg, 1.401 mM) in DCM (5 ml), triethyl amine (585 μl, 4.20 mM) was added. The reaction mixture was then stirred at 0° C. for 5 min. 4-methylbenzene-1-sulfonyl chloride (320 mg, 1.681 mM) was added to the reaction mixture which was further stirred for 2 h. The reaction mixture was then concentrated to obtain a crude product which was purified by column chromatography to afford the title compound (1,1-dioxidotetrahydro-2H-thiopyran-4-yl)methyl 4-methylbenzenesulfonate (249 mg) as a white solid. Yield: 55.8%; ¹H NMR (CDCl₃, 300 MHz): δ 7.81 (d, J=8.1 Hz, 2H), 7.40 (d, J=8.1 Hz, 2H), 3.92 (m, 4H), 3.36 (t, J=6.5 Hz, 2H), 2.47 (s, 3H), 1.97-1.94 (m, 1H), 1.62 (d, J=12 Hz, 2H), 1.35-1.23 (m, 2H); MS: m/z 341 (M+Na).

### Step 1b

### Ethyl 2-(3-(4-((4′-((1,1-dioxidotetrahydro-2H-thiopyran-4-yl)methoxy)-2′,6′-dimethyl-[1,1′-biphenyl]-3-yl)methoxy)phenyl)oxetan-3-yl)acetate

To a stirred solution of ethyl 2-(3-(4-((4′-hydroxy-2′,6′-dimethyl-[1,1′-biphenyl]-3-yl)methoxy)phenyl)oxetan-3-yl)acetate (compound of Step 1c of Example 39, 175 mg, 0.392 mM) and (1,1-dioxidotetrahydro-2H-thiopyran-4-yl)methyl 4-methylbenzenesulfonate (125 mg, 0.392 mM, compound of Step 1a) dissolved in DMF (5 ml), cesium carbonate (255 mg, 0.784 mM) was added. The reaction mixture was then stirred at 80° C. for 4 h. The reaction mixture was quenched with water and extracted with ethyl acetate. The crude product obtained was further purified by column chromatography to afford the title compound ethyl 2-(3-(4-((4′-((1,1-dioxidotetrahydro-2H-thiopyran-4-yl)methoxy)-2′,6′-dimethyl-[1,1′-biphenyl]-3-yl)methoxy)phenyl)oxetan-3-yl)acetate (175 mg) as a white solid. Yield: 75%; ¹H NMR (CDCl₃, 300 MHz): δ 7.47-7.42 (m, 2H), 7.18 (s, 1H), 7.11 (s, d, J=8.7 Hz, 3H), 6.96 (d, J=8.7 Hz, 2H), 6.65 (s, 2H), 5.10 (s, 2H), 5.03 (d, J=6.1 Hz, 2H), 4.87 (d, J=6.1 Hz, 2H), 4.15 (q, J=6.1 Hz, 2H), 3.90 (s, 2H), 3.18-3.06 (m including s 3.10, 6H), 2.32-2.28 (m, 2H), 2.09-2.06 (m, 3H), 2.00 (s, 6H), 1.13 (t, J=8.7 Hz, 3H); MS: m/z 593 (M+).

### Example 45

### 2-(3-(4-((4′-((1,1-Dioxidotetrahydro-2H-thiopyran-4-yl)methoxy)-2′,6′-dimethyl-[1,1′-biphenyl]-3-yl)methoxy)phenyl)oxetan-3-yl)acetic acid (Compound 45)

To a solution of ethyl 2-(3-(4-((4′-((1,1-dioxidotetrahydro-2H-thiopyran-4-yl)methoxy)-2′,6′-dimethyl-[1,1′-biphenyl]-3-yl)methoxy)phenyl)oxetan-3-yl)acetate (compound of Step 1b of Example 44, 145 mg, 0.245 mM) in THF:MeOH (4:1) (4 ml), aqueous LiOH (979 μl, 1.468 mM) was added. The reaction mixture was stirred at RT for 4 h and the solvent was removed under reduced pressure. The reaction mixture was neutralized with saturated NH₄Cl and extracted with ethyl acetate. The organic layer was washed with brine, dried over Na₂SO₄ and concentrated to afford the title compound 2-(3-(4-((4′-((1,1-dioxidotetrahydro-2H-thiopyran-4-yl)methoxy)-2′,6′-dimethyl-[1,1′-biphenyl]-3-yl)methoxy)phenyl)oxetan-3-yl)acetic acid (110 mg) as a white solid. Yield: 80%; ¹H NMR (DMSO-d₆, 300 MHz): δ 12.8 (s, 1H, OH), 7.47-7.42 (m, 2H), 7.21-7.14 (m, 3H), 7.06 (d, J=6.9 Hz, 1H), 6.98 (d, J=8.7 Hz, 2H), 6.70 (s, 2H), 5.13 (s, 2H), 4.76-4.70 (m, 4H), 3.90 (s, 2H), 3.18-3.04 (m, 4H), 2.98 (s, 2H), 2.16-2.06 (m, 3H), 1.90 (s, 6H), 1.82-1.70 (m, 2H); MS: m/z 565 (M+1).

### Example 46

### Ethyl 2-(3-(4-((2′,6′-dimethyl-4′-((tetrahydrofuran-2-yl)methoxy)-[1,1′-biphenyl]-3-yl) methoxy)phenyl)oxetan-3-yl)acetate (Compound 46)

### Step 1a

### Synthesis of Tetrahydrofuran-2-yl)methyl 4-methylbenzenesulfonate

To a stirred solution of (tetrahydrofuran-2-yl)methanol (500 mg, 4.90 mM) in DCM (5 ml), triethyl amine (1486 mg, 14.69 mM) was added and the reaction mixture was stirred for 5 min at 0° C. 4-methylbenzene-1-sulfonyl chloride (1120 mg, 5.87 mM) was added to the reaction mixture which was further stirred for 2 h. The reaction mixture was concentrated to obtain a crude product which was purified by column chromatography to afford the title compound tetrahydrofuran-2-yl)methyl 4-methylbenzenesulfonate (856 mg). Yield: 68.2%; ¹H NMR (CDCl₃, 300 MHz): δ 7.83 (d, J=8.1 Hz, 2H), 7.37 (d, J=8.1 Hz, 2H), 4.12-3.99 (m, 3H), 3.81-3.71 (m, 2H), 2.46 (s, 3H), 2.00-1.84 (m, 3H), 1.71-1.62 (m, 1H); MS: m/z 279 (M+Na).

### Step 1b

### Ethyl 2-(3-(4-((2′,6′-dimethyl-4′-((tetrahydrofuran-2-yl)methoxy)-[1,1′-biphenyl]-3-yl) methoxy)phenyl)oxetan-3-yl)acetate

To a stirred solution of ethyl 2-(3-(4-((4′-hydroxy-2′,6′-dimethyl-[1,1′-biphenyl]-3-yl)methoxy)phenyl)oxetan-3-yl)acetate (compound of Step 1c of Example 39, 150 mg, 0.585 mM) and (tetrahydrofuran-2-yl)methyl 4-methylbenzenesulfonate (314 mg, 0.702 mM, compound of Step 1a) dissolved in DMF (5 ml), cesium carbonate (381 mg, 1.170 mM) was added. The reaction mixture was stirred at 60° C. for 4 h. The reaction mixture was quenched with water, extracted with ethyl acetate and purified by column chromatography (30% ethyl acetate in hexane) to afford the title compound ethyl 2-(3-(4-((2′,6′-dimethyl-4′-((tetrahydrofuran-2-yl)methoxy)-[1,1′-biphenyl]-3-yl)methoxy)phenyl)oxetan-3-yl)acetate (170 mg) as a colourless liquid. Yield: 54.7%; ¹H NMR (CDCl₃, 300 MHz): δ 7.44-7.56 (m, 2H), 7.19 (s, 1H), 7.11 (d, J=8.4 Hz, 3H), 6.97 (d, J=8.5 Hz, 2H), 6.70 (s, 2H), 5.11 (s, 2H), 5.00 (d, J=6.1 Hz, 2H), 4.87 (d, J=6.1 Hz, 2H), 4.32-4.28 (m, 1H), 4.05-3.96 (m, 5H), 3.89-3.84 (m, 1H), 3.10 (s, 2H), 2.13-2.05 (m, 2H), 2.00 (s, 6H), 1.85-1.82 (m, 2H), 1.13 (t, J=8.7 Hz, 3H); MS: m/z 553 (M+Na).

### Example 47

### 2-(3-(4-((2′,6′-Dimethyl-4′-((tetrahydrofuran-2-yl)methoxy)-[1,1′-biphenyl]-3-yl) methoxy)phenyl)oxetan-3-yl)acetic acid (Compound 47)

Ethyl 2-(3-(4-((2′,6′-dimethyl-4′-((tetrahydrofuran-2-yl)methoxy)-[1,1′-biphenyl]-3-yl)methoxy)phenyl)oxetan-3-yl)acetate (compound of Step 1b of Example 46, 135 mg, 0.254 mM) was dissolved in a mixture of THF (4 ml) and MeOH (1 ml) and aqueous LiOH monohydrate (1018 μl, 1.526 mM) was added to the reaction mixture. The reaction mixture was stirred for 6 h and quenched with saturated NH₄Cl. The mixture was extracted with ethyl acetate and the organic layer was washed with brine, dried over Na₂SO₄ and concentrated to afford the title compound 2-(3-(4-((2′,6′-dimethyl-4′-((tetrahydrofuran-2-yl)methoxy)-[1,1′-biphenyl]-3-yl)methoxy)phenyl)oxetan-3-yl)acetic acid (80 mg) as a white solid. Yield: 62.6%; ¹H NMR (DMSO-d₆, 300 MHz): δ 12.8 (s, 1H), 7.44-7.40 (m, 2H), 7.21-7.18 (m, 3H), 7.07 (d, J=7.2 Hz, 1H), 6.96 (d, J=8.7 Hz, 2H), 6.69 (s, 2H), 5.14 (s, 2H), 4.74 (s, 4H), 4.15-4.10 (m, 1H), 3.95-3.93 (m, 2H), 3.80-3.67 (m, 2H), 3.50-3.32 (m, 2H), 3.01 (s, 2H), 2.09-2.05 (m, s, 1.91, 7H), 1.80-1.74 (m, 1H); MS: m/z 525 (M+Na).

### Example 48

### R)-ethyl 2-(3-(4-((2′,6′-dimethyl-4′-((tetrahydrofuran-3-yl)methoxy)-[1,1′-biphenyl]-3-yl)methoxy)phenyl)oxetan-3-yl)acetate (Compound 48

### Step 1a

### Synthesis of (R)-(tetrahydrofuran-3-yl)methyl 4-methylbenzenesulfonate

To a solution of (R)-(tetrahydrofuran-3-yl)methanol (500 mg, 4.90 mM) in DCM (10 ml), triethyl amine (1486 mg, 14.69 mM). The reaction mixture was stirred for 5 min at 0° C. followed by the addition of 4-methylbenzene-1-sulfonyl chloride (1120 mg, 5.87 mM) and DMAP (1 mg). The reaction mixture was further stirred for 2 h, concentrated to obtain a crude product which was purified by column chromatography to afford the title compound (R)-(tetrahydrofuran-3-yl)methyl 4-methylbenzenesulfonate (925 mg) as a white solid. Yield: 73.7%; ¹H NMR (CDCl₃, 300 MHz): δ 7.81 (d, J=8.1 Hz, 2H), 7.38 (d, J=8.1 Hz, 2H), 3.95-3.90 (m, 2H), 3.81-3.84 (m, 3H), 3.53-3.48 (m, 1H), 2.62-2.56 (m, 1H), 2.47 (s, 3H), 2.07-1.96 (m, 1H), 1.60-1.51 (m, 1H); MS: m/z 256 (M+).

### Step 1b

### (R)-ethyl 2-(3-(4-((2′,6′-dimethyl-4′-((tetrahydrofuran-3-yl)methoxy)-[1,1′-biphenyl]-3-yl)methoxy)phenyl)oxetan-3-yl)acetate

To a stirred solution of ethyl 2-(3-(4-((4′-hydroxy-2′,6′-dimethyl-[1,1′-biphenyl]-3-yl)methoxy)phenyl)oxetan-3-yl)acetate (compound of Step 1c of Example 39, 175 mg, 0.392 mM) and (R)-(tetrahydrofuran-3-yl)methyl 4-methylbenzenesulfonate (121 mg, 0.470 mM, compound of Step 1a) dissolved in DMF (5 ml), cesium carbonate (255 mg, 0.784 mM) was added. The reaction mixture was stirred at 60° C. for 2 h. The reaction mixture was quenched with water, extracted with ethyl acetate and purified by column chromatography to afford the title compound (R)-ethyl 2-(3-(4-((2′,6′-dimethyl-4′-((tetrahydrofuran-3-yl)methoxy)-[1,1′-biphenyl]-3-yl)methoxy)phenyl)oxetan-3-yl)acetate (165 mg) as a viscous liquid. Yield: 79%; ¹H NMR (CDCl₃, 300 MHz): δ 7.44-7.56 (m, 2H), 7.19 (s, 1H), 7.11 (d, J=8.7 Hz, 3H), 6.96 (d, J=8.7 Hz, 2H), 6.67 (s, 2H), 5.10 (s, 2H), 5.00 (d, J=6.1 Hz, 2H), 4.87 (d, J=6.2 Hz, 2H), 4.05-3.57 (m, 8H), 3.10 (s, 2H), 2.79-2.74 (m, 1H), 2.12-2.10 (m, 1H), 2.00 (s, 6H), 1.80-1.65 (m, 1H), 1.13 (t, J=8.7 Hz, 3H); MS: m/z 553 (M+Na).

### Example 49

### R)-2-(3-(4-((2′,6′-Dimethyl-4′-((tetrahydrofuran-3-yl)methoxy)-[1,1′-biphenyl]-3-yl)methoxy)phenyl)oxetan-3-yl)acetic acid (Compound 49

(R)-ethyl 2-(3-(4-((2′,6′-dimethyl-4′-((tetrahydrofuran-3-yl)methoxy)-[1,1′-biphenyl]-3-yl)methoxy)phenyl)oxetan-3-yl)acetate (compound of Step 1b of Example 48, 244 mg, 0.460 mM) was dissolved in a mixture of THF (4 ml) and MeOH (1 ml) followed by the addition of aqueous LiOH monohydrate (1839 μl, 2.76 mM). The reaction mixture was stirred for 6 h and quenched with saturated NH₄Cl. The reaction mixture was then extracted with ethyl acetate. The organic layer was washed with brine, dried over Na₂SO₄ and concentrated to afford the title compound (R)-2-(3-(4-((2′,6′-dimethyl-4′-((tetrahydrofuran-3-yl)methoxy)-[1,1′-biphenyl]-3-yl) methoxy)phenyl)oxetan-3-yl)acetic acid (189 mg) as a white solid. Yield: 82%; ¹H NMR (DMSO-d₆, 300 MHz): δ 12.8 (s, 1H), 7.44-7.40 (m, 2H), 7.20-7.15 (m, 3H), 7.06 (d, J=8.1 Hz, 1H), 6.96 (d, J=8.7 Hz, 2H), 6.67 (s, 2H), 5.15 (s, 2H), 4.73 (s, 4H), 3.86-3.50 (m, 6H), 3.10 (s, 2H), 2.64-2.62 (m, 1H), 2.00-1.96 (m, 1H), 1.90 (s, 6H), 1.68-1.62 (m, 1H); MS: m/z 525 (M+Na).

### Example 50

### Ethyl 2-(3-(4-((2′,6′-dimethyl-4′-((3-methyloxetan-3-yl)methoxy)-[1,1′-biphenyl]-3-yl)methoxy)phenyl)oxetan-3-yl)acetate (Compound 50)

### Step 1a

### Synthesis of 3-Methyloxetan-3-yl)methyl 4-methylbenzenesulfonate

To a solution of (3-methyloxetan-3-yl)methanol (1 g, 9.79 mM) in DCM (15 ml), triethylamine (2.71 ml, 19.58 mM) was added at 0° C. followed by the addition of 4-methylbenzene-1-sulfonyl chloride (1.867 g, 9.79 mM). The reaction mixture was stirred at RT for 3 h to 5 h. The reaction mixture was then quenched with water, extracted with ethyl acetate and purified by column chromatography to afford the title compound (3-methyloxetan-3-yl)methyl 4-methylbenzenesulfonate (1.875 g) as a white solid. Yield: 74.7%; ¹H NMR (DMSO-d₆, 300 MHz): δ 7.82 (d, J=8.1 Hz, 2H), 7.51 (d, J=8.1 Hz, 2H), 4.25 (d, J=5.7 Hz, 2H), 4.19 (d, J=6.0 Hz, 2H), 4.11 (s, 2H), 2.43 (s, 3H), 1.18 (s, 3H); MS (ESI): m/z 279.0 (M+Na).

### Step 1b

### Ethyl 2-(3-(4-((2′,6′-dimethyl-4′-((3-methyloxetan-3-yl)methoxy)-[1,1′-biphenyl]-3-yl)methoxy)phenyl)oxetan-3-yl)acetate

To a stirred solution of ethyl 2-(3-(4-((4′-hydroxy-2′,6′-dimethyl-[1,1′-biphenyl]-3-yl)methoxy)phenyl)oxetan-3-yl)acetate (compound of Step 1c of Example 39, 800 mg, 1.792 mM) and (3-methyloxetan-3-yl)methyl 4-methylbenzenesulfonate (459 mg, 1.792 mM, compound of Step 1a) dissolved in DMF (15 ml), cesium carbonate (518 mg, 2.69 mM) was added. The reaction mixture was stirred at 80° C. for 2 h to 5 h. The reaction mixture was quenched with water, extracted with ethyl acetate and purified by column chromatography to afford the title compound ethyl 2-(3-(4-((2′,6′-dimethyl-4′-((3-methyloxetan-3-yl)methoxy)-[1,1′-biphenyl]-3-yl)methoxy)phenyl)oxetan-3-yl)acetate (843 mg) as a pale white semisolid. Yield: 88.7%; ¹H NMR (CDCl₃, 300 MHz): δ 7.47-7.41 (m, 2H), 7.19 (s, 1H), 7.19-7.09 (m, 3H), 6.95 (d, J=8.4 Hz, 2H), 6.72 (s, 2H), 5.11 (s, 2H), 4.99 (d, J=6.0 Hz, 2H), 4.86 (d, J=6.0 Hz, 2H), 4.66 (d, J=5.7 Hz, 2H), 4.48 (d, J=5.7 Hz, 2H), 4.10-3.98 (m, 4H), 3.10 (s, 2H), 2.01 (s, 6H), 1.46 (s, 3H), 1.13 (t, J=6.9 Hz, 3H); MS: m/z 531.1 (M+1), 553.0 (M+Na).

### Example 51

### 2-(3-(4-((2′,6′-Dimethyl-4′-((3-methyloxetan-3-yl)methoxy)-[1,1′-biphenyl]-3-yl) methoxy)phenyl)oxetan-3-yl)acetic acid (Compound 51)

To a solution of ethyl 2-(3-(4-((2′,6′-dimethyl-4′-((3-methyloxetan-3-yl)methoxy)-[1,1′-biphenyl]-3-yl)methoxy)phenyl)oxetan-3-yl)acetate (compound of Step 1b of Example 50, 750 mg, 1.413 mM) in THF:MeOH (4:1) (10 ml), aqueous LiOH monohydrate (4711 μl, 7.07 mM) was added. The reaction mixture was stirred at RT for 4 h and the solvent was removed under reduced pressure. The reaction mixture was neutralized with saturated ammonium chloride mixture and then extracted with ethyl acetate. The organic layer was washed with brine, dried over Na₂SO₄ and concentrated to afford the title compound 2-(3-(4-((2′,6′-dimethyl-4′-((3-methyloxetan-3-yl)methoxy)-[1,1′-biphenyl]-3-yl)methoxy)phenyl)oxetan-3-yl)acetic acid (578 mg, 1.149 mM) as a white solid. Yield: 81.3%; ¹H NMR (DMSO-d₆, 300 MHz): δ 12.12 (bs, 1H), 7.48-7.42 (m, 3H), 7.20-7.04 (m, 3H), 6.97 (d, J=8.7 Hz, 2H), 6.74 (s, 2H), 5.14 (s, 2H), 4.73 (s, 4), 4.48 (d, J=5.7 Hz, 2H), 4.31 (d, J=5.7 Hz, 2H), 4.04 (s, 2H), 3.00 (s, 2H), 1.91 (s, 6H), 1.36 (s, 3H); MS (ESI): m/z 503.4 (M+1), 525.1 (M+Na).

### Example 52

### Ethyl 2-(3-(4-((4′-((1,1-dioxidotetrahydrothiophen-3-yl)methoxy)-2′,6′-dimethyl-[1,1′-biphenyl]-3-yl)methoxy)phenyl)oxetan-3-yl)acetate (Compound 52)

### Step 1a

### Synthesis of 3-(Hydroxymethyl)tetrahydrothiophene 1,1-dioxide

To a solution of tetrahydrothiophene-3-carboxylic acid 1,1-dioxide (302 mg, 1.839 mM) in THF (20 ml) at −10° C., N-methyl morpholine (202 μl, 1.839 mM) was added. The reaction mixture was stirred for 1 min followed by the addition of ethyl chloroformate (200 mg, 1.839 mM) dropwise. The reaction mixture was stirred at −10° C. for 15 min, filtered through celite and the filtrate was added dropwise via syringe to a mixture of NaBH₄ borohydride (139 mg, 3.68 mM) in water (10 ml) at 5° C. The reaction mixture was further stirred at ambient temperature for 2 h. The reaction mixture was quenched with saturated, aqueous NH₄Cl (10 ml) and diluted with ethyl acetate (10 ml). The aqueous layer was extracted with ethyl acetate. The organic layer was dried over anhydrous Na₂SO₄, filtered and concentrated under reduced pressure to afford the title compound 3-(hydroxymethyl)tetrahydrothiophene 1,1-dioxide (156 mg) which was used for the next step without purification. Yield: 56.5%.

### Step 1b

### Synthesis of (1,1-Dioxidotetrahydrothiophen-3-yl)methyl 4-methylbenzenesulfonate

To a solution of 3-(hydroxymethyl)tetrahydrothiophene 1,1-dioxide (156 mg, 1.039 mM, compound of Step 1a) in DCM (10 ml), DMAP (2 mg, 1.039 mM) and p-toluene sulfonylchloride (198 mg, 1.039 mM) were added. The reaction mixture was stirred at 0° C., triethyl amine (0.289 ml, 2.077 mM) was added to the reaction mixture which was further stirred at RT for 1 h. The starting material was removed under reduced pressure. The crude compound obtained was purified by column chromatography to afford the title compound (1,1-dioxidotetrahydrothiophen-3-yl)methyl 4-methylbenzenesulfonate (165 mg) as a white solid. Yield: 52.2%; ¹H NMR (CDCl₃, 300 MHz): δ 7.81 (d, J=8.4 Hz, 2H), 7.40 (d, J=8.4 Hz, 2H), 4.14-4.02 (m, 2H), 3.22-3.14 (m, 2H), 3.09-2.99 (m, 1H), 2.84-2.73 (m, 2H), 2.48 (s, 3H), 2.34-2.30 (m, 1H), 2.00-1.93 (m, 1H); MS: m/z 327 (M+Na).

### Step 1c

### Ethyl 2-(3-(4-((4′-((1,1-dioxidotetrahydrothiophen-3-yl)methoxy)-2′,6′-dimethyl-[1,1′-biphenyl]-3-yl)methoxy)phenyl)oxetan-3-yl)acetate

To a stirred solution of ethyl 2-(3-(4-((4′-hydroxy-2′,6′-dimethyl-[1,1′-biphenyl]-3-yl)methoxy)phenyl)oxetan-3-yl)acetate (compound of Step 1c of Example 39, 100 mg, 0.224 mM) and (1,1-dioxidotetrahydrothiophen-3-yl)methyl 4-methylbenzenesulfonate (68.2 mg, 0.224 mM, compound of Step 1b) dissolved in DMF (5 ml), cesium carbonate (146 mg, 0.448 mM) was added and stirred at 80° C. for 4 h. The reaction mixture was quenched with water, extracted with ethyl acetate and purified by column chromatography to afford the title compound ethyl 2-(3-(4-((4′-((1,1-dioxidotetrahydrothiophen-3-yl)methoxy)-2′,6′-dimethyl-[1,1′-biphenyl]-3-yl)methoxy)phenyl)oxetan-3-yl)acetate (110 mg). Yield: 83.3%; ¹H NMR (CDCl₃, 300 MHz): δ 7.47-7.42 (m, 2H), 7.18-7.09 (m, 4H), 6.95 (d, J=8.7 Hz, 2H), 6.66 (s, 2H), 5.11 (s, 2H), 4.98 (d, J=5.7 Hz, 2H), 4.86 (d, J=6.0 Hz, 2H), 4.06-3.98 (m, 4H), 3.37-3.25 (m, 2H), 3.25-2.97 (m, 5H), 2.46-2.44 (m, 1H), 2.26-2.06 (m, 1H), 2.00 (s, 6H), 1.13 (t, J=7.2 Hz, 3H); MS (ESI): m/z 578.9 (M+1).

### Example 53

### 2-(3-(4-((4′-((1,1-Dioxidotetrahydrothiophen-3-yl)methoxy)-2′,6′-dimethyl-[1,1′-biphenyl]-3-yl)methoxy)phenyl)oxetan-3-yl)acetic acid (Compound 53)

To a solution of ethyl 2-(3-(4-((4′-((1,1-dioxidotetrahydrothiophen-3-yl)methoxy)-2′,6′-dimethyl-[1,1′-biphenyl]-3-yl)methoxy)phenyl)oxetan-3-yl)acetate (compound of Step 1c of Example 52, 70 mg, 0.121 mM) in THF:MeOH (4:1) (2 ml), aqueous LiOH monohydrate (403 μl, 0.605 mM) was added. The reaction mixture was stirred at RT for 4 h and the solvent was removed under reduced pressure. The reaction mixture was neutralized with saturated NH₄Cl and extracted with ethyl acetate. The organic layer was washed with brine, dried over Na₂SO₄ and concentrated to afford the title compound 2-(3-(4-((4′-((1,1-dioxidotetrahydro thiophen-3-yl)methoxy)-2′,6′-dimethyl-[1,1′-biphenyl]-3-yl)methoxy)phenyl)oxetan-3-yl)acetic acid (60.1 mg) as a white solid. Yield: 87.6%; ¹H NMR (CDCl₃, 300 MHz): δ 12.10 (bs, 1H), 7.45-7.42 (m, 2H), 7.20-7.04 (m, 4H), 6.97 (d, J=8.4 Hz, 2H), 6.71 (s, 2H), 5.14 (s, 2H), 4.73 (s, 4H), 4.04 (d, J=5.7 Hz, 2H), 3.24-3.11 (m, 3H), 3.00 (s, 2H), 2.97-2.90 (m, 3H), 2.36-2.31 (m, 1H), 1.91 (s, 6H); MS (ESI): m/z 551.0 (M+1), 548.9 (M−1).

### Example 54

### Ethyl 2-(3-(4-((4′-((3-(hydroxymethyl)oxetan-3-yl)methoxy)-2′,6′-dimethyl-[1,1′-biphenyl]-3-yl)methoxy)phenyl)oxetan-3-yl)acetate (Compound 54)

To a stirred solution of ethyl 2-(3-(4-((4′-hydroxy-2′,6′-dimethyl-[1,1′-biphenyl]-3-yl)methoxy)phenyl)oxetan-3-yl)acetate (compound of Step 1c of Example 39, 105 mg, 0.235 mM) and (3-(bromomethyl)oxetan-3-yl)methanol (42.6 mg, 0.235 mM) dissolved in DMF (5 ml), cesium carbonate (91 mg, 0.470 mM) was added. The reaction mixture was stirred at 60° C. for 2 h. The reaction mixture was quenched with water, extracted with ethyl acetate and purified by column chromatography to afford the title compound ethyl 2-(3-(4-((4′-((3-(hydroxymethyl)oxetan-3-yl)methoxy)-2′,6′-dimethyl-[1,1′-biphenyl]-3-yl)methoxy)phenyl)oxetan-3-yl)acetate (120 mg). Yield: 91%; ¹H NMR (DMSO-d₆, 300 MHz): δ 7.47-7.41 (m, 2H), 7.14-7.04 (m, 4H), 6.96 (d, J=8.4 Hz, 2H), 6.74 (s, 2H), 5.14 (s, 2H), 4.98 (bs, 1H), 4.74 (s, 4H), 4.41 (s, 4H), 4.13 (s, 2H), 3.92-3.83 (m, 2H), 3.71-3.69 (m, 2H), 3.07 (s, 2H), 1.91 (s, 6H), 1.02 (t, J=6.9 Hz, 3H); MS: m/z 547.1 (M+1), 569.1 (M+Na).

### Example 55

### 2-(3-(4-((4′-((3-(Hydroxymethyl)oxetan-3-yl)methoxy)-2′,6′-dimethyl-[1,1′-biphenyl]-3-yl)methoxy)phenyl)oxetan-3-yl)acetic acid (Compound 55)

To a solution of ethyl 2-(3-(4-((4′-((3-(hydroxymethyl)oxetan-3-yl)methoxy)-2′,6′-dimethyl-[1,1′-biphenyl]-3-yl)methoxy)phenyl)oxetan-3-yl)acetate (compound of Example 54, 70 mg, 0.128 mM) in THF:MeOH (4:1) (5 ml), LiOH monohydrate (427 μl, 0.640 mM). The reaction mixture was stirred at RT for 2-3 h and the solvent was removed under reduced pressure. The reaction mixture was then neutralized with saturated NH₄Cl and extracted with ethyl acetate. The organic layer was washed with brine, dried over Na₂SO₄ and concentrated to afford the title compound 2-(3-(4-((4′-((3-(hydroxymethyl)oxetan-3-yl)methoxy)-2′,6′-dimethyl-[1,1′-biphenyl]-3-yl) methoxy)phenyl)oxetan-3-yl)acetic acid (60 mg). Yield: 88%; ¹H NMR (DMSO-d₆, 300 MHz): δ 12.11 (bs, 1H), 7.45-7.42 (m, 2H), 7.20-7.15 (m, 3H), 7.07-6.96 (m, 3H), 6.74 (s, 2H), 5.14 (s, 2H), 4.99 (bs, 1H), 4.73 (s, 4H), 4.41 (s, 4H), 4.13 (s, 2H), 3.71-3.69 (m, 2H), 3.00 (s, 2H), 1.91 (s, 6H); MS (ESI): 519.1 (M+1), 541.0 (M+Na).

### Example 56

### Ethyl 2-(3-(4-((4′-((1,1-dioxidotetrahydro-2H-thiopyran-4-yl)oxy)-2′,6′-dimethyl-[1,1′-biphenyl]-3-yl)methoxy)phenyl)oxetan-3-yl)acetate (Compound 56)

### Step 1a

### Synthesis of 4-Bromo-3,5-dimethylphenol

To a solution of dihydro-2H-thiopyran-4(3H)-one (530 mg, 4.56 mM) in methanol, NaBH₄ (207 mg, 5.47 mM) was added. The reaction mixture was stirred at RT and quenched with saturated NH₄Cl. The reaction mixture was then extracted with ethyl acetate. The organic layer was washed with brine, dried over Na₂SO₄ and concentrated to obtain a crude product which was purified by column chromatography to afford the title compound tetrahydro-2H-thiopyran-4-ol (446 mg). Yield: 83%; ¹H NMR (CDCl₃, 300 MHz): δ 3.66 (s, 1H), 2.76-2.57 (m, 4H), 2.16-2.14 (m, 2H), 1.76-1.59 (m, 2H); Ms: m/z 118 (M+).

### Step 1b

### Synthesis of 4-(4-Bromo-3,5-dimethylphenoxy)tetrahydro-2H-thiopyran

To a stirring solution of 4-bromo-3,5-dimethylphenol (710 mg, 3.53 mM), tetrahydro-2H-thiopyran-4-ol (501 mg, 4.24 mM, compound of Step 1a) and PPh₃ (2316 mg, 8.83 mM) in anhydrous DCM (10 ml), DIAD (1785 mg, 8.83 mM) was added under Ar atmosphere. The reaction was warmed at RT and stirred for 16 h to 18 h. The reaction mixture was then concentrated under reduced pressure to afford the crude product which was purified by column chromatography to afford the title compound 4-(4-bromo-3,5-dimethylphenoxy)tetrahydro-2H-thiopyran (320 mg), and compound used for next step without purification. Yield: 30.1%.

### Step 1c

### Synthesis of 4-(4-Bromo-3,5-dimethylphenoxy)tetrahydro-2H-thiopyran 1,1-dioxide

4-(4-Bromo-3,5-dimethylphenoxy)tetrahydro-2H-thiopyran (150 mg, 0.498 mM, compound of Step 1b) was dissolved in methanol (10 ml) and reacted with oxone (611 mg, 0.996 mM) in water (10 ml). The reaction mixture was stirred at RT for 6 h and quenched with the addition of saturated NaHCO₃. The reaction mixture was then extracted with ethylacetate. The organic layer was washed with brine, dried over Na₂SO₄ and concentrated under reduced pressure. The crude product obtained was purified by column chromatography to afford the title compound 4-(4-bromo-3,5-dimethylphenoxy)tetrahydro-2H-thiopyran 1,1-dioxide (80 mg). Yield: 48.2%; ¹H NMR (CDCl₃, 300 MHz): δ 6.76 (s, 2H), 4.68 (s, 1H), 3.52 (t, J=12.2 Hz, 2 Hz), 2.97 (d, J=8.2 Hz, 2H), 2.65 (s, 6H), 2.50-2.34 (m, 4H). MS: m/z 333 (M+).

### Step 1d

### Ethyl 2-(3-(4-((4′-((1,1-dioxidotetrahydro-2H-thiopyran-4-yl)oxy)-2′,6′-dimethyl-[1,1′-biphenyl]-3-yl)methoxy)phenyl)oxetan-3-yl)acetate

To a mixture of ethyl 2-(3-(4-((3-(4,4,5,5-tetramethyl-1,3,2-dioxaborolan-2-yl)benzyl)oxy)phenyl)oxetan-3-yl)acetate (compound of Step 1b of Example 39, 250 mg, 0.553 mM), 4-(4-bromo-3,5-dimethylphenoxy)tetrahydro-2H-thiopyran 1,1-dioxide (compound of Step 1c, 203 mg, 0.608 mM), and K₂CO₃ (229 mg, 1.658 mM) in a mixture of dioxane (4 ml) and water (1 ml) degassed for 5 min with Ar, Pd(PPh₃)₄ (38.3 mg, 0.033 mM) was added. The reaction mixture was heated in a microwave for 10 min at 115° C. and concentrated under reduced pressure. The crude product obtained was purified by column chromatography (eluted with 20% ethyl acetate in petroleum ether) to afford the title compound ethyl 2-(3-(4-((4′-((1,1-dioxidotetrahydro-2H-thiopyran-4-yl)oxy)-2′,6′-dimethyl-[1,1′-biphenyl]-3-yl)methoxy)phenyl)oxetan-3-yl)acetate (220 mg) as a colourless viscous liquid. Yield: 68.8%; ¹H NMR (CDCl₃, 300 MHz): δ 7.54-7.39 (m, 2H), 7.19 (s, 1H), 7.12-7.08 (m, 3H), 6.96 (d, J=6.9 Hz, 2H), 6.69 (s, 2H), 5.10 (s, 2H), 4.99 (d, J=6.0 Hz, 2H), 4.87 (d, J=6.9 Hz, 2H), 4.68 (s, 1H), 4.10 (q, J=6.8 Hz, 2H), 3.46 (t, J=11.1 Hz, 2H), 3.10 (s, 2H), 2.98 (bd, J=12.6 Hz, 2H), 2.54 (bd, J=12.0 Hz, 2H), 2.38 (t, J=12.9 Hz, 2H), 2.00 (s, 6H), 1.14 (t, J=12.9 Hz, 3H); MS (ESI): m/z 579.9 (M+1).

### Example 57

### 2-(3-(4-((4′-((1,1-Dioxidotetrahydro-2H-thiopyran-4-yl)oxy)-2′,6′-dimethyl-[1,1′-biphenyl]-3-yl)methoxy)phenyl)oxetan-3-yl)acetic acid (Compound 57)

To a solution of ethyl 2-(3-(4-((4′-((1,1-dioxidotetrahydro-2H-thiopyran-4-yl)oxy)-2′,6′-dimethyl-[1,1′-biphenyl]-3-yl)methoxy)phenyl)oxetan-3-yl)acetate (compound of Step 1d of Example 56, 62 mg, 0.107 mM) in THF:MeOH (4:1) (4 ml), aqueous Lithium hydroxide monohydrate (429 μl, 0.643 mM) was added. The mixture was stirred at RT for 4 h and the solvent was removed under reduced pressure. The reaction mixture was then neutralized with saturated NH₄Cl and extracted with ethyl acetate. The organic layer was washed with brine, dried over Na₂SO₄ and concentrated to afford the title compound 2-(3-(4-((4′-((1,1-dioxidotetrahydro-2H-thiopyran-4-yl)oxy)-2′,6′-dimethyl-[1,1′-biphenyl]-3-yl)methoxy)phenyl)oxetan-3-yl)acetic acid (56 mg) as a white solid. Yield: 95%; ¹H NMR (DMSO-d₆, 300 MHz): δ 12.13 (bs, 1H), 7.48-7.42 (m, 2H), 7.20-7.10 (m, 3H), 7.08 (d, J=6.9 Hz, 1H), 6.98 (d, J=6.9 Hz, 2H), 6.80 (s, 2H), 5.13 (s, 2H), 4.73 (s, 4H), 3.18-3.12 (m, 4H), 3.00 (s, 2H), 2.21-2.18 (m, 4H), 2.36-2.31 (m, 1H), 1.91 (s, 6H); MS: m/z 551.9 (M+1).

### Example 58

### Ethyl 2-(3-(4-((4′-(cyclopentyloxy)-2′,6′-dimethyl-[1,1′-biphenyl]-3-yl)methoxy)phenyl)oxetan-3-yl)acetate (Compound 58)

To a stirred solution of ethyl 2-(3-(4-((4′-hydroxy-2′,6′-dimethyl-[1,1′-biphenyl]-3-yl)methoxy)phenyl)oxetan-3-yl)acetate (Compound of Step 1c of Example 39, 100 mg, 0.224 mM) and cesium carbonate (1.45 g, 7.52 mM) in dry DMF (5 ml), bromocyclopentane (33.4 mg, 0.224 mM) was added at RT under nitrogen atmosphere. The reaction mixture was stirred at 80° C. for 2 h, quenched with water and extracted with ethyl acetate, dried over Na₂SO₄ and concentrated to afford the title compound ethyl 2-(3-(4-((4′-(cyclopentyloxy)-2′,6′-dimethyl-[1,1′-biphenyl]-3-yl)methoxy)phenyl)oxetan-3-yl)acetate (102 mg) as a pale yellow semisolid. The title compound was used for the next step without purification. Yield: 88%.

### Example 59

### 2-(3-(4-((4′-(Cyclopentyloxy)-2′,6′-dimethyl-[1,1′-biphenyl]-3-yl)methoxy)phenyl)oxetan-3-yl)acetic acid (Compound 59)

To a solution of ethyl 2-(3-(4-((4′-(cyclopentyloxy)-2′,6′-dimethyl-[1,1′-biphenyl]-3-yl)methoxy)phenyl)oxetan-3-yl)acetate (compound of Example 58, 40.0 mg, 0.078 mM) in THF:MeOH (4:1) (1 ml), lithium hydroxide hydrate (3.26 mg, 0.078 mM) was added. The reaction mixture was stirred at RT for 1-2 h and the solvent was removed under reduced pressure. The reaction mixture was further neutralized with saturated NH₄Cl and extracted with ethyl acetate. The organic layer was washed with brine, dried over Na₂SO₄ and concentrated to obtain the crude product which was purified by flash column chromatography (eluted with 30% ethyl acetate in petroleum ether) to afford the title compound 2-(3-(4-((4′-(cyclopentyloxy)-2′,6′-dimethyl-[1,1′-biphenyl]-3-yl)methoxy)phenyl)oxetan-3-yl)acetic acid (32 mg). Yield: 82%; ¹H NMR (CDCl₃, 300 MHz): δ 12.10 (bs, 1H), 7.47-7.41 (m, 2H), 7.21-7.06 (m, 3H), 6.98 (d, J=8.4 Hz, 2H), 6.64 (s, 2H), 5.14 (s, 2H), 4.81-4.74 (m, 5H), 3.00 (s, 2H), 1.99 (s, 6H), 1.71-1.58 (m, 8H); MS: m/z 487.1 (M+1), 585.8 (M−1).

### Example 60

### Ethyl 2-(3-(4-((2′-chloro-4′-hydroxy-[1,1′-biphenyl]-3-yl)methoxy)phenyl)oxetan-3-yl)acetate (Compound 60)

To a degassed solution of ethyl 2-(3-(4-((3-bromobenzyl)oxy)phenyl)oxetan-3-yl)acetate (compound of Step 1a of Example 39, 250 mg, 0.617 mM), (2-chloro-4-hydroxyphenyl) boronic acid (128 mg, 0.740 mM) and potassium carbonate (213 mg, 1.542 mM) in dioxane (4 ml) and water (1 ml), palladium tetrakistriphenylphosphine (35.6 mg, 0.031 mM) was added and the reaction mixture was heated in microwave at 115° C. for 10 min. The reaction mixture was concentrated and purified by column chromatography to obtain the compound ethyl 2-(3-(4-((2′-chloro-4′-hydroxy-[1,1′-biphenyl]-3-yl)methoxy)phenyl)oxetan-3-yl)acetate (220 mg, 0.486 mM) as a pale yellow solid. Yield: 79%; ¹H NMR (CDCl₃, 300 MHz): δ 7.59-7.40 (m, 4H), 7.22 (d, J=8.1 Hz, 1H), 7.13 (d, J=8.1 Hz, 3H), 6.96 (d, J=8.1 Hz, 2H), 6.82 (d, J=8.1 Hz, 2H), 6.04 (s, OH), 5.10 (s, 2H), 5.03 (d, J=6.0 Hz, 2H), 4.89 (d, J=6.0 Hz, 2H), 4.11 (q, J=6.90 Hz, 2H), 3.12 (s, 2H), 1.14 (t, J=6.90 Hz, 3H); LCMS (m/z): 475 (M+Na).

### Example 61

### Ethyl 2-(3-(4-((2′-chloro-4′-(3-(methylsulfonyl)propoxy)-[1,1′-biphenyl]-3-yl)methoxy)phenyl)oxetan-3-yl)acetate (Compound 61)

To the stirred solution of ethyl 2-(3-(4-((2′-chloro-4′-hydroxy-[1,1′-biphenyl]-3-yl)methoxy)phenyl)oxetan-3-yl)acetate (compound of Example 60, 405 mg, 0.894 mM) and 3-(methylsulfonyl)propyl 4-methylbenzenesulfonate (314 mg, 1.073 mM) dissolved in DMF (5 ml), cesium carbonate (583 mg, 1.788 mM) was added and stirred at 60° C. for 2 h. The reaction mixture was quenched with water and extracted with ethyl acetate, concentrated and purified by column chromatography to obtain the compound, ethyl 2-(3-(4-((2′-chloro-4′-(3-(methylsulfonyl)propoxy)-[1,1′-biphenyl]-3-yl)methoxy)phenyl)oxetan-3-yl)acetate (465 mg, 0.810 mM). Yield: 91%; ¹H NMR (300 MHz, CDCl₃) δ: 7.48-7.42 (m, 4H), 7.27 (d, J=2.5 Hz, 1H), 7.13 (d, 1H), 7.07 (d, J=8.3 Hz, 2H), 6.98 (d, J=2.1 Hz, 2H), 6.89 (dd, J=8.3 Hz, 2.5 Hz, 1H), 5.11 (s, 2H), 5.01 (d, J=6.0 Hz, 2H), 4.87 (d, J=6.0 Hz, 2H), 4.18 (t, J=5.3 Hz, 2H), 4.05 (q, J=5.3 Hz, 2H), 3.28 (t, J=7.2 Hz, 2H), 3.11 (s, 3H), 2.99 (s, 2H), 2.39-2.35 (m, 2H), 1.13 (t, J=7.2 Hz, 3H); MS: m/z: 573 (M⁺).

### Example 62

### 2-(3-(4-((2′-Chloro-4′-(3-(methylsulfonyl)propoxy)-[1,1′-biphenyl]-3-yl)methoxy)phenyl)oxetan-3-yl)acetic acid (Compound 62)

To a solution of ethyl 2-(3-(4-((2′-chloro-4′-(3-(methylsulfonyl)propoxy)-[1,1′-biphenyl]-3-yl)methoxy)phenyl)oxetan-3-yl)acetate (compound of Example 61, 791 mg, 1.380 mM) in 5 ml of THF:MeOH (4:1) aqueous lithium hydroxide monohydrate (5521 μl, 8.28 mM) was added and the mixture was allowed to stir at RT for 4 h. Solvent was removed and the reaction mixture was neutralized with saturated ammonium chloride and extracted with ethyl acetate. The organic layer was washed with brine, dried and concentrated to obtain 2-(3-(4-((2′-chloro-4′-(3-(methylsulfonyl)propoxy)-[1,1′-biphenyl]-3-yl)methoxy)phenyl)oxetan-3-yl)acetic acid (680 mg, 1.248 mM) as a white solid. Yield: 90%; ¹H NMR (300 MHz, DMSO-d₆): δ 12.12 (s, 1H), 7.46 (bs, 3H), 7.36-7.32 (m, 2H), 7.22-7.13 (m, 3H), 7.09-6.95 (m, 3H), 5.14 (s, 2H), 4.74 (s, 4H), 4.16 (t, J=5.7 Hz, 2H), 3.33-3.26 (m, 2H), 3.03 (s, 3H), 3.01 (s, 2H), 2.20-2.10 (m, 2H); MS (m/z): 545 (M+).

### Example 63

### Ethyl 2-(3-(4-((2′-chloro-4′-((3-methyloxetan-3-yl)methoxy)-[1,1′-biphenyl]-3-yl) methoxy)phenyl)oxetan-3-yl)acetate (Compound 63)

To the stirred solution of ethyl 2-(3-(4-((2′-chloro-4′-hydroxy-[1,1′-biphenyl]-3-yl)methoxy)phenyl)oxetan-3-yl)acetate (compound of Example 60, 100 mg, 0.221 mM) and (3-methyloxetan-3-yl)methyl 4-methylbenzenesulfonate[70 mg, 0.273 mM; prepared by reacting (3-methyloxetan-3-yl)methanol with 4-methylbenzene-1-sulfonyl chloride] dissolved in DMF (5 ml), cesium carbonate (144 mg, 0.442 mM) was added and stirred at 80° C. for 4 h. The reaction mixture was quenched with water and extracted with ethyl acetate, concentrated and purified by column chromatography to obtain ethyl 2-(3-(4-((2′-chloro-4′-((3-methyloxetan-3-yl)methoxy)-[1,1′-biphenyl]-3-yl)methoxy)phenyl)oxetan-3-yl)acetate as colorless viscous liquid (109 mg, 0.203 mM). Yield: 92%; ¹H NMR (300 MHz, CDCl₃) δ: 7.49-7.40 (m, 4H), 7.31 (s, 1H), 7.13-7.08 (m, 3H), 6.99 (d, J=8.7 Hz, 2H), 6.94 (dd, J=8.7 Hz, 1.5 Hz, 1H), 5.11 (s, 2H), 5.01 (d, J=5.4 Hz, 2H), 4.87 (d, J=5.7 Hz, 2H), 4.66 (d, J=6.0 Hz, 2H), 4.50 (d, J=5.7 Hz, 2H), 4.07 (s, 2H), 4.10 (q, J=7.2 Hz, 2H), 3.11 (s, 2H), 1.47 (s, 3H), 1.11 (t, J=7.2 Hz, 3H); MS: m/z 559 (M+Na).

### Example 64

### 2-(3-(4-((2′-Chloro-4′-((3-methyloxetan-3-yl)methoxy)-[1,1′-biphenyl]-3-yl)methoxy)phenyl)oxetan-3-yl)acetic acid (Compound 64)

To a solution of ethyl 2-(3-(4-((2′-chloro-4′-((3-methyloxetan-3-yl)methoxy)-[1,1′-biphenyl]-3-yl)methoxy)phenyl)oxetan-3-yl)acetate (compound of Example 63, 87 mg, 0.162 mM) in 5 ml of THF:MeOH (4:1) aqueous lithium hydroxide monohydrate (648 μl, 0.972 mM) was added and the mixture was stirred at RT for 4 h. Solvent was removed and the reaction mixture was neutralized with saturated ammonium chloride. The reaction mixture was extracted with ethyl acetate. The organic layer was washed with brine, dried and concentrated to obtain 2-(3-(4-((2′-chloro-4′-((3-methyloxetan-3-yl)methoxy)-[1,1′-biphenyl]-3-yl)methoxy)phenyl)oxetan-3-yl)acetic acid (60 mg, 0.118 mM) as white solid. Yield: 72.8%; ¹H NMR (300 MHz, DMSO-d₆) δ: 11.68 (s, 1H), 7.46-7.40 (bm, 3H), 7.35 (d, J=8.1 Hz, 2H), 7.23-7.11 (m, 2H), 7.09-7.00 (m, 2H), 6.98 (d, J=8.7 Hz, 2H), 5.11 (s, 2H), 4.74 (s, 4H), 4.50 (d, J=5.4 Hz, 2H), 4.32 (d, J=6.0 Hz, 2H), 4.12 (d, J=5.7 Hz, 2H), 3.01 (s, 2H), 1.37 (s, 3H); MS: m/z 508 (M+).

### Example 65

### Ethyl 2-(3-(4-((2′-chloro-4′-((3-(hydroxymethyl)oxetan-3-yl)methoxy)-[1,1′-biphenyl]-3-yl)methoxy)phenyl)oxetan-3-yl)acetate (Compound 65)

To a stirred solution of ethyl 2-(3-(4-((2′-chloro-4′-hydroxy-[1,1′-biphenyl]-3-yl)methoxy)phenyl)oxetan-3-yl)acetate (compound of Example 60, 102 mg, 0.225 mM) and (3-(bromomethyl)oxetan-3-yl)methanol (53.0 mg, 0.293 mM) in DMF (5 ml), cesium carbonate (147 mg, 0.450 mM) was added and stirred at 60° C. for 2 h. The reaction mixture was quenched with water and extracted with ethyl acetate. Organic layer was washed with brine, dried and concentrated. The crude compound was purified by column chromatography to obtain ethyl 2-(3-(4-((2′-chloro-4′-((3-(hydroxymethyl)oxetan-3-yl)methoxy)-[1,1′-biphenyl]-3-yl)methoxy)phenyl)oxetan-3-yl)acetate (45 mg, 0.081 mM) as colorless liquid. Yield: 36%; ¹H NMR (300 MHz, CDCl₃) δ: 7.49-7.41 (m, 4H), 7.31 (d, J=8.4 Hz, 1H), 7.14-7.04 (m, 3H), 6.96-6.92 (m, 3H), 5.11 (s, 2H), 5.01 (d, J=6.4 Hz, 2H), 4.87 (d, J=6.4 Hz, 2H), 4.61 (s, 4H), 4.30 (s, 2H), 4.15 (s, 2H), 3.98 (q, J=6.4 Hz, 2H), 3.11 (s, 2H), 1.02 (t, J=6.9 Hz, 3H); MS: (m/z) 553 (M+).

### Example 66

### 2-(3-(4-((2′-Chloro-4′-((3-(hydroxymethyl)oxetan-3-yl)methoxy)-[1,1′-biphenyl]-3-yl)methoxy)phenyl)oxetan-3-yl)acetic acid (Compound 66)

To a solution of ethyl 2-(3-(4-((2′-chloro-4′-((3-(hydroxymethyl)oxetan-3-yl)methoxy)-[1,1′-biphenyl]-3-yl)methoxy)phenyl)oxetan-3-yl)acetate (compound of Example 65, 60 mg, 0.108 mM) in 5 ml of THF:MeOH (4:1) lithium hydroxide hydrate (434 μl, 0.651 mM) was added and the mixture was stirred at RT for 2-3 h. Solvent was removed and the reaction mixture was neutralized with saturated ammonium chloride. The mixture was then extracted with ethyl acetate. The organic layer was washed with brine, dried and concentrated to obtain 2-(3-(4-((2′-chloro-4′-((3-(hydroxymethyl)oxetan-3-yl)methoxy)-[1,1′-biphenyl]-3-yl)methoxy)phenyl)oxetan-3-yl)acetic acid (25 mg, 0.048 mM) as a white solid. Yield: 43.9%; ¹H NMR (300 MHz, CDCl₃) δ: 12.88 (s, OH), 7.49-7.41 (m, 5H), 7.13-7.08 (m, 3H), 6.98-6.90 (m, 3H), 5.10 (s, 2H), 4.99 (d, J=6.4 Hz, 2H), 4.85 (d, J=6.4 Hz, 2H), 4.60 (s, 4H), 4.28 (s, 2H), 4.05 (s, 2H), 3.15 (s, 2H), 1.8 (bs, OH); MS: (m/z) 525 (M+1).

### Example 67

### Ethyl 2-(3-(4-((2′-chloro-4′-((1,1-dioxidotetrahydrothiophen-3-yl)methoxy)-[1,1′-biphenyl]-3-yl)methoxy)phenyl)oxetan-3-yl)acetate (Compound 67)

To the stirred solution of ethyl 2-(3-(4-((2′-chloro-4′-hydroxy-[1,1′-biphenyl]-3-yl)methoxy)phenyl)oxetan-3-yl)acetate (compound of Example 60,100 mg, 0.221 mM) and (1,1-dioxidotetrahydrothiophen-3-yl)methyl 4-methylbenzenesulfonate (compound of Step 1b of Example 52, 67.2 mg, 0.221 mM) dissolved in DMF (3 ml), cesium carbonate (144 mg, 0.442 mM) was added and stirred at 80° C. for 4 h. The reaction mixture was quenched with water and extracted with ethyl acetate, concentrated and purified by column chromatography to obtain the pure compound ethyl 2-(3-(4-((2′-chloro-4′-((1,1-dioxidotetrahydrothiophen-3-yl)methoxy)-[1,1′-biphenyl]-3-yl) methoxy)phenyl)oxetan-3-yl)acetate (90 mg, 0.154 mM) as colorless viscous liquid. Yield: 69.7%; ¹H NMR (300 MHz, CDCl₃): δ 7.48-7.40 (m, 4H), 7.31 (s, 1H), 7.13 (d, J=8.4 Hz, 2H), 7.02 (d, J=1.8 Hz, 1H), 6.98 (d, J=8.7 Hz, 2H), 6.89 (dd, J=8.7, 1.5 Hz, Hz, 1H), 5.11 (s, 2H), 5.00 (d, J=5.5 Hz, 2H), 4.87 (d, J=6.1 Hz, 2H), 4.15-3.99 (m, 4H), 3.38-3.25 (m, 2H), 3.20-2.90 (m, including s at 3.07, 5H), 2.47-2.44 (m, 1H), 2.26-2.15 (m, 1H), 1.30 (t, J=8.7 Hz, 3H); MS: (m/z) 585 (M+).

### Example 68

### 2-(3-(4-((2′-Chloro-4′-((1,1-dioxidotetrahydrothiophen-3-yl)methoxy)-[1,1′-biphenyl]-3-yl)methoxy)phenyl)oxetan-3-yl)acetic acid (Compound 68)

To a solution of ethyl 2-(3-(4-((2′-chloro-4′-((1,1-dioxidotetrahydrothiophen-3-yl)methoxy)-[1,1′-biphenyl]-3-yl)methoxy)phenyl)oxetan-3-yl)acetate (compound of Example 67, 87 mg, 0.149 mM) in 5 ml of THF:MeOH (4:1), aqueous lithium hydroxide mono hydrate (595 μl, 0.892 mM) was added and the mixture was stirred at RT for 4 h. Solvent was removed and the reaction mixture was neutralized with saturated ammonium chloride. The mixture was then extracted with ethyl acetate. The organic layer was washed with brine, dried and concentrated to obtain the title compound, 2-(3-(4-((2′-chloro-4′-((1,1-dioxidotetrahydrothiophen-3-yl)methoxy)-[1,1′-biphenyl]-3-yl) methoxy)phenyl)oxetan-3-yl)acetic acid (57 mg) as white solid. Yield: 68%; ¹H NMR (300 MHz, DMSO-d₆) δ: 12.10 (bs, 1H), 7.45-7.35 (m, 5H), 7.20-7.18 (m, 3H), 7.12-7.00 (m, 3H), 5.13 (s, 2H), 4.73 (s, 4H), 4.12 (d, J=5.7 Hz, 2H), 3.00-2.80 (m including s at 2.30, 7H), 2.32-2.28 (m, 1H), 1.93-1.90 (m, 1H); MS: m/z 556.0 (M+).

### Example 69

### Ethyl 2-(3-(4-((2′-chloro-4′-((1,1-dioxidotetrahydro-2H-thiopyran-4-yl)methoxy)-[1,1′-biphenyl]-3-yl)methoxy)phenyl)oxetan-3-yl)acetate (Compound 69)

To the stirred solution of ethyl 2-(3-(4-((2′-chloro-4′-hydroxy-[1,1′-biphenyl]-3-yl)methoxy)phenyl)oxetan-3-yl)acetate (compound of Example 60, 336 mg, 0.742 mM) and (1,1-dioxidotetrahydro-2H-thiopyran-4-yl)methyl 4-methylbenzenesulfonate (compound of Step 1a of Example 44, 236 mg, 0.742 mM) dissolved in DMF (5 ml), cesium carbonate (483 mg, 1.484 mM) was added and stirred at 80° C. for 4 h. The reaction mixture was quenched with water and extracted with ethyl acetate, concentrated and purified by column chromatography to obtain the title compound, ethyl 2-(3-(4-((2′-chloro-4′-((1,1-dioxidotetrahydro-2H-thiopyran-4-yl)methoxy)-[1,1′-biphenyl]-3-yl) methoxy)phenyl)oxetan-3-yl)acetate (350 mg, 0.584 mM) as white solid. Yield: 79%; ¹H NMR (500 MHz, CDCl₃): δ 7.48-7.40 (m, 4H), 7.30 (s, 1H), 7.13 (d, J=8.7 Hz, 2H), 7.02 (d, J=2.5 Hz, 1H), 6.98 (d, J=8.7 Hz, 2H), 6.88 (d, J=8.7 Hz, 1H), 5.11 (s, 2H), 5.00 (d, J=5.5 Hz, 2H), 4.87 (d, J=6.1 Hz, 2H), 4.15 (q, J=6.1 Hz, 2H), 3.92 (s, 2H), 3.18 (d, J=13.5 Hz, 2H), 3.11 (d, J=13.5 Hz, 2H), 2.97 (t, J=13.5 Hz, 2H), 2.33 (d, J=10.5 Hz, 2H), 2.08-2.06 (m, 3H), 1.13 (t, J=8.7 Hz, 3H); MS: (m/z) 599 (M+)

### Example 70

### 2-(3-(4-((2′-Chloro-4′-((1,1-dioxidotetrahydro-2H-thiopyran-4-yl)methoxy)-[1,1′-biphenyl]-3-yl)methoxy)phenyl)oxetan-3-yl)acetic acid (Compound 70)

Ethyl 2-(3-(4-((2′-chloro-4′-((1,1-dioxidotetrahydro-2H-thiopyran-4-yl)methoxy)-[1,1′-biphenyl]-3-yl)methoxy)phenyl)oxetan-3-yl)acetate (compound of Example 69, 370 mg, 0.618 mM) was dissolved in a mixture of THF (4 ml) and MeOH (1 ml) and aqueous lithium hydroxide monohydrate (2470 μl, 3.71 mM) was added to it and the mixture was stirred for 6 h. The reaction mixture was quenched with saturated ammonium chloride and extracted with ethyl acetate. The organic layer was washed with brine, dried and concentrated to obtain the title compound, 2-(3-(4-((2′-chloro-4′-((1,1-dioxidotetrahydro-2H-thiopyran-4-yl)methoxy)-[1,1′-biphenyl]-3-yl)methoxy)phenyl)oxetan-3-yl)acetic acid (23 mg, 0.040 mM) as white solid. Yield: 6.52%; ¹H NMR (300 MHZ, DMSO-d₆): δ 12.0 (s, 1H, OH), 7.45 (bs, 3H), 7.38-7.31 (m, 2H), 7.22-7.16 (m, 3H), 7.03-6.98 (m, 3H), 5.14 (s, 2H), 4.74 (s, 4H), 3.98 (d, J=5.1 Hz, 2H), 3.19 (t, J=14 2 Hz, 2H), 3.05 (t, J=14 2 Hz, 2H), 3.01 (s, 2H), 2.16-2.12 (m, 3H), 1.82-1.70 (m, 2H); MS: (m/z) 593 (M+Na).

### Example 71

### Ethyl 2-(3-(4-((2′-chloro-4′-((tetrahydro-2H-pyran-4-yl)methoxy)-[1,1′-biphenyl]-3-yl) methoxy)phenyl)oxetan-3-yl)acetate (Compound 71)

To a stirred solution of ethyl 2-(3-(4-((2′-chloro-4′-hydroxy-[1,1′-biphenyl]-3-yl)methoxy)phenyl)oxetan-3-yl)acetate (compound of Example 60,100 mg, 0.221 mM) and (tetrahydro-2H-pyran-4-yl)methyl4-methylbenzenesulfonate (compound of Step 1a of Example 42, 71.6 mg, 0.265 mM) dissolved in DMF (5 ml), cesium carbonate (144 mg, 0.442 mM) was added and stirred at 60° C. for 2 h. The reaction mixture was quenched with water and extracted with ethyl acetate, concentrated and purified by column chromatography to obtain the title compound, ethyl 2-(3-(4-((2′-chloro-4′-((tetrahydro-2H-pyran-4-yl)methoxy)-[1,1′-biphenyl]-3-yl)methoxy)phenyl)oxetan-3-yl)acetate (80 mg, 0.145 mM). Yield: 65.8%; ¹H NMR (300 MHz, CDCl₃): δ 7.49-7.41 (m, 4H), 7.26-7.25 (m, 1H), 7.13 (d, J=8.4 Hz, 2H), 7.02 (bs, 1H), 6.98 (d, J=8.4 Hz, 2H), 6.89 (dd, J=8.4 Hz, 2.5 Hz, 1H), 5.11 (s, 2H), 5.01 (d, J=5.7 Hz, 2H), 4.87 (d, J=5.7 Hz, 2H), 4.10-3.98 (m, 4H), 3.86 (d, J=5.7 Hz, 2H), 3.47 (t, J=11.38 Hz, 2H), 3.11 (s, 2H), 1.81 (d, J=12.3 Hz, 2H), 1.58-1.47 (m, 3H), 1.13 (t, J=6.5 Hz, 3H); MS: (m/z) 552 (M+).

### Example 72

### 2-(3-(4-((2′-Chloro-4′-((tetrahydro-2H-pyran-4-yl)methoxy)-[1,1′-biphenyl]-3-yl) methoxy)phenyl)oxetan-3-yl)acetic acid (Compound 72)

To a solution of ethyl 2-(3-(4-((2′-chloro-4′-((tetrahydro-2H-pyran-4-yl)methoxy)-[1,1′-biphenyl]-3-yl)methoxy)phenyl)oxetan-3-yl)acetate (compound of Example 71, 88 mg, 0.160 mM) in 5 ml of THF:MeOH (4:1) aqueous lithium hydroxide hydrate (40.2 mg, 0.958 mM) was added and the mixture was stirred at RT for 4 h. Solvent was removed, and the reaction mixture was neutralized with saturated ammonium chloride, extracted with ethyl acetate. The organic layer was washed with brine, dried and concentrated to obtain 2-(3-(4-((2′-chloro-4′-((tetrahydro-2H-pyran-4-yl)methoxy)-[1,1′-biphenyl]-3-yl)methoxy)phenyl)oxetan-3-yl)acetic acid (65 mg, 0.124 mM) as white solid. Yield: 78%; ¹H NMR (300 MHz, DMSO-d₆): δ 12.8 (s, 1H); 7.45 (s, 3H), 7.33-7.30 (m, 2H), 7.22-7.13 (m, 3H), 7.00-6.97 (m, 3H), 5.14 (s, 2H), 4.74 (s, 4H), 3.91-3.87 (m, 4H), 3.31 (t, J=6.5 Hz, 2H), 3.00 (s, 2H), 2.1-1.99 (m, 1H), 1.70 (d, J=11.8 Hz, 2H), 1.34-1.24 (m, 2H); MS: (m/z) 523 (M+).

### Example 73

### Ethyl 2-(3-(4-((4′-hydroxy-[1,1′-biphenyl]-3-yl) methoxy)phenyl)oxetan-3-yl)acetate (Compound 73)

To a solution of ethyl 2-(3-(4-((3-bromobenzyl)oxy)phenyl)oxetan-3-yl)acetate (compound of Step 1a of Example 39, 300 mg, 074 mM), (4-hydroxyphenyl) boronic acid (153 mg, 1.11 mM) in DMF:H₂O (2 ml:0.2 ml) sodium carbonate (157 mg, 1.48 mM) was added. To the resulting solution PdCl₂(PPh₃)₂ (26 mg, 0.037 mM) was added and the mixture was heated at 120° C. for 10 min in microwave. The reaction mixture was diluted with ethyl acetate (100 ml) and water (10 ml). The organic layer was separated and washed with brine, dried and concentrated. The crude compound was purified by column chromatography to obtain the compound ethyl 2-(3-(4-((4′-hydroxy-[1,1′-biphenyl]-3-yl) methoxy)phenyl)oxetan-3-yl)acetate (125 mg, 0.263 mM). Yield: 36%; ¹H NMR (300 MHz, CDCl₃): δ 9.56 (s, 1H), 7.64 (s, 1H), 7.54-7.44 (m, 3H), 7.42 (d, J=7.8 Hz, 1H), 7.35 (d, J=7.2 Hz, 1H), 7.18 (d, J=8.4 Hz, 2H), 7.01 (d, J=8.4 Hz, 2H), 6.86 (d, J=8.1 Hz, 2H), 5.14 (s, 2H), 4.75 (s, 4H), 3.93-3.86 (q, J=6.9, Hz, 2H), 3.07 (s, 2H), 1.04 (t, J=6.9 Hz, 3H); MS: (m/z) 441 (M+Na).

### Example 74

### Ethyl 2-(3-(4-((4′-(cyclobutylmethoxy)-[1,1′-biphenyl]-3-yl)methoxy)phenyl)oxetan-3-yl)acetate (Compound 74)

To the stirred solution of ethyl 2-(3-(4-((4′-hydroxy-[1,1′-biphenyl]-3-yl)methoxy)phenyl)oxetan-3-yl)acetate (compound of Example 73, 50 mg, 0.119 mM) and (bromomethyl)cyclobutane (0.020 ml, 0.179 mM) dissolved in DMF (5 ml), cesium carbonate (97 mg, 0.299 mM) was added and stirred at RT for 2 h. The reaction mixture was quenched with water and extracted with ethyl acetate, concentrated and purified by column chromatography to obtain the pure compound ethyl 2-(3-(4-((2′-chloro-4′-(3-(methylsulfonyl)propoxy)-[1,1′-biphenyl]-3-yl)methoxy)phenyl)oxetan-3-yl)acetate (27 mg, 0.055 mM). Yield: 46%; ¹H NMR (300 MHz, CDCl₃): δ 7.62 (s, 1H), 7.55 (d, J=8.4 Hz, 3H), 7.47-7.42 (t, J=7.5, 15 Hz, 1H), 7.38 (d, J=6.9 Hz, 1H), 7.13 (d, J=8.4 Hz, 2H), 7.00 (d, J=6.6 Hz, 4H), 5.11 (s, 2H), 5.01 (d, J=5.7 Hz, 2H), 4.87 (d, J=6 Hz, 2H), 4.05-3.98 (m, 4H), 3.11 (s, 2H), 2.83-2.79 (m, 1H), 2.23-2.19 (m, 2H), 2.02-1.94 (m, 4H), 1.15-1.11 (t, J=6.9, 14.1 Hz, 3H); MS: (m/z) 509 (M+Na).

### Example 75

### 2-(3-(4-((4′-(Cyclobutylmethoxy)-[1,1′-biphenyl]-3-yl)methoxy)phenyl)oxetan-3-yl)acetic acid (Compound 75)

To a solution of ethyl 2-(3-(4-((4′-(cyclobutylmethoxy)-[1,1′-biphenyl]-3-yl)methoxy)phenyl)oxetan-3-yl)acetate (compound of Example 74, 74 mg, 0.015 mM) in 5 ml of THF:MeOH (4:1), lithium hydroxide hydrate (38 mg, 0.912 mM) was added and the mixture was stirred at RT for 4 h. Solvent was removed and the reaction mixture was neutralized with saturated ammonium chloride and extracted with ethyl acetate, dried and concentrated to obtain the compound 2-(3-(4-((4′-(cyclobutylmethoxy)-[1,1′-biphenyl]-3-yl)methoxy)phenyl)oxetan-3-yl)acetic acid (35 mg, 0.076 mM). Yield: 50%; ¹H NMR (300 MHz, CDCl₃): δ 12.12 (s, 1H), 7.68 (s, 2H), 7.58 (s, 2H), 7.44 (d, J=16.2 Hz, 2H), 7.20 (s, 2H), 7.02 (s, 4H), 5.15 (s, 2H), 4.79 (s, 4H), 4.00 (s, 2H), 3.01 (s, 2H), 2.73 (s, 2H), 2.09 (s, 2H), 1.99-1.88 (m, 2H), 1.24-1.17 (m, 1H); MS: (m/z) 457 (M−1).

### Example 76

### Ethyl 2-(3-(4-((2′-methyl-4′-(3-(methylsulfonyl)propoxy)-[1,1′-biphenyl]-3-yl)methoxy)phenyl)oxetan-3-yl)acetate (Compound 76)

### Step 1a

### Synthesis of 4′-Hydroxy-2′-methyl-[1,1′-biphenyl]-3-carbaldehyde

To a solution of 4-bromo-3-methylphenol (300 mg. 1.604 mM) and (3-formylphenyl)boronic acid (361 mg, 2.406 mM) in DMF:H₂O (2 ml:0.2 ml), sodium carbonate (340 mg, 3.21 mM) was added. To the resulting solution PdCl₂(PPh₃)₂ (56 mg, 0.08 mM) was added and the mixture was heated at 120° C. for 10 min in microwave. The reaction mixture was diluted with ethyl acetate (100 ml) and water (10 ml), organic layer was separated and washed with brine, dried and concentrated. The crude compound was purified by column chromatography to obtain the compound 4′-hydroxy-2′-methyl-[1,1′-biphenyl]-3-carbaldehyde (68 mg, 0.320 mM). Yield: 20%; ¹H NMR (300 MHz, DMSO-d₆): δ 10.07 (s, 1H), 7.86-7.83 (m, 2H), 7.59 (d, J=4.2 Hz, 2H), 7.14 (d, J=8.1 Hz, 2H), 6.80-6.75 (m, 2H), 2.25 (s, 3H); MS: (m/z) 213 (M+1).

### Step 1b

### Synthesis of 3′-(Hydroxymethyl)-2-methyl-[1,1′-biphenyl]-4-ol

To a solution of 4′-hydroxy-2′-methyl-[1,1′-biphenyl]-3-carbaldehyde (compound of Step 1a, 50 mg, 0.236 mM) in methanol (2 ml), sodium borohydride (11 mg, 0.283 mM) was added at 0° C. and stirred. The reaction mixture was quenched by adding aqueous ammonium chloride, diluted with ethyl acetate (100 ml), organic layer was separated and washed with brine, dried and concentrated. The crude compound was purified by column chromatography to obtain the compound 3′-(hydroxymethyl)-2-methyl-[1,1′-biphenyl]-4-ol (50 mg, 0.226 mM). Yield: 96%; ¹H NMR (300 MHz, DMSO-d₆): δ 9.33 (s, 1H), 7.36-7.31 (m, 1H), 7.25 (s, 1H), 7.23 (d, J=6.6 Hz, 1H), 7.14 (d, J=7.2 Hz, 1H), 7.00 (d, J=8.1 Hz, 1H), 6.68 (d, J=5.7 Hz, 1H), 6.63 (s, 1H), 5.20-5.16 (t, J=5.4, 11.1 Hz, 1H), 4.53 (d, J=5.7 Hz, 2H), 2.15 (s, 3H); MS: (m/z) 237 (M+Na).

### Step 1c

### Synthesis of (2′-Methyl-4′-(3-(methylsulfonyl)propoxy)-[1,1′-biphenyl]-3-yl) methanol

To a solution of 3′-(hydroxymethyl)-2-methyl-[1,1′-biphenyl]-4-ol (compound of Step 1b, 50 mg, 0.233 mM) and 3-(methylsulfonyl)propyl 4-methylbenzenesulfonate (75 mg, 0.257 mM) in anhydrous DMF (2 ml), cesium carbonate (76 mg, 0.233 mM) was added at RT. The reaction mixture was stirred at RT for 2 h. Reaction was then quenched by addition of water (5 ml) and allowed to stir for 10 min and then extracted with ethyl acetate. The organic layer was washed with brine, dried and concentrated. The residue was purified by flash column chromatography (silica gel, 40% ethyl acetate in n-hexane) to obtain the compound ethyl 2-(3-(4-((2′-methyl-4′-(3-(methylsulfonyl)propoxy)-[1,1′-biphenyl]-3-yl) methoxy)phenyl)oxetan-3-yl) acetate (98 mg, 0.170 mM) as colorless oil. Yield: 99%; ¹H NMR (300 MHz, CDCl₃): δ 7.44-7.39 (m, 1H), 7.39-7.31 (m, 2H), 7.25 (d, J=7.5 Hz, 1H), 7.18 (d, J=8.4 Hz, 1H), 6.81 (d, J=5.4 Hz, 1H), 6.77 (s, 1H), 4.76 (d, J=4.8 Hz, 2H), 4.18-4.14 (t, J=5.4, 11.1 Hz, 2H), 3.32-3.27 (t, J=15.6, 15.3 Hz, 2H), 2.98 (s, 3H), 2.40-2.35 (m, 2H), 2.27 (s, 3H), 1.70 (t, J=5.4, 11.1 Hz, 1H); MS: (m/z) 357 (M+Na).

### Step 1d

### Synthesis of 3′-(Bromomethyl)-2-methyl-4-(3-(methylsulfonyl)propoxy)-1,1′-biphenyl

To (2′-methyl-4′-(3-(methylsulfonyl)propoxy)-[1,1′-biphenyl]-3-yl)methanol (compound of Step 1c, 170 mg, 0.508 mM) in DCM at 0° C., PBr₃ (138 mg, 0.508 mM) was added and stirred for 1 h. Saturated NaHCO₃ was added and extracted with DCM, dried and concentrated to obtain the compound 3′-(bromomethyl)-2-methyl-4-(3-(methylsulfonyl)propoxy)-1,1′-biphenyl (150 mg, 0.378 mM). ¹HNMR (300 MHz, DMSO-d₆): δ 7.41-7.39 (m, 3H), 7.25 (s, 1H), 7.14 (d, J=8.4 Hz, 1H), 6.89 (s, 1H), 6.86 (d, J=8.1 Hz, 1H), 4.75 (s, 2H), 4.13-4.10 (t, J=6, 11.7 Hz, 2H), 3.28-3.26 (m, 2H), 3.03 (s, 3H), 2.21 (s, 3H), 2.18-2.15 (m, 2H); MS: (m/z) 420 (M+Na).

### Step 1e

### Ethyl 2-(3-(4-((2′-methyl-4′-(3-(methylsulfonyl)propoxy)-[1,1′-biphenyl]-3-yl)methoxy)phenyl)oxetan-3-yl)acetate (Compound 76)

To a solution of ethyl 2-(3-(4-hydroxyphenyl)oxetan-3-yl)acetate (compound of Step 1b of Example 1, 40 mg, 0.169 mM) and 3′-(bromomethyl)-2-methyl-4-(3-(methylsulfonyl)propoxy)-1,1′-biphenyl (compound of Step 1d, 60 mg, 0.152 mM) in anhydrous DMF (2 ml), cesium carbonate (110 mg, 0.339 mM) was added at RT. The reaction mixture was stirred at RT for 2 h. Reaction was then quenched with addition of water (5 ml) and allowed to stir for 10 min, and then extracted with ethyl acetate. The organic layer was washed with brine, dried and concentrated. The residue was purified by flash column chromatography (silica gel, 40% ethyl acetate in n-hexane) to obtain the compound ethyl 2-(3-(4-((2′-methyl-4′-(3-(methylsulfonyl)propoxy)-[1,1′-biphenyl]-3-yl)methoxy)phenyl)oxetan-3-yl)acetate (98 mg, 0.170 mM) as colorless oil. Yield: 99%; ¹H NMR (300 MHz, DMSO-d₆): δ 7.41 (s, 1H), 7.36 (s, 1H), 7.27 (d, J=6.3 Hz, 1H), 7.18-7.11 (m, 3H), 7.00 (d, J=8.4 Hz, 2H), 6.88-6.83 (m, 2H), 6.72 (d, J=7.8 Hz, 1H), 5.14 (s, 2H), 4.75 (s, 4H), 4.11-4.01 (q, J=6.9 Hz, 2H), 3.90 (d, J=7.2 Hz, 2H), 3.26 (s, 3H), 3.08 (s, 2H), 3.03 (s, 2H), 2.18 (s, 3H), 1.99 (s, 2H), 1.04 (t, J=6.6 Hz, 3H); MS: (m/z) 553 (M+1).

### Example 77

### 2-(3-(4-((2′-Methyl-4′-(3-(methylsulfonyl)propoxy)-[1,1′-biphenyl]-3-yl)methoxy)phenyl)oxetan-3-yl)acetic acid (Compound 77)

To a solution of ethyl 2-(3-(4-((2′-methyl-4′-(3-(methylsulfonyl)propoxy)-[1,1′-biphenyl]-3-yl)methoxy)phenyl)oxetan-3-yl)acetate (compound of Example 76, 76 mg, 0.138 mM) in 5 ml of THF:MeOH (4:1), lithium hydroxide hydrate (34 mg, 5.78 mM) was added and the mixture was stirred at RT for 6 h. Solvent was removed and the reaction mixture was neutralized with saturated ammonium chloride and extracted with ethyl acetate, dried and concentrated to obtain the compound 2-(3-(4-((2′-methyl-4′-(3-(methylsulfonyl)propoxy)-[1,1′-biphenyl]-3-yl)methoxy)phenyl)oxetan-3-yl)acetic acid (35 mg, 0.059 mM). Yield: 43%; ¹H NMR (300 MHz, DMSO-d₆): δ 12.18 (s, 1H), 7.43-7.34 (m, 2H), 7.25 (d, J=6.3 Hz, 1H), 7.20 (d, J=8.4 Hz, 2H), 7.13 (d, J=8.1 Hz, 2H), 6.99 (d, J=8.4 Hz, 2H), 6.88 (s, 1H), 6.86 (d, J=8.4 Hz, 1H), 5.12 (s, 2H), 4.73 (s, 4H), 4.12-4.08 (t, J=6 Hz, 2H), 3.29 (s, 2H), 2.82-2.80 (m, 2H), 3.01 (s, 5H), 2.17 (s, 3H); MS: (m/z) 525 (M+1).

### Example 78

### Ethyl 2-(3-(4-((3′,5′-dimethyl-4′-(3-(methylsulfonyl)propoxy)-[1,1′-biphenyl]-3-yl)methoxy)phenyl)oxetan-3-yl)acetate (Compound 78)

### Step 1a

### Synthesis of 4′-Hydroxy-3′,5′-dimethyl-[1,1′-biphenyl]-3-carbaldehyde

To a solution of 4-bromo-2,6-dimethylphenol (300 mg, 1.492 mM) and (3-formylphenyl)boronic acid (268 mg, 1.791 mM) in DMF:H₂O (2 ml: 0.2 ml), sodium carbonate (285 mg, 2.69 mM) was added. To the resulting solution PdCl₂(PPh₃)₂ (21 mg, 0.030 mM) was added and the mixture was heated at 120° C. for 10 min in microwave. The reaction mixture was diluted with ethyl acetate (100 ml) and water (10 ml), organic layer was separated and washed with brine, dried and concentrated. The crude compound was purified by column chromatography to obtain the compound 4′-hydroxy-3′,5′-dimethyl-[1,1′-biphenyl]-3-carbaldehyde (41 mg, 0.154 mM). Yield: 10%; ¹H NMR (300 MHz, DMSO-d₆): δ 10.13 (d, J=17.4 Hz, 1H), 8.48 (s, 1H), 8.11 (s, 1H), 7.94 (d, J=7.8 Hz, 1H), 7.81 (d, J=7.5 Hz, 1H), 7.65 (t, J=7.8 Hz, 1H), 7.33 (s, 2H), 2.24 (s, 6H); MS: (m/z) 227 (M+1).

### Step 1b

### Synthesis of 3′,5′-Dimethyl-4′-(3-(methylsulfonyl)propoxy)-[1,1′-biphenyl]-3-carbaldehyde

A mixture of 4′-hydroxy-3′,5′-dimethyl-[1,1′-biphenyl]-3-carbaldehyde (compound of Step 1a, 50 mg, 0.221 mM), 3-(methylsulfonyl)propyl 4-methylbenzenesulfonate (78 mg, 0.265 mM) and cesium carbonate (108 mg, 0.331 mM) in DMF was stirred for 2 h. The reaction mixture was concentrated. The crude compound was purified by column chromatography to obtain the compound 3′,5′-dimethyl-4′-(3-(methylsulfonyl)propoxy)-[1,1′-biphenyl]-3-carbaldehyde (55 mg) as colorless liquid. Yield: 71%; ¹HNMR (300 MHz, CDCl₃): δ 10.08 (s, 1H), 8.15 (s, 1H), 7.98 (d, J=7.8 Hz, 1H), 7.87 (d, J=7.5 Hz, 1H), 7.81 (d, J=8.1 Hz, 1H), 7.68-7.63 (t, J=7.5, 15 Hz, 1H), 7.51 (d, J=8.1 Hz. 1H), 3.91-3.87 (t, J=6, 12 Hz, 2H), 3.40-3.37 (m, 2H), 3.04 (s, 3H), 2.31 (s, 6H), 2.20-2.15 (m, 2H); MS: (m/z) 369 (M+Na).

### Step 1c

### Synthesis of (3′,5′-Dimethyl-4′-(3-(methylsulfonyl)propoxy)-[1,1′-biphenyl]-3-yl) methanol

A mixture of 3′,5′-dimethyl-4′-(3-(methylsulfonyl)propoxy)-[1,1′-biphenyl]-3-carbaldehyde (compound of Step 1b, 45 mg, 0.130 mM) and NaBH₄ (6 mg, 0.156 mM) in dry methanol was stirred for 1 h. The reaction mixture was concentrated and the crude compound was purified by column chromatography to obtain the compound (3′,5′-dimethyl-4′-(3-(methylsulfonyl)propoxy)-[1,1′-biphenyl]-3-yl) methanol (35 mg, 0.092 mM). Yield: 71%; ¹H NMR (300 MHz, CDCl₃): δ 7.81 (d, J=7.8 Hz, 1H), 7.54-7.50 (m, 1H), 7.47-7.45 (d, J=8.4 Hz, 1H), 7.39-7.37 (d, J=7.5 Hz, 1H), 7.31 (s, 1H), 7.27 (d, J=7.2 Hz, 1H), 5.23-5.19 (t, J=5.7, 10.8 Hz, 2H), 4.55 (d, J=5.4 Hz, 2H), 4.14-4.10 (m, 1H), 3.89-3.85 (t, J=5.7, 11.4 Hz, 2H), 3.39 (s, 3H), 3.04 (s, 6H), 2.96-2.90 (m, 2H); MS: (m/z) 371 (M+Na).

### Step 1d

### Synthesis of 3′-(Bromomethyl)-3,5-dimethyl-4-(3-(methylsulfonyl)propoxy)-1,1′-biphenyl

To a solution of (2′-methyl-4′-(3-(methylsulfonyl)propoxy)-[1,1′-biphenyl]-3-yl) methanol (compound of Step 1c, 170 mg, 0.508 mM) in DCM at 0° C., PBr₃ (138 mg, 0.508 mM) was added and stirred for 1 h. Saturated NaHCO₃ was added to the reaction mixture, extracted with DCM, dried and concentrated to obtain the compound 3′-(bromomethyl)-2-methyl-4-(3-(methylsulfonyl)propoxy)-1,1′-biphenyl (150 mg, 0.378 mM); ¹H NMR (300 MHz, CDCl₃): δ 7.70 (s, 1H), 7.57 (d, J=6 Hz, 1H), 7.45-7.40 (m, 2H), 7.34 (s, 2H), 4.76 (s, 2H), 3.90 (t, J=11.4 Hz, 2H), 3.40-3.37 (m, 2H), 3.05 (s, 3H), 2.29 (s, 6H), 2.20-2.18 (m, 2H); MS: (m/z) 420 (M+Na).

### Step 1e

### Ethyl 2-(3-(4-((3′,5′-dimethyl-4′-(3-(methylsulfonyl)propoxy)-[1,1′-biphenyl]-3-yl) methoxy)phenyl)oxetan-3-yl)acetate (Compound 78)

To a solution of ethyl 2-(3-(4-hydroxyphenyl)oxetan-3-yl)acetate (compound of Step 1b of Example 1, 40 mg, 0.169 mM) and 3′-(bromomethyl)-3,5-dimethyl-4-(3-(methyl sulfonyl)propoxy)-1,1′-biphenyl (compound of Step 1d, 62 mg, 0.152 mM) in anhydrous DMF (2 ml), cesium carbonate (110 mg, 0.339 mM) was added at RT. The reaction mixture was stirred at RT for 2 h and then quenched by addition of 5 ml water, stirred for 10 min, extracted with ethyl acetate. The organic layer was washed with brine, dried and concentrated to obtain the compound ethyl 2-(3-(4-((3′,5′-dimethyl-4′-(3-(methyl sulfonyl)propoxy)-[1,1′-biphenyl]-3-yl)methoxy)phenyl)oxetan-3-yl)acetate (92 mg, 0.162 mM) as colorless oil. Yield: 96%; ¹H NMR (300 MHz, DMSO-d₆): δ 7.69 (s, 1H), 7.58 (d, J=7.2 Hz, 1H), 7.47-7.38 (m, 2H), 7.35 (s, 2H), 7.23 (d, J=8.4 Hz, 2H), 7.02 (d, J=8.4 Hz, 2H), 5.14 (s, 2H), 4.74 (s, 4H), 3.89-3.86 (q, J=6.9 Hz, 2H), 3.45-3.40 (m, 4H), 3.05 (s, 3H), 3.01-2.97 (m, 2H), 2.29 (s, 6H), 2.18 (s, 2H), 1.23 (t, J=6.6 Hz, 3H).

### Example 79

### 2-(3-(4-((3′,5′-Dimethyl-4′-(3-(methylsulfonyl)propoxy)-[1,1′-biphenyl]-3-yl)methoxy)phenyl)oxetan-3-yl)acetic acid (Compound 79)

To a solution of ethyl 2-(3-(4-((3′,5′-dimethyl-4′-(3-(methylsulfonyl)propoxy)-[1,1′-biphenyl]-3-yl)methoxy)phenyl)oxetan-3-yl)acetate (compound of Example 78, 92 mg, 0.162 mM) in 10 ml of THF:MeOH (4:1), lithium hydroxide hydrate (649 μl, 0.974 mM) was added, and the mixture was stirred at RT over-night. Solvent was removed, the reaction mixture was neutralized with saturated ammonium chloride and extracted with ethyl acetate dried and concentrated to get the compound 2-(3-(4-((3′,5′-dimethyl-4′-(3-(methylsulfonyl)propoxy)-[1,1′-biphenyl]-3-yl)methoxy)phenyl)oxetan-3-yl)acetic acid (50 mg, 0.088 mM). Yield: 54%; ¹H NMR (300 MHz, DMSO-d₆): δ 12.12 (s, 1H), 7.69 (s, 1H), 7.58 (d, J=7.2 Hz, 1H), 7.47-7.38 (m, 2H), 7.35 (s, 2H), 7.23 (d, J=8.4 Hz, 2H), 7.02 (d, J=8.4 Hz, 2H), 5.14 (s, 2H), 4.74 (s, 4H), 3.89-3.86 (t, J=5.7 Hz, 2H), 3.45-3.40 (m, 2H), 3.05 (s, 3H), 3.01-2.97 (m, 2H), 2.29 (s, 6H), 2.18 (s, 2H); MS: (m/z) 539 (M+1).

### Example 80

### Ethyl 2-(3-(4-((3′-methoxy-4′-(3-(methylsulfonyl)propoxy)-[1,1′-biphenyl]-3-yl) methoxy)phenyl)oxetan-3-yl)acetate (Compound 80)

To a solution of ethyl 2-(3-(4-((4′-hydroxy-3′-methoxy-[1,1′-biphenyl]-3-yl)methoxy)phenyl)oxetan-3-yl)acetate (40 mg, 0.089 mM; prepared by the method analogous to method described in Step 1d of Example 1) and 3-(methyl sulfonyl)propyl 4-methylbenzenesulfonate (28.7 mg, 0.098 mM) in anhydrous DMF (2 ml), cesium carbonate (58 mg, 0.178 mM) was added at RT. The reaction mixture was stirred at RT for 2 h and then quenched by addition of 5 ml water, stirred for 10 min and extracted with ethyl acetate. The organic layer was washed with brine, dried and concentrated. The residue was purified by flash column chromatography (silica gel, 40% ethyl acetate in n-hexane) to obtain the compound ethyl 2-(3-(4-((3′-methoxy-4′-(3-(methylsulfonyl)propoxy)-[1,1′-biphenyl]-3-yl)methoxy)phenyl)oxetan-3-yl)acetate (50 mg, 0.087 mM) as colorless oil. Yield: 98%; ¹H NMR (300 MHz, DMSO-d₆): δ 7.72 (s, 1H), 7.62 (d, J=7.2 Hz, 1H), 7.47 (d, J=7.5 Hz, 1H), 7.42-7.38 (t, J=6.9 Hz, 1H), 7.24 (s, 1H), 7.18-7.16 (m, 3H), 7.07 (d, J=8.1, 1H), 7.02 (d, J=8.4 Hz, 2H), 5.15 (s, 2H), 4.75 (s, 4H), 4.14-4.10 (t, J=6 Hz, 2H), 3.93-3.88 (m, 2H), 3.86 (s, 3H), 3.28-3.26 (m, 2H), 3.08 (s, 2H), 3.03 (s, 3H), 2.15 (s, 2H), 1.04 (t, J=7.2 Hz, 3H); MS: (m/z) 569 (M+Na).

### Example 81

### 2-(3-(4-((3′-Methoxy-4′-(3-(methylsulfonyl)propoxy)-[1,1′-biphenyl]-3-yl)methoxy)phenyl)oxetan-3-yl)acetic acid (Compound 81)

To a solution of ethyl 2-(3-(4-((3′-methoxy-4′-(3-(methylsulfonyl)propoxy)-[1,1′-biphenyl]-3-yl)methoxy)phenyl)oxetan-3-yl)acetate (compound of Example 80, 45 mg, 0.079 mM) in 5 ml of THF:MeOH (4:1), lithium hydroxide hydrate (317 μl, 0.475 mM) was added and the mixture was stirred at RT over-night. Solvent was removed, the reaction mixture was neutralized with saturated ammonium chloride and extracted with ethyl acetate, dried and concentrated to obtain the compound 2-(3-(4-((3′-methoxy-4′-(3-(methylsulfonyl)propoxy)-[1,1′-biphenyl]-3-yl)methoxy)phenyl)oxetan-3-yl)acetic acid (26 mg, 0.045 mM). Yield: 56%; ¹H NMR (300 MHz, DMSO-d₆): δ 12.12 (s, 1H), 7.73 (s, 1H), 7.63 (d, J=7.2 Hz, 1H), 7.48-7.38 (m, 2H), 7.24 (d, J=4.2 Hz, 2H), 7.20 (s, 2H), 7.07 (s, 1H), 7.05-6.99 (m, 2H), 5.15 (s, 2H), 4.75 (s, 4H), 4.14-4.10 (t, J=6 Hz, 2H), 3.86 (s, 3H), 3.28-3.24 (m, 2H), 3.03 (s, 3H), 3.01-2.97 (m, 2H), 2.27-2.16 (m, 2H); MS: (m/z) 563 (M+Na).

### Example 82

### Ethyl 2-(3-(4-((4′-(methylthio)-[1,1′-biphenyl]-3-yl)methoxy)phenyl)oxetan-3-yl)acetate (Compound 82)

To a solution of ethyl 2-(3-(4-((3-bromobenzyl)oxy)phenyl)oxetan-3-yl)acetate (compound of Step 1a of Example 39, 50 mg, 0.123 mM) and (4-(methylthio)phenyl)boronic acid (24.8 mg, 0.148 mM) in dioxane (4 ml) and water (1 ml), potassium carbonate (42.6 mg, 0.308 mM) was added and the mixture was degassed with argon for 10 min. To the resulting solution palladium tetrakistriphenylphosphine (7 mg, 6.17 mM) was added and the mixture was heated at 80° C. for 2 h. The reaction mixture was diluted with 50 ml ethyl acetate and 10 ml water and filtered through celite. From the filtrate, organic layer was separated and washed with brine, dried and concentrated. The crude compound was purified by column chromatography to obtain the compound ethyl 2-(3-(4-((4′-(methylthio)-[1,1′-biphenyl]-3-yl)methoxy)phenyl)oxetan-3-yl)acetate (36 mg, 0.080 mM) as white solid. Yield: 65%; ¹HNMR (300 MHz, DMSO-d₆): δ 7.72 (s, 1H), 7.64-7.61 (m, 3H), 7.47-7.43 (m, 2H), 7.37 (d, J=8.1 Hz, 2H), 7.18 (d, J=8.4 Hz, 2H), 7.02 (d, J=8.4 Hz, 2H), 5.16 (s, 2H), 4.75 (s, 4H), 3.90 (q, J=7.2 Hz, 2H), 3.08 (s, 2H), 2.25 (s, 3H), 1.04 (t, J=6.9 Hz, 3H); MS: (m/z) 449 (M+1).

### Example 83

### 2-(3-(4-((4′-(Methylthio)-[1,1′-biphenyl]-3-yl)methoxy)phenyl)oxetan-3-yl)acetic acid (Compound 83)

To a solution of ethyl 2-(3-(4-((4′-(methylthio)-[1,1′-biphenyl]-3-yl) methoxy)phenyl)oxetan-3-yl)acetate (compound of Example 82, 50 mg, 0.111 mM) in 5 ml of THF:MeOH (4:1), lithium hydroxide hydrate (0.372 ml, 0.557 mM) was added and the mixture was stirred at RT over-night. Solvent was removed, the reaction mixture was neutralized with saturated ammonium chloride and extracted with ethyl acetate, dried and concentrated to obtain the compound 2-(3-(4-((4′-(methylthio)-[1,1′-biphenyl]-3-yl)methoxy)phenyl)oxetan-3-yl)acetic acid (40 mg, 0.093 mM). Yield: 83%; ¹H NMR (300 MHz, DMSO-d₆): δ 12.10 (s, 1H), 7.73 (s, 1H), 7.65-7.62 (m, 3H), 7.50-7.41 (m, 2H), 7.37 (d, J=8.1 Hz, 2H), 7.23 (d, J=8.4 Hz, 2H), 7.02 (d, J=8.1 Hz, 2H), 5.16 (s, 2H), 4.75 (s, 4H), 3.01 (s, 2H), 2.25 (s, 3H); MS: (m/z) 459 (M+K).

### Example 84

### Ethyl 2-(3-(4-((4′-(butylthio)-[1,1′-biphenyl]-3-yl)methoxy)phenyl)oxetan-3-yl)acetate (Compound 84)

To a solution of ethyl 2-(3-(4-((3-bromobenzyl)oxy)phenyl)oxetan-3-yl)acetate (compound of Step 1a of Example 39, 200 mg, 0.493 mM) and (4-(butylthio)phenyl)boronic acid (124 mg, 0.592 mM) in dioxane (4 ml) and water (1 ml), potassium carbonate (171 mg, 1.234 mM) was added and the mixture was degassed with argon for 10 min. To the resulting solution palladium tetrakistriphenylphosphine (28.5 mg, 0.025 mM) was added and the mixture was heated at 120° C. for 10 min in microwave. The reaction mixture was diluted with ethyl acetate (100 ml) and water (10 ml) and filtered through celite. From the filtrate, organic layer was separated and washed with brine, dried and concentrated. The crude compound was purified by column chromatography to obtain the compound ethyl 2-(3-(4-((4′-(butylthio)-[1,1′-biphenyl]-3-yl)methoxy)phenyl)oxetan-3-yl)acetate (167 mg, 0.340 mM) as white solid. Yield: 69%; ¹H NMR (300 MHz, DMSO-d₆): δ 7.72 (s, 1H), 7.63-7.61 (d, J=8.1 Hz, 3H), 7.50-7.47 (m, 1H), 7.43-7.38 (m, 3H), 7.18 (d, J=8.1 Hz, 2H), 7.02 (d, J=8.4 Hz, 2H), 5.16 (s, 2H), 4.75 (s, 4H), 3.90 (q, J=7.2 Hz, 2H), 3.07 (s, 2H), 3.03 (t, J=6.9 Hz, 2H), 1.59-1.56 (m, 2H), 1.43-1.41 (m, 2H) 1.04 (t, J=7.2 Hz, 3H), 0.92 (t, J=7.2 Hz, 3H); MS: (m/z) 491 (M+1).

### Example 85

### 2-(3-(4-((4′-(Butylthio)-[1,1′-biphenyl]-3-yl)methoxy)phenyl)oxetan-3-yl)acetic acid (Compound 85)

To a solution of ethyl 2-(3-(4-((4′-(butylthio)-[1,1′-biphenyl]-3-yl)methoxy)phenyl)oxetan-3-yl)acetate (compound of Example 84, 50 mg, 0.102 mM) in 5 ml of THF:MeOH (4:1), lithium hydroxide hydrate (0.340 ml, 0.510 mM) was added and the mixture was stirred at RT over-night. Solvent was removed, the reaction mixture was neutralized with saturated ammonium chloride and extracted with ethyl acetate, dried and concentrated to obtain the compound 2-(3-(4-((4′-(butylthio)-[1,1′-biphenyl]-3-yl)methoxy)phenyl)oxetan-3-yl)acetic acid (37 mg, 0.076 mM). Yield: 74.8%; ¹H NMR (300 MHz, DMSO-d₆): δ 12.11 (s, 1H), 7.72 (s, 1H), 7.63 (d, J=7.8 Hz, 3H), 7.50 (d, J=7.8 Hz, 1H), 7.43 (t, J=7.8 Hz, 3H), 7.22 (d, J=8.4 Hz, 2H), 7.02 (d, J=8.1 Hz, 2H), 5.15 (s, 2H), 4.74 (s, 4H), 3.00-2.98 (m, 4H), 1.61-1.58 (m, 2H), 1.46-1.38 (m, 2H), 0.91 (t, J=7.2 Hz, 3H); MS: (m/z) 463 (M+1).

### Example 86

### Ethyl 2-(3-(4-((4′-(3-(methylsulfonyl)propoxy)-3′-(trifluoromethyl)-[1,1′-biphenyl]-3-yl)methoxy)phenyl)oxetan-3-yl)acetate (Compound 86)

### Step 1a

### Synthesis of 4′-Hydroxy-3′-(trifluoromethyl)-[1,1′-biphenyl]-3-carbaldehyde

To a solution of 4-bromo-2-(trifluoromethyl) phenol (1 g, 4.15 mM), (3-formylphenyl) boronic acid (747 mg, 4.98 mM) in DMF: H₂O (2 ml:0.2 ml), sodium carbonate (792 mg, 7.47 mM) was added. To the resulting solution PdCl₂(PPh₃)₂ (58 mg, 0.083 mM) was added and the mixture was heated at 120° C. for 10 min in microwave. The reaction mixture was diluted with ethyl acetate (100 ml) and water (10 ml), organic layer was separated and washed with brine, dried and concentrated. The crude compound was purified by column chromatography to obtain the compound 4′-hydroxy-3′-(trifluoromethyl)-[1,1′-biphenyl]-3-carbaldehyde (615 mg, 2.078 mM). Yield: 50%; ¹H NMR (300 MHz, DMSO-d₆): δ 10.83 (s, 1H), 10.09 (s, 1H), 8.18 (s, 1H), 8.01 (d, J=7.5 Hz, 1H), 7.88 (d, J=9 Hz, 3H), 7.70 (t, J=7.8 Hz, 1H), 7.17 (d, J=8.1 Hz, 1H); MS: (m/z) 265 (M+K).

### Step 1b

### Synthesis of 3′-(Hydroxymethyl)-3-(trifluoromethyl)-[1,1′-biphenyl]-4-ol

To a solution of 4′-hydroxy-3′-(trifluoromethyl)-[1,1′-biphenyl]-3-carbaldehyde (Step 1a, 200 mg, 0.751 mM) in 10 ml of dry methanol, NaBH₄ (34 mg, 0.902 mM) was added and the mixture was stirred at RT for 1 h. Solvent was removed, residue was extracted with ethyl acetate, dried and concentrated to obtain the compound 3′-(hydroxymethyl)-3-(trifluoromethyl)-[1,1′-biphenyl]-4-ol (179 mg, 0.660 mM). Yield: 88%; ¹H NMR (300 MHz, DMSO-d₆): δ 10.68 (s, 1H), 7.77 (s, 1H), 7.74 (d, J=5.7 Hz, 1H), 7.56 (s, 1H), 7.50 (d, J=7.5 Hz, 1H), 7.42 (t, J=7.5 Hz, 1H), 7.29 (d, J=7.2 Hz, 1H), 7.13 (d, J=8.4 Hz, 1H), 5.24 (t, J=5.4 Hz, 1H), 4.57 (d, J=5.4 Hz, 2H); MS: (m/z) 267 (M−1).

### Step 1c

### Synthesis of (4′-(3-(Methylsulfonyl)propoxy)-3′-(trifluoromethyl)-[1,1′-biphenyl]-3-yl) methanol

To a solution of 3′-(hydroxymethyl)-3-(trifluoromethyl)-[1,1′-biphenyl]-4-ol (compound of Step 1b, 100 mg, 0.373 mM) in DMF (2 ml), Cs₂CO₃ (121 mg, 0.373 mM) was added, followed by addition of 3-(methylsulfonyl) propyl 4-methylbenzenesulfonate (120 mg, 0.410 mM) and allowed to stir at RT for 2 h. The reaction mixture was quenched with water and extracted with ethyl acetate, concentrated and purified by column chromatography to obtain the pure compound (4′-(3-(methylsulfonyl)propoxy)-3′-(trifluoromethyl)-[1,1′-biphenyl]-3-yl) methanol (122.5 mg, 0.302 mM). Yield: 81%; ¹H NMR (300 MHz, DMSO-d₆): δ 7.94 (d, J=8.4 Hz, 1H), 7.83 (s, 1H), 7.61 (s, 1H), 7.55 (d, J=7.5 Hz, 1H), 7.44 (d, J=7.5 Hz, 1H), 7.38-7.30 (m, 2H), 5.27 (t, J=5.4 Hz, 1H), 4.58 (d, J=5.4 Hz, 2H), 4.31 (t, J=5.7 Hz, 2H), 3.28-3.23 (m, 2H), 3.03 (s, 3H), 2.22 (t, J=7.5 Hz, 2H); MS: (m/z) 411 (M+Na).

### Step 1d

### Synthesis of 3′-(Bromomethyl)-4-(3-(methylsulfonyl)propoxy)-3-(trifluoromethyl)-1,1′-biphenyl

To a solution of (4′-(3-(methylsulfonyl)propoxy)-3′-(trifluoromethyl)-[1,1′-biphenyl]-3-yl) methanol (compound of Step 1c, 100 mg, 0.257 mM) in DCM, PBr₃ (77 mg, 0.286 mM) was added and the mixture was allowed to stir at RT for 30 min. The reaction mixture was diluted with ethylacetate (100 ml) and water (10 ml), organic layer was separated and washed with brine, dried and concentrated, to obtain the compound 3′-(bromomethyl)-4-(3-(methylsulfonyl)propoxy)-3-(trifluoromethyl)-1,1′-biphenyl (89.6 mg, 0.199 mM). Yield: 69%; ¹H NMR (300 MHz, DMSO-d₆): δ 7.96 (d, J=8.1 Hz, 1H), 7.86 (s, 1H), 7.79 (s, 1H), 7.64 (s, 1H), 7.45 (s, 2H), 7.40 (d, J=8.7 Hz, 1H), 4.30 (s, 2H), 3.28-3.23 (m, 2H), 3.03 (s, 3H), 2.20 (m, 2H), 1.23 (s, 2H); MS: (m/z) 474 (M+1).

### Step 1e

### Ethyl 2-(3-(4-((4′-(3-(methylsulfonyl)propoxy)-3′-(trifluoromethyl)-[1,1′-biphenyl]-3-yl)methoxy)phenyl)oxetan-3-yl)acetate (Compound 86)

To a solution of ethyl 2-(3-(4-hydroxyphenyl)oxetan-3-yl)acetate (compound of Step 1b, Example 1, 45 mg, 0.190 mM) and 3′-(bromomethyl)-4-(3-(methylsulfonyl)propoxy)-3-(trifluoromethyl)-1,1′-biphenyl (compound of Step 1d, 77 mg, 0.171 mM) in anhydrous DMF (2 ml), cesium carbonate (124 mg, 0.381 mM) was added at RT. The reaction mixture was stirred at RT for 2 h. Reaction was then quenched with addition of 5 ml water and allowed to stir for 10 min, and then extracted with ethyl acetate. The organic layer was washed with brine, dried and concentrated. The residue was purified by flash column chromatography (silica gel; 30% ethyl acetate in n-hexane) to obtain the compound ethyl 2-(3-(4-((4′-(3-(methylsulfonyl)propoxy)-3′-(trifluoromethyl)-[1,1′-biphenyl]-3-yl)methoxy)phenyl)oxetan-3-yl)acetate (59 mg, 0.097 mM) as colorless oil. Yield: 51%; ¹H NMR (300 MHz, DMSO-d₆): δ 7.96 (d, J=8.4 Hz, 1H), 7.86 (s, 1H), 7.76 (s, 1H), 7.66 (d, J=6.9 Hz, 1H), 7.51-7.46 (m, 2H), 7.39 (d, J=8.4 Hz, 1H), 7.19 (d, J=8.4 Hz, 2H), 7.02 (d, J=8.4 Hz, 2H), 5.17 (s, 2H), 4.75 (s, 4H), 4.31 (t, J=5.7 Hz, 2H), 3.93 (q, J=6.9 Hz, 2H), 3.28-3.23 (m, 2H), 3.08 (s, 2H), 3.03 (s, 3H), 2.20 (s, 2H), 1.04 (t, J=6.9 Hz, 3H); MS: m/z 607 (M+1).

### Example 87

### 2-(3-(4-((4′-(3-(Methylsulfonyl)propoxy)-3′-(trifluoromethyl)-[1,1′-biphenyl]-3-yl)methoxy)phenyl)oxetan-3-yl)acetic acid (Compound 87)

To a solution of ethyl 2-(3-(4-((4′-(3-(methylsulfonyl)propoxy)-3′-(trifluoromethyl)-[1,1′-biphenyl]-3-yl)methoxy)phenyl)oxetan-3-yl)acetate (compound of Example 86, 50 mg, 0.082 mM) in 5 ml of THF:MeOH (4:1), lithium hydroxide hydrate (0.275 ml, 0.412 mM) was added and the mixture was stirred at RT over-night. Solvent was removed, and the reaction mixture was neutralized with saturated ammonium chloride, extracted with ethyl acetate, dried and concentrated to obtain the compound 2-(3-(4-((4′-(3-(methylsulfonyl)propoxy)-3′-(trifluoromethyl)-[1,1′-biphenyl]-3-yl)methoxy)phenyl)oxetan-3-yl)acetic acid (35 mg, 0.055 mM). Yield: 67%; ¹H NMR (300 MHz, DMSO-d₆): δ 12.12 (s, 1H), 7.97 (d, J=8.1 Hz, 1H), 7.87 (s, 1H), 7.77 (s, 1H), 7.66 (d, J=6.6 Hz, 1H), 7.49-7.46 (m, 2H), 7.39 (d, J=8.4 Hz, 1H), 7.23 (d, J=8.7 Hz, 2H), 7.02 (d, J=8.1 Hz, 2H), 5.16 (s, 2H), 4.74 (s, 4H), 4.29 (t, J=5.7 Hz, 2H), 3.28-3.26 (m, 2H), 3.03 (s, 2H), 3.02 (s, 3H), 2.20 (s, 2H); MS (m/z): δ 01 (M+Na).

### Example 88

### Ethyl 2-(3-(4-((4′-(isopropylthio)-[1,1′-biphenyl]-3-yl) methoxy)phenyl)oxetan-3-yl)acetate (Compound 88)

To a solution of ethyl 2-(3-(4-((3-bromobenzyl)oxy)phenyl)oxetan-3-yl)acetate (compound of Step 1a, Example 39, 200 mg, 0.493 mM) and (4-(isopropylthio)phenyl)boronic acid (116 mg, 0.592 mM) in dioxane (4 ml) and water (1 ml), potassium carbonate (171 mg, 1.234 mM) was added and the mixture was degassed with argon for 10 min. To the resulting solution palladium tetrakistriphenylphosphine (28.5 mg, 0.025 mM) was added and the mixture was heated at 120° C. for 10 min in microwave. The reaction mixture was diluted with ethyl acetate (100 ml) and water (10 ml), and the organic layer was separated and washed with brine, dried and concentrated. The crude compound was purified by column chromatography to obtain the compound ethyl 2-(3-(4-((4′-(isopropylthio)-[1,1′-biphenyl]-3-yl)methoxy)phenyl)oxetan-3-yl)acetate (122 mg, 0.259 mM) as colorless oil. Yield: 51%; ¹H NMR (300 MHz, DMSO-d₆): δ 7.74 (s, 2H), 7.65 (d, J=7.8 Hz, 2H), 7.50-7.44 (m, 4H), 7.18 (d, J=8.1 Hz, 2H), 7.02 (d, J=8.1 Hz, 2H), 5.17 (s, 2H), 4.75 (s, 4H), 3.92 (q, J=6.6 Hz, 2H), 3.57-3.53 (m, 1H), 3.08 (s, 2H), 1.28 (s, 3H), 1.26 (s, 3H), 1.04 (t, J=6.9 Hz, 3H); MS: (m/z) 477 (M+1).

### Example 89

### 2-(3-(4-((4′-(Isopropylthio)-[1,1′-biphenyl]-3-yl)methoxy)phenyl)oxetan-3-yl)acetic acid (Compound 89)

To a solution of ethyl 2-(3-(4-((4′-(isopropylthio)-[1,1′-biphenyl]-3-yl)methoxy)phenyl)oxetan-3-yl)acetate (compound of Example 88, 50 mg, 0.105 mM) in 5 ml of THF:MeOH (4:1), lithium hydroxide hydrate (0.350 ml, 0.525 mM) was added and the mixture was stirred at RT over-night. Solvent was removed, and the reaction mixture was neutralized with saturated ammonium chloride and extracted with ethyl acetate, dried and concentrated to obtain the compound 2-(3-(4-((4′-(isopropylthio)-[1,1′-biphenyl]-3-yl)methoxy)phenyl)oxetan-3-yl)acetic acid (40 mg, 0.086 mM). Yield: 82%; ¹H NMR (300 MHz, DMSO-d₆): δ 12.12 (s, 1H), 7.74 (s, 1H), 7.65-7.63 (m, 3H), 7.50-7.44 (m, 4H), 7.22 (d, J=8.4 Hz, 2H), 7.02 (d, J=8.4 Hz, 2H), 5.15 (s, 2H), 4.75 (s, 4H), 3.57-3.50 (m, 1H), 3.01 (s, 2H), 1.27 (s, 3H), 1.25 (s, 3H); MS: (m/z) 449 (M+1).

### Example 90

### Ethyl 2-(3-(4-((5-methyl-2-phenyloxazol-4-yl)methoxy)phenyl)oxetan-3-yl)acetate (Compound 90)

The title compound was prepared in an analogous manner as the compound 1 of Example 1 involving reaction of ethyl 2-(3-(4-hydroxyphenyl)oxetan-3-yl)acetate (compound of Step 1b of Example 1) with 4-(chloromethyl)-5-methyl-2-phenyloxazole. Yield: 77%; ¹H NMR (300 MHz, CDCl₃): δ 8.03 (d, J=6.0 Hz, 2H), 7.47-7.40 (m, 3H), 7.13 (d, J=8.4 Hz, 2H), 7.02 (d, J=8.4 Hz, 2H), 5.01 (d including s at 4.99, J=6 Hz, 4H), 4.87 (d, J=6 Hz, 2H), 4.03 (q, J=6 Hz, 2H), 3.11 (s, 2H), 2.45 (s, 3H), 1.13 (t, J=6.2 Hz, 3H); MS: m/z 408 (M+1).

### Example 91

### 2-(3-(4-((5-Methyl-2-phenyloxazol-4-yl)methoxy)phenyl)oxetan-3-yl)acetic acid (Compound 91)

The title compound was prepared in an analogous manner as the compound 2 of Example 2. Compound 91 was obtained by hydrolyzing the compound of Example 90. Yield: 59.7%; ¹H NMR (300 MHz, DMSO-d₆): δ 12.8 (s, 1H), 7.94 (d, J=6.0 Hz, 2H), 7.52-7.50 (m, 3H), 7.24 (d, J=8.4 Hz, 2H), 7.02 (d, J=8.4 Hz, 2H), 4.98 (s, 2H), 4.75 (s, J=6 Hz, 2H), 3.01 (s, 2H), 2.44 (s, 3H); MS: m/z 380 (M+1).

### Example 92

### Ethyl 2-(3-(4-((2′,6′-dimethyl-4′-(3-(methylsulfonyl)propoxy)-[1,1′-biphenyl]-3-yl)methoxy)phenyl)azetidin-3-yl)acetate (Compound 92)

### Step 1a

### Synthesis of tert-butyl 3-(2-Ethoxy-2-oxoethylidene)azetidine-1-carboxylate

To a suspension of sodium hydride (0.056 g, 2.337 mM) in THF (10 ml) ethyl 2-(diethoxyphosphoryl)acetate (0.524 g, 2.337 mM) was added at 0° C. The reaction mixture was stirred for 30 min. To the resulting mixture tert-butyl 3-oxoazetidine-1-carboxylate (0.2 g, 1.168 mM) in THF (2 ml) was added dropwise. The reaction mixture was stirred for 2 h. Reaction mixture was quenched with addition of water and extracted with ethyl acetate. The organic layer was washed with brine, dried and concentrated. Crude product was purified by column chromatography (silica gel; 10% ethyl acetate in petether) to obtain tert-butyl 3-(2-ethoxy-2-oxoethylidene)azetidine-1-carboxylate (160 mg, 0.663 mM). Yield: 56.8%; ¹H NMR (300 MHz, CDCl₃): δ 5.78 (bs, 1H), 4.84-4.83 (m, 2H), 4.61-4.60 (m, 2H), 4.18 (q, J=6.89, 2H), 1.47 (s, 9H), 1.28 (t, J=6.89, 3H); MS: (m/z): 242 (M+1), 264 (M+Na).

### Step 1b

### Synthesis of tert-butyl 3-(2-Ethoxy-2-oxoethyl)-3-(4-hydroxyphenyl)azetidine-1-carboxylate

A mixture of tert-butyl 3-(2-ethoxy-2-oxoethylidene)azetidine-1-carboxylate (compound of Step 1a, 50 mg, 0.207 mM), (4-hydroxyphenyl)boronic acid (57.2 mg, 0.414 mM), chloro(1,5-cyclooctadiene)rhodium(I) dimer (4.09 mg, 8.29 μM), potassium hydroxide (0.276 ml, 0.414 mM) in THF (1 ml) and dioxane (1 ml) was heated in microwave at 100° C. for 10 min and then the reaction mixture was extracted with ethyl acetate. Organic layer was washed with brine, dried and concentrated. Crude product was purified by column chromatography (silica gel; 10% ethyl acetate in pet ether) to obtain tert-butyl 3-(2-ethoxy-2-oxoethyl)-3-(4-hydroxyphenyl)azetidine-1-carboxylate (35 mg, 0.104 mM) as off white solid. Yield: 50.4%; ¹H NMR (300 MHz, DMSO-d₆): δ 7.04 (d, J=8.1 Hz, 2H), 6.79 (d, J=8.1 Hz, 2H), 6.24 (bs, 1H), 4.24-4.20 (m, 4H), 4.03 (q, J=6.90 Hz, 2H), 2.93 (s, 2H), 1.17 (s, 9H), 1.14 (t, J=6.90 Hz, 3H); MS: (m/z) 336 (M+1), 358 (M+Na).

### Step 1c

### Synthesis of tert-butyl 3-(4-((2′,6′-Dimethyl-4′-(3-(methylsulfonyl)propoxy)-[1,1′-biphenyl]-3-yl) methoxy)phenyl)-3-(2-ethoxy-2-oxoethyl) azetidine-1-carboxylate

To a solution of tert-butyl 3-(2-ethoxy-2-oxoethyl)-3-(4-hydroxy phenyl)azetidine-1-carboxylate (compound of Step 1b, 80 mg, 0.239 mM) in anhydrous DMF, cesium carbonate (155 mg, 0.477 mM) was added followed by addition of 3′-(bromomethyl)-2,6-dimethyl-4-(3-(methylsulfonyl)propoxy)-1,1′-biphenyl (preparation is described in Example 9, 88 mg, 0.215 mM) at RT. The reaction mixture was stirred at RT for 2 h. Reaction was then quenched by addition of water 5 ml and allowed to stir for 10 min and extracted with ethyl acetate. The organic layer was washed with brine, dried and concentrated. The residue was purified by flash column chromatography to obtain tert-butyl 3-(4-((2′,6′-dimethyl-4′-(3-(methyl sulfonyl)propoxy)-[1,1′-biphenyl]-3-yl)methoxy)phenyl)-3-(2-ethoxy-2-oxoethyl) azetidine-1-carboxylate (124 mg, 0.185 mM). Yield: 78%; ¹H NMR (300 MHz, CDCl₃): δ 7.45-7.41 (m, 2H), 7.18-7.15 (m, 3H), 7.07 (s, 1H), 6.97 (d, J=8.1 Hz, 2H), 6.70 (s, 2H), 5.14 (s, 2H), 4.08 (s, 4H), 4.03 (s, 3H), 3.91-3.88 (q, J 7.2, Hz, 2H), 3.03 (s, 2H), 2.94 (s, 2H), 2.27-2.13 (m, 2H), 1.90 (s, 6H), 1.37 (s, 9H), 1.24 (s, 2H), 1.05 (t, J=6.9 Hz, 3H); MS: (m/z) 688 (M+Na).

### Step 1d

### Synthesis of 2-(1-(tert-Butoxycarbonyl)-3-(4-((2′,6′-dimethyl-4′-(3-(methylsulfonyl)propoxy)-[1,1′-biphenyl]-3-yl)methoxy)phenyl)azetidin-3-yl)acetic acid

To a solution of tert-butyl 3-(4-((2′,6′-dimethyl-4′-(3-(methylsulfonyl)propoxy)-[1,1′-biphenyl]-3-yl)methoxy)phenyl)-3-(2-ethoxy-2-oxoethyl)azetidine-1-carboxylate (compound of Step 1c, 100 mg, 0.150 mM) in 4 ml of THF:MeOH (4:1), lithium hydroxide hydrate (05 ml, 0.751 mM) was added and the mixture was stirred at RT for 4 h. Solvent was removed and the reaction mixture was neutralized using saturated ammonium chloride. The mixture was the extracted with ethyl acetate. The organic layer was washed with brine, dried and concentrated to obtain 2-(1-(tert-butoxycarbonyl)-3-(4-((2′,6′-dimethyl-4′-(3-(methylsulfonyl)propoxy)-[1,1′-biphenyl]-3-yl)methoxy)phenyl) azetidin-3-yl)acetic acid (60 mg, 0.093 mM). Yield: 62%; ¹H NMR (300 MHz, CDCl₃): δ 12.13 (s, 1H), 7.45-7.41 (m, 2H), 7.21-7.15 (m, 3H), 7.07 (d, J=6.6 Hz, 1H), 6.98 (d, J=8.1 Hz, 2H), 6.70 (s, 2H), 5.14 (s, 2H), 4.08 (s, 4H), 4.01 (s, 2H), 3.27-3.25 (m, 2H), 3.03 (s, 3H), 2.88 (s, 2H), 2.14 (bs, 2H), 1.91 (s, 6H), 1.37 (s, 9H); MS: (m/z) 660 (M+Na).

### Step 1e

### Ethyl 2-(3-(4-((2′,6′-dimethyl-4′-(3-(methylsulfonyl)propoxy)-[1,1′-biphenyl]-3-yl) methoxy)phenyl)azetidin-3-yl)acetate (Compound 92)

To a solution of tert-butyl 3-(4-((2′,6′-dimethyl-4′-(3-(methylsulfonyl)propoxy)-[1,1′-biphenyl]-3-yl)methoxy)phenyl)-3-(2-ethoxy-2-oxoethyl)azetidine-1-carboxylate (compound of Step 1c, 600 mg, 0.901 mM) in anhydrous DCM, 2,2,2-trifluoroacetic acid (11 ml, 9.01 mM) was added at 0° C. The reaction mixture was stirred at RT for 1 h. Reaction was then quenched with addition of NaHCO₃ solution and allowed to stir for 10 min and then extracted with DCM. The organic layer was washed with brine, dried and concentrated. The residue was purified by flash column chromatography to obtain ethyl 2-(3-(4-((2′,6′-dimethyl-4′-(3-(methylsulfonyl)propoxy)-[1,1′-biphenyl]-3-yl) methoxy)phenyl)azetidin-3-yl)acetate (490 mg, 0.813 mM). Yield: 90%; ¹H NMR (300 MHz, DMSO-d₆): δ 7.45-7.41 (m, 2H), 7.15 (s, 2H), 7.09-7.06 (m, 2H), 6.95 (d, J=8.1 Hz, 2H), 6.70 (s, 2H), 5.76 (s, 1H), 5.13 (s, 2H), 4.08 (m, 2H), 3.90-3.83 (q, J=6.9 Hz, 2H), 3.78 (d, J=7.2 Hz, 2H), 3.68 (d, J=7.5 Hz, 2H), 3.27 (s, 2H), 3.03 (s, 3H), 2.96 (s, 2H), 2.27-2.14 (m, 2H), 1.91 (s, 6H), 1.02-0.97 (t, J=6.9, 13.8 Hz, 3H); MS: (m/z) 566 (M+1).

### Example 93

### Ethyl 2-(3-(4-((2′,6′-dimethyl-4′-(3-(methylsulfonyl)propoxy)-[1,1′-biphenyl]-3-yl) methoxy)phenyl)-1-(methylsulfonyl)azetidin-3-yl)acetate (Compound 93)

To the stirred solution of ethyl 2-(3-(4-((2′,6′-dimethyl-4′-(3-(methylsulfonyl)propoxy)-[1,1′-biphenyl]-3-yl)methoxy)phenyl)azetidin-3-yl)acetate (compound of Example 92, 140 mg, 0.247 mM) and triethylamine (125 mg, 1.237 mM) in DCM (5 ml) methanesulfonyl chloride (34.0 mg, 0.297 mM) was added and allowed to stir at RT for 2 h. The reaction mixture was quenched with water and extracted with ethyl acetate and purified by column chromatography to give the pure compound of ethyl 2-(3-(4-((2′,6′-dimethyl-4′-(3-(methylsulfonyl)propoxy)-[1,1′-biphenyl]-3-yl)methoxy)phenyl)-1-(methylsulfonyl) azetidin-3-yl)acetate (100 mg, 0.515 mM). Yield: 61%; ¹H NMR (300 MHz, DMSO-d₆): δ 7.45-7.41 (m, 2H), 7.22-7.16 (m, 3H), 7.07 (d, J=6.9 Hz, 1H), 6.99 (d, J=8.4 Hz, 2H), 6.70 (s, 2H), 5.15 (s, 2H), 4.07-4.04 (m, 4H), 3.91 (q, J=6.9, 14.1 Hz, 2H), 3.27 (s, 2H), 3.03 (s, 3H), 3.00 (s, 3H), 2.14 (s, 2H), 1.91 (s, 6H), 1.23-1.20 (m, 2H), 1.17-1.15 (m, 2H), 1.05 (t, J=7.2 Hz, 3H); MS: (m/z) 644 (M+1).

### Example 94

### 2-(3-(4-((2′,6′-Dimethyl-4′-(3-(methylsulfonyl)propoxy)-[1,1′-biphenyl]-3-yl)methoxy)phenyl)-1-(methylsulfonyl)azetidin-3-yl)acetic acid (Compound 94)

To a solution of ethyl ethyl 2-(3-(4-((2′,6′-dimethyl-4′-(3-(methyl sulfonyl)propoxy)-[1,1′-biphenyl]-3-yl)methoxy)phenyl)-1-(methylsulfonyl) azetidin-3-yl)acetate (compound of Example 93, 93 mg, 0.144 mM) in 5 ml of THF:MeOH (4:1) lithium hydroxide hydrate (578 μl, 0.867 mM) was added and the mixture was stirred at RT for 4 h. Solvent was removed and the reaction mixture was neutralized with saturated ammonium chloride and extracted with ethyl acetate, dried over sodium sulphate to get the compound 2-(3-(4-((2′,6′-dimethyl-4′-(3-(methylsulfonyl)propoxy)-[1,1′-biphenyl]-3-yl)methoxy)phenyl)-1-(methylsulfonyl) azetidin-3-yl)acetic acid (68 mg, 0.110 mM). Yield: 76%; ¹H NMR (300 MHz, DMSO-d₆): δ 12.18 (s, 1H), 7.46-7.42 (m, 2H), 7.26 (d, J=8.4 Hz, 2H), 7.16 (s, 1H), 7.08 (d, J=6.9 Hz, 1H), 7.00 (d, J=8.4 Hz, 2H), 6.70 (s, 2H), 5.14 (s, 2H), 4.10-4.03 (m, 4H), 3.27 (s, 2H), 3.03 (s, 3H), 2.98 (s, 3H), 2.94 (s, 2H), 2.14-2.10 (m, 2H), 1.91 (s, 6H), 1.23-1.20 (m, 2H); MS: (m/z) 616 (M+1).

### Example 95

### Ethyl 2-(1-acetyl-3-(4-((2′,6′-dimethyl-4′-(3-(methylsulfonyl)propoxy)-[1,1′-biphenyl]-3-yl)methoxy)phenyl)azetidin-3-yl)acetate (Compound 95)

To the stirred solution of ethyl 2-(3-(4-((2′,6′-dimethyl-4′-(3-(methylsulfonyl)propoxy)-[1,1′-biphenyl]-3-yl)methoxy)phenyl)azetidin-3-yl)acetate (compound of Example 92, 50 mg, 0.088 mM) and triethylamine (0.061 ml, 0.442 mM) in DCM (5 ml) acetyl chloride (7.63 mg, 0.097 mM) was added and stirred at RT for 2 h. Reaction mixture was quenched with water and extracted with ethyl acetate, concentrated and purified by column chromatography to obtain ethyl 2-(1-acetyl-3-(4-((2′,6′-dimethyl-4′-(3-(methylsulfonyl)propoxy)-[1,1′-biphenyl]-3-yl) methoxy)phenyl)azetidin-3-yl)acetate (37 mg, 0.061 mM). Yield: 69%; ¹H NMR (300 MHz, CDCl₃): δ 7.45-7.41 (m, 2H), 7.25-7.23 (m, 2H), 7.21-7.16 (m, 2H), 6.99 (d, J=8.4 Hz, 2H), 6.70 (s, 2H), 5.14 (s, 2H), 4.33 (s, 2H), 4.08 (s, 2H), 3.91-3.89 (q, J=6.9 Hz, 2H), 3.25 (s, 3H), 3.03 (s, 3H), 2.14 (m, 2H), 1.91 (s, 6H), 1.76 (s, 2H), 1.23 (s, 2H), 1.05-1.01 (t, J=7.2, 14.1 Hz, 3H); MS: (m/z) 608 (M+1).

### Example 96

### 2-(1-Acetyl-3-(4-((2′,6′-dimethyl-4′-(3-(methylsulfonyl)propoxy)-[1,1′-biphenyl]-3-yl)methoxy)phenyl)azetidin-3-yl)acetic acid (Compound 96)

To a solution of ethyl 2-(1-acetyl-3-(4-((2′,6′-dimethyl-4′-(3-(methyl sulfonyl)propoxy)-[1,1′-biphenyl]-3-yl)methoxy)phenyl)azetidin-3-yl)acetate (compound of Example 95, 30 mg, 0.049 mM) in 2 ml of THF:MeOH (4:1) lithium hydroxide hydrate (197 μl, 0.296 mM) was added and the mixture was stirred at RT for 2-3 h. Solvent was removed and the reaction mixture was neutralized with saturated ammonium chloride and extracted with ethyl acetate, dried and concentrated to obtain the compound 2-(1-acetyl-3-(4-((2′,6′-dimethyl-4′-(3-(methylsulfonyl)propoxy)-[1,1′-biphenyl]-3-yl)methoxy)phenyl)azetidin-3-yl)acetic acid (20 mg, 0.035 mM. Yield: 70%; ¹H NMR (300 MHz, CDCl₃): δ 12.14 (s, 1H), 7.45-7.41 (m, 2H), 7.23 (d, J=7.8 Hz, 2H), 7.15-7.05 (m, 2H), 6.98 (d, J=7.8 Hz, 2H), 6.70 (s, 2H), 5.13 (s, 2H), 4.32 (s, 2H), 4.08-4.036 (m, 3H), 3.97 (d, J=9.3 Hz, 2H), 3.24 (s, 4H), 3.02 (s, 3H), 2.91 (s, 2H), 2.13-2.00 (m, 2H), 1.90 (s, 6H); MS: (m/z) 602 (M+1).

### Example 97

### Ethyl 2-(3-(3-fluoro-4-((4′-(trifluoromethyl)-[1,1′-biphenyl]-3-yl)methoxy)phenyl)oxetan-3-yl)acetate (Compound 97)

### Step 1a

### Synthesis of Ethyl 2-(3-(3-fluoro-4-hydroxyphenyl)oxetan-3-yl)acetate

To a solution of Rh(COD)₂Cl₂ (69.74 mg, 1.243 mM) in dioxane (5 ml), potassium hydroxide (394 mg, 7.03 mM) was added and the yellow solution formed was stirred at room temperature for 15 min. To this, (3-fluoro-4-hydroxyphenyl)boronic acid (1097 mg, 7.03 mM) was added followed by ethyl 2-(oxetan-3-ylidene)acetate (compound of Step 1a of Example 1, 500 mg, 3.52 mM) dissolved in dioxane, and the reaction mixture was stirred at room temperature for 10-12 h. Reaction mixture was filtered through celite and extracted using ethyl acetate, concentrated and purified by column chromatography to obtain the compound ethyl 2-(3-(3-fluoro-4-hydroxyphenyl)oxetan-3-yl)acetate (190 mg) as brown solid. Yield: 21.05%; ¹H NMR (300 MHz, CDCl₃) δ: 7.34-7.21 (m, 3H), 7.13 (d, J=8.4 Hz, 2H), 6.93 (d, J=8.4 Hz, 2H), 5.05 (s, 2H), 5.00 (d, J=6.0 Hz, 2H), 4.86 (d, J=6.0 Hz, 2H), 4.02 (q, J=7.2 Hz, 2H), 3.11 (s, 2H), 1.14 (t, J=7.2 Hz, 3H); MS: (e/z) 451.8 (M+Na).

### Step 1b

### Ethyl 2-(3-(3-fluoro-4-((4′-(trifluoromethyl)-[1,1′-biphenyl]-3-yl)methoxy)phenyl)oxetan-3-yl)acetate (Compound 97)

To a solution of 3-(bromomethyl)-4′-(trifluoromethyl)-1,1′-biphenyl (compound of Step 1c″ of Example 1, 94 mg, 0.299 mM) and ethyl 2-(3-(3-fluoro-4-hydroxyphenyl)oxetan-3-yl)acetate (compound of Step 1a, 76 mg, 0.299 mM) dissolved in dry DMF (2 ml), cesium carbonate (57.7 mg, 0.299 mM) was added and stirred at room temperature under nitrogen atmosphere. The reaction mixture was quenched with water and extracted with ethyl acetate, dried, concentrated and purified by column chromatography to obtain the compound ethyl 2-(3-(3-fluoro-4-((4′-(trifluoromethyl)-[1,1′-biphenyl]-3-yl)methoxy)phenyl)oxetan-3-yl)acetate (122 mg) as colourless thick liquid. Yield: 83%; ¹H NMR (300 MHz, CDCl₃) δ: 7.81-7.69 (m, 5H), 7.57-7.50 (m, 3H), 7.04-6.97 (m, 2H), 6.88 (d, J=8.1 Hz, 1H), 5.21 (s, 2H), 4.95 (d, J=6.0 Hz, 2H), 4.85 (d, J=6.0 Hz, 2H), 4.03 (q, J=6.9 Hz, 2H), 3.10 (s, 2H), 1.14 (t, J=7.2 Hz, 3H); MS: (e/z) 489.2 (M+1), 511.2 (M+Na).

### Example 98

### 2-(3-(3-Fluoro-4-((4′-(trifluoromethyl)-[1,1′-biphenyl]-3-yl)methoxy)phenyl)oxetan-3-yl)acetic acid (Compound 98)

To a solution of ethyl 2-(3-(3-fluoro-4-((4′-(trifluoromethyl)-[1,1′-biphenyl]-3-yl)methoxy)phenyl)oxetan-3-yl)acetate (compound of Example 97, 100 mg, 0.205 mM) in 4 ml of THF:MeOH (4:1), lithium hydroxide hydrate (682 μl, 1.024 mM) was added and the mixture was stirred at room temperature for 2-3 h. After completion of the reaction, solvent was evaporated and washed with acetonitrile and neutralized with saturated ammonium chloride and extracted with ethyl acetate. The organic layer was washed with brine, dried and concentrated to obtain the compound 2-(3-(3-fluoro-4-((4′-(trifluoromethyl)-[1,1′-biphenyl]-3-yl)methoxy)phenyl)oxetan-3-yl) acetic acid (78 mg) as white solid. Yield: 87%; ¹H NMR (300 MHz, DMSO-d₆) δ: 12.18 (bs, 1H), 7.91 (d, J=8.1 Hz, 2H), 7.84 (d, J=5.4 Hz, 3H), 7.73 (d, J=6.3 Hz, 1H), 7.55 (d, J=5.7 Hz, 2H), 7.29-7.17 (m, 2H), 7.06 (d, J=8.1 Hz, 1H), 5.26 (s, 2H), 4.73 (s, 4H), 3.04 (s, 2H); MS: (e/z) 461.0 (M+1), 483.1 (M+Na).

### Example 99

### Ethyl 2-(3-(4-((4-fluoro-3-(trifluoromethoxy)benzyl)oxy)phenyl)oxetan-3-yl)acetate (Compound 99)

To a solution of ethyl 2-(3-(4-hydroxyphenyl)oxetan-3-yl)acetate (compound of Step 1b of Example 1, 80 mg, 0.339 mM) and 4-(bromomethyl)-1-fluoro-2-(trifluoromethoxy)benzene (92 mg, 0.339 mM) dissolved in dry DMF (2 ml), cesium carbonate (220 mg, 1.140 mM) was added and stirred at room temperature for 2 h under nitrogen atmosphere. Reaction mixture was quenched with water and extracted with ethyl acetate, dried, concentrated and purified by column chromatography to obtain the compound ethyl 2-(3-(4-((4-fluoro-3-(trifluoromethoxy)benzyl)oxy)phenyl)oxetan-3-yl)acetate (118 mg) as pale yellow thick liquid. Yield: 79%; ¹H NMR (300 MHz, CDCl₃) δ: 7.42-7.36 (m, 2H), 7.22 (d, J=8.7 Hz, 1H), 7.13 (d, J=8.4 Hz, 2H), 6.94 (d, J=8.4 Hz, 2H), 5.03 (s, 2H), 5.00 (d, J=6.0 Hz, 2H), 4.86 (d, J=6.0 Hz, 2H), 4.02 (q, J=7.2 Hz, 2H), 3.11 (s, 2H), 1.14 (t, J=7.2 Hz, 3H); MS: (e/z) 452.4 (M+Na).

### Example 100

### 2-(3-(4-((4-Fluoro-3-(trifluoromethoxy)benzyl)oxy)phenyl)oxetan-3-yl)acetic acid (Compound 100)

To a solution of ethyl 2-(3-(4-((4-fluoro-3-(trifluoromethoxy)benzyl)oxy)phenyl)oxetan-3-yl)acetate (compound of Example 99, 118 mg, 0.275 mM) in 4 ml of THF:MeOH (4:1), lithium hydroxide hydrate (57.8 mg, 1.377 mM) was added and the mixture was stirred at room temperature for 2-3 h. After completion of the reaction, solvent was evaporated and washed with acetonitrile, neutralized with saturated ammonium chloride and extracted with ethyl acetate. The organic layer was washed with brine, dried and concentrated to obtain the compound 2-(3-(4-((4-fluoro-3-(trifluoromethoxy)benzyl)oxy)phenyl)oxetan-3-yl)acetic acid (100 mg) as white solid. Yield: 89%; ¹H NMR (300 MHz, DMSO-d₆) δ: 12.14 (bs, 1H), 7.66 (d, J=7.2 Hz, 1H), 7.55 (d, J=7.8 Hz, 2H), 7.22 (d, J=8.4 Hz, 2H), 6.99 (d, J=8.4 Hz, 2H), 5.12 (s, 2H), 4.75 (s, 4H), 3.02 (s, 2H); MS: (e/z) 401.1 (M+1).

### Example 101

### Ethyl 2-(3-(4-((3-fluorobenzyl)oxy)phenyl)oxetan-3-yl)acetate (Compound 101)

To the stirred solution of 1-(chloromethyl)-3-fluorobenzene (500 mg, 3.46 mM) and ethyl 2-(3-(4-hydroxyphenyl)oxetan-3-yl)acetate (compound of Step 1 b of Example 1, 817 mg, 3.46 mM) dissolved in DMF (20 ml), cesium carbonate (1686 mg, 8.74 mM) was added and stirred at 80° C. for 2-3 h. The reaction mixture was quenched with water, extracted with ethyl acetate, concentrated and purified by column chromatography to obtain the compound ethyl 2-(3-(4-((3-fluorobenzyl)oxy)phenyl)oxetan-3-yl)acetate (992 mg) as pale yellow thick liquid. Yield: 83.3%; ¹H NMR (300 MHz, DMSO-d₆) δ: 7.39-7.32 (m, 2H), 7.21.7.00 (m, 4H), 6.94 (d, J=8.4 Hz, 2H), 5.06 (s, 2H), 4.99 (d, J=6.0 Hz, 2H), 4.86 (d, J=6.0 Hz, 2H), 4.02 (q, J=6.0 Hz, 2H), 3.11 (s, 2H), 1.13 (t, J=7.2 Hz, 3H); MS: (e/z) 367.0 (M+Na).

### Example 102

### 2-(3-(4-((3-Fluorobenzyl)oxy)phenyl)oxetan-3-yl)acetic acid (Compound 102)

To a solution of ethyl 2-(3-(4-((3-fluorobenzyl)oxy)phenyl)oxetan-3-yl)acetate (compound of Example 101, 100 mg, 0.290 mM) in 5 ml of THF:MeOH (4:1), lithium hydroxide hydrate (968 μl, 1.452 mM) was added and the reaction mixture was stirred at room temperature for 2-3 h. After completion of the reaction, solvent was evaporated and washed with ethyl acetate, neutralized with saturated ammonium chloride and extracted with ethyl acetate. The organic layer was washed with brine, dried and concentrated to obtain the compound 2-(3-(4-((3-fluorobenzyl)oxy)phenyl)oxetan-3-yl)acetic acid (83.5 mg) as white solid. Yield: 87%; ¹H NMR (300 MHz, DMSO-d₆) δ: 12.12 (bs, 1H), 7.47-7.40 (m, 1H), 7.29-7.13 (m, 5H), 6.98 (d, J=8.7 Hz, 2H), 5.11 (s, 2H), 4.74 (s, 4H), 3.01 (s, 2H); MS: (e/z) 317.0 (M+1).

### Example 103

### Ethyl 2-(3-(4-((2-fluoro-5-(trifluoromethoxy)benzyl)oxy)phenyl)oxetan-3-yl)acetate (Compound 103)

To a stirred solution of 2-(bromomethyl)-1-fluoro-4-(trifluoromethoxy)benzene (83 mg, 0.304 mM) and ethyl 2-(3-(4-hydroxyphenyl)oxetan-3-yl)acetate (compound of Step 1b of Example 1, 72 mg, 0.305 mM) dissolved in DMF (3 ml), cesium carbonate (58.8 mg, 0.305 mM) was added under nitrogen atmosphere, at room temperature and stirred. After completion of the reaction, the reaction mixture was quenched with saturated ammonium chloride and extracted with ethyl acetate, dried, concentrated and purified to obtain the compound ethyl 2-(3-(4-((2-fluoro-5-(trifluoromethoxy)benzyl)oxy)phenyl)oxetan-3-yl)acetate (112 mg) as white solid. Yield: 81%; ¹H NMR (300 MHz, CDCl₃) δ: 7.41-7.43 (m, 1H), 7.10-7.19 (m, 4H), 6.97 (d, J=8.4 Hz, 2H), 5.13 (s, 2H), 5.00 (d, J=6.0 Hz, 2H), 4.87 (d, J=6.0 Hz, 2H), 4.02 (q, J=7.2 Hz, 2H), 3.12 (s, 2H), 1.14 (t, J=7.2 Hz, 3H); MS: (e/z) 451.8 (M+Na).

### Example 104

### 2-(3-(4-((2-Fluoro-5-(trifluoromethoxy)benzyl)oxy)phenyl)oxetan-3-yl)acetic acid (Compound 104)

To a solution of ethyl 2-(3-(4-((2-fluoro-5-(trifluoromethoxy)benzyl)oxy)phenyl)oxetan-3-yl)acetate (compound of Example 103, 72 85 mg, 0.198 mM) in 4 ml of THF:MeOH (4:1), lithium hydroxide hydrate (661 μl, 0.992 mM) was added and the mixture was stirred at room temperature for 2-3 h. After completion of the reaction, solvent was evaporated, washed with acetonitrile and neutralized with saturated ammonium chloride and extracted with ethyl acetate. The organic layer was washed with brine, dried and concentrated to obtain the compound 2-(3-(4-((2-fluoro-5-(trifluoromethoxy)benzyl)oxy)phenyl)oxetan-3-yl)acetic acid (67.3 mg) as white solid. Yield: 84%; ¹H NMR (300 MHz, DMSO-d₆) δ: 12.15 (bs, 1H), 7.61 (bs, 1H), 7.45-739 (m, 2H), 7.23 (d, J=8.4 Hz, 2H), 7.02 (d, J=8.4 Hz, 2H), 5.15 (s, 2H), 4.75 (s, 4H), 3.02 (s, 2H); MS: (e/z) 401.1 (M+1), 423.1 (M+Na).

### Example 105

### Ethyl 2-(3-(4-((3-(5-methoxypyridin-3-yl)benzyl)oxy)phenyl)oxetan-3-yl)acetate (Compound 105)

To a solution of ethyl 2-(3-(4-((3-bromobenzyl)oxy)phenyl)oxetan-3-yl)acetate (compound of Step 1a of Example 39,120 mg, 0.296 mM) and 3-methoxy-5-(4,4,5,5-tetramethyl-1,3,2-dioxaborolan-2-yl)pyridine (84 mg, 0.355 mM) in 5 ml dioxane:water (4:1), potassium carbonate (82 mg, 0.592 mM) was added and the mixture was degassed with argon for 3 min. To the resulting solution, palladium tetrakistriphenylphosphine (17.10 mg, 0.015 mM) was added and the mixture was heated at 110° C. for 10 min in microwave. After completion of the reaction, reaction mixture was extracted with ethyl acetate, dried, concentrated and purified by column chromatography to obtain the compound ethyl 2-(3-(4-((3-(5-methoxypyridin-3-yl)benzyl)oxy)phenyl)oxetan-3-yl)acetate (96 mg) as colourless thick liquid. Yield: 73.6%; ¹HNMR (300 MHz, CDCl₃) δ: 8.49 (s, 1H), 8.30 (s, 1H), 7.83 (s, 1H), 7.71 (d, J=8.7 Hz, 1H), 7.63 (s, 1H), 7.55-7.53 (m, 2H), 7.18 (d, J=8.4 Hz, 2H), 7.01 (d, J=8.4 Hz, 2H), 5.18 (s, 2H), 4.75 (s, 4H), 3.91-3.86 (m, 5H), 3.08 (s, 2H), 1.02 (t, J=6.0 Hz, 3H); MS (e/z): 434.5 (M+1).

### Example 106

### 2-(3-(4-((3-(5-Methoxypyridin-3-yl)benzyl)oxy)phenyl)oxetan-3-yl)acetic acid (Compound of 106)

To a solution of ethyl 2-(3-(4-((3-(5-methoxypyridin-3-yl)benzyl)oxy)phenyl)oxetan-3-yl)acetate (compound of Example 105, 85 mg, 0.196 mM) in 3 ml of THF:MeOH (4:1), lithium hydroxide hydrate (654 μl, 0.980 mM) was added and the mixture was stirred at room temperature for 2-3 h. After completion of the reaction, solvent was evaporated and washed with ethyl acetate, neutralized with saturated ammonium chloride and extracted with ethyl acetate. The organic layer was washed with brine, dried and concentrated to obtain the compound 2-(3-(4-((3-(5-methoxypyridin-3-yl)benzyl)oxy)phenyl)oxetan-3-yl)acetic acid (68 mg) as white solid. Yield: 83%; ¹HNMR (300 MHz, DMSO-d₆) δ: 12.15 (bs, 1H), 8.50 (s, 1H), 8.30 (s, 1H), 7.84 (s, 1H), 7.72 (d, J=8.7 Hz, 1H), 7.64 (s, 1H), 7.55-7.51 (m, 2H), 7.22 (d, J=8.1 Hz, 2H), 7.02 (d, J=8.1 Hz, 2H), 5.17 (s, 2H), 4.75 (s, 4H), 3.91 (s, 3H), 3.01 (s, 2H); MS: (e/z) 406.4 (M+1).

### Example 107

### Ethyl 2-(3-(4-((3-(2-morpholinopyrimidin-5-yl)benzyl)oxy)phenyl)oxetan-3-yl)acetate (Compound 107)

To a solution of ethyl 2-(3-(4-((3-bromobenzyl)oxy)phenyl)oxetan-3-yl)acetate (compound of Step 1a of Example 39, 120 mg, 0.296 mM) and 4-(5-(4,4,5,5-tetramethyl-1,3,2-dioxaborolan-2-yl)pyrimidin-2-yl)morpholine (103 mg, 0.355 mM) in 4 ml dioxane:water (4:1), potassium carbonate (82 mg, 0.592 mM) was added and the mixture was degassed with argon for 3 min. To the resulting solution palladium tetrakistriphenylphosphine (17.10 mg, 0.015 mM) was added and the mixture was heated at 110° C. for 10 min in microwave. After completion of the reaction, reaction mixture was extracted with ethyl acetate, dried, concentrated and purified by column chromatography to obtain the compound ethyl 2-(3-(4-((3-(2-morpholinopyrimidin-5-yl)benzyl)oxy)phenyl)oxetan-3-yl)acetate (104 mg), as white solid. Yield: 71.4%; ¹HNMR (300 MHz, CDCl₃) δ: 8.74 (s, 2H), 7.72 (s, 1H), 7.62 (d, J=7.2 Hz, 1H), 7.50-7.40 (m, 2H), 7.18 (d, J=8.4 Hz, 2H), 7.00 (d, J=8.4 Hz, 2H), 5.14 (s, 2H), 4.75 (s, 4H), 3.90 (q, J=7.2 Hz, 2H), 3.75 (d, J=4.5 Hz, 4H), 3.67 (d, J=4.5 Hz, 4H), 3.08 (s, 2H), 1.02 (t, J=7.2 Hz, 3H); MS: (e/z) 490.4 (M+1).

### Example 108

### 2-(3-(4-((3-(2-Morpholinopyrimidin-5-yl)benzyl)oxy)phenyl)oxetan-3-yl)acetic acid (Compound 108)

To a solution of ethyl 2-(3-(4-((3-(2-morpholinopyrimidin-5-yl)benzyl)oxy)phenyl)oxetan-3-yl)acetate (compound of Example 107, 80 mg, 0.163 mM) in 4 ml of THF:MeOH (4:1), lithium hydroxide hydrate (545 μl, 0.817 mM) was added and the mixture was stirred at room temperature for 2-3 h. After completion of the reaction, solvent was evaporated and washed with ethyl acetate, neutralized with saturated ammonium chloride and extracted with ethyl acetate. The organic layer was washed with brine, dried and concentrated to obtain compound 2-(3-(4-((3-(2-morpholinopyrimidin-5-yl)benzyl)oxy)phenyl)oxetan-3-yl)acetic acid (68 mg) as white solid. Yield: 83%; ¹HNMR (300 MHz, DMSO-d₆) δ: 11.94 (bs, 1H), 7.64-8.74 (s, 1H), 7.73 (s, 1H), 8.30 (s, 1H), 7.62 (d, J=7.2 Hz, 1H), 7.50-7.41 (m, 2H), 7.22 (d, J=8.4 Hz, 2H), 7.02 (d, J=8.4 Hz, 2H), 5.13 (s, 2H), 4.74 (s, 4H), 3.75 (d, J=4.2 Hz, 4H), 3.69 (d, J=4.2 Hz, 4H), 3.01 (s, 2H); MS: (e/z) 462.2 (M+1).

### Example 109

### Ethyl 2-(3-(4-((3-(6-(3-(methylsulfonyl)propoxy)pyridin-3-yl)benzyl)oxy)phenyl)oxetan-3-yl)acetate (Compound 109)

### Step 1a

### Synthesis of Ethyl 2-(3-(4-((3-(6-hydroxypyridin-3-yl)benzyl)oxy)phenyl)oxetan-3-yl)acetate

To a solution of ethyl 2-(3-(4-((3-bromobenzyl)oxy)phenyl)oxetan-3-yl)acetate (compound of Step 1a of Example 39, 900 mg, 2.221 mM) and (6-hydroxypyridin-3-yl)boronic acid (463 mg, 3.33 mM) in 4 ml dioxane:water (4:1) potassium carbonate (767 mg, 5.55 mM) was added and the mixture was degassed with argon for 2-3 min. To the resulting solution palladium tetrakistriphenylphophine (154 mg, 0.133 mM) was added and the mixture was heated at 110° C. for 10 min in microwave. After completion of the reaction, reaction mixture was quenched with water and extracted with ethyl acetate, dried, concentrated and purified by column chromatography on silica gel to obtain the compound ethyl 2-(3-(4-((3-(6-hydroxypyridin-3-yl)benzyl)oxy)phenyl)oxetan-3-yl)acetate (820 mg) as colourless thick liquid. Yield: 88%; ¹HNMR (300 MHz, DMSO-d₆) δ: 11.85 (bs, 1H), 7.85-7.81 (m, 2H) 7.71 (s, 1H), 7.63-7.41 (m, 1H), 7.52-7.34 (m, 2H), 7.17 (d, J=8.4 Hz, 2H), 6.99 (d, J=8.4 Hz, 2H), 6.44 (d, J=9.6 Hz, 1H), 5.11 (s, 2H), 4.75 (s, 4H), 3.99 (q, J=7.2 Hz, 2H), 3.07 (s, 2H), 1.01 (t, J=7.2 Hz, 3H); MS (e/z): 420.2 (M+1).

### Step 1b

### Ethyl 2-(3-(4-((3-(6-(3-(methylsulfonyl)propoxy)pyridin-3-yl)benzyl)oxy)phenyl)oxetan-3-yl)acetate (Compound 109)

To a solution of ethyl 2-(3-(4-((3-(6-hydroxypyridin-3-yl)benzyl)oxy)phenyl)oxetan-3-yl)acetate (compound of Step 1a, 40 mg, 0.095 mM) and 3-(methylsulfonyl)propyl 4-methylbenzenesulfonate (30.7 mg, 0.105 mM) dissolved in DMF (3 ml), cesium carbonate (62.07 mg, 0.322 mM) was added and stirred at 80° C. for 2 h. After completion of the reaction, the reaction mixture was quenched with water and extracted with ethyl acetate, concentrated and purified by column chromatography to obtain the compound ethyl 2-(3-(4-((3-(6-(3-(methylsulfonyl) propoxy)pyridin-3-yl)benzyl)oxy)phenyl)oxetan-3-yl)acetate (42 mg) as pale yellow semisolid. Yield: 81%; ¹HNMR (300 MHz, DMSO-d₆) δ: 8.47 (s, 1H), 8.04-8.01 (m, 1H) 7.73 (s, 1H), 7.63-742 (m, 1H), 7.52-7.34 (m, 2H), 7.17 (d, J=8.4 Hz, 2H), 6.99 (d, J=8.4 Hz, 2H), 6.93 (d, J=8.7 Hz, 1H), 5.15 (s, 2H), 4.75 (s, 4H), 4.40 (t, J=6.0 Hz, 2H), 3.99 (q, J=6.9 Hz, 2H), 3.30-3.25 (m, 2H), 3.07 (s, 2H), 3.02 (s, 3H), 2.19-1.198 (m, 2H), 1.01 (t, J=6.9 Hz, 3H); MS: (e/z) 540.2 (M+1).

### Example 110

### 2-(3-(4-((3-(6-(3-(Methylsulfonyl)propoxy)pyridin-3-yl)benzyl)oxy)phenyl)oxetan-3-yl)acetic acid (Compound 110)

To a solution of ethyl 2-(3-(4-((3-(6-(3-(methylsulfonyl)propoxy)pyridin-3-yl)benzyl)oxy)phenyl)oxetan-3-yl)acetate (compound of Example 109, 15 mg, 0.028 mM) in 2 ml of THF:MeOH (4:1), lithium hydroxide hydrate (93 μl, 0.139 mM) was added and the mixture was stirred at room temperature for 2-3 h. After completion of the reaction, solvent was evaporated and washed with ethyl acetate, neutralized with saturated ammonium chloride and extracted with ethyl acetate. The organic layer was washed with brine, dried and concentrated to obtain the compound 2-(3-(4-((3-(6-(3-(methylsulfonyl)propoxy)pyridin-3-yl)benzyl)oxy)phenyl)oxetan-3-yl)acetic acid (10.42 mg) as white solid. Yield: 73.3%; ¹HNMR (300 MHz, CDCl₃) δ: 8.00 (s, 1H), 7.85-7.82 (m, 1H) 7.48-740 (m, 4H), 7.17 (d, J=8.4 Hz, 2H), 6.96 (d, J=8.4 Hz, 2H), 6.83 (d, J=8.7 Hz, 1H), 5.20 (s, 2H), 5.02 (d, J=6.0 Hz, 2H), 4.85 (d, J=6.0 Hz, 2H), 4.45 (t, J=5.7 Hz, 2H), 3.27-3.21 (m, 4H), 2.97 (s, 4H), 2.43-2.35 (m, 2H); MS: (e/z) 512.5 (M+1).

### Example 111

### Ethyl 2-(3-(4-((4′-(isopentyloxy)-2′,6′-dimethyl-[1,1′-biphenyl]-3-yl)methoxy)phenyl)oxetan-3-yl)acetate (Compound 111)

To a solution of ethyl 2-(3-(4-((4′-hydroxy-2′,6′-dimethyl-[1,1′-biphenyl]-3-yl)methoxy)phenyl)oxetan-3-yl)acetate (compound of Step 1c of Example 39, 82 mg, 0.184 mM) and isopentyl 4-methylbenzenesulfonate (57.9 mg, 0.239 mM) dissolved in dry DMF (3 ml), cesium carbonate (34.6 mg, 0.179 mM) was added and stirred at 80° C. under nitrogen atmosphere for 2-3 h. After completion of the reaction, the reaction mixture was quenched with water and extracted with ethyl acetate, dried, concentrated and purified by column chromatography to get the pure compound ethyl 2-(3-(4-((4′-(isopentyloxy)-2′,6′-dimethyl-[1,1′-biphenyl]-3-yl)methoxy)phenyl) oxetan-3-yl)acetate (76 mg) as pale yellow thick liquid. Yield: 78.1%; ¹H NMR (300 MHz, CDCl₃) δ: 7.47-7.38 (m, 2H), 7.19 (s, 1H), 7.13-7.08 (m, 3H), 6.99-6.89 (m, 2H), 6.70 (s, 2H), 5.13 (bs, 1H), 5.11 (s, 2H), 4.99 (d, J=6.0 Hz, 2H), 4.86 (d, J=6.0 Hz, 2H), 4.13-3.98 (m, 6H), 3.10 (s, 2H), 2.03 (s, 6H); MS: (e/z) 491.0 (M+1), 513.0 (M+Na).

### Example 112

### 2-(3-(4-((4′-(Isopentyloxy)-2′,6′-dimethyl-[1,1′-biphenyl]-3-yl)methoxy)phenyl)oxetan-3-yl)acetic acid (Compound 112)

To a solution of ethyl 2-(3-(4-((4′-(isopentyloxy)-2′,6′-dimethyl-[1,1′-biphenyl]-3-yl)methoxy)phenyl)oxetan-3-yl)acetate (compound of Example 111, 80 mg, 0.155 mM) in 4 ml of THF:MeOH (4:1), lithium hydroxide hydrate (774 μl, 0.774 mM) was added and the mixture was stirred at room temperature for 2-3 h. After completion of the reaction, solvent was evaporated and washed with acetonitrile, neutralized with saturated ammonium chloride and extracted with ethyl acetate. The organic layer was washed with brine, dried and concentrated to obtain the compound 2-(3-(4-((4′-(isopentyloxy)-2′,6′-dimethyl-[1,1′-biphenyl]-3-yl)methoxy)phenyl)oxetan-3-yl)acetic acid (61.4 mg) as off-white solid. Yield: 80%; ¹H NMR (300 MHz, DMSO-d₆) δ: 12.10 (bs, 1H), 7.43-7.37 (m, 2H), 7.19 (s, 1H), 7.13-7.10 (m, 3H), 6.95 (d, J=8.4 Hz, 2H), 6.67 (s, 2H), 5.13 (s, 2H), 4.98 (d, J=6.0 Hz, 2H), 4.85 (d, J=6.0 Hz, 2H), 4.17-4.10 (m, 1H), 4.01 (t, J=6.6 Hz, 2H), 3.15 (s, 2H), 1.99 (s, 6H), 1.73-1.67 (m, 2H), 0.99 (d, J=6.6 Hz, 6H); MS: (m/z) 489.2 (M+1), 511.0 (M+Na).

### Example 113

### Ethyl 2-(3-(4-((4′-((1,3-difluoropropan-2-yl)oxy)-2′,6′-dimethyl-[1,1′-biphenyl]-3-yl)methoxy)phenyl)oxetan-3-yl)acetate (Compound 113)

To a solution of ethyl 2-(3-(4-((4′-hydroxy-2′,6′-dimethyl-[1,1′-biphenyl]-3-yl)methoxy)phenyl)oxetan-3-yl)acetate (compound of Step 1c of Example 39, 100 mg, 0.224 mM) and 1,3-difluoropropan-2-yl 4-methylbenzenesulfonate (61.6 mg, 0.246 mM) dissolved in dry DMF (7 ml), cesium carbonate (146 mg, 0.448 mM) was added and stirred at 80° C. under nitrogen atmosphere for 2-3 h. After completion of the reaction, the reaction mixture was quenched with water and extracted with ethyl acetate, dried, concentrated and purified by column chromatography to obtain the compound ethyl 2-(3-(4-((4′-((1,3-difluoropropan-2-yl)oxy)-2′,6′-dimethyl-[1,1′-biphenyl]-3-yl)methoxy)phenyl)oxetan-3-yl)acetate (105 mg) as pale yellow thick liquid. Yield: 88%; ¹H NMR (300 MHz, CDCl₃) δ: 7.48-7.42 (m, 2H), 7.19 (s, 1H), 7.12-7.09 (m, 3H), 6.96 (d, J=8.4 Hz, 2H), 6.74 (s, 2H), 5.11 (s, 2H), 4.99 (d, J=5.7 Hz, 2H), 4.86 (d, J=5.7 Hz, 2H), 4.78 (s, 2H), 4.70-4.63 (m, 4H), 4.02 (q, J=7.2 Hz, 2H), 7.01 (s, 2H), 2.00 (s, 6H), 1.14 (t, J=7.2 Hz, 3H); MS: (m/z) 525.0 (M+1), 547.0 (M+Na).

### Example 114

### 2-(3-(4-((4′-((1,3-Difluoropropan-2-yl)oxy)-2′,6′-dimethyl-[1,1′-biphenyl]-3-yl)methoxy)phenyl)oxetan-3-yl)acetic acid (Compound 114)

To a solution of ethyl 2-(3-(4-((4′-((1,3-difluoropropan-2-yl)oxy)-2′,6′-dimethyl-[1,1′-biphenyl]-3-yl)methoxy)phenyl)oxetan-3-yl)acetate (compound of Example 113, 67 mg, 0.128 mM) in 3 ml of THF:MeOH (4:1), lithium hydroxide hydrate (426 μl, 0.639 mM) was added and the mixture was stirred at room temperature for 2-3 h. After completion of the reaction, solvent was evaporated and washed with acetonitrile, neutralized with saturated ammonium chloride and extracted with ethyl acetate. The organic layer was washed with brine, dried and concentrated to obtain the compound 2-(3-(4-((4′-((1,3-difluoropropan-2-yl)oxy)-2′,6′-dimethyl-[1,1′-biphenyl]-3-yl)methoxy)phenyl)oxetan-3-yl)acetic acid (48 mg) as white solid. Yield: 82.7%; ¹H NMR (300 MHz, DMSO-d₆) δ: 12.12 (bs, 1H), 7.46-7.42 (m, 2H), 7.21-7.18 (m, 3H), 7.07 (d, J=6.9 Hz, 2H), 6.95 (d, J=6.9 Hz, 2H), 6.81 (s, 2H), 5.14 (s, 2H), 4.81-4.74 (m, 8H), 3.12 (s, 2H), 1.91 (s, 6H); MS: (m/z) 497.2 (M+1), 519.1 (M+Na).

### Example 115

### Ethyl 2-(3-(4-((2′,6′-dimethyl-4′-(neopentyloxy)-[1,1′-biphenyl]-3-yl)methoxy)phenyl)oxetan-3-yl)acetate (Compound 115)

To a solution of ethyl 2-(3-(4-((4′-hydroxy-2′,6′-dimethyl-[1,1′-biphenyl]-3-yl)methoxy)phenyl)oxetan-3-yl)acetate (compound of Step 1c of Example 39, 150 mg, 0.336 mM) and neopentyl 4-methylbenzenesulfonate (81 mg, 0.336 mM) dissolved in dry DMF (8 ml), cesium carbonate (130 mg, 0.672 mM) was added and stirred at 80° C. under nitrogen atmosphere for 2-3 h. After completion of the reaction, the reaction mixture was quenched with water and extracted with ethyl acetate, dried, concentrated and purified by column chromatography to obtain the compound ethyl 2-(3-(4-((2′,6′-dimethyl-4′-(neopentyloxy)-[1,1′-biphenyl]-3-yl) methoxy)phenyl)oxetan-3-yl)acetate (154 mg) as pale yellow thick liquid. Yield: 88.3%; ¹H NMR (300 MHz, CDCl₃) δ: 7.44-7.40 (m, 2H), 7.19 (s, 1H), 7.14 (d, J=8.4 Hz, 3H), 6.95 (d, J=8.4 Hz, 2H), 6.69 (s, 2H), 5.11 (s, 2H), 4.99 (d, J=5.7 Hz, 2H), 4.86 (d, J=5.7 Hz, 2H), 4.02 (q, J=6.9 Hz, 2H), 3.62 (s, 2H), 3.10 (s, 2H), 2.00 (s, 6H), 1.14 (t, J=7.2 Hz, 3H), 1.06 (s, 9H); MS: (m/z) 525.0 (M+1), 547.0 (M+Na).

### Example 116

### 2-(3-(4-((2′,6′-Dimethyl-4′-(neopentyloxy)-[1,1′-biphenyl]-3-yl)methoxy)phenyl)oxetan-3-yl)acetic acid (Compound 116)

To a solution of ethyl 2-(3-(4-((2′,6′-dimethyl-4′-(neopentyloxy)-[1,1′-biphenyl]-3-yl)methoxy)phenyl)oxetan-3-yl)acetate (compound of Example 115, 30 mg, 0.058 mM) in 2 ml of THF:MeOH (4:1), lithium hydroxide hydrate (12.18 mg, 0.290 mM) was added and the mixture was stirred at room temperature for 2-3 h. After completion of the reaction, solvent was evaporated and washed with acetonitrile, neutralized with saturated ammonium chloride and extracted with ethyl acetate. The organic layer was washed with brine, dried and concentrated to obtain the compound 2-(3-(4-((2′,6′-dimethyl-4′-(neopentyloxy)-[1,1′-biphenyl]-3-yl)methoxy)phenyl)oxetan-3-yl)acetic acid (24.33 mg) as white solid. Yield: 84.8%; ¹H NMR (300 MHz, DMSO-d₆) δ: 12.12 (bs, 1H), 7.45-7.38 (m, 2H), 7.17 (t, J=6.9 Hz, 3H), 7.05 (d, J=6.9 Hz, 1H), 6.97 (d, J=6.9 Hz, 2H), 6.68 (s, 2H), 5.14 (s, 2H), 4.73 (s, 4H), 3.61 (s, 2H), 3.00 (s, 2H), 1.90 (s, 6H), 1.00 (s, 9H); MS: (m/z) 489.0 (M+1), 511.0 (M+Na).

### Example 117

### Ethyl 2-(3-(4-((4′-(2-methoxyethoxy)-2′,6′-dimethyl-[1,1′-biphenyl]-3-yl)methoxy)phenyl)oxetan-3-yl)acetate (Compound 117)

To a stirred solution of ethyl 2-(3-(4-((4′-hydroxy-2′,6′-dimethyl-[1,1′-biphenyl]-3-yl)methoxy)phenyl)oxetan-3-yl)acetate (compound of Step 1c of Example 39, 80 mg, 0.179 mM) and 1-bromo-2-methoxyethane (32.4 mg, 0.233 mM) dissolved in DMF (3 ml), sodium hydride (5.59 mg, 0.233 mM) was added under nitrogen atmosphere at 0° C. and stirred at room temperature. After completion of the reaction the reaction mixture was quenched with saturated ammonium chloride and extracted with ethyl acetate, dried, concentrated and purified to obtain the compound ethyl 2-(3-(4-((4′-(2-methoxyethoxy)-2′,6′-dimethyl-[1,1′-biphenyl]-3-yl)methoxy)phenyl)oxetan-3-yl)acetate (42 mg) pale yellow thick liquid. Yield: 43.7%; ¹H NMR (300 MHz, DMSO-d₆) δ: 7.44-7.41 (m, 2H), 7.16-7.07 (m, 4H), 6.97 (d, J=8.1 Hz, 2H), 6.7 (s, 2H), 5.14 (s, 2H), 4.75 (bs, 4H), 4.08 (bs, 2H), 3.89 (q, J=6.9 Hz, 2H), 3.65 (bs, 2H), 3.31 (s, 3H), 1.91 (s, 6H), 1.02 (t, J=6.9 Hz, 3H); MS: (e/z) 505.2 (M+1), 527.2 (M+Na).

### Example 118

### 2-(3-(4-((4′-(2-Methoxyethoxy)-2′,6′-dimethyl-[1,1′-biphenyl]-3-yl)methoxy)phenyl)oxetan-3-yl)acetic acid (Compound 118)

To a solution of ethyl 2-(3-(4-((4′-(2-methoxyethoxy)-2′,6′-dimethyl-[1,1′-biphenyl]-3-yl)methoxy)phenyl)oxetan-3-yl)acetate (compound of Example 117, 28 mg, 0.055 mM) in 4 ml of THF:MeOH (4:1), lithium hydroxide hydrate (277 μl, 0.277 mM) was added and the mixture was stirred at room temperature for 2-3 h. After completion of the reaction, solvent was evaporated and washed with acetonitrile, neutralized with saturated ammonium chloride and extracted with ethyl acetate. The organic layer was washed with brine, dried and concentrated to obtain the compound as 2-(3-(4-((4′-(2-methoxyethoxy)-2′,6′-dimethyl-[1,1′-biphenyl]-3-yl)methoxy)phenyl)oxetan-3-yl)acetic acid (22 mg) off-white semisolid. Yield: 79%; ¹H NMR (300 MHz, DMSO-d₆) δ: 12.12 (bs, 1H). 7.42 (bs, 2H), 7.18 (bs, 2H), 7.07-6.99 (m, 2H), 6.70 (s, 2H), 5.14 (s, 2H), 4.74 (bs, 4H), 4.09 (bs, 2H), 3.65 (bs, 2H), 3.17 (bs, 2H), 3.01 (s, 2H), 1.91 (s, 6H); MS: (e/z): 477.2 (M+1), 599.1 (M+Na).

### Example 119

### Ethyl 2-(3-(4-((4′-((3-(methoxymethyl)oxetan-3-yl)methoxy)-2′,6′-dimethyl-[1,1′-biphenyl]-3-yl)methoxy)phenyl)oxetan-3-yl)acetate (Compound 119)

To a stirred suspension of sodium hydride (5.55 mg, 0.231 mM) in DMF (3 ml) at 0° C., ethyl 2-(3-(4-((4′-((3-(hydroxymethyl)oxetan-3-yl)methoxy)-2′,6′-dimethyl-[1,1′-biphenyl]-3-yl)methoxy)phenyl)oxetan-3-yl)acetate (compound of Example 54, 115 mg, 0.210 mM) was added and stirred at room temperature for 5 min. To this reaction mixture, iodomethane (0.020 ml, 0.316 mM) was added and stirred at the same temperature for 1 h. After completion of reaction, the reaction mixture was quenched with water and extracted with ethyl acetate, dried, and concentrated to obtain the compound ethyl 2-(3-(4-((4′-((3-(methoxymethyl)oxetan-3-yl)methoxy)-2′,6′-dimethyl-[1,1′-biphenyl]-3-yl)methoxy)phenyl)oxetan-3-yl)acetate (107 mg) as pale yellow semisolid. Yield: 91%; ¹H NMR (300 MHz, CDCl₃) δ: 7.47-7.39 (m, 2H), 7.19 (s, 1H), 7.10 (d, J=8.4 Hz, 3H), 6.95 (d, J=8.7 Hz, 2H), 6.72 (s, 2H), 5.11 (s, 2H), 4.99 (d, J=6.0 Hz, 2H), 4.86 (d, J=5.7 Hz, 2H), 4.64-4.56 (m, 4H), 4.20 (s, 2H), 4.02 1.02 (q, J=6.9 Hz, 2H) 3.75 (s, 2H), 3.42 (s, 3H), 3.10 (s, 2H), 2.01 (s, 6H), 1.13 (d, J=6.0 Hz, 3H); MS: (m/z) 583.2 (M+Na).

### Example 120

### 2-(3-(4-((4′-((3-(Methoxymethyl)oxetan-3-yl)methoxy)-2′,6′-dimethyl-[1,1′-biphenyl]-3-yl)methoxy)phenyl)oxetan-3-yl)acetic acid (Compound 120)

To a solution of ethyl 2-(3-(4-((4′-((3-(methoxymethyl)oxetan-3-yl)methoxy)-2′,6′-dimethyl-[1,1′-biphenyl]-3-yl)methoxy)phenyl)oxetan-3-yl)acetate (compound of Example 119, 760 mg, 0.107 mM) in 4 ml of THF:MeOH (4:1), lithium hydroxide hydrate (357 μl, 0.535 mM) was added and the mixture was stirred at room temperature for 2-3 h. After completion of the reaction, solvent was evaporated and washed with acetonitrile, neutralized with saturated ammonium chloride and extracted with ethyl acetate. The organic layer was washed with brine, dried and concentrated to obtain the compound 2-(3-(4-((4′-((3-(methoxymethyl)oxetan-3-yl)methoxy)-2′,6′-dimethyl-[1,1′-biphenyl]-3-yl)methoxy)phenyl)oxetan-3-yl)acetic acid (47 mg) as off-white solid. Yield: 82%; ¹H NMR (300 MHz, DMSO-d₆) δ: 12.11 (s, 1H), 7.48-7.42 (m, 2H), 7.20-7.15 (m, 3H), 7.07-6.96 (m, 3H), 6.74 (s, 2H), 5.14 (s, 2H), 5.06 (s, 4H), 4.42-4.43 (m, 4H), 4.14 (m, 2H), 3.64 (s, 2H), 3.00 (s, 2H), 3.42 (s, 3H), 1.98 (s, 6H); MS: (m/z) 555.0 (M+Na).

### Example 121

### Ethyl 2-(3-(4-(((4′-((1,1-dioxidotetrahydrothiophen-3-yl)methoxy)-2′,6′-dimethyl-[1,1′-biphenyl]-3-yl)methyl)amino)phenyl)oxetan-3-yl)acetate (Compound of 121)

To a solution of ethyl 2-(3-(4-(((4′-hydroxy-2′,6′-dimethyl-[1,1′-biphenyl]-3-yl)methyl)amino)phenyl)oxetan-3-yl)acetate (prepared by the analogous method described for the preparation of compound of Step 1c of Example 39, 85 mg, 0.191 mM) and (1,1-dioxidotetrahydrothiophen-3-yl)methyl 4-methylbenzenesulfonate (compound of Step 1b of Example 52, 58.1 mg, 0.191 mM) dissolved in DMF (5 ml), cesium carbonate (124 mg, 0.643 mM) was added and stirred at 80° C. for 2 h. After completion of the reaction, the reaction mixture was quenched with water and extracted with ethyl acetate, concentrated and purified by column chromatography to obtain the compound ethyl 2-(3-(4-(((4′-((1,1-dioxidotetrahydrothiophen-3-yl)methoxy)-2′,6′-dimethyl-[1,1′-biphenyl]-3-yl)methyl)amino)phenyl)oxetan-3-yl) acetate (91 mg) as pale yellow solid. Yield: 77%; ¹HNMR (300 MHz, CDCl₃) δ: 7.45-7.40 (m, 2H), 7.18 (s, 1H), 7.11-6.93 (m, 5H), 6.65-6.59 (m, 2H), 5.10 (s, 2H), 4.99-4.55 (m, 2H), 4.87-4.82 (m, 4H), 4.37 (s, 2H), 4.15-3.90 (m, 4H), 3.37-3.30 (m, 1H), 3.18-2.96 (m, 3H), 2.46-2.44 (m, 1H), 2.23-2.16 (m, 1H), 1.98 (s, 6H), 1.14 (t, J=7.2 Hz, 3H); MS: (m/z) 578.2 (M+1).

### Example 122

### 2-(3-(4-(((4′-((1,1-Dioxidotetrahydrothiophen-3-yl)methoxy)-2′,6′-dimethyl-[1,1′-biphenyl]-3-yl)methyl)amino)phenyl)oxetan-3-yl)acetic acid (Compound 122)

To a solution of ethyl 2-(3-(4-(((4′-((1,1-dioxidotetrahydrothiophen-3-yl)methoxy)-2′,6′-dimethyl-[1,1′-biphenyl]-3-yl)methyl)amino)phenyl)oxetan-3-yl) acetate (compound of Example 121, 28 mg, 0.048 mM) in 4 ml of THF:MeOH (4:1), lithium hydroxide hydrate (162 μl, 0.242 mM) was added and the mixture was stirred at room temperature for 2-3 h. After completion of reaction, solvent was evaporated and washed with acetonitrile, neutralized with saturated ammonium chloride and extracted with ethyl acetate. The organic layer was washed with brine, dried and concentrated to obtain the compound 2-(3-(4-(((4′-((1,1-dioxidotetrahydrothiophen-3-yl)methoxy)-2′,6′-dimethyl-[1,1′-biphenyl]-3-yl)methyl)amino)phenyl)oxetan-3-yl) acetic acid (22 mg) as white solid. Yield: 76%; ¹H NMR (300 MHz, DMSO-d₆) δ: 12.04 (s, 1H), 7.39-7.29 (m, 2H), 7.03 (s, 1H), 6.93 (d, J=8.1, Hz, 2H), 6.68 (s, 2H), 6.52 (d, J=8.4 Hz, 2H), 6.20 (t, J=6.1 Hz, 1H), 4.70-4.65 (m, 4H), 4.29 (d, J=4.8 Hz, 2H), 4.02 (d, J=6.0 Hz, 2H), 3.26-3.06 (m, 4H), 2.96-2.83 (m, 5H), 2.39-2.30 (m, 1H), 1.86 (s, 6H); MS: (m/z) 550.1 (M+1).

### Pharmacological Assays

The pharmacological activity of the compounds as GPR40 agonists can be confirmed by a number of pharmacological assays known in the art. The exemplified pharmacological assay, given below, has been carried out with the compounds of the present invention synthesized in the above Examples.

### Example 123

### Inositol Phosphate Accumulation Assay

The inositol phosphate accumulation assay was performed to characterise the GPR40 agonist activity of the compounds of the present invention.

The assay was carried out in accordance with the method substantially as described in Diabetes, 2008, 57(8):2211-2219 and PLoS One, 2011, 6(11):e27270.

a) Generation of FFAR1 (GPR40) CHOK1 Clone

A stable FFAR1 (GPR40) CHOK1 clone was used to determine the GPR40 agonist activity of the test compounds (compounds of the present invention synthesized in the above Examples). The stable FFAR1 (GPR40) CHOK1 clone expressing recombinant human GPR40 was generated according to the procedure described herein below.

Full-length human GPR40 cDNA (Accession Number: NM_{—}005303) was cloned into mammalian expression vector (pReceiver) and was stably transfected into (Chinese Hamster Ovary) CHOK1 cells using Amaxa technology. 2 μg of pReceiver hGPR40 were transfected into 1×10⁶ CHOK1 cells in 6 well plates. The cells were split into three 100 mm cell culture plates on the second day and geneticin (800 μg/ml) was added to the cell culture on the third day. The selection medium comprising Ham's F-12 K supplemented with FBS (10%) and geneticin (800 μg/ml) was changed every three days until colonies were formed. The colonies isolated were further purified (single cell cloning) after 14 days to obtain pure isogenic single cell homogenous population of cells expressing the GPR40 receptor protein on the cell surface. GPR40 receptor expression on cell surface was measured by flow cytometry. The in-house transgenic cell line (clone) created was labeled as FFAR1 (GPR40) CHOK1 clone.

b) Determination of Intracellular Inositol Phosphate Release

FFAR1 (GPR40) CHOK1 cells were suspended in culture medium comprising Ham's F-12 K supplemented with FBS (10%) and geneticin (800 μg/ml). Cells were seeded at a density of 2×10⁴ cells per well in a 384 well tissue culture plate and cultured overnight. The medium was discarded and the cells were further resuspended in a stimulation buffer comprising HEPES (10 mM), glucose (5.5 mM), CaCl₂ (1 mM), NaCl (150 mM), KCl (4.2 mM), MgCl₂ (0.5 mM) and LiCl (50 mM) having a pH of 7.6. The test compounds (representative compounds of Formula (I)) 10 mM stock were prepared in DMSO and subsequently log fold dilution of the test compounds were carried out in the stimulation buffer. Various concentrations of the test compounds and the standard DMSO solution were added to each well. The plates were further incubated at 37° C., 5% CO₂ incubator for 1 h. The final concentration of the test compounds in the each well varied from 1 μM to 10 μM. The DMSO concentration in the assay was 0.1% or less. After incubation, lysis reagent and anti-Tb conjugate were added to each well. The intracellular Inositol Phosphate release and accumulation in the test compounds were measured by the binding ability of the anti-Tb conjugate with the inositol phosphate inherently produced in each well as compared with the inositol phosphate coupled to dye d2 added externally to each well. The plates were then read using Perkin Elmer (Envision) plate reader and the fluorescence signal was captured. The EC₅₀ values for the test compounds were calculated from the non linear regression sigmoidal curve graphs plotted between the concentrations of the test compounds and the fluorescence intensity. The EC₅₀ values for the test compounds are given in Table 1.

TABLE 1

| Example No. | EC₅₀ in nM |
|---|---|
| 4 | ++ |
| 6 | ++ |
| 8 | ++ |
| 10 | +++ |
| 12 | + |
| 14 | + |
| 16 | + |
| 18 | ++ |
| 22 | + |
| 24 | + |
| 28 | ++ |
| 30 | + |
| 32 | + |
| 41 | +++ |
| 43 | +++ |
| 45 | +++ |
| 47 | +++ |
| 49 | +++ |
| 51 | + |
| 53 | +++ |
| 55 | +++ |
| 57 | ++ |
| 59 | + |
| 62 | +++ |
| 66 | +++ |
| 68 | +++ |
| 70 | +++ |
| 72 | +++ |
| 75 | + |
| 77 | +++ |
| 79 | ++ |
| 81 | + |
| 83 | ++ |
| 85 | + |
| 87 | + |
| 89 | ++ |
| 94 | + |
| 96 | + |
| 98 | + |
| 102 | + |
| 106 | + |
| 108 | +++ |

| | |
|---|---|
| Symbol | EC₅₀ range class |
| +++ | <100 nM |
| ++ | >100 nM but <500 nM |
| + | >500 nM |

c) Conclusion:

The EC₅₀ values determined for the test compounds by the inositol phosphate accumulation assay is indicative of GPR40 agonist activity of the compounds of the present invention.

1-25. (canceled)

26. A compound of Formula (I); 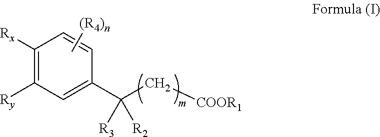
wherein,
R₁ is hydrogen or (C₁-C₆) alkyl;
R₂ and R₃ together form a saturated or a partially unsaturated 3- to 9-membered heterocyclyl ring containing one or two heteroatoms selected from O, N or S; or R₂ and R; together form a saturated or a partially unsaturated (C₄-C₈)cycloalkyl ring;
R₄ at each occurrence is independently selected from the group consisting of hydrogen, (C₁-C₆)alkyl, halogen, halo(C₁-C₆)alkyl, hydroxy, —O(C₁-C₆)alkyl, (C₈-C₁₀)aryl, amino, cyano, nitro, —C(O)R₉ and —S(O)ₚR₆;
Rₓ and R_{y} are independently selected from A-CH(R₇)—X and R₅; provided that at least one of Rₓ and R_{y} is A-CH(R₇)—X;
R₅ is hydrogen, (C₁-C₆)alkyl, halogen, halo(C₁-C₆) alkyl, hydroxy, —O(C₁-C₆)alkyl, (C₆-C₁₀)aryl, amino, cyano, nitro, —C(O)R₉ and —S(O)ₚR₆;
R₆ is hydrogen, (C₁-C₆)alkyl or amino;
R₇ is hydrogen or (C₁-C₆)alkyl;
X is O, NR₈ or S;
R₈ is hydrogen, (C₁-C₆) alkyl, (C₃-C₈)cycloalkyl, (C₆-C₁₀)aryl, heterocyclyl, heteroaryl, cyano, —C(O)(C₁-C₆)alkyl, —C(O)O(C₁-C₆)alkyl, —C(O)NH₂ or —S(O)ₚR₆; wherein R₆ is as defined above;
R₉ is (C₁-C₆)alkyl, —O(C₁-C₆)alkyl, hydroxy or amino;
A is (C₃-C₆)cycloalkyl, (C₆-C₁₀)aryl, heterocyclyl, heteroaryl, 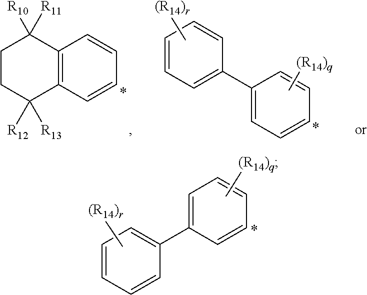
R₁₀, R₁₁, R₁₂ and R₁₃ are independently selected from hydrogen or (C₁-C₆) alkyl; or R₁₀ and R₁₁ can together form a (C₃-C₈)cycloalkyl ring and R₁₂ and R₁₃ are hydrogen; or
R₁₂ and R₁₃ can together form a (C₃-C₈)cycloalkyl ring and R₁₀ and R₁₁ are hydrogen;
R₁₄ at each occurrence is independently selected from the group consisting of hydrogen, (C₁-C₆) alkyl, halogen, halo(C₁-C₆)alkyl, hydroxy, —O(C₁-C₆)alkyl, —O(C₃-C₆)cycloalkyl, —O(C₁-C₆)alkyl-heterocyclyl, —O-heterocyclyl, halo(C₁-C₆)alkoxy, —O(C₁-C₆)alkyl-S(O)ₚR₆, (C₆-C₁₀)aryl, amino, cyano, nitro, —C(O)R₉, —S(O)ₚR₆, —(CH₂)ₛNR₁₅R₁₆ and —X(CH₂)ₛNR₁₅R₁₆;
R₁₅ and R₁₆ are independently selected from the group consisting of hydrogen, (C₁-C₆)alkyl and —(CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4;
t is an integer from 1 to 4;
* indicates the point of attachment to —CH of CH(R₇)—X;
wherein,
(C₁-C₆)alkyl is unsubstituted or substituted with one or more groups independently selected from the group consisting of (C₁-C₆)alkyl, (C₂-C₈)alkenyl, (C₂-C₈)alkynyl, halogen, halo(C₁-C₆)alkyl, hydroxy, —O(C₁-C₆)alkyl, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆;
—O(C₁-C₆)alkyl is unsubstituted or substituted with one or more groups independently selected from the group consisting of (C₁-C₆)alkyl, (C₃-C₈)cycloalkyl, heterocyclyl, hydroxy, halogen, amino, cyano, —(C₁-C₆)alkyl-S(O)ₚR₆, —S(O)ₚR₆, —NR₁₅R₁₆ and —(CH₂)ₛNR₁₅R₁₆;
(C₆-C₁₀)aryl is unsubstituted or substituted with one or more groups independently selected from the group consisting of (C₁-C₆)alkyl, (C₂-C₈)alkenyl, (C₂-C₈)alkynyl, halogen, halo(C₁-C₆)alkyl, hydroxy, —O(C₁-C₆)alkyl, halo(C₁-C₆)alkoxy, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆;
heterocyclyl is a 3- to 9-membered ring, which is unsubstituted or substituted with one or more groups independently selected from the group consisting of (C₁-C₁₀)alkyl, (C₂-C₈)alkenyl, (C₂-C₈)alkynyl, halogen, halo(C₁-C₆)alkyl, hydroxy, —O(C₁-C₆)alkyl, halo(C₁-C₆)alkoxy, (C₃-C₈)cycloalkyl, (C₆-C₁₀)aryl, heterocyclyl, heteroaryl, amino, cyano, nitro, —(C₁-C₆)alkyl-OH, (C₁-C₆)alkyl-O—(C₁-C₆)alkyl, —C(O)R₉ and —O(C₁-C₆)alkyl-S(O)ₚR₆;
heteroaryl is a 3- to 10-membered ring, which is unsubstituted or substituted with one or more groups independently selected from the group consisting of (C₁-C₆)alkyl, (C₂-C₃)alkenyl, (C₂-C₈) alkynyl, halogen, halo(C₁-C₆)alkyl, hydroxy, —O(C₁-C₆)alkyl, halo(C₁-C₆)alkoxy, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆;
halogen is chlorine, bromine, iodine or fluorine;
or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

27. A compound according to claim 26 of the Formula (Ia); 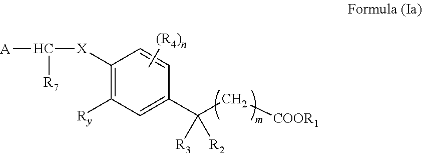
wherein,
R₁, R₂, R₃, R₄, R₇, R_{y}, A, X, m and n are as defined in claim 26;
or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

28. A compound according to claim 26;
wherein,
R₁ is hydrogen or (C₁-C₆) alkyl;
R₂ and R₃ together form a saturated or a partially unsaturated 3- to 9-membered heterocyclyl ring containing one or two heteroatoms independently selected from O, N and S;
R₄ at each occurrence is independently selected from the group consisting of hydrogen, (C₁-C₆) alkyl, halogen, halo(C₁-C₆)alkyl, hydroxy, —O(C₁-C₆)alkyl, amino, cyano, —C(O)R₉ and —S(O)ₚR₆;
R_{y} is R₅;
R₅ is hydrogen, (C₁-C₆)alkyl, halogen, halo(C₁-C₆)alkyl, hydroxy, —O(C₁-C₆)alkyl, amino, cyano, —C(O)R₉ or —S(O)ₚR₆;
R₆ is hydrogen, (C₁-C₆) alkyl or amino;
R₇ is hydrogen or (C₁-C₆)alkyl;
X is O, NR₈ or S;
R₈ is selected from the group consisting of hydrogen, (C₁-C₆) alkyl, —C(O)(C₁-C₆)alkyl, —C(O)O(C₁-C₆)alkyl, —C(O)NH₂ and —S(O)ₚR₆,
R₉ is selected from the group consisting of (C₁-C₆) alkyl, —O(C₁-C₆)alkyl, hydroxy and amino;
A is (C₆-C₁₀)aryl, heteroaryl, 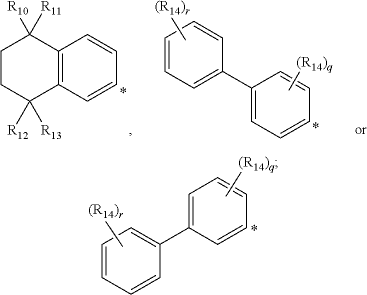
R₁₀, R₁₁, R₁₂, and R₁₃ are independently selected from hydrogen or (C₁-C₆) alkyl; or R₁₀ and R₁₁ can together form a (C₃-C₈) cycloalkyl ring and R₁₂ and R₁₃ are hydrogen; or
R₁₂ and R₁₃ can together form a (C₃-C₈) cycloalkyl ring and R₁₂ and R₁₃ are hydrogen;
R₁₄ at each occurrence is independently selected from the group consisting of hydrogen, (C₁-C₆) alkyl, halogen, halo(C₁-C₆)alkyl, hydroxy, —O(C₁-C₆)alkyl, —O(C₃-C₈)cycloalkyl, halo(C₁-C₆)alkoxy, —O(C₁-C₆)alkyl-S(O)ₚR₆, —O(C₁-C₆)alkyl-heterocyclyl, —O-heterocyclyl, (C₆-C₁₀)aryl, amino, cyano, nitro, —C(O)R₉, —S(O)ₚR₆, —(CH₂)ₛNR₁₅R₁₆ and —X(CH₂)ₛNR₁₅R₁₆;
R₁₅ and R₁₆ are independently selected from the group consisting of hydrogen, (C₁-C₆)alkyl and —(CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4;
t is an integer from 1 to 4;
* indicates the point of attachment to —CH of CH(R₇)—X;
wherein,
(C₁-C₆)alkyl is unsubstituted or substituted with one or more groups independently selected from the group consisting of (C₁-C₆)alkyl, halogen, halo(C₁-C₆)alkyl, hydroxy, —O(C₁-C₆)alkyl, (C₃-C₈)cycloalkyl, (C₆-C₁₀)aryl, heterocyclyl, heteroaryl, amino, cyano, nitro, —C(O)R₉ and —O(C₁-C₁₀)alkyl-S(O)ₚR₆;
—O(C₁-C₆)alkyl is unsubstituted or substituted with one or more groups independently selected from the group consisting of (C₁-C₆)alkyl, (C₃-C₈)cycloalkyl, heterocyclyl, hydroxy, halogen, amino, cyano, —(C₁-C₆)alkyl-S(O)₉R₆, —S(O)ₚR₆, —NR₁₅R₁₆ and —(CH₂)NR₁₅R₁₆;
(C₆-C₁₀)aryl is unsubstituted or substituted with one or more groups independently selected from the group consisting of (C₁-C₆)alkyl, halogen, halo(C₁-C₆)alkyl, hydroxy, —O((C₁-C₆) alkyl, halo(C₁-C₆)alkoxy, (C₆-C₁₀)aryl, heteroaryl, amino, cyano, nitro, —C(O)R₉ and —O(C₁-C₆)alkyl-S(O)ₚR₆;
heterocyclyl is a 3 to 9-membered ring, which is unsubstituted or substituted with one or more groups independently selected from the group consisting of (C₁-C₆)alkyl, halogen, halo(C₁-C₆)alkyl, hydroxy, —O(C₁-C₆) alkyl, halo(C₁-C₆)alkoxy, (C₆-C₁₀)aryl, amino, cyano, nitro, —(C₁-C₆)alkyl-OH, (C₁-C₆)alkyl-O—(C₁-C₆)alkyl and —O(C₁-C₆)alkyl-S(O)ₚR₆;
heteroaryl is a 3- to 10-membered ring, which is unsubstituted or substituted with one or more groups independently selected from the group consisting of (C₁-C₆)alkyl, halogen, hydroxy, —O(C₁-C₆) alkyl, halo(C₁-C₆)alkoxy, (C₁-C₁₀)aryl, heteroaryl, heterocyclyl, amino, cyano, nitro, —C(O)R₉ and —O(C₁-C₆)alkyl-S(O)ₚR₆;
halogen is chlorine, bromine, iodine or fluorine;
or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

29. A compound according to claim 26;
wherein,
R₁ is hydrogen or (C₁-C₆) alkyl;
R₂ and R₃ together form a saturated or a partially unsaturated 3- to 9-membered heterocyclyl ring containing one or two oxygen atoms;
R₄ at each occurrence is independently selected from the group consisting of hydrogen, (C₁-C₆) alkyl, halogen, halo(C₁-C₆)alkyl, hydroxy, —O(C₁-C₆)alkyl, amino, cyano, —C(O)R and —S(O)ₚR₆;
R_{y} is R₅;
R₅ is hydrogen, (C₁-C₆) alkyl, halogen, halo(C₁-C₆)alkyl, hydroxy, —O(C₁-C₆)alkyl, amino, cyano, —C(O)R₉ or —S(O)ₚR₆;
R₆ is hydrogen, (C₁-C₆) alkyl or amino;
R₇ is hydrogen or (C₁-C₆)alkyl;
X is O;
R₉ is (C₁-C₄) alkyl, —O(C₁-C₆)alkyl, hydroxy or amino;
A is 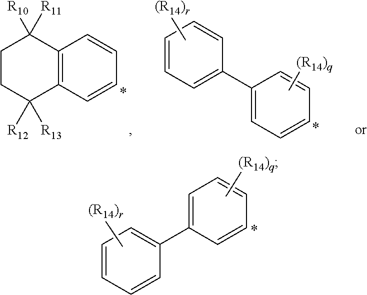
R₁₀, R₁₁, R₁₂ and R₁₁ are independently selected from hydrogen or (C₁-C₆) alkyl; or R₁₀ and R₁₁ can together form a (C₃-C₈)cycloalkyl ring and R₁₂ and R₁₃ are hydrogen; or
R₁₂ and R₁₃ can together form a (C₃-C₈)cycloalkyl ring and R₁₂ and R₁₃ are hydrogen;
R₁₄ at each occurrence is independently selected from the group consisting of hydrogen, (C₁-C₆) alkyl, halogen, halo(C₁-C₆)alkyl, hydroxy, —O(C₁-C₆)alkyl, —O(C₃-C₈)cycloalkyl, halo(C₁-C₆)alkoxy, —O(C₁-C₆)alkyl-S(O)ₚR₆, —O(C₁-C₆)alkyl-heterocyclyl, —O-heterocyclyl, (C₆-C₁₀)aryl, amino, cyano, nitro, —C(O)R₉, —S(O)ₚR₉, —(CH₂)ₛNR₁₅R₁₆ and —X(CH₂)ₛNR₁₅R₁₆;
R₁₅ and R₁₆ are independently selected from the group consisting of hydrogen, (C₁-C₆)alkyl and (CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4;
t is an integer from 1 to 4;
* indicates the point of attachment to —CH of CH(R₇)—X;
wherein,
(C₁-C₆)alkyl is unsubstituted or substituted with one or more groups independently selected from the group consisting of (C₁-C₆)alkyl, halogen, halo(C₁-C₆)alkyl, hydroxy, O(C₁-C₆)alkyl, (C₃-C₈)cycloalkyl, (C₆-C₁₀)aryl, heterocyclyl, heteroaryl, amino, cyano, nitro, C(O)R₉ and O(C₁-C₆)alkyl-S(O)ₚR₆;
—O(C₁-C₆)alkyl is unsubstituted or substituted with one or more groups independently selected from the group consisting of (C₁-C₆)alkyl, (C₃-C₈)cycloalkyl, heterocyclyl, hydroxy, halogen, amino, cyano, (C₁-C₆)alkyl-S(O)ₚR₆, —S(O)ₚR₆; —NR₁₅R₁₆ and —(CH₂)ₛNR₁₅R₁₆;
(C₆-C₁₀)aryl is unsubstituted or substituted with one or more groups independently selected from the group consisting of (C₁-C₆)alkyl, halogen, halo(C₁-C₆)alkyl, hydroxy, O(C₁-C₆) alkyl, halo(C₁-C₆)alkoxy, (C₆-C₁₀)aryl, heteroaryl, amino, cyano, nitro, C(O)R₉ and O(C₁-C₆)alkyl-S(O)ₚR₆;
heterocyclyl is a 3- to 9-membered ring, which is unsubstituted or substituted with one or more groups independently selected from the group consisting of (C₁-C₆)alkyl, halogen, halo(C₁-C₆)alkyl, hydroxy, O(C₁-C₆)alkyl, halo(C₁-C₆)alkoxy, (C₆-C₁₀)aryl, amino, cyano, nitro, (C₁-C₆)alkyl-OH, (C₁-C₁₀)alkyl-O—(C₁-C₆)alkyl and O(C₁-C₆)alkyl-S(O)ₚR₆;
heteroaryl is a 3- to 10-membered ring, which is unsubstituted or substituted with one or more groups independently selected from the group consisting of (C₁-C₆)alkyl, halogen, halo(C₁-C₆)alkyl, hydroxy, O(C₁-C₆) alkyl, halo(C₁-C₆)alkoxy, (C₆-C₁₀)aryl, heteroaryl, amino, cyano, nitro, C(O)R₉ and O(C₁-C₆)alkyl-S(O)ₚR₆;
halogen is chlorine, bromine, iodine or fluorine;
or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

30. A compound according to claim 26;
wherein,
R₁ is hydrogen or (C₁-C₆)alkyl;
R₂ and R₃ together form a saturated or a partially unsaturated 3- to 9-membered heterocyclyl ring containing one or two oxygen atoms;
R₄ at each occurrence is independently selected from the group consisting of hydrogen, (C₁-C₆)alkyl, halogen, halo(C₁-C₆)alkyl, hydroxy, —O(C₁-C₆)alkyl, amino, cyano, —C(O)R₉ and —S(O)ₚR₆;
R_{y} is R₅;
R₅ is hydrogen, (C₁-C₆) alkyl, halogen, halo(C₁-C₆)alkyl, hydroxy, —O(C₁-C₆)alkyl, amino, cyano, —C(O)R₉ or —S(O)ₚR₆;
R₆ is hydrogen, (C₁-C₆) alkyl or amino;
R₇ is hydrogen or (C₁-C₆)alkyl;
X is O;
R₉ is (C₁-C₄) alkyl, —O(C₁-C₆)alkyl, hydroxy or amino;
A is 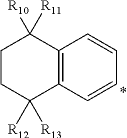
R₁₀, R₁₁, R₁₂ and R₁₃ represent (C₁-C₆) alkyl;
R₁₅ and R₁₆ are independently selected from the group consisting of hydrogen, (C₁-C₆)alkyl and —(CH₂)ₜOH;
n is an integer from 1 to 3;
m is an integer from 0 to 4;
p is an integer from 0 to 2;
s is an integer from 1 to 4;
t is an integer from 1 to 4;
* indicates the point of attachment to —CH of CH(R₇)—X;
wherein,
(C₁-C₆)alkyl is unsubstituted or substituted with one or more groups independently selected from the group consisting of (C₁-C₆)alkyl, halogen, halo(C₁-C₆)alkyl, hydroxy, —O(C₁-C₆) alkyl, amino, cyano, nitro, —C(O)R₉ and —O(C₁-C₆)alkyl-S(O)ₚR₆;
—O(C₁-C₆)alkyl is unsubstituted or substituted with one or more groups independently selected from the group consisting of hydroxy, halogen, amino, —(C₁-C₆)alkyl-S(O)ₚR₆, —S(O)ₚR₆, —NR₁₅R₁₆ and —(CH₂)ₛNR₁₅R₁₆;
halogen is chlorine, bromine, iodine or fluorine;
or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

31. A compound according to claim 26;
wherein,
R₁ is hydrogen or (C₁-C₆)alkyl;
R₂ and R₃ together form a saturated or a partially unsaturated 3- to 9-membered heterocyclyl ring containing one or two oxygen atoms,
R₄ at each occurrence is independently selected from the group consisting of hydrogen, (C₁-C₆)alkyl, halogen, halo(C₁-C₆)alkyl, hydroxy, —O(C₁-C₆)alkyl, amino, cyano, —C(O)R₉ and —S(O)ₚR₆;
R_{y} is R₅;
R₅ is hydrogen, (C₁-C₆) alkyl, halogen, halo(C₁-C₆)alkyl, hydroxy, —O(C₁-C₆)alkyl, amino, cyano, —C(O)R₉ or —S(O)ₚR₆;
R₆ is hydrogen, (C₁-C₆)alkyl or amino;
R₇ is hydrogen or (C₁-C₆)alkyl;
X is O;
R₉ is (C₁-C₆) alkyl, O(C₁-C₆)alkyl, hydroxy or amino;
A is 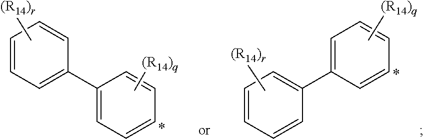
R₁₄ at each occurrence is independently selected from the group consisting of hydrogen, (C₁-C₆)alkyl, halogen, halo(C₁-C₆)alkyl, —O(C₁-C₆)alkyl, halo(C₁-C₆)alkoxy, —O(C₁-C₆)alkyl-S(O)ₚR₆, —O(C₃-C₈)cycloalkyl, —O(C₁-C₆)alkyl-heterocyclyl, —O-heterocyclyl, cyano, —S(O)ₚR₆, —(CH₂)ₛNR₁₅R₁₆ and —X(CH₂)ₛNR₁₅R₁₆;
R₁₅ and R₁₆ are independently selected from the group consisting of hydrogen, (C₁-C₆)alkyl and —(CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4,
t is an integer from 1 to 4;
* indicates the point of attachment to —CH of CH(R₇)—X;
wherein,
(C₁-C₆)alkyl is unsubstituted or substituted with one or more groups independently selected from the group consisting of (C₁-C₆)alkyl, halogen, halo(C₁-C₆)alkyl, hydroxy, —O(C₁-C₆) alkyl, (C₆-C₁₀)aryl, amino, cyano, nitro, —C(O)R₉ and —O(C₁-C₆)alkyl-S(O)ₚR₆;
—O(C₁-C₆)alkyl is unsubstituted or substituted with one or more groups independently selected from the group consisting of (C₁-C₆)alkyl, (C₃-C₈)cycloalkyl, heterocyclyl, hydroxy, halogen, cyano, —(C₁-C₆)alkyl-S(O)ₚR₆, —S(O)ₚR₆, —NR₁₅R₁₆ and —(CH₂)ₛNR₁₅R₁₆;
heterocyclyl is a 3- to 9-membered ring, which is unsubstituted or substituted with one or more groups independently selected from the group consisting of (C₁-C₆)alkyl, halogen, halo(C₁-C₆)alkyl, hydroxy, —O(C₁-C₆)alkyl, halo(C₁-C₆)alkoxy, (C₆-C₁₀)aryl, amino, cyano, nitro, —(C₁-C₆)alkyl-OH, (C₁-C₆)alkyl-O—(C₁-C₆)alkyl and —O(C₁-C₆)alkyl-S(O)ₚR₆;
halogen is chlorine, bromine, iodine or fluorine;
or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

32. A compound according to claim 26;
wherein,
R₁ is hydrogen or (C₁-C₆) alkyl;
R₂ and R₃ together form a saturated or a partially unsaturated 3- to 9-membered heterocyclyl ring containing one or two oxygen atoms;
R₄ at each occurrence is independently selected from the group consisting of hydrogen, (C₁-C₆)alkyl, halogen, halo(C₁-C₆)alkyl, hydroxy, —O(C₁-C₆)alkyl, amino, cyano, —C(O)R₉ and —S(O)ₚR₆;
R_{y} is R₅;
R₅ is hydrogen, (C₁-C₆) alkyl, halogen, halo(C₁-C₆)alkyl, hydroxy, —O(C₁-C₆)alkyl, amino, cyano, —C(O)R₉ or —S(O)ₚR₆;
R₆ is hydrogen, (C₁-C₆)alkyl or amino;
R₇ is hydrogen or (C₁-C₆)alkyl;
X is O;
R₉ is (C₁-C₆)alkyl, O(C₁-C₆)alkyl, hydroxy or amino;
A is 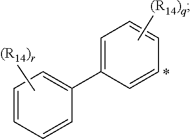
R₁₄ at each occurrence is independently selected from the group consisting of hydrogen, (C₁-C₆)alkyl, halogen, halo(C₁-C₆)alkyl, —O(C₁-C₆)alkyl, halo(C₁-C₆)alkoxy, —O(C₁-C₆)alkyl-S(O)ₚR₆, —O(C₃-C₈)cycloalkyl, —O(C₁-C₆)alkyl-heterocyclyl, —O-heterocyclyl, cyano, —S(O)ₚR₆, —(CH₂)ₛNR₁₅R₁₆ and —X(CH₂)NR₁₅R₁₆;
R₁₅ and R₁₆ are independently selected from the group consisting of hydrogen, (C₁-C₆)alkyl and —(CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4;
t is an integer from 1 to 4;
* indicates the point of attachment to —CH of CH(R₇)—X;
wherein,
(C₁-C₆)alkyl is unsubstituted or substituted with one or more groups independently selected from the group consisting of (C₁-C₆)alkyl, halogen, halo(C₁-C₆)alkyl, hydroxy, amino, cyano, nitro, —C(O)ₚR₉ and —O(C₁-C₆)alkyl-S(O)ₚR₆;
—O(C₁-C₆)alkyl is unsubstituted or substituted with one or more groups independently selected from the group consisting of (C₁-C₆)alkyl, (C₃-C₈)cycloalkyl, heterocyclyl, hydroxy, halogen, amino, cyano, —(C₁-C₆)alkyl-S(O)ₚR₆, —S(O)ₚR₆, —NR₁₅R₁₆, and —(CH₂)ₛNR₁₅R₁₆;
heterocyclyl is a 3- to 9-membered ring, which is unsubstituted or substituted with one or more groups independently selected from the group consisting of (C₁-C₆)alkyl, halogen, halo(C₁-C₆)alkyl, hydroxy, —O(C₁-C₆) alkyl, halo(C₁-C₆)alkoxy, (C₆-C₁₀)aryl, amino, —(C₁-C₆)alkyl-OH, —(C₁-C₆)alkyl-O—(C₁-C₆)alkyl and —O((C₁-C₆)alkyl-S(O)ₚR₆;
halogen is chlorine, bromine, iodine or fluorine;
or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

33. A compound according to claim 26,
wherein,
R₁ is hydrogen or (C₁-C₆) alkyl;
R₂ and R₃ together form a saturated or a partially unsaturated 3- to 9-membered heterocyclyl ring containing one or two oxygen atoms;
R₄ at each occurrence is independently selected from the group consisting of hydrogen, (C₁-C₆)alkyl, halogen, halo(C₁-C₆)alkyl, hydroxy, —O(C₁-C₆)alkyl, amino, cyano, —C(O)R₉ and —S(O)ₚR₆;
R_{y} is R₅;
R₅ is hydrogen, (C₁-C₆) alkyl, halogen, halo(C₁-C₆)alkyl, hydroxy, —O(C₁-C₆)alkyl, amino, cyano, —C(O)R₉ or —S(O)ₚR₆;
R₆ is hydrogen, (C₁-C₆)alkyl or amino;
R₇ is hydrogen or (C₁-C₆)alkyl;
X is O;
R₉ is (C₁-C₄) alkyl, —O(C₁-C₆)alkyl, hydroxy or amino;
A is (C₆-C₁₀)aryl or heteroaryl;
n is an integer from 1 to 3;
m is an integer from 0 to 4;
p is an integer from 0 to 2;
wherein,
(C₁-C₆)alkyl is unsubstituted or substituted with one or more groups independently selected from the group consisting of (C₁-C₆)alkyl, halogen, halo(C₁-C₆)alkyl, hydroxy, amino, cyano, nitro, —C(O)R₉ and —O(C₁-C₆)alkyl-S(O)ₚR₆;
—O(C₁-C₆)alkyl is unsubstituted or substituted with one or more groups independently selected from the group consisting of (C₁-C₆)alkyl, (C₃-C₈)cycloalkyl, heterocyclyl, hydroxy, halogen, amino, cyano, —(C₁-C₆)alkyl-S(O)ₚR₆, —S(O)ₚR₆, —NR₁₅R₁₆ and —(CH₂)ₛNR₁₅R₁₆;
(C₆-C₁₀)aryl is unsubstituted or substituted with one or more groups independently selected from the group consisting of (C₁-C₆)alkyl, halogen, halo(C₁-C₆)alkyl, hydroxy, —O(C₁-C₆) alkyl, halo(C₁-C₆)alkoxy, heteroaryl, amino, cyano, nitro, —C(O)R₉ and —O(C₁-C₆)alkyl-S(O)ₚR₆;
heterocyclyl is a 3- to 9-membered ring, which is unsubstituted or substituted with one or more groups independently selected from the group consisting of (C₁-C₆)alkyl, halogen, halo(C₁-C₆)alkyl, hydroxy, —O(C₁-C₆) alkyl, halo(C₁-C₆)alkoxy, (C₆-C₁₀)aryl, amino, —(C₁-C₆)alkyl-OH, (C₁-C₆)alkyl-O—(C₁-C₆)alkyl and —O(C₁-C₆)alkyl-S(O)ₚR₆;
heteroaryl is a 3- to 10-membered ring, which is unsubstituted or substituted with one or more groups independently selected from the group consisting of (C₁-C₆)alkyl, halogen, halo(C₁-C₆)alkyl, hydroxy, —O(C₁-C₆) alkyl, halo(C₁-C₆)alkoxy, amino, cyano, nitro, (C₆-C₁₀)aryl, heterocyclyl, —C(O)R₉ and —O(C₁-C₆)alkyl-S(O)ₚR₆;
halogen is chlorine, bromine, iodine or fluorine;
or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

34. A compound according to claim 26;
wherein,
R₁ is hydrogen or (C₁-C₆) alkyl;
R₂ and R₃ together form a saturated or a partially unsaturated 3- to 9-membered heterocyclyl ring containing one or two oxygen atoms;
R₄ at each occurrence is independently selected from the group consisting of hydrogen, (C₁-C₆)alkyl, halogen, halo(C₁-C₆)alkyl, hydroxy, —O(C₁-C₆)alkyl, amino, cyano, —C(O)R₉ and —S(O)ₚR₆;
R_{y} is R₅;
R₅ is hydrogen, (C₁-C₆) alkyl, halogen, halo(C₁-C₆)alkyl, hydroxy, —O(C₁-C₆)alkyl, amino, cyano, —C(O)R₉ or —S(O)ₚR₆;
R₆ is hydrogen, (C₁-C₆) alkyl or amino;
R₇ is hydrogen or (C₁-C₆)alkyl;
X is NR₆;
R₈ is hydrogen or (C₁-C₆)alkyl,
R₉ is (C₁-C₄) alkyl, —O(C₁-C₆)alkyl, hydroxy or amino;
A is 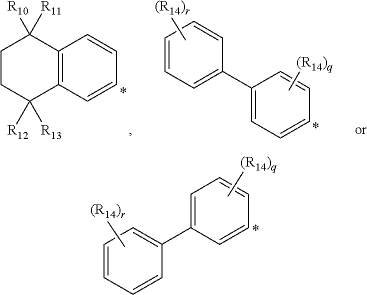
R₁₀, R₁₁, R₁₂ and R₁₃ are independently selected from hydrogen or (C₁-C₆) alkyl; or R₁₀ and R₁₁ can together form a (C₁-C₆)cycloalkyl ring and R₁₂ and R₁₃ are hydrogen; or
R₁₂ and R₁₃ can together form a (C₃-C₈) cycloalkyl ring and R₁₂ and R₁₃ are hydrogen;
R₁₄ at each occurrence is independently selected from the group consisting of hydrogen, (C₁-C₆) alkyl, halogen, halo(C₁-C₆)alkyl, hydroxy, —O(C₁-C₆)alkyl, halo(C₁-C₆)alkoxy, —O(C₁-C₆)alkyl-S(O)ₚR₆, —O(C₃-C₈)cycloalkyl, —O(C₁-C₆)alkyl-heterocyclyl, —O-heterocyclyl, (C₆-C₁₀)aryl, amino, cyano, nitro, —C(O)R₉, —S(O)ₚR₆, —(CH₂)ₛNR₁₅R₁₆ and —X(CH₂)ₛNR₁₅R₁₆;
R₁₅ and R₁₆ are independently selected from the group consisting of hydrogen, (C₁-C₆)alkyl and —(CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4;
t is an integer from 1 to 4;
* indicates the point of attachment to —CH of CH(R₇)—X;
wherein,
(C₁-C₆)alkyl is unsubstituted or substituted with one or more groups independently selected from the group consisting of (C₁-C₆)alkyl, halogen, halo(C₁-C₆)alkyl, hydroxy, O(C₁-C₆)alkyl, (C₃-C₈)cycloalkyl, (C₆-C₁₀)aryl, heterocyclyl, heteroaryl, amino, cyano, nitro, C(O)R₉ and O(C₁-C₆)alkyl-S(O)ₚR₆;
—O(C₁-C₆)alkyl is unsubstituted or substituted with one or more groups independently selected from the group consisting of (C₁-C₆)alkyl, (C₃-C₈)cycloalkyl, heterocyclyl, hydroxy, halogen, amino, cyano, —(C₁-C₆)alkyl-S(O)ₚR₆, —S(O)ₚR₆, —NR₁₅R₁₆ and —(CH₂)ₛNR₁₅R₁₆;
(C₆-C₁₀)aryl is unsubstituted or substituted with one or more groups independently selected from the group consisting of (C₁-C₆)alkyl, halogen, halo(C₁-C₆)alkyl, hydroxy, —O(C₁-C₆) alkyl, halo(C₁-C₆)alkoxy, (C₆-C₁₀)aryl, heteroaryl, amino, cyano, nitro, —C(O)R₉ and —O(C₁-C₆)alkyl-S(O)ₚR₆;
heterocyclyl is a 3- to 9-membered ring, which is unsubstituted or substituted with one or more groups independently selected from the group consisting of (C₁-C₆)alkyl, halogen, halo(C₁-C₆)alkyl, hydroxy, —O(C₁-C₆) alkyl, halo(C₁-C₆)alkoxy, (C₆-C₁₀)aryl, amino, cyano, nitro, —(C₁-C₆)alkyl-OH, (C₁-C₆)alkyl-O—(C₁-C₆)alkyl and —O(C₁-C₆)alkyl-S(O)ₚR₆;
heteroaryl is a 3- to 10-membered ring, which is unsubstituted or substituted with one or more groups independently selected from the group consisting of (C₁-C₆)alkyl, halogen, halo(C₁-C₆)alkyl, hydroxy, —O(C₁-C₆) alkyl, halo(C₁-C₆)alkoxy, (C₆-C₁₀)aryl, heteroaryl, amino, cyano, nitro, —C(O)R₉ and —O(C₁-C₆)alkyl-S(O)ₚR₆;
halogen is chlorine, bromine, iodine or fluorine;
or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

35. A compound according to claim 26;
wherein,
R₁ is hydrogen or (C₁-C₆) alkyl;
R₂ and R₃ together form a saturated or a partially unsaturated 3- to 9-membered heterocyclyl ring containing one or two oxygen atoms;
R₄ at each occurrence is independently selected from the group consisting of hydrogen, (C₁-C₆)alkyl, halogen, halo(C₁-C₆)alkyl, hydroxy, —O(C₁-C₆)alkyl, amino, cyano, —C(O)R₉ and —S(O)ₚR₆;
R_{y} is R₅;
R₅ is hydrogen, (C₁-C₆) alkyl, halogen, halo(C₁-C₆)alkyl, hydroxy, —O(C₁-C₆)alkyl, amino, cyano, —C(O)R₉ or —S(O)ₚR₆;
R₆ is hydrogen, (C₁-C₆) alkyl or amino;
R₇ is hydrogen or (C₁-C₆)alkyl;
X is NR₈;
R₈ is hydrogen or (C₁-C₆) alkyl;
R₉ is (C₁-C₆) alkyl, —O(C₁-C₆)alkyl, hydroxy or amino,
A is 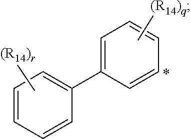
R₁₄ at each occurrence is independently selected from the group consisting of hydrogen, (C₁-C₆) alkyl, halogen, halo(C₁-C₆)alkyl, —O(C₁-C₆)alkyl, halo(C₁-C₆)alkoxy, —O(C₁-C₆)alkyl-S(O)ₚR₆, —O(C₃-C₈)cycloalkyl, —O(C₁-C₆)alkyl-heterocyclyl, —O-heterocyclyl, cyano, —S(O)ₚR₆, —(CH₂)NR₁₅R₁₆ and —X(CH₂)ₛNR₁₅R₁₆;
R₁₅ and R₁₆ are independently selected from the group consisting of hydrogen, (C₁-C₆)alkyl and —(CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4;
t is an integer from 1 to 4;
* indicates the point of attachment to —CH of CH(R₇)—X;
wherein,
(C₁-C₆)alkyl is unsubstituted or substituted with one or more groups independently selected from the group consisting of (C₁-C₆)alkyl, halogen, halo(C₁-C₆)alkyl, hydroxy, amino, cyano, nitro, —C(O)R₉ and —O(C₁-C₆)alkyl-S(O)ₚR₆;
—O(C₁-C₆)alkyl is unsubstituted or substituted with one or more groups independently selected from the group consisting of (C₁-C₆)alkyl, (C₁-C₆)cycloalkyl, heterocyclyl, hydroxy, halogen, amino, cyano, —(C₁-C₆)alkyl-S(O)ₚR₆, —S(O)ₚR₆, —NR₁₅R₁₆ and —(CH₂)NR₁₅R₁₆;
heterocyclyl is a 3- to 9-membered ring, which is unsubstituted or substituted with one or more groups independently selected from the group consisting of (C₁-C₆)alkyl, halogen, halo(C₁-C₆)alkyl, hydroxy, —O(C₁-C₆)alkyl, halo(C₁-C₆)alkoxy, (C₆-C₁₀)aryl, amino, —(C₁-C₆)alkyl-OH, (C₁-C₆)alkyl-O—(C₁-C₆)alkyl and —O(C₁-C₆)alkyl-S(O)ₚR₆;
halogen is chlorine, bromine, iodine or fluorine;
or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

36. A compound according to claim 26;
wherein,
R₁ is hydrogen or (C₁-C₆)alkyl;
R₂ and R₃ together form a saturated or a partially unsaturated 3- to 9-membered heterocyclyl ring containing one or two nitrogen or sulfur atoms when the heteroatom is N, it may be substituted with a group selected from the group consisting of hydrogen, (C₁-C₆)alkyl, —C(O)(C₁-C₆)alkyl and —S(O)₂(C₁-C₆)alkyl;
R₄ at each occurrence is independently selected from the group consisting of hydrogen, (C₁-C₆) alkyl, halogen, halo(C₁-C₆)alkyl, hydroxy, —O(C₁-C₆)alkyl, amino, cyano, —C(O)R₉ and —S(O)ₚR₆;
R_{y} is R₅;
R₅ is hydrogen, (C₁-C₆)alkyl, halogen, halo(C₁-C₆)alkyl, hydroxy, —O(C₁-C₆)alkyl, amino, cyano, —C(O)R₉ or —S(O)ₚR₆;
R₆ is hydrogen, (C₁-C₄) alkyl or amino;
R₇ is hydrogen or (C₁-C₆)alkyl;
X is O,
R₉ is (C₁-C₄) alkyl, —O(C₁-C₆)alkyl, hydroxy or amino;
A is (C₆-C₁₀)aryl, heteroaryl, 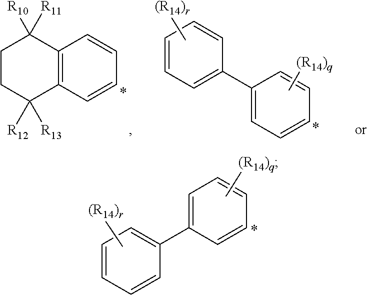
R₁₀, R₁₁, R₁₂ and R₁₃ are independently selected from hydrogen or (C₁-C₆) alkyl; or R₁₀ and R₁₁ can together form a (C₃-C₈) cycloalkyl ring and R₁₂ and R₁₃ are hydrogen; or
R₁₂ and R₁₃ can together form a (C₃-C₈) cycloalkyl ring and R₁₂ and R₁₃ are hydrogen;
R₁₄ at each occurrence is independently selected from the group consisting of hydrogen, (C₁-C₆)alkyl, halogen, halo(C₁-C₆)alkyl, hydroxy, —O(C₁-C₆)alkyl, halo(C₁-C₆)alkoxy, —O(C₁-C₆)alkyl-S(O)ₚR₆, —O(C₃-C₈)cycloalkyl, —O(C₁-C₆)alkyl-heterocyclyl, —O-heterocyclyl, (C₆-C₁₀)aryl, amino, cyano, nitro, —C(O)R₉, —S(O)ₚR₆, —(CH₂)ₛNR₁₅R₁₆ and —X(CH₂)ₛNR₁₅R₁₆;
R₁₅ and R₁₆ are independently selected from the group consisting of hydrogen, (C₁-C₆)alkyl and —(CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4;
is an integer from 1 to 4;
* indicates the point of attachment to —CH of CH(R₇)—X;
wherein,
(C₁-C₆)alkyl is unsubstituted or substituted with one or more groups independently selected from the group consisting of (C₁-C₆)alkyl, halogen, halo(C₁-C₆)alkyl, hydroxy, —O(C₁-C₆)alkyl, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆;
—O(C₁-C₆)alkyl is unsubstituted or substituted with one or more groups independently selected from the group consisting of (C₁-C₆)alkyl, (C₃-C₈)cycloalkyl, heterocyclyl, hydroxy, halogen, amino, cyano, —(C₁-C₆)alkyl-S(O)ₚR₆, —S(O)ₚR₆, —NR₁₅R₁₆ and —(CH₂)ₛNR₁₅R₁₆;
(C₆-C₁₀)aryl is unsubstituted or substituted with one or more groups independently selected from the group consisting of (C₁-C₆)alkyl, halogen, halo(C₁-C₆)alkyl, hydroxy, —O(C₁-C₆)alkyl, halo(C₁-C₆)alkoxy, (C₆-C₁₀)aryl, heteroaryl, amino, cyano, nitro, —C(O)R₉ and —O(C₁-C₆)alkyl-S(O)ₚR₆;
heterocyclyl is a 3- to 9-membered ring, which is unsubstituted or substituted with one or more groups independently selected from the group consisting of (C₁-C₆)alkyl, halogen, halo(C₁-C₆)alkyl, hydroxy, —O(C₁-C₆) alkyl, halo(C₁-C₆)alkoxy, (C₆-C₁₀)aryl, amino, cyano, nitro, —C(O)R₉, —(C₁-C₆)alkyl-OH, (C₁-C₆)alkyl-O—(C₁-C₆)alkyl and —O(C₁-C₆)alkyl-S(O)ₚR₆;
heteroaryl is a 3- to 10-membered ring, which is unsubstituted or substituted with one or more groups independently selected from the group consisting of (C₁-C₆)alkyl, halogen, halo(C₁-C₆)alkyl, hydroxy, —O(C₁-C₆) alkyl, halo(C₁-C₆)alkoxy, (C₆-C₁₀)aryl, heteroaryl, amino, cyano, nitro, —C(O)R₉ and —O(C₁-C₆)alkyl-S(O)ₚR₆;
halogen is chlorine, bromine, iodine or fluorine;
or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

37. A compound according to claim 26;
wherein,
R₁ is hydrogen or (C₁-C₆) alkyl;
R₂ and R₃ together form a saturated or a partially unsaturated (C₄-C₈) cycloalkyl ring;
R₄ at each occurrence is independently selected from the group consisting of hydrogen, (C₁-C₆) alkyl, halogen, halo(C₁-C₆)alkyl, hydroxy, —O(C₁-C₆)alkyl, amino, cyano, —C(O)R₉ and —S(O)ₚR₆;
R_{y} is R₅;
R₅ is hydrogen, (C₁-C₆) alkyl, halogen, halo(C₁-C₆)alkyl, hydroxy, —O(C₁-C₆)alkyl, amino, cyano, —C(O)R₉ or —S(O)ₚR₆;
R₆ is hydrogen, (C₁-C₆) alkyl or amino;
R₇ is hydrogen or (C₁-C₆)alkyl;
X is O, NR₈ or S,
R₈ is hydrogen, (C₁-C₆; alkyl, —C(O)(C₁-C₆)alkyl, —C(O)O(C₁-C₆)alkyl, —C(O)NH₂ or —S(O)ₚR₆;
R₉ is (C₁-C₄) alkyl, —O(C₁-C₆)alkyl, hydroxy or amino;
A is (C₆-C₁₀)aryl, heteroaryl, 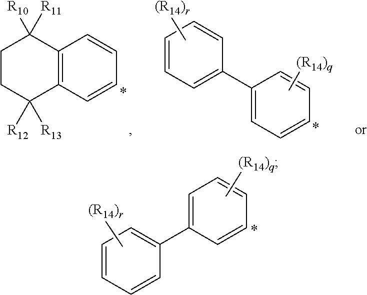
R₁₀, R₁₁, R₁₂ and R₁₃ are independently selected from hydrogen or (C₁-C₆) alkyl; or R₁₀ and R₁₁ can together form a (C₃-C₈)cycloalkyl ring and R₁₂ and R₁₃ are hydrogen; or
R₁₂ and R₁₃ can together form a (C₃-C₈) cycloalkyl ring and R₁₂ and R₁₃ are hydrogen,
R₁₄ at each occurrence is independently selected from the group consisting of hydrogen, (C₁-C₆) alkyl, halogen, halo(C₁-C₆)alkyl, hydroxy, —O(C₁-C₆)alkyl, halo(C₁-C₆)alkoxy, —O(C₁-C₆)alkyl-S(O)ₚR₆, —O(C₃-C₈)cycloalkyl, —O(C₁-C₆)alkyl-heterocyclyl, —O-heterocyclyl, (C₆-C₁₀)aryl, amino, cyano, nitro, —C(O)R₉, —S(O)ₚR₆, —(CH₂)ₛNR₁₅R₁₆ and —X(CH₂)ₛNR₁₅R₁₆;
R₁₅ and R₁₆ are independently selected from the group consisting of hydrogen, (C₁-C₆)alkyl and —(CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4;
t is an integer from 1 to 4;
* indicates the point of attachment to —CH of CH(R₇)—X;
wherein,
(C₁-C₆)alkyl is unsubstituted or substituted with one or more groups independently selected from the group consisting of (C₁-C₆)alkyl, halogen, halo(C₁-C₆)alkyl hydroxy, —O(C₁-C₆)alkyl, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆;
—O(C₁-C₆)alkyl is unsubstituted or substituted with one or more groups independently selected from the group consisting of (C₁-C₆)alkyl, hydroxy, halogen, amino, cyano, —(C₁-C₆)alkyl-S(O)ₚR₉, —S(O)ₚR₆, —NR₁₅R₁₆ and —(CH₂)ₛNR₁₅R₁₆;
(C₆-C₁₀)aryl is unsubstituted or substituted with one or more groups independently selected from the group consisting of (C₁-C₆)alkyl, halogen, halo(C₁-C₆)alkyl, hydroxy, —O(C₁-C₆) alkyl, halo(C₁-C₆)alkoxy, (C₆-C₁₀)aryl, heteroaryl, amino, cyano, nitro, —C(O)R₉ and —O(C₁-C₆)alkyl-S(O)ₚR₆;
heterocyclyl is a 3- to 9-membered ring, which is unsubstituted or substituted with one or more groups independently selected from the group consisting of (C₁-C₆)alkyl, halogen, halo(C₁-C₆)alkyl, hydroxy, —O(C₁-C₆) alkyl, halo(C₁-C₆)alkoxy, (C₆-C₁₀)aryl, amino, cyano, nitro, —C(O)R₉, —(C₁-C₆)alkyl-OH, (C₁-C₆)alkyl-O—(C₁-C₆)alkyl and —O(C₆-C₁₀)alkyl-S(O)ₚR₆;
heteroaryl is a 3- to 10-membered ring, which is unsubstituted or substituted with one or more groups independently selected from the group consisting of (C₁-C₆)alkyl, halogen, halo(C₁-C₆)alkyl, hydroxy, —O(C₁-C₆) alkyl, halo(C₁-C₆)alkoxy, (C₆-C₁₀)aryl, heteroaryl, amino, cyano, nitro, —C(O)R₉ and —O(C₁-C₆)alkyl-S(O)ₚR₆;
halogen is chlorine, bromine, iodine or fluorine;
or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

38. A compound according to claim 26 of the Formula (Ib), 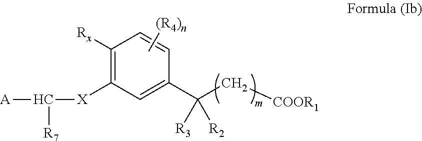
wherein,
R₁ is hydrogen or (C₁-C₆) alkyl;
R₂ and R₃ together form a saturated or a partially unsaturated 3- to 9-membered heterocyclyl ring containing one or two heteroatoms selected from O, N or S; or
R₂ and R₃ together form a saturated or a partially unsaturated (C₄-C₈) cycloalkyl ring;
R₄ at each occurrence is independently selected from the group consisting of hydrogen, (C₁-C₆) alkyl, halogen, halo(C₁-C₆)alkyl, hydroxy, —O(C₁-C₆)alkyl, (C₆-C₁₀)aryl, amino, cyano, nitro, —C(O)R₉ and —S(O)ₚR₆;
Rₓ is A-CH(R₇)—X or R₅;
R₅ is hydrogen, (C₁-C₆) alkyl, halogen, halo(C₁-C₆)alkyl, hydroxy, —O(C₁-C₆)alkyl, (C₆-C₁₀)aryl, amino, cyano, nitro, —C(O)R₉ or —S(O)ₚR₆;
R₆ is hydrogen, (C₁-C₆) alkyl or amino;
R₇ is hydrogen or (C₁-C₆)alkyl;
X is O, NR₈ or S;
R₈ is hydrogen, (C₁-C₆) alkyl, (C₃-C₈)cycloalkyl, (C₆-C₁₀)aryl, heterocyclyl, heteroaryl, cyano, —C(O)(C₁-C₆)alkyl, —C(O)O(C₁-C₆)alkyl, —C(O)NH₂ or —S(O)ₚR₆; wherein R₆ is as defined above;
R₉ is (C₁-C₆) alkyl, —O(C₁-C₆)alkyl, hydroxy or amino;
A is (C₃-C₈)cycloalkyl, (C₆-C₁₀)aryl, heterocyclyl, heteroaryl, 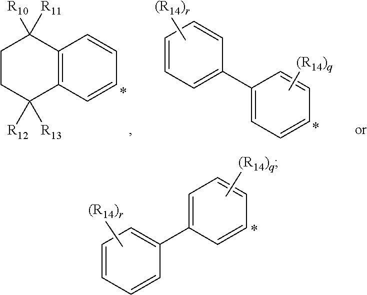
R₁₀, R₁₁, R₁₂ and R₁₃ are independently selected from hydrogen and (C₁-C₆)alkyl; or R₁₀ and R₁₁ can together form a (C₃-C₈)cycloalkyl ring and R₁₂ and R₁₃ are hydrogen; or
R₁₂ and R₁₃ can together form a (C₃-C₈) cycloalkyl ring and R₁₀ and R₁₁ are hydrogen;
R₁₄ at each occurrence is independently selected from the group consisting of hydrogen, (C₁-C₆) alkyl, halogen, halo(C₁-C₆)alkyl, hydroxy, —O(C₁-C₆)alkyl, halo(C₁-C₆)alkoxy, —O(C₁-C₆)alkyl-S(O)ₚR₆, —O(C₃-C₈)cycloalkyl, —O(C₁-C₆)alkyl-heterocyclyl, —O-heterocyclyl, (C₆-C₁₀)aryl, amino, cyano, nitro, —C(O)R₉, —S(O)ₚR₆, —(CH₂)NR₁₅R₁₆ and —X(CH₂)ₛNR₁₅R₁₆;
R₁₅ and R₁₆ are independently selected from the group consisting of hydrogen, (C₁-C₆)alkyl and —(CH₂)ₜOH;
n is an integer from 1 to 3;
m is an integer from 0 to 4;
p is an integer from 0 to 2;
q is an integer from 1 to 4;
r is an integer from 1 to 5;
s is an integer from 1 to 4;
t is an integer from 1 to 4;
* indicates the point of attachment to —CH of CH(R₇)—X;
wherein,
(C₁-C₆)alkyl is unsubstituted or substituted with one or more groups independently selected from the group consisting of (C₁-C₆)alkyl, (C₂-C₈) alkenyl, (C₂-C₈) alkynyl, halogen, halo(C₁-C₆)alkyl, hydroxy, —O(C₁-C₆)alkyl, (C₃-C₈)cycloalkyl, (C₆-C₁₀)aryl, heterocyclyl, heteroaryl, amino, cyano, nitro, —C(O)R₉ and —O(C₁-C₆)alkyl-S(O)ₚR₆;
—O(C₁-C₆)alkyl is unsubstituted or substituted with one or more groups independently selected from the group consisting of (C₁-C₆)alkyl, (C₃-C₈)cycloalkyl, heterocyclyl, hydroxy, halogen amino, cyano, —(C₁-C₆)alkyl-S(O)ₚR₆, —S(O)ₚR₆, —NR₁₅R₁₆ and —(CH₂)ₛNR₁₅R₁₆;
(C₆-C₁₀)aryl is unsubstituted or substituted with one or more groups independently selected from the group consisting of (C₁-C₆)alkyl, (C₂-C₈) alkenyl, (C₂-C₈) alkynyl, halogen, halo(C₁-C₆)alkyl, hydroxy, —O(C₁-C₆) alkyl, halo(C₁-C₆)alkoxy, (C₃-C₆)cycloalkyl, (C₆-C₁₀)aryl, heterocyclyl, heteroaryl, amino, cyano, nitro, —C(O)R₉ and —O(C₁-C₆)alkyl-S(O)ₚR₆;
heterocyclyl is a 3- to 9-membered ring, which is unsubstituted or substituted with one or more groups independently selected from the group consisting of (C₁-C₆)alkyl, (C₂-C₈)alkenyl, (C₂-C₈)alkynyl, halogen, halo(C₁-C₆)alkyl, hydroxy, —O(C₁-C₆) alkyl, halo(C₁-C₆)alkoxy, (C₃-C₆)cycloalkyl, (C₆-C₁₀)aryl, heterocyclyl, heteroaryl, amino, cyano, nitro, —C(O)R₉, —(C₁-C₆)alkyl-OH, (C₁-C₆)alkyl-O—(C₁-C₆)alkyl and —O(C₁-C₆)alkyl-S(O)ₚR₆;
heteroaryl is a 3- to 10-membered ring, which is unsubstituted or substituted with one or more groups independently selected from the group consisting of (C₁-C₆)alkyl, (C₂-C₈) alkenyl, (C₂-C₈)alkynyl, halogen, halo(C₁-C₆)alkyl, hydroxy, —O(C₁-C₆) alkyl, halo(C₁-C₆)alkoxy, (C₃-C₆)cycloalkyl, (C₆-C₁₀)aryl, heterocyclyl, heteroaryl, amino, cyano, nitro, —C(O)R₉ and —O(C₁-C₆)alkyl-S(O)ₚR₆; wherein R₆, R₉, and p are as defined above;
halogen is chlorine, bromine, iodine or fluorine;
or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

39. A compound according to claim 26, wherein the compound is:
Ethyl 2-(3-(4-((4′-(trifluoromethyl)biphenyl-3-yl)methoxy)phenyl)oxetan-3-yl)acetate;
2-(3-(4-((4′-(Trifluoromethyl)biphenyl-3-yl)methoxy)phenyl)oxetan-3-yl)acetic acid;
Ethyl 2-(3-(4-([1,1′-biphenyl]-3-ylmethoxy)phenyl)oxetan-3-yl)acetate;
2-(3-(4-([1,1′-Biphenyl]-3-ylmethoxy)phenyl)oxetan-3-yl)acetic acid;
Ethyl 2-(3-(4-((2′-cyano-[1,1′-biphenyl]-4-yl)methoxy)phenyl)oxetan-3-yl)acetate;
2-(3-(4-((2′-Cyano-([1,1′-biphenyl]-4-yl)methoxy)phenyl)oxetan-3-yl)acetic acid;
Ethyl 2-(3-(4-([1,1′-biphenyl]-4-ylmethoxy)phenyl)oxetan-3-yl)acetate;
2-(3-(4-([1,1′-Biphenyl]-4-yl)methoxy)phenyl)oxetan-3-yl)acetic acid;
Ethyl 2-(3-(4-((2′,6′-dimethyl-4′-(3-(methylsulfonyl)propoxy)-[1,1′-biphenyl]-3-yl)methoxy)phenyl)oxetan-3-yl)acetate;
2-(3-(4-((2′,6′-Dimethyl-4′-(3-(methylsulfonyl)propoxy)-[1,1′-biphenyl]-3-yl)methoxy)phenyl)oxetan-3-yl)acetic acid;
Ethyl 2-(3-(4-([1,1′-biphenyl]-3-ylmethoxy)-3-fluorophenyl)oxetan-3-yl)acetate;
2-(3-(4-([1,1′-Biphenyl]-3-ylmethoxy)-3-fluorophenyl)oxetan-3-yl)acetic acid;
Ethyl 2-(3-(4-([1,1′-biphenyl]-4-ylmethoxy)-3-fluorophenyl)oxetan-3-yl)acetate;
2-(3-(4-(t 1,1′-Biphenyl-4-ylmethoxy)-3-fluorophenyl)oxetan-3-yl)acetic acid;
Ethyl 2-(3-(4-((2′,6′-dimethyl-4′-(3-(methylsulfonyl)propoxy)-[1,1′-biphenyl]-3-yl)methoxy)-3-fluorophenyl)oxetan-3-yl)acetate;
2-(3-(4-((2′,6-Dimethyl-4′-(3-(methylsulfonyl)propoxy)-[1,1′-biphenyl]-3-yl)methoxy)-3-fluorophenyl)oxetan-3-yl)acetic acid;
Ethyl 2-(3-(4-((5,5,8,8-tetramethyl-5,6,7,6-tetrahydronaphthalen-2-yl)methoxy)phenyl)oxetan-3-yl)acetate;
2-(3-(4-((5,5,5,8-Tetramethyl-5,6,7,8-tetrahydronaphthalen-2-yl)methoxy)phenyl)oxetan-3-yl)acetic acid,
Ethyl 2-(3-fluoro-4-((5,5,8,8-tetramethyl-5,6,7,8-tetrahydronaphthalen-2-yl)methoxy)phenyl)oxetan-3-yl)acetate;
2-(3-(3-Fluoro-4-((5,5,8,8-tetramethyl-5,6,7,8-tetrahydronaphthalen-2-yl)methoxy)phenyl)oxetan-3-yl)acetic acid;
Ethyl 2-(3-(4-((4-methoxy-3-(trifluoromethyl)benzyl)oxy)phenyl)oxetan-3-yl)acetate;
2-(3-(4(4-Methoxy-3-(trifluoromethyl)benzyl)oxy)phenyl)oxetan-3-yl)acetic acid;
Ethyl 2-(3-(4-((2-methyl-5-(trifluoromethyl)benzyl)oxy)phenyl)oxetan-3-yl)acetate;
2-(3-(4-(2-Methyl-5-(trifluoromethyl)benzyl)oxy)phenyl)oxetan-3-yl)acetic acid;
Ethyl 2-(3-(4-((2-methoxy-5-(trifluoromethyl)benzyl)oxy)phenyl)oxetan-3-yl)acetate;
2-(3-(4-(2-Methoxy-5-(trifluoromethyl)benzyl)oxy)phenyl)oxetan-3-yl)acetic acid;
Ethyl 2-(3-(4-((4-methyl-3-(trifluoromethyl)benzyl)oxy)phenyl)oxetan-3-yl)acetate;
2-(3-(4-((4-Methyl-3-(trifluoromethyl)benzyl)oxy)phenyl)oxetan-3-yl)acetic acid;
Ethyl 2-(3-(4(3-methoxy-4-(trifluoromethyl)benzyl)oxy)phenyl)oxetan-3-Yl)acetate;
2-(3-(4-((3-Methoxy-4-(trifluoromethyl)benzyl)oxy)phenyl)oxetan-3-yl)acetic acid;
Ethyl 2-(3-(4-(3-fluoro-4-(trifluoromethyl)benzyl)oxy)phenyl)oxetan-3-yl)acetate;
2-(3-(4-((3-Fluoro-4-(trifluoromethyl)benzyl)oxy)phenyl)oxetan-3-yl)acetic acid;
Ethyl 2-3(4-((3-fluoro-5-(trifluoromethoxy)benzyl)oxy)phenyl)oxetan-3-yl)acetic acid;
2-(3-(4-((3-Fluoro-5-(trifluoromethoxy)benzyl)oxy)phenyl)oxetan-3-yl)acetic acid;
Ethyl 2-(3-(4-((3-fluoro-4-(trifluoromethoxy)benzyl)oxy)phenyl)oxetan-3-yl)acetate,
2-(3-(4-((3-Fluoro-4-(trifluoromethoxy)benzyl)oxy)phenyl)oxetan-3-yl)acetic acid;
Ethyl 2-(3-(4-((2-fluoro-3-(trifluoromethyl)benzyl)oxy)phenyl)oxetan-3-yl)acetate;
2-(3-(4-((2-Fluoro-3-(trifluoromethyl)benzyl)oxy)phenyl)oxetan-3-yl)acetic acid;
Ethyl 2-(3-(4-((4′-hydroxy-2′,6′-dimethyl-[1,1′-biphenyl]-3-yl)methoxy)phenyl)oxetan;
Ethyl 2-(3-(4-((2′,6′-dimethyl-4-((tetrahydrofuran-3-yl)methoxy)-[1,1′-biphenyl]-3-yl)methoxy)phenyl)oxetan-3-yl)acetate;
2-(3-(4-((2′,6′-Dimethyl-4′-((tetrahydrofuran-3-yl)methoxy)-[1,1′-biphenyl]-3-yl)methoxy)phenyl)oxetan-3-yl)acetic acid,
Ethyl 2-(3-(4-((2′,6′-dimethyl-4′-((tetrahydro-2H-pyran-4-yl)methoxy)-[1,1′-biphenyl]-3-yl)methoxy)phenyl)oxetan-3-yl)acetate;
2-(3-(4-((2′,6′-Dimethyl-4′((tetrahydro-2H-pyran-4-yl)methoxy)-[1,1′-biphenyl]-3-yl)methoxy)phenyl)oxetan-3-yl)acetic acid;
Ethyl 2-(2-(4-((4′-((1,1-dioxidotetrahydro-2H-thiopyran-4-yl)methoxy)-2′,6′-dimethyl-[1,1′-biphenyl]-3-yl)methoxy)phenyl)oxetan-3-yl)acetate;
2-(3-(4-((4′-((1,1-Dioxidotetrahydro-2H-thiopyran-4-yl)methoxy)-2′,6′-dimethyl-[1,1′-biphenyl]-3-yl)methoxy)phenyl)oxetan-3-yl)acetic acid;
Ethyl 2-(3-(4-((2,6′-dimethyl-4′-((tetrahydrofuran-2-yl)methoxy)-[1,1′-biphenyl]-3-yl)methoxy)phenyl)oxetan-3-yl)acetate;
2-(3-(4-((2,6′-Dimethyl-4′-((tetrahydrofuran-2-yl)methoxy)-[1,1′-biphenyl]-3-yl)methoxy)phenyl)oxetan-3-yl)acetic acid;
(R)-ethyl 2-(3-(4-((2′,6′-dimethyl-4′-((tetrahydrofuran-3-yl)methoxy)-[1,1]-biphenyl-3-yl)methoxy)phenyl)oxetan-3-yl)acetate;
(R)-2-(3-(4-((2′,6′-dimethyl-4′-((tetrahydrofuran-3-yl)methoxy)-[1,1′-biphenyl]-3-yl)methoxy)phenyl)oxetan-3-yl)acetic acid;
Ethyl 2-(3-(4-((2′,6′-dimethyl-4-((3-methyloxetan-3-yl)methoxy)-[1,1′-biphenyl]-3-yl)methoxy)phenyl)oxetan-3-yl)acetate;
2-(3-(4-((2′,6′-Dimethyl-4′-((3-methyloxetan-3-yl)methoxy)-[1,1′-biphenyl]-3-yl)methoxy)phenyl)oxetan-3-yl)acetic acid;
Ethyl 2-(3-(4-((4′-((1,1-dioxidotetrahydrothiophen-3-yl)methoxy)-2′,6′-dimethyl-[1,1′-biphenyl]-3-yl)methoxy)phenyl)oxetan-3-yl)acetate;
2-(3-(4-((4′-((1,1-Dioxidotetrahydrothiophen-3-yl)methoxy)-2,6-dimethyl-[1,1′-biphenyl]-3-yl)methoxy)phenyl)oxetan-3-yl)acetic acid;
Ethyl 2-(3-(4-((4′-((3-(hydroxymethyl)oxetan-3-yl)methoxy)-2′,6′-dimethyl-[1,1′-biphenyl]-3-yl)methoxy)phenyl)oxetan-3-yl)acetate;
2-(3-(4-((4′-((3-(Hydroxymethyl)oxetan-3-yl)methoxy)-2′,6′-dimethyl-[1,1′-biphenyl]-3-yl)methoxy)phenyl)oxetan-3-yl)acetic acid;
Ethyl 2-(3-(4-((4′-((1,1-dioxidotetrahydro-2H-thiopyran-4-yl)oxy)-2′,6′-dimethyl-[1,1′-biphenyl]-3-yl)methoxy)phenyl)oxetan-3-yl)acetate;
2-(3-(4-((4′-((1,1-Dioxidotetrahydro-2H-thiopyran-4-yl)oxy)-2′,6′-dimethyl-[1,1′-biphenyl]-3-yl)methoxy)phenyl)oxetan-3-yl)acetic acid;
Ethyl 2-(3-(4-((4′-(cyclopentyloxy)-2′,6′-dimethyl-[1,1′-biphenyl]-3-yl)methoxy)phenyl)oxetan-3-yl)acetate;
2-(3-(4-((4′-(Cyclopentyloxy)-2′,6′-dimethyl-[1,1′-biphenyl]-3-yl)methoxy)phenyl)oxetan-3-yl) acetic acid;
Ethyl 2-(3-(4-((2′-chloro-4′-hydroxy-[1,1′-biphenyl]-3-yl)methoxy)phenyl)oxetan-3-yl)acetate;
Ethyl 2-(3-(4-((2′-chloro-4′-(3-(methylsulfonyl)propoxy)-[1,1′-biphenyl]-3-yl)methoxy)phenyl)oxetan-3-yl)acetate;
2-(3-(4-((2′-Chloro-4′-(3-(methylsulfonyl)propoxy)-[1,1′-biphenyl]-3-yl)methoxy)phenyl)oxetan-3-yl)acetic acid;
Ethyl 2-(3-(4-((2′-chloro-4′-((3-methyloxetan-3-yl)methoxy)-[1,1′-biphenyl]-3-yl)methoxy)phenyl)oxetan-3-yl)acetate;
2-(3-(4-((2′-Chloro-4′-((3-methyloxetan-3-yl)methoxy)-[1,1′-biphenyl]-3-yl)methoxy)phenyl)oxetan-3-yl)acetic acid;
Ethyl 2-(3-(4-((2′-chloro-4′-((3-(hydroxymethyl)oxetan-3-yl)methoxy)-[1,1′-biphenyl]-3-yl)methoxy)phenyl)oxetan-3-yl)acetate;
2-(3-(4-((2-Chloro-4′-((3-(hydroxymethyl)oxetan-3-yl)methoxy)-[1,1′-biphenyl]-3-yl)methoxy)phenyl)oxetan-3-yl)acetic acid;
Ethyl 2-(3-(4-((2′-Chloro-4′-((1,1-dioxidotetrahydrothiophen-3-yl)methoxy)-[1,1′-biphenyl]-3-yl)methoxy)phenyl)oxetan-4 yl)acetate;
2-(3-(4-((2′-Chloro-4′-((1,1-dioxidotetrahydrothiophen-3-yl)methoxy)-[1,1′-biphenyl]-3-yl)ethoxy)phenyl)oxetan-3-yl)acetic acid;
Ethyl 2-(3-(4-(2′-chloro-4′-((1,1-dioxidotetrahydro-2H-thiopyran-4-yl)methoxy)-[1,1′-biphenyl]-3-yl)methoxy)phenyl)oxetan-3-yl)acetate;
2-(3-(4-((2′-Chloro-4′-((1,1-dioxidotetrahydro-2H-thiopyran-4-yl)methoxy)-[1,1′-biphenyl]-3-yl)methoxy)phenyl)oxetan-3-yl)acetic acid,
Ethyl 2-(3-(4-((2′-chloro-4′-((tetrahydro-2H-pyran-4yl)methoxy)-[1,1′-biphenyl]-3-yl)methoxy)phenyl)oxetan-3-yl)acetate;
2-(3-(4-((2′-Chloro-4′-((tetrahydro-2H-pyran-4-yl)methoxy)-[1,1′-biphenyl]-3-yl)methoxy)phenyl)oxetan-3-yl)acetic acid;
Ethyl 2-(3-(4-((4′-hydroxy-[1,1′-biphenyl]-3-yl)methoxy)phenyl)oxetan-3-yl)acetate;
Ethyl 2-(3-(4-((4′-(cyclobutylmethoxy)-[1,1′-biphenyl]-3-yl methoxy)phenyl)oxetan-3-yl)acetate;
2-(3-(4-((4′-(Cyclobutylmethoxy)-[1,1′-biphenyl]-3-yl)methoxy)phenyl)oxetan-3-yl)acetic acid;
Ethyl 2-(3-(4-((2-methyl-4′-(3-(methylsulfonyl)propoxy)-[1,1′-biphenyl]-3-yl)methoxy)phenyl)oxetan-3-yl)acetate;
2-(3-(4-((2′-Methyl-4′-(3-(methylsulfonyl)propoxy-[1,1′-biphenyl]-3-yl)methoxy)phenyl)oxetan-3-yl)acetic acid;
Ethyl 2-(3-(4-((3,5′-dimethyl-4′-(3-(methylsulfonyl)propoxy)-[1,1′-biphenyl]-3-yl)methoxy)phenyl)oxetan-3-yl)acetate;
2-(3-(4-((3′,5′-Dimethyl-4′-(3-(methylsulfonyl)propoxy)-[1,1′-biphenyl]-3-yl)methoxy)phenyl)oxetan-3-yl)acetic acid;
Ethyl 2-(3-(4-((3′-methoxy-4′-(3-(methylsulfonyl)propoxy)-[1,1′-biphenyl]-3-yl)methoxy)phenyl)oxetan-3-yl)acetate;
2-(3-(4-((3′-Methoxy-4′-(3-(methylsulfonyl)propoxy)-[1,1′-biphenyl]-3-yl)methoxy)phenyl)oxetan-3-yl)acetic acid;
Ethyl 2-(3-(4-((4′-(methylthio)-[1,1′-biphenyl]-3-yl)methoxy)phenyl)oxetan-3-yl)acetate;
2-(3-(4-((4′-(Methylthio)-[1,1′-biphenyl]-3-yl)methoxy)phenyl)oxetan-3-yl)acetic acid;
Ethyl 2-(3-(4-((4′-(butylthio)-[1,1′-biphenyl]-3-yl)methoxy)phenyl)oxetan-3-yl)acetate;
2-(3-(4-((4′-(Butylthio)-[1,1′-biphenyl]-3-yl)methoxy)phenyl)oxetan-3-yl)acetic acid;
Ethyl 2-(3-(4-((4′-(3-(methylsulfonyl)propoxy)-3′-(trifluoromethyl)-[1,1′-biphenyl]-3-yl)methoxy)phenyl)oxetan-3-yl)acetate;
2-(3-(4-((4′-(3-Methylsulfonyl)propoxy)-3′-(trifluoromethyl)-[1,1′-biphenyl]-3-yl)methoxy)phenyl)oxetan-3-yl)acetic acid;
Ethyl 2-(3-(4-((4′-(isopropylthio)-[1,1′-biphenyl]-3-yl)methoxy)phenyl)oxetan-3-yl)acetate;
2-(3-(4-((4′-(Isopropylthio)-[1,1′-biphenyl]-3-yl)methoxy)phenyl)oxetan-3-yl)acetic acid;
Ethyl 2-(3-(4-((5-methyl-2-phenyloxazol-4-yl)methoxy)phenyl)oxetan-3-yl)acetate;
2-(3-(4-((5-Methyl-2-phenyloxazol-4-yl)methoxy)phenyl)oxetan-3-yl)acetic acid;
Ethyl 2-(3-(4 ((2′,6′-dimethyl-4′-(3-(methylsulfonyl)propoxy)-[1,1′-biphenyl]-3-yl)methoxy)phenyl)azetidin-3-yl)acetate;
Ethyl 2-(3-(4-((2,6′-dimethyl-4′-(3-(methylsulfonyl)propoxy)-[1,1]-biphenyl)-3-yl)methoxy)phenyl)-1-(methylsulfonyl)azetidin-3-yl)acetate;
2-(3-(4-((2′,6′-Dimethyl-4′-(3-(methylsulfonyl)propoxy)-[1,1′-biphenyl]-3-yl)methoxy)phenyl)-1-(methylsulfanyl)azetidin-3-yl)acetic acid;
Ethyl 2-(1-acetyl-3-(4-((2′,6′-dimethyl-4′-(3-(methylsulfonyl)propoxy)-[1,1′-biphenyl]-3-yl)methoxy)phenyl)azetidin-3-yl)acetate;
2-(1-Acetyl-3-(4-((2′,6′-dimethyl-4′-(3-(methylsulfonyl)propoxy)-[1,1′-biphenyl]-3-yl)methoxy)phenyl)azetidin-3-yl)acetic acid;
Ethyl 2-(3-(3-fluoro-4-((4′-(trifluoromethyl)-[1,1′-biphenyl]-3-yl)methoxy)phenyl)oxetan-3-yl)acetate;
2-(3-(3-Fluoro-4-((4′-(trifluoromethyl)-[1,1′-biphenyl]-3-yl)methoxy)phenyl)oxetan-3-yl)acetic acid;
Ethyl 2-(3-(4-((4-fluoro-3-(trifluoromethoxy)benzyl)oxy)phenyl)oxetan-3-yl)acetate;
2-(3-(4-((4-Fluoro-3-(trifluoromethoxy)benzyl)oxy)phenyl)oxetan-3-yl)acetic acid;
Ethyl 2-(3-(4-((3-fluorobenzyl)oxy)phenyl)oxetan-3-yl)acetate;
2-(3-(4-((3-Fluorobenzyl)oxy)phenyl)oxetan-3-yl)acetic acid;
Ethyl 2-(3-(4-((2-fluoro-5-(trifluoromethoxy)benzyl)oxy)phenyl)oxetan-3-yl)acetate;
2-(3-(4-((2-Fluoro-5-(trifluoromethoxy)benzyl)oxy)phenyl)oxetan-3-yl)acetic acid;
Ethyl 2-(3-(4-((3-(5-methoxypyridin-3-yl)benzyl)oxy)phenyl)oxetan-3-yl)acetate;
2-(3-(1-((3-(5-Methoxypyridin-3-yl)benzyl)oxy)phenyl)oxetan-3-yl)acetic acid;
Ethyl 2-(3-(4-((3-(2-morpholinopyrimidin-5-yl)benzyl)oxy)phenyl)oxetan-3-yl)acetate;
2-(3-(4-((3-(2-Morpholinopyrimidin-5-yl)benzyl)oxy)phenyl)oxetan-3-yl)acetic acid;
Ethyl 2-(3-(4-((3-(6-(3-(methylsulfonyl)propoxy)pyridin-3-yl)benzyl)oxy)phenyl)oxetan-3-yl)acetate;
2-(3-(4-((3-(6-(3-(Methylsulfonyl)propoxy)pyridin-3-yl)benzyl)oxy)phenyl)oxetan 3-yl)acetic acid;
Ethyl 2-(3-(4-((4′-(isopentyloxy)-2′,6′-dimethyl-[1,1′-biphenyl]-3-yl)methoxy)phenyl)oxetan-3-yl)acetate;
2-(3-(4-((4′-(Isopentyloxy-2′,6′-dimethyl-[1,1′-biphenyl]-3-yl)methoxy)phenyl)oxetan-3-yl)acetic acid;
Ethyl 2-(3-(4-((4′-((1,3-difluoropropan-2-yl)oxy)-2′,6′-dimethyl-[1,1′-biphenyl]-3yl)methoxy)phenyl)oxetan-3-yl)acetate;
2-(3-(4-((4′-((1,3-Difluoropropan-2-yl)oxy)-2,6′-dimethyl-[1,1′-biphenyl]-3-yl)methoxy)phenyl)oxetan-3-yl)acetic acid,
Ethyl 2-(3-(4-((2′,6-dimethyl-4′-(neopentyloxy)-[1,1′-biphenyl]-3-yl)methoxy)phenyl)oxetan-3-yl)acetate;
2-(3-(4-((2′,6′-Dimethyl-4′-(neopentyloxy)-[1,1′-biphenyl]-3yl)methoxy)phenyl)oxetan-3-yl)acetic acid;
Ethyl 2-(3-(4-4(4′-(2-methoxyethoxy)-2′,6′-dimethyl-[1,1′-biphenyl]-3-yl)methoxy)phenyl)oxetan-3-yl)acetate,
2-(3-(4-((4′-(2-Methoxyethoxy)-2′,6′-dimethyl-[1,1′-biphenyl]-3-yl)methoxy)phenyl)oxetan-3-yl)acetic acid;
Ethyl 2-(3-(4-((4′-((3-(methoxymethyl)oxetan-3-yl)methoxy)-2′,6′-dimethyl-[1,1′-biphenyl]-3-yl)methoxy)phenyl)oxetan-3-yl)acetate;
2-(3-(4-((4′-((3-(Methoxymethyl)oxetan-3-yl)methoxy)-2′,6′-dimethyl-[1,1′-biphenyl]-3-yl)methoxy)phenyl)oxetan-3-yl)acetic acid;
Ethyl 2-(3-(4-(((4′-((1,1-dioxidotetrahydrothiophen-3-yl)methoxy)-2′,6′-dimethyl-[1,1′-biphenyl]-3-yl)methyl)amino)phenyl)oxetan-3-yl)acetate,
2-(3(4-((4-((1,1-Dioxidotetrahydrothiophen-3-yl)methoxy)-2′,6′-dimethyl-[1,1′-biphenyl]-3-yl)methyl)amino)phenyl)oxetan-3-yl)acetic acid;
or an isotopic form or a stereoisomer or a tautomer or a pharmaceutically acceptable salt, a pharmaceutically acceptable solvate, a prodrug, a polymorph, N-oxide, S-oxide or a carboxylic acid isostere thereof.

40. A pharmaceutical composition comprising a therapeutically effective amount of the compound of formula (I) as defined in claim 26, or an isotopic form or a stereoisomer or a tautomer or a pharmaceutically acceptable salt or a pharmaceutically acceptable solvate thereof, and a pharmaceutically acceptable excipient.

41. A method for the treatment of a disease or a condition mediated by GPR40, comprising administering to a subject in need thereof a therapeutically effective amount of a compound according to claim 26, or an isotopic form, or a stereoisomer or a tautomer or a pharmaceutically acceptable salt or a pharmaceutically acceptable solvate thereof.

42. A method according to claim 41, wherein the disease or condition mediated by GPR40 is selected from the group consisting of diabetes, obesity, hyperglycemia, glucose intolerance, insulin resistance, hyperinsulinemia, hypercholesterolemia, hypertension, hyperlipoproteinemia, hyperlipidemia, hypertriglyceridemia, dyslipidemia, metabolic syndrome, syndrome X, cardiovascular disease, atherosclerosis, kidney disease, polycystic ovary syndrome, ketoacidosis, thrombotic disorders, nephropathy, diabetic neuropathy, diabetic retinopathy, sexual dysfunction, fatty liver development, dermatopathy, dyspepsia, hypoglycemia, cancer, edema and pancreatic beta cell degeneration.

43. A method according to claim 42, wherein the disease or condition mediated by GPR40 is selected from the group consisting of diabetes, obesity, insulin resistance, hyperglycemia, glucose intolerance, hypercholesterolemia, hypertriglyceridemia, dyslipidemia, hyperlipoproteinemia, hyperinsulinemia, atherosclerosis, diabetic neuropathy, diabetic retinopathy, metabolic syndrome, syndrome X, hypertension and pancreatic beta cell degeneration.

44. A method according to claim 43, wherein the disease or condition mediated by GPR40 is selected from the group consisting of diabetes, obesity, insulin resistance, hyperglycemia, glucose intolerance, metabolic syndrome, syndrome X and pancreatic beta cell degeneration.

45. A method according to claim 44, wherein the disease or condition mediated by GPR40 is diabetes, wherein the diabetes is Type 2 diabetes.
